(19) United States
(12) Patent Application Publication
(10) Pub. No.: US 2020/0129570 A1
(43) Pub. Date: Apr. 30, 2020

(54) Specific Bacterial Species and Metabolite That Improves Immune Checkpoint Inhibitor Therapy Efficacy

(71) Applicant: BOARD OF REGENTS, THE UNIVERSITY OF TEXAS SYSTEM, Austin, TX (US)

(72) Inventors: Arthur E. Frankel, Mobile, AL (US); Andrew Y. Koh, Southlake, TX (US)

(21) Appl. No.: 16/617,704

(22) PCT Filed: Jun. 1, 2018
(86) PCT No.: PCT/US2018/035564

§ 371 Date: Nov. 27, 2019

Related U.S. Application Data
(60) Provisional application No. 62/514,209, filed on Jun. 2, 2017.
(60) Provisional application No. 62/552,120, filed on Aug. 30, 2017.

(51) Int. Cl. A61K 35/742; C07K 16/28; A23L 33/135; A61K 31/192

(52) U.S. Cl. CPC A61K 35/742; C07K 16/2818; A61K 45/06; A61K 31/192; A23L 33/135

(57) ABSTRACT

The present invention includes compositions and methods for increasing an efficacy of an immune checkpoint inhibitor comprising: identifying a human patient in need of treatment for a melanoma; providing the human patient with an effective amount of the immune checkpoint inhibitor; and providing the human patient with at least one of: a probiotic bacteria, a prebiotic agent, or a xenobiotic agent, in the amount is effective to increase the potency of the immune checkpoint inhibitor against the melanoma.

### TECHNICAL FIELD OF THE INVENTION

The present invention relates in general to the field of cancer therapy, and more particularly, to specific bacterial species and metabolite that improves immune checkpoint inhibitor therapy efficacy.

### BACKGROUND OF THE INVENTION

Without limiting the scope of the invention, its background is described in connection with checkpoint inhibitors.

Immune checkpoint inhibitor therapy, ICT, achieves durable remissions in up to half of patients with metastatic melanoma (Larkin et al. NEJM 2015). It is still unclear what host factors modulate response to ICT. Preclinical mouse studies with B16 melanoma demonstrated that ICT response was dependent on the presence of specific commensal gut bacteria (Vetizou et al. Science 2015; Sivan et al. Science 2015). In mice, these specific gut bacteria induced the maturation of dendritic cells (DCs) and T-cells needed for effective ICT.

For example, Vetizou et al., in a publication entitled Anticancer immunotherapy by CTLA-4 blockade relies on the gut microbiota,” Science, 27 Nov. 2015 • Vol. 350 (6264), 1079-1084, and International Patent Application Publication No. WO2016/063263, filed Oct. 23, 2015, compared the relative therapeutic efficacy of a CTLA-4-specific 9D9 Ab against established MCA205 sarcomas in mice housed in specific pathogen-free (SPF) versus germ-free (GF) conditions. They further addressed the impact of the gut microbiota on the incidence and severity of intestinal lesions induced by CTLA-4 Ab treatment. The address the clinical relevance of the findings in mice, the authors analyzed the gut microbiome before and after treatment with ipilimumab in 25 individuals with metastatic myeloma. The gut microbiome was clustered into three groups, Alloprevotella or Prevotella driving the first cluster and distinct Bacteroides spp., driving clusters two and three. A fecal gut microbe transplantation was performed from patients with metastatic myeloma into mice with a metastatic myeloma. It was found that ipilimumab modified the abundance of immunogenic Bacteroides spp. in the gut, which in turn affects its anticancer efficacy. It was also found that intestinal reconstitution with Bacteroides fragilis and Burkholderia cepacia reduced histopathological signs of colitis induced by CTLA-4 blockade. The authors conclude that, “The geodistribution of Bf [Bacteroides fragilis] in the mucosal layer of the intestine . . . and its association with Burkholderiales—recognized through the pyrin-caspase-1 inflammasome (17) and synergizing with TLR2/TLR4 signaling pathways . . . —may account for the immunomodulatory effects of CTLA-4 Ab.” Id. at 1082. In their patent application, International Patent Application Publication No. WO 2016/063263, these authors further state that the invention also pertains to the use of vancomycin or penicillin to modulate the gut microbiota to potentiate the anticancer effects of anti-CTLA4 molecules.

Thus, a need remains for identifying agents that improve the effect of immune checkpoint antagonists in human myeloma patients.

### SUMMARY OF THE INVENTION

In one embodiment, the present invention includes a method of increasing the efficacy of an immune checkpoint inhibitor comprising: identifying a human patient in need of treatment for a melanoma; providing the human patient with an effective amount of the immune checkpoint inhibitor; and providing the human patient with at least one of: a probiotic bacteria, a prebiotic agent, or a xenobiotic agent, in the amount is effective to increase the potency of the immune checkpoint inhibitor against the melanoma. In one aspect, the immune checkpoint inhibitor is selected from at least one of a CTLA-4 inhibitor or a PD1 inhibitor. In another aspect, the human patient does not have a BRAF mutation. In another aspect, the immune checkpoint inhibitor is selected from at least one of ipilimumab, nivolumab, ipilimumab plus nivolumab, or pembrolizumab. In another aspect, the probiotic bacteria are selected from at least one of Alistipes shahii, Bacteroides caccae, Rumminococcaceae sp., Streptococcus mutans, or Dorea formicigenerans. In another aspect, the probiotic bacteria is Dorea formicigeneran and is provided to enhance the therapeutic effect of pembrolizumab (PD1). In another aspect, the probiotic bacteria are at least one of: Bacteroides thetaiotamicron, Alistepes shahii, Ruminococcaceae, or Streptococcus mutans, and the probiotic bacteria are provided to enhance the therapeutic effect of a combination of ipilimumab and nivolumab. In another aspect, the xenobiotic agent is an anacardic acid, a 15:2 anacardic acid, or active derivatives thereof. In another aspect, the patient is further provided with an effective amount of the probiotic and an anacardic acid in an amount sufficient to enhance the therapeutic effect of the immune checkpoint inhibitor selected from at least one of ipilimumab, nivolumab, ipilimumab plus nivolumab, or pembrolizumab. In another aspect, the method further comprises providing a probiotic further comprising one or more bacteria selected from at least one of: Methanobrevibacter smithii, Bacteroides thetaiotamicron, Lactobacillus plantarum, or Eubacterium limosum, in an amount sufficient to enhance the activity of the immune checkpoint inhibitor selected from at least one of ipilimumab, nivolumab, ipilimumab plus nivolumab, or pembrolizumab. In another aspect, the melanoma is metastatic melanoma. In another aspect, the melanoma is resistant or refractory to the immune checkpoint inhibitor. In another aspect, the patient did not receive a concurrent antibiotic or a probiotic therapy.

In one aspect, the probiotic bacteria that increase the effectiveness of a combination of the immune checkpoint inhibitors ipilimumab plus nivolumab is selected from at least one of: Faecalibacterium prausnitzii, Holdemania filiformis or Bacteroides thetaiotamicron. In another aspect, the probiotic bacteria that increase the effectiveness of ipilimumab plus nivolumab is Dorea formicigenerans. In another aspect, the probiotic bacteria that increase the effectiveness of the immune checkpoint inhibitor pembrolizumab is selected from at least one of: Bacteroides caccae or Streptococcus parasanguinis.

In another embodiment, the present invention includes a method of identifying a patient that will respond to therapy with an immune checkpoint inhibitor comprising: identifying a subject in need of treatment with the immune checkpoint inhibitor for the treatment of a melanoma; obtaining a biological sample from the patient that comprises gut intestinal flora; and determining whether the intestinal flora comprises at least one of Faecalibacterium prausnitzii, Holdemania filiformis, Bacteroides thetaiotamicron, Dorea formicigenerans, Bacteroides caccae or Streptococcus parasanguinis, wherein the presence of at least one of the Faecalibacterium prausnitzii, Holdemania filiformis, Bacteroides thetaiotamicron, Dorea formicigenerans, Bacteroides caccae or Streptococcus parasanguinis, is indicative that the melanoma will respond to the immune checkpoint inhibitor, or will have an improved response to the immune checkpoint inhibitor. In one aspect, the method further comprises providing the patient with an amount of Alistipes shahii, Bacteroides caccae, Rumminococcaceae sp., or Streptococcus mutans probiotics sufficient to enhance the response to the immune checkpoint inhibitor. In another aspect, the biological sample is a fecal sample. In another aspect, the presence of the probiotic bacteria is determined by at least one of metagenomic shotgun sequencing of bacteria or metabolic LC-MS analysis of metabolites. In another aspect, the patient does not have a BRAF mutation. In another aspect, the immune checkpoint inhibitor is selected from at least one of CTLA-4 inhibitor, a PD1 inhibitor, ipilimumab, nivolumab, ipilimumab plus nivolumab, or pembrolizumab. In another aspect, the probiotic bacteria are selected from at least one of Faecalibacterium prausnitzii, Holdemania filiformis, Bacteroides thetaiotamicron, Dorea formicigenerans, Bacteroides caccae or Streptococcus parasanguinis. In another aspect, the presence of Dorea formicigeneran is indicative of enhanced therapeutic effect by pembrolizumab. In another aspect, the presence of at least one of: Bacteroides thetaiotamicron, Faecalibacterium prausnitzii, or Holdemania filiformis is indicative of an enhanced therapeutic effect by ipilimumab plus nivolumab. In another aspect, the presence of Bacteroides caccae or Streptococcus parasanguinis is indicative of enhanced therapeutic effect by any combination of immune checkpoint inhibitor therapy. In another aspect, the presence of 15:2 anacardic acid in the biological sample is indicative of enhanced therapeutic effect by the immune checkpoint inhibitor. In another aspect, the patient is a human patient. In another aspect, the melanoma is metastatic melanoma. In another aspect, the melanoma is resistant or refractory to the immune checkpoint inhibitor. In another aspect, the patient did not receive a concurrent antibiotic or a probiotic therapy. In one aspect, the probiotic bacteria that increase the effectiveness of a combination of the immune checkpoint inhibitors ipilimumab plus nivolumab is selected from at least one of: Faecalibacterium prausnitzii, Holdemania filiformis or Bacteroides thetaiotamicron. In another aspect, the probiotic bacteria that increase the effectiveness of pembrolizumab is Dorea formicigenerans. In another aspect, the probiotic bacteria that increase the effectiveness of any of the immune checkpoint inhibitor therapies is selected from at least one of: Bacteroides caccae or Streptococcus parasanguinis.

In another embodiment, the present invention includes a composition that increases the efficacy of an immune checkpoint inhibitor comprising: a first composition comprising at least one of a probiotic or prebiotic composition or an anacardic acid; wherein the first composition is capable of increasing the efficacy of the immune checkpoint inhibitor; and a second composition comprising a therapeutically effective amount of the immune checkpoint inhibitor, wherein the immune checkpoint inhibitor is selected from at least one of ipilimumab, nivolumab, ipilimumab plus nivolumab, or pembrolizumab. In one aspect, the first and second compositions are provided in a single formulation, are provided concurrently, or are provided separately. In another aspect, the patient does not have a BRAF mutation. In another aspect, the first composition further comprises at least one of Methanobrevibacter smithii, Bacteroides thetaiotamicron, Lactobacillus plantarum, Eubacterium limosum, Bacteroides caccae, Streptococcus parasanguinis, Dorea formicigenerans, Faecalibacterium prausnitzii, Holdemania filiformis or Bacteroides thetaiotamicron. In another aspect, the first composition comprises Dorea formicigeneran, when the second composition comprises pembrolizumab (PD1). In another aspect, the first composition comprises at least one of: Bacteroides caccae or Streptococcus parasanguinis, when the second composition comprises at least one of ipilimumab, nivolumab, ipilimumab plus nivolumab, or pembrolizumab. In another aspect, the first composition comprises at least one of: Faecalibacterium prausnitzii, Holdemania filiformis or Bacteroides thetaiotamicron, when the second composition is ipilimumab and nivolumab. In another aspect, the first composition consists essentially of Faecalibacterium prausnitzii, Holdemania filiformis or Bacteroides thetaiotamicron when the second composition is ipilimumab and nivolumab. In another aspect, the first composition consists essentially of Dorea formicigeneran when the second composition is pembrolizumab. In another aspect, the probiotic bacteria is selected from the responsive bacteria in Tables 2A to 2D having a relative response of 1.0 or greater.

In another embodiment, the present invention includes a pharmaceutical composition comprising at least one of an isolated probiotic bacteria in an amount effective to increase the effectiveness of an immune checkpoint inhibitor. In one aspect, the composition consists essentially of Bacteroides caccae and Streptococcus parasanguinis. In another aspect, the composition consists essentially of Faecalibacterium prausnitzii, Holdemania filiformis and Bacteroides thetaiotamicron. In another aspect, the composition consists essentially of Dorea formicigenerans. In one aspect, the immune checkpoint inhibitor is selected from at least one of ipilimumab, nivolumab, ipilimumab plus nivolumab, or pembrolizumab.

In yet another embodiment, the present invention includes a method of increasing the efficacy of an immune checkpoint inhibitor comprising: identifying a human patient in need of treatment for a melanoma; providing the human patient with an effective amount of the immune checkpoint inhibitor; and providing the human patient with at least one of a targeted antibacterial agent in the amount effective to increase the potency of the immune checkpoint inhibitor against the melanoma. In one aspect, the antibacterial agent is directed against at least one of actinobacteria, coriobacteriaceae, coriobacteriales, bacteroides eggerthii, parvimonas micrs, parvimonas, Bifidobacterium dentium, or actinomyces viscosues. In another aspect, the antibacterial agent is directed against at least one of lactobacillaceae, lactobacillus, acidaminococcaceae, anaerococcus, atopobium parvulum, anaerococcus vaginalis, peptoniphilus or lactobacillus gasseri. In one aspect, the antibacterial agent is a bacteriophage. In one aspect, the antibacterial agent is an antimicrobial CRISP-Cas system agent

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show the relative abundance of gut bacterial taxa as determined by MetaPhlAn analysis of MSS data generated from fecal specimens collected from melanoma patients prior to receiving either ipilimumab/nivolumab or pembrolizumab. Differential taxonomic abundance was analyzed by linear discriminate analysis coupled with effect size measurements (LEfSe) projected as a histogram (FIG. 1A, FIG. 1C, FIG. 1E) or cladrogram (FIG. 1B, FIG. 1D, FIG. 1F). FIGS. 1A and 1B are for all immune checkpoint inhibitors (all ICT), FIGS. 1C and 1D for a combination therapy of ipilimumab and nivolumab, and FIGS. 1E and 1F for pembrolizumab. All listed bacterial groups were significantly (P<0.05, Kruskal-Wallis test) enriched for their respective groups (responder vs progressive).

FIGS. 2A and 2B shows non-targeted metabolomics analysis of stool metabolites from adult melanoma patients prior to treatment with immune checkpoint therapy with: FIG. 2A ipilimumab/nivolumab, and in FIG. 2B pembrolizumab, performed at Metabolon with four separate UHPLC-MS/MS methods. Data were log transformed and mean-centered. The heatmaps show the normalized relative abundances of stool metabolites comparing responders to those with progressive disease (q<0.05, unpaired t test with Welch's correction followed by FDR correction). Orange colors indicate relative abundances above and blue indicate relative abundances below the mean of all samples.

FIG. 3 shows a study schema with I, ipilimumab. N, nivolumab. P, pembrolizumab.

FIG. 4 shows a hierarchical clustering of species abundances shown as dendrogram plots with paired samples marked in red color. Samples without numbers were non-melanoma patient fecal specimens assayed identically.

FIGS. 5A and 5B show histograms of bacterial species' abundance versus response for FIG. 5A. Alistepes shahii in IN patients and, FIG. 5B. Dorea Formicigenerans in P patients.

FIG. 6 is a scattergram of response versus 15:2 anacardic acid levels. Green dots represent patients who also report ≥weekly cashew consumption.

FIG. 7 shows the structure of 15:2 anacardic acid of use with the present invention.

FIG. 8 shows an outline of in vivo studies and expected outcomes of the present invention.

FIG. 9 shows an outline of the detailed in vivo studies using the present invention.

FIG. 10 is a graph that shows the results of the precision probiotic therapy on Taconic C57BL/6 (versus a control probiotic) that promotes the activity of immune checkpoint therapy of the present invention.

FIG. 11 is a graph that shows the results of the precision probiotic therapy on Taconic C57BL/6 prolongs survival in mice with melanoma that are treated with ICT compared to mice treated with a control probiotic.

FIG. 12 is a graph that shows the results of the precision probiotic therapy on Jackson C57BL/6 (versus a control probiotic) that promotes the activity of immune checkpoint therapy against melanoma using the present invention.

FIG. 13 is a graph that shows the results of the precision probiotic therapy on Jackson C57BL/6 prolongs survival in mice with melanoma that are treated with ICT compared to mice treated with a control probiotic.

FIG. 14 is a graph that shows that Jackson C57BL/6 mice have decreased relative abundance of Clostridial firmicutes (which include F. prausnitzii, H. filiformis) compared to Taconic C57BL/6 Mice.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “immune checkpoint inhibitor” refers to agents that block immune checkpoints resulting in the enhancement of antigen-specific T cell responses. Non-limiting examples of immune checkpoints includes agents that target, e.g., PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitor(s) for use with the present invention include but are not limited to: ipilimumab, nivolumab, ipilimumab plus nivolumab, or pembrolizumab.

The probiotic bacteria, prebiotic agents, and/or xenobiotics for use with the present invention can be provided in a variety of dosage forms. For example, e.g., tablets, capsules, pills, powders, granules, elixirs, tinctures, suspensions, syrups, and emulsions may be used to provide the probiotic bacteria, prebiotic agents, and/or xenobiotics of the present invention to a patient in need of therapy for cancer with the immune checkpoint inhibitor(s). The immune checkpoint inhibitor(s) and the probiotic bacteria, prebiotic agents, and/or xenobiotics can be provided concurrently, in the same dosage form, at the same time thorough the same or different routes, one after the other, or separately in location and/or time.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference, and the like, relevant portions incorporated herein by reference.

For example, the probiotic bacteria, prebiotic agents, and/or xenobiotics may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

In one embodiment, gelatin capsules (gelcaps) may include the probiotic bacteria, prebiotic agents, and/or xenobiotics, and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the probiotic bacteria, prebiotic agents, and/or xenobiotics may be adapted for oral administration. Examples of suitable liquid dosage forms include powders, tablets, gelcaps, solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, emulsifying agents, suspending agents, diluents, sweeteners, thickeners, and melting agents, mixtures thereof, and the like, that do not affect the viability of the probiotic bacteria or enhance the viability of the probiotic bacteria.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. Suitable pharmaceutical carriers are described in Remington's Pharmaceutical Sciences, Mack Publishing Company, a standard reference text in this field, relevant portions incorporated herein by reference.

The inventors of the present invention sought to determine whether specific gut microbiota and/or microbial metabolites are associated with improved response to ICT in melanoma patients. The present invention includes the use of specific gut microbial species and a novel plant xenobiotic enhanced ICT clinical efficacy in melanoma patients. This is the first prospective study of the effects of human gut microbiota and metabolites on immune checkpoint inhibitor (ICT) response in human metastatic melanoma patients. Whereas many melanoma patients can exhibit profound response to ICT, there are fewer options for patients failing ICT-particularly with BRAF-wild-type disease. In preclinical studies, specific mouse gut microbial species promoted regression of melanoma bearing mice. The present inventors conducted a study of the effects of pretreatment gut microbiota and metabolites on ICT RECIST response in 39 metastatic melanoma patients treated with ipilimumab (I), nivolumab (N), ipilimumab plus nivolumab (IN) or pembrolizumab (P). IN yielded 54% responses and 8% stable disease; P achieved 23% responses and 23% stable disease. Responding patients for all therapies were enriched for Bacteroides coccae. Among IN responders, the gut microbiome was enriched for Bacteroides thetaiotamicron, Alistepes shahii, Ruminococcaceae and Streptococcus mutans. Among P responders, the microbiome was enriched for Dorea formicogenerans. Shotgun metabolomics revealed high levels of anacardic acid in ICT responders. Based on these studies it was found that certain bacteria species and xenobiotics had ICT enhancing activity.

The present inventors recognized that intestinal tract bacteria, collectively known as the gut microbiota, can influence and control host immune responses. In preclinical mouse models, the composition of the host gut microbiota was found to affect ICT response [7-9]. Germ-free or antibiotic treated tumor bearing mice did not respond to immune therapy [7]. Bifidobacterium fed B16 melanoma bearing mice showed increased tumor dendritic cell anti-tumor immune gene expression and enhanced anti-PD-L1 immunotherapy response [8]. In one study, gut Bacteroides thetaiotamicron or Bacteroides fragilis were necessary for anti-CTLA4 antibody anti-B16 melanoma in vivo efficacy [9]. Further, dendritic cells (DCs) and T cells mixed with either of these Bacteroides species in vitro increased T cell interferon γ production and in vivo tumor growth inhibition. In all the above studies, the gut bacteria induced maturation of anti-melanoma DCs and T cells.

The present invention is the first to study metastatic melanoma patients initiating ICT. Patients were stratified for type of immunotherapy. Fecal samples were collected and analyzed. The present inventors demonstrate herein the ICT efficacy in 39 metastatic melanoma patients and correlate clinical responses with gut taxome profiles, gut metabolite levels, and patient dietary and antibiotic histories.

Patients and Methods. The study design was a single-site, correlative study of the effects of gut microbiota and metabolites on ICT efficacy in 39 patients. The study was approved by the University of Texas Southwestern Medical Center Institutional Review Board (IRB # STU012016-056). The study was conducted in accordance with the Declaration of Helsinki. The inventors enrolled patients with a histologic diagnosis of unresectable or metastatic melanoma that were scheduled to begin ICT and able to collect stool specimens, store them in a freezer and deliver them to the University of Texas Southwestern Cancer Center facility. In addition, patients had to have measurable disease by Response Evaluation Criteria in Solid Tumors (RECIST) v1.1. Tumor sizes were evaluated within 4 weeks prior to beginning therapy by exams, CT scans, and/or MRIs. Therapy involved outpatient ipilimumab 3 mg/kg IV every three weeks for four doses (I), nivolumab 1 mg/kg IV with ipilimumab 3 mg/kg IV every three weeks for four doses followed by nivolumab alone at 240 mg IV every two weeks (IN), nivolumab alone at 240 mg IV every two weeks (N), or pembrolizumab 2 mg/kg IV every three weeks (P). After informed consent, stool samples were collected and immediately frozen until transferred to the University of Texas Southwestern Medical Center at pretreatment and various times post-therapy. Repeat exams and scans were obtained every 2-3 months. Demographics, antibiotic use, and probiotic exposure were recorded for each patient. Patient response, stable disease and progression were evaluated by RECIST v1.1 criteria as reported [10].

Fecal specimens collected at patient's homes and immediately frozen were transferred in Styrofoam containers with cold packs to the clinic and immediately transferred to −80° C. freezer until assayed. Thawed 200 mg fecal aliquots were suspended in 0.71 mL extraction buffer consisting of 200 mM NaCl, 200 mM Tris pH 7.9, 20 mM EDTA, 6% SDS and 0.5 mL Ambion phenol/chloroform/isoamyl alcohol added. Cells were lysed by bead-beating with 0.1 mm diameter Biospec zirconia/silica beads and centrifuged at 20,000×g for 3 minutes. Supernatants were reextracted with phenol/chloroform/isoamyl alcohol, adjusted to 200 mM sodium acetate, and nucleic acids precipitated with equal volume isopropyl alcohol followed by an ethanol wash. Crude DNA extracts were treated with Qiagen RNAse A and column-purified with the Qiagen PCR purification kit. DNA was assayed for purity by UV spectroscopy and DNA concentrations quantified by the Life Technologies Quan-iT PicoGreen dsDNA assay. Metagenomic shotgun sequencing (MSS) data was generated by pair-end sequencing of the fecal gDNA with 100 bp reads on an Illumina HiSeq 2000. Raw MSS data was quality controlled using NGS-QC (www.nipgr.res.in/ngsqctoolkit.html), and human sequences removed with the NCBI BMTagger Human Contamination Screening Tool (ftp.ncbi.nlm.nih.gov/pub/agarwala/bmtagger/). Then Metagenomic bacterial taxonomic analysis was performed on the filtered sequences with the computational tool MetaPhlAn. Finally, linear discriminate analysis coupled with effect size measurements—LefSe was done to quantitate differential taxonomic abundance between responders and non-responders as previously reported by our laboratory [11].

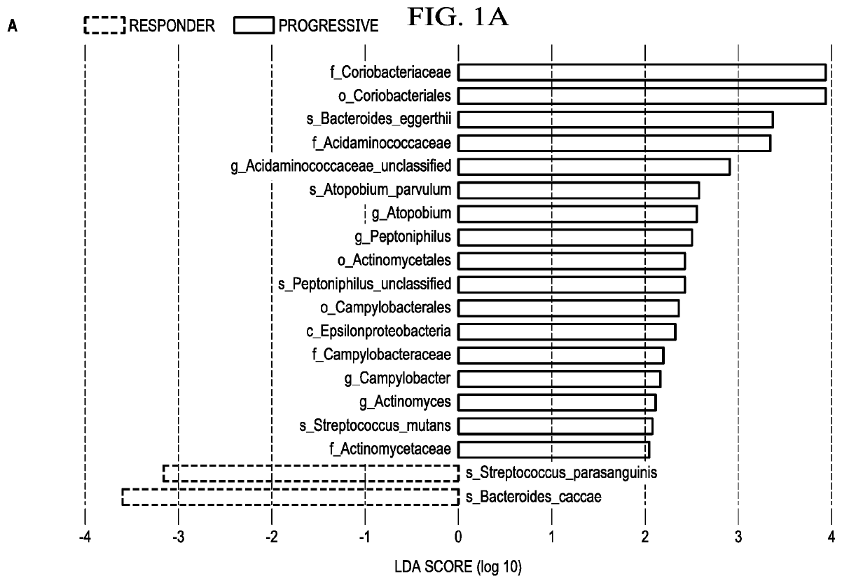

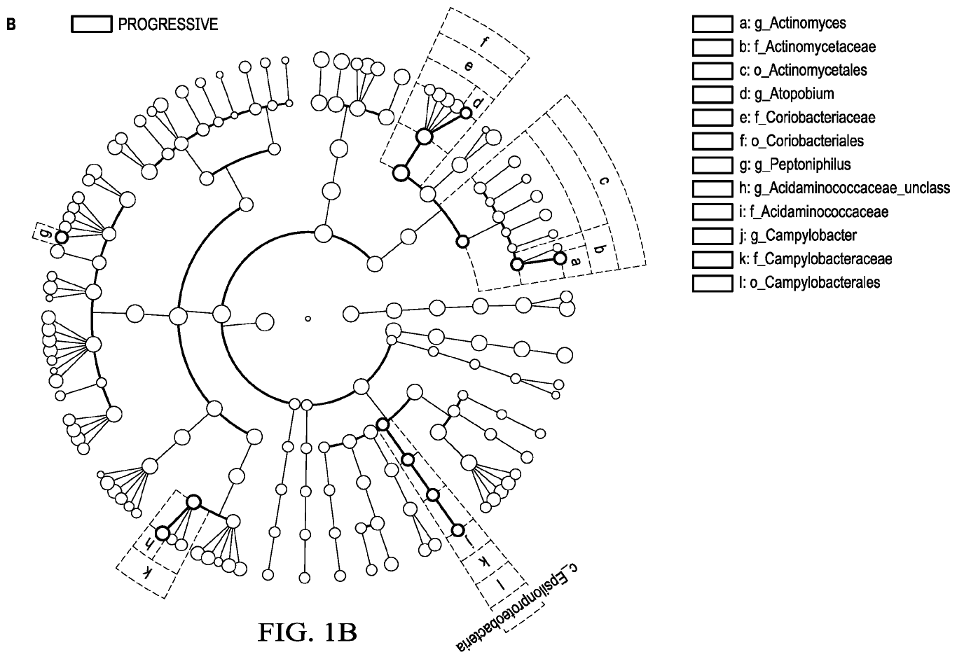

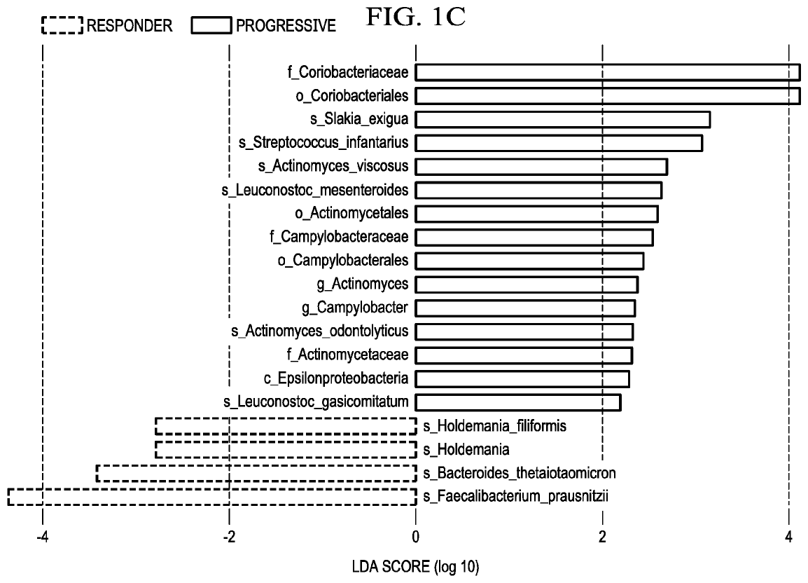

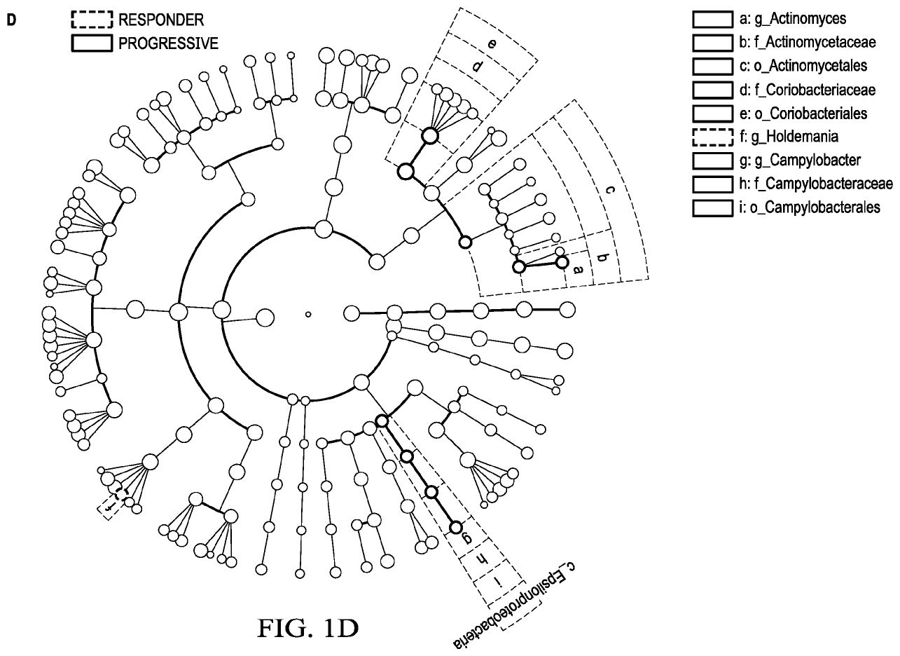

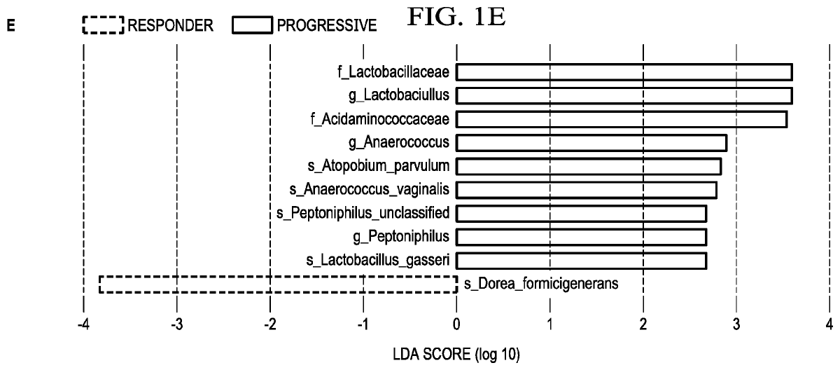

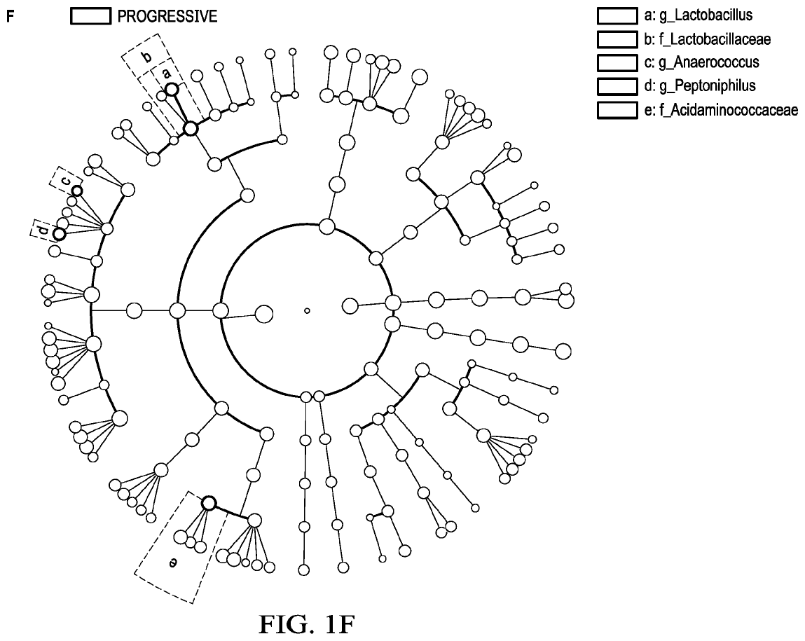

FIGS. 1A to 1D show the relative abundance of gut bacterial taxa as determined by MetaPhlAn analysis of MSS data generated from fecal specimens collected from melanoma patients prior to receiving either ipilimumab/nivolumab or pembrolizumab. Differential taxonomic abundance was analyzed by linear discriminate analysis coupled with effect size measurements (LEfSe) projected as a histogram (FIG. 1A, FIG. 1C) or cladrogram (FIG. 1B, FIG. 1D). All listed bacterial groups were significantly (P<0.05, Kruskal-Wallis test) enriched for their respective groups (responder vs progressive).

Patient gut pathway abundance was calculated by mapping the MSS sequences to the Kyoto Encyclopedia of Genes and Genomes (KEGG) Database using USEARCH and the computational tools sHUMAnN and LEfSE [12]. Pathway abundance was compared for responders and stable disease patients versus progressors.

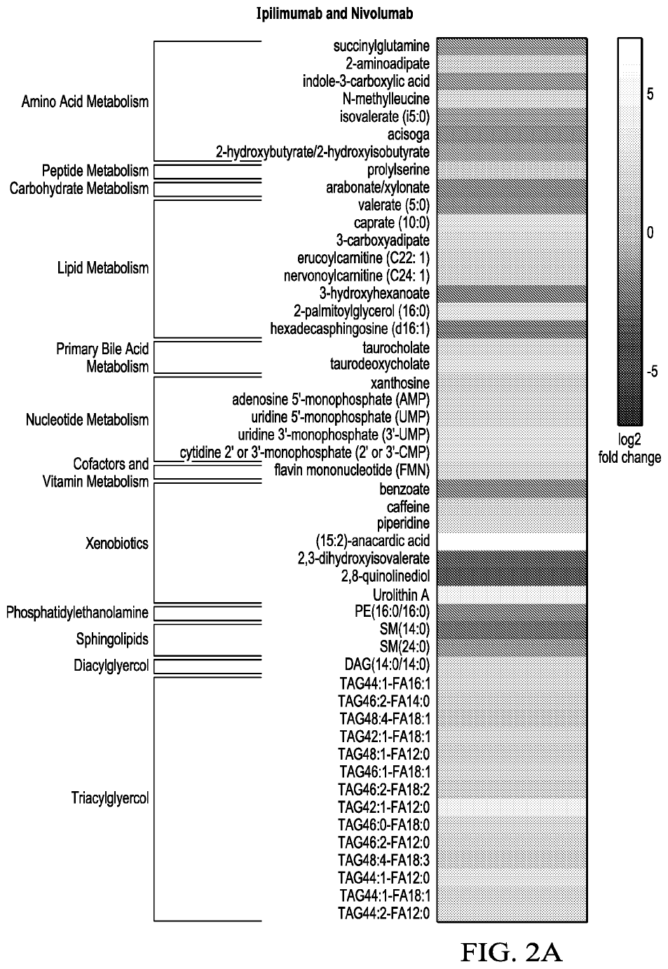

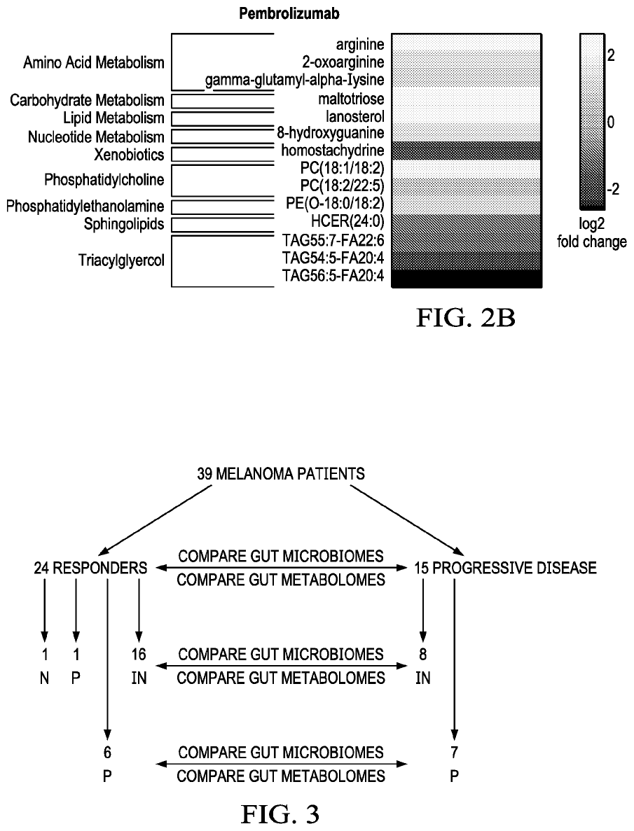

Gut microbial and food metabolites were also identified and quantitated by ultrahigh-performance liquid chromatography/mass spectrometry (UHPLC/MS) at Metabolon, Inc. (Durham, N.C.) as previously described [13]. Briefly, fecal samples were lyophilized and subjected to methanol extraction then split into aliquots for analysis for UHPLC/MS in the positive (two methods), negative, and polar ion mode, followed by normalization to account for differential volume extracted. Compounds were identified by automated comparison to reference chemical library entries with subsequent visual inspection for quality control as previously described [14]. For statistical analyses and data display, any missing values were assumed to be below the limits of detection; these values were imputed with the compound minimum (minimum value imputation). Statistical tests were performed in ArrayStudio (Omicsoft) or “R” to compare data between experimental groups; p<0.05 was considered significant. An estimate of the false discovery rate (q-value) was also calculated to take into account the multiple comparison that normally occur in metabolomics-based studies, with q<0.05 used as an indication of high confidence in a result. FIGS. 2A and 2B shows non-targeted metabolomics analysis of stool metabolites from adult melanoma patients prior to treatment with immune checkpoint therapy with: FIG. 2A ipilimumab/nivolumab, and in FIG. 2B pembrolizumab, performed at Metabolon with four separate UHPLC-MS/MS methods. Data were log transformed and mean-centered. The heatmaps show the normalized relative abundances of stool metabolites comparing responders to those with progressive disease (q<0.05, unpaired t test with Welch's correction followed by FDR correction). Orange colors indicate relative abundances above and blue indicate relative abundances below the mean of all samples.

Patient histories were interrogated for consumption of foods enriched for identified plant xenobiotic in excess in ICT non-progressors based on metametabolomics.

Thirty-nine metastatic melanoma patients were enrolled, consented, provided pretreatment fecal samples, underwent ICT, and had follow-up exams and scans (Table 1). There were 30 males and 9 females. Median age was 66 years with a range of 37 years to 92 years. Sites of metastases were single organ in 22 patients including 11 lung, 4 nodes, 4 liver, 2 SQ and 1 bone. There were two sites of metastases in 14 patients including 3 lung/liver, 3 lung/nodes, 3 nodes/SQ, 2 nodes/bone, and one each with lung/SQ, SQ/adrenal and lung/adrenal. At least three organ sites were involved in 3 patients-lung/liver/nodes, lung/nodes/SQ and lung/liver/bone, respectively. Twenty-three patients showed RECIST response or stable disease to ICT (Table 1 and FIG. 3). IN achieved 13/24 (54%) RECIST responders and 2/24 (8%) stable disease patients 8% stable disease. P treatment led to 3/13 (23%) RECIST responders and 3/13 (23% stable disease patients. One patient each with I alone and N alone responded.

TABLE 1
Patient Characteristics

| | | | | ICT Therapy | | |
|---|---|---|---|---|---|---|
| | | | | (I = ipilumumab, | Change in | |
| Patient | | | | N = nivoimab, | tumor size | RECIST |
| Identifier | Sex | Age | Site of Metastases | P = pembrolizumab) | (%) | Category |
| P7 | M | 63 | Lung | IN | −4 | Stable |
| P8 | M | 70 | Lung | IN | −55 | Response |
| P10 | M | 75 | Lung | IN | −83 | Response |
| P14 | F | 69 | Lung | IN | −60 | Response |
| P16 | M | 80 | Lung, Nodes | P | 53 | Progression |
| P17 | M | 68 | Nodes | N | −70 | Response |
| P22 | M | 64 | Lung, Liver | P | 81 | Progression |
| P23 | M | 76 | Lung | IN | −55 | Response |
| P24 | M | 44 | Nodes | IN | 136 | Progressiion |
| P25 | F | 60 | SQ | IN | −100 | Response |
| P28 | F | 68 | Lung, Liver | IN | 85 | Progression |
| P30 | M | 54 | Lung, Liver | IN | 100 | Progression |
| P32 | F | 57 | Nodes, Bone | IN | 100 | Progression |
| P33 | F | 74 | Nodes | P | −36 | Response |
| P34 | M | 57 | Liver | IN | −66 | Response |
| P35 | M | 63 | Nodes | IN | −30 | Response |
| P39 | M | 48 | Nodes, SQ | P | −68 | Response |
| P42 | M | 67 | SQ | P | 100 | Progression |
| P44 | F | 63 | Nodes | P | −27 | Stable |
| P45 | M | 43 | Lung, Nodes, SQ | P | −14 | Stable |
| P46 | M | 68 | SQ, Adrenal | IN | 116 | Progression |
| P48 | M | 86 | Lung | P | 0 | Stable |
| P49 | M | 84 | Lung, Liver, Nodes | P | 125 | Progression |
| P52 | M | 41 | Bone | IN | 67 | Progression |
| P53 | M | 74 | Lung, Adrenal | IN | 100 | Progression |
| P54 | M | 79 | Nodes, SQ | IN | 100 | Progression |
| P55 | F | 37 | SQ, Adrenal | IN | −100 | Response |
| P56 | M | 66 | Lung, Nodes | P | 46 | Progression |
| P57 | M | 70 | Liver | I | −100 | Response |
| P58 | M | 52 | Nodes | P | −48 | Response |
| P59 | M | 78 | Lung | IN | −10 | Stable |
| P61 | M | 58 | Lung | IN | −40 | Response |
| P63 | M | 63 | Nodes, SQ | IN | −100 | Response |
| P64 | F | 77 | Lung, Liver, Bone | P | 100 | Progression |
| P65 | M | 69 | Liver | P | 131 | Progression |
| P66 | M | 80 | Lung, Nodes | IN | 61 | Progression |
| P67 | M | 83 | Lung | IN | −34 | Response |
| P68 | M | 92 | Lung | IN | −34 | Response |
| P69 | F | 55 | Lung | IN | −87 | Response |

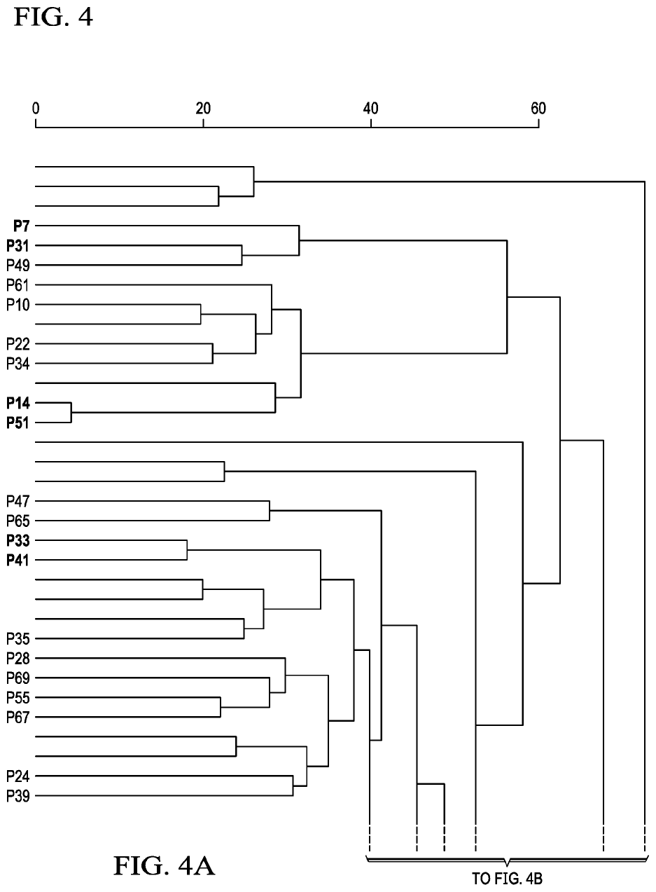

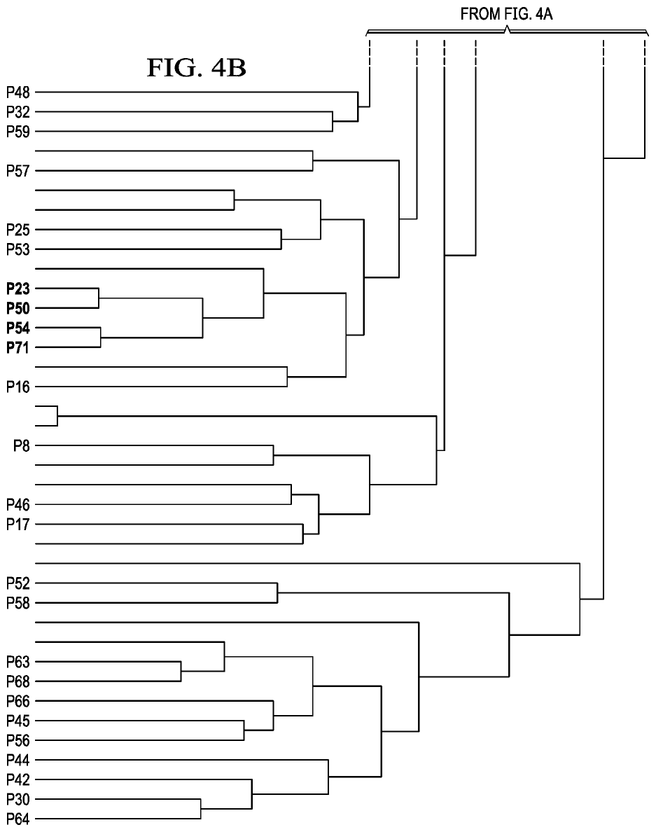

Taxonomic profiles showed stability of results on repeated patient sampling in five patients based on matched hierarchical clustering of species abundances among samples (FIG. 4).

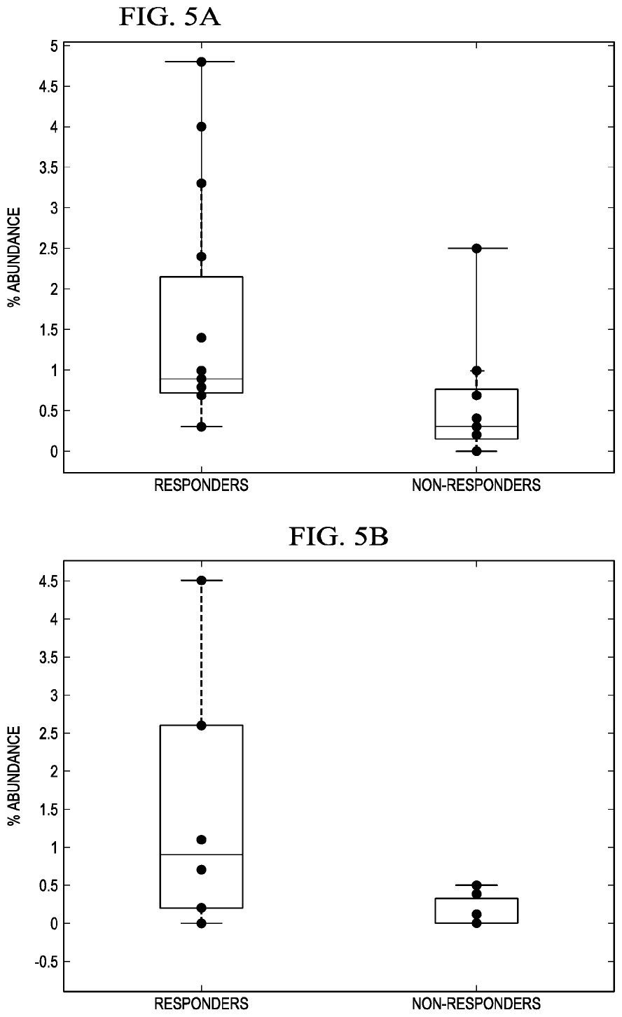

Taxonomic profiles of pretreatment samples from ICT treated patients revealed significant differences in abundance of bacterial species between responder and progressors (FIGS. 1A-1D and FIGS. 5A-5B). FIGS. 5A and 5B show histograms of bacterial species' abundance versus response for FIG. 5A. Alistepes shahii in IN patients and, FIG. 5B. Dorea Formicigenerans in P patients. Among all treated patients, responder microbiomes were significantly enriched with Bacteroides caccae (p=0.034; linear discriminant analysis coupled with effect size measurements, LEfSe; Kruskal-Wallis test) compared to those with progression. Among those patients treated only with IN, responder microbiomes were enriched with Bacteroides thetaiotamicron (p=0.017), Alistipes shahii (p=0.022), the Firmicutes family Ruminococcaceae (p=0.025), and Streptococcus mutans (p=0.039). Among those patients treated only with P, responder microbiomes were enriched with Dorea Formicigenerans (p=0.045).

Next, the inventors determined if antibiotic use during ICT modified gut microbiomes and affect response. Only three patients on the study received systemic antibiotics immediately before or during the treatment course. Patient #7 received a two weeks course of ceftriaxone prior to therapy. Patient #22 received two weeks of ciprofloxacin, vancomycin and metronidazole after two ICT cycles. Patient #44 received a course of nitrofurantoin after four ICT cycles. Only patient #23 took daily doses of the probiotic Lactobacillus rhamnosus. The taxome profile on this patient did not show any detectable Lactobacillus rhamnosus. No particular response or toxicity was linked to antibiotic or probiotic exposure.

Functional pathway enrichment based on KEGG analysis did not yield statistically significant differences between responders and progressors for any of the treatment groups.

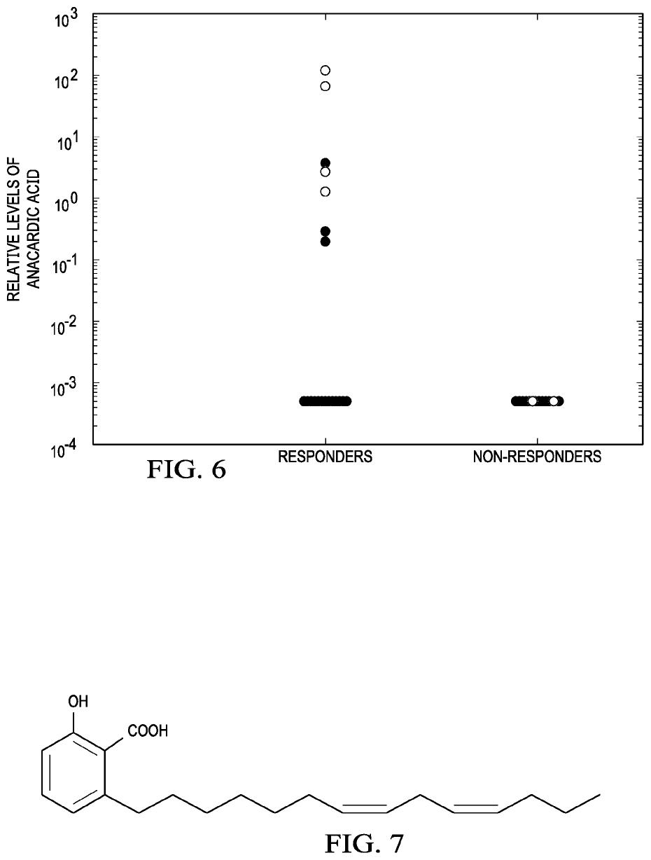

Metabolomic analysis of the patient fecal samples showed differences in metabolites between responders and non-responders (FIGS. 2A, 2B and 6). IN responders had increased levels of anacardic acids; P responders showed, to a lesser degree, increased levels of arginine.

Based on the discovery of a food metabolite increased in responders, we expanded our patient histories to include queries regarding regular consumption of plant-related products with high levels of anacardic acid including cashews. Five of six patients with high 15:2 anacardic acid levels versus two of thirty-three evaluable patients with low 15:2 anacardic acid levels consumed cashews at least weekly (significant with P<0.0001 chi-squared test with Yates continuity correction).

It was found that the general disease control rate (response plus stable disease) observed in the study for both IN and P was consistent with published results. Hodi and colleagues report a 59% response rate and 13% stable disease rate in a phase 2 study of IN [15], and Wolchok and coworkers achieved a 44% response rate and 8% stable disease rate in a phase 1 study of IN [16]. These numbers are close to the findings herein of a 54% response rate and 8% stable disease rate, and suggest the patient cohort and treatment management used herein was consistent with others.

The present invention stated with snapshots of patient gut microbiomes pretreatment, which may or may not reflect potential variations in taxome distribution over days-months of therapy. However, in repeat testing on five patients, there was evidence for relative stability of the microbiome based on matched hierarchical clustering of species abundances among the patient samples. These results are consistent with those of Gordon and Relman [17, 18]. Individual patient gut bacterial community composition remains stable for months unless perturbed by antibiotics or disease or dramatic diet change.

The present invention includes the first report of human gut microbiota in melanoma patients treated with combination anti-PD1 and anti-CTLA4 immunotherapy (IN) as well as anti-PD1 therapy alone (P). The findings of distinct predictive microbes for human ICT response for IN and P is unique. Six clades of bacteria involved in modulating ICT response were found. Bacteroides species have been previously reported to enhance anti-CTLA4 immune checkpoint efficacy in mice [9]. From the preclinical work, these bacteria are presumed to directly contact and stimulate host dendritic cells and T cells via pathogen-associated molecular patterns—PAMPs. The clinical findings herein differ significantly from prior studies, and show an increase of Bacteroides caccae, Bacteroides thetaiotamicron, and Alistipes shahii in anti-CTLA4 plus anti-PD1 responders. In addition, it was found that firmicutes of the Clostridiales order—Ruminococcaceae, Dorea formicigenerans and of the Lactobacillales order—Streptococcus mutans were associated with anti-CTLA4 plus anti-PD1 clinical efficacy.

The detailed molecular mechanism for immune enhancement by any of the bacteria in humans remains unknown. By way of explanation, and in no way a limitation of the present invention, in vitro studies with dendritic cells have implicated some of these bacterial species with immune modulation. Streptococcus mutans activates dendritic cells (DCs) in vitro [22]. In part, the Streptococcus mutans' Wall-associated protein A (WapA) stimulates DC TLR4-induced NF-κB pathway [23]. Bacteroides thetaiotamicron releases 10 nm-80 nm outer membrane vesicles (OMVs) that contain mucin-degrading glycosidase hydrolases and sulfatases [24]. These enzymes degrade the gut mucin and permit the OMVs to reach and be phagocytosed by DCs. The OMVs also contain toxins, adhesins and enzymes that trigger DC activation. Bacteroides caccae and thetaiotamicron are Gram-negative organisms with surface lipopolysaccharide (LPS) that stimulate DCs in a TLR4-dependent manner [25]. Similarly, the Gram-negative Alistipes shahii induces TLR4-signaling and DC production of TNFα improving the response of murine tumors to CpG-oligonucleotides and platinum [7]. The six identified response related bacteria merit in vitro testing with human peripheral blood mononuclear cells in mixed lymphocyte reactions (MLRs). When bacterial species are combined with ipilimumab and nivolumab in the MLR and secretion of interleukin-2 and interferon γ measured in the supernatant, the results provide an indirect assay of DC activation [26).

The present inventors found that there was little if any effect of patient exposure to systemic antibiotics or probiotics. For example, the lack of effect of the probiotic on patient #23 is consistent with a baseline stable commensal community that inhibits overgrowth of the probiotic organism [28, 29]. No particular response or toxicity was linked to antibiotic or probiotic exposure in this study.

The present inventors performed metabolomics on all 39 patient samples. Surprisingly, among thousands of measured metabolites, the most dramatic correlation with response was seen with a plant xenobiotic-15:2 anacardic acid (FIG. 7). This compound is an alkyl derivative of salicylic acid and produced in the nutshell of cashews [30]. Anacardic acids stimulate neutrophils and macrophages [31, 32]. Similar to the effects of particular bacterial species, the activation of macrophages/dendritic cells may enhance T cell recruitment to tumor metastases and, consequently, enhance ICT. Recently, cashew and other tree nut consumption were linked to reduced colon cancer recurrence in an epidemiologic study [33]. The present inventors found a correlation between cashew consumption and patient fecal anacardic acid levels.

Tables 2A to 2D show additional results from the present invention. A value greater than or equal to 1.0 is indicative of a response.

TABLE 2A

| Rank | Name | P1 | P2 | P3 | P4 | P5 | P6 | P7 | P8 | P9 |
|---|---|---|---|---|---|---|---|---|---|---|
| k | Archaea | 0.0 | 0.0 | 0.0 | 0.0 | 0.0 | 0.0 | 4.8 | 0.0 | 0.0 |
| p | Euryarchaeota | 0.0 | 0.0 | 0.0 | 0.0 | 0.0 | 0.0 | 4.8 | 0.0 | 0.0 |
| c | Methanobacteria | 0.0 | 0.0 | 0.0 | 0.0 | 0.0 | 0.0 | 4.8 | 0.0 | 0.0 |
| o | Methanobacteriales | 0.0 | 0.0 | 0.0 | 0.0 | 0.0 | 0.0 | 4.8 | 0.0 | 0.0 |
| f | Methanobacteriaceae | 0.0 | 0.0 | 0.0 | 0.0 | 0.0 | 0.0 | 4.8 | 0.0 | 0.0 |
| g | Methanobrevibacter | 0.0 | 0.0 | 0.0 | 0.0 | 0.0 | 0.0 | 4.8 | 0.0 | 0.0 |
| s | Methanobrevibacter_smithii | 0.0 | 0.0 | 0.0 | 0.0 | 0.0 | 0.0 | 4.8 | 0.0 | 0.0 |
| k | Bacteria | 100.0 | 100.0 | 100.0 | 100.0 | 100.0 | 100.0 | 95.2 | 100.0 | 100.0 |
| p | Actinobacteria | 19.5 | 8.9 | 0.2 | 2.7 | 2.1 | 3.5 | 12.0 | 3.2 | 16.7 |
| c | Actinobacteria | 19.5 | 8.9 | 0.2 | 2.7 | 2.1 | 3.5 | 12.0 | 3.2 | 16.7 |
| o | Actinomycetales | 9.0 | 0.0 | 0.1 | 0.1 | 0.0 | 0.0 | 0.0 | 0.0 | 0.0 |
| f | Corynebacteriaceae | 9.0 | 0.0 | 0.0 | 0.0 | 0.0 | 0.0 | 0.0 | 0.0 | 0.0 |
| g | Corynebacterium | 9.0 | 0.0 | 0.0 | 0.0 | 0.0 | 0.0 | 0.0 | 0.0 | 0.0 |
| s | Corynebacterium_amycolatum | 8.3 | 0.0 | 0.0 | 0.0 | 0.0 | 0.0 | 0.0 | 0.0 | 0.0 |
| s | Corynebacterium_striatum | 0.0 | 0.0 | 0.0 | 0.0 | 0.0 | 0.0 | 0.0 | 0.0 | 0.0 |
| o | Bifidobacteriales | 7.2 | 4.2 | 0.0 | 0.1 | 0.0 | 2.2 | 9.4 | 2.0 | 16.1 |
| f | Bifidobacteriaceae | 7.2 | 4.2 | 0.0 | 0.1 | 0.0 | 2.2 | 9.4 | 2.0 | 16.1 |
| g | Bifidobacterium | 7.2 | 4.2 | 0.0 | 0.1 | 0.0 | 2.2 | 9.4 | 2.0 | 16.1 |
| s | Bifidobacterium_adolescentis | 6.4 | 0.0 | 0.0 | 0.0 | 0.0 | 0.0 | 0.0 | 0.0 | 0.0 |
| s | Bifidobacterium_animalis | 0.0 | 0.0 | 0.0 | 0.0 | 0.0 | 1.5 | 0.0 | 0.0 | 12.1 |
| s | Bifidobacterium_bifidum | 0.0 | 0.0 | 0.0 | 0.0 | 0.0 | 0.0 | 0.0 | 0.0 | 0.0 |
| s | Bifidobacterium_breve | 0.0 | 0.0 | 0.0 | 0.0 | 0.0 | 0.6 | 0.0 | 0.0 | 3.9 |
| s | Bifidobacterium_catenulatum | 0.0 | 0.0 | 0.0 | 0.0 | 0.0 | 0.0 | 0.0 | 0.0 | 0.0 |
| s | Bifidobacterium_dentium | 0.0 | 0.2 | 0.0 | 0.0 | 0.0 | 0.1 | 0.0 | 0.0 | 0.1 |
| s | Bifidobacterium_longum | 0.8 | 4.0 | 0.0 | 0.1 | 0.0 | 0.0 | 9.4 | 1.2 | 0.0 |
| s | Bifidobacterium_pseudocatenulatum | 0.0 | 0.0 | 0.0 | 0.0 | 0.0 | 0.0 | 0.0 | 0.7 | 0.0 |
| o | Coriobacteriales | 3.2 | 4.6 | 0.0 | 2.6 | 2.1 | 1.3 | 2.5 | 1.2 | 0.6 |
| f | Coriobacteriaceae | 3.2 | 4.6 | 0.0 | 2.6 | 2.1 | 1.3 | 2.5 | 1.2 | 0.6 |
| g | Collinsella | 0.0 | 4.4 | 0.0 | 0.0 | 2.0 | 0.0 | 0.0 | 0.9 | 0.0 |
| s | Collinsella_aerofaciens | 0.0 | 4.4 | 0.0 | 0.0 | 2.0 | 0.0 | 0.0 | 0.9 | 0.0 |
| s | Collinsella_intestinalis | 0.0 | 0.0 | 0.0 | 0.0 | 0.0 | 0.0 | 0.0 | 0.0 | 0.0 |
| g | Eggerthella | 2.3 | 0.3 | 0.0 | 1.1 | 0.0 | 1.1 | 1.4 | 0.0 | 0.6 |
| s | Eggerthella_lenta | 2.3 | 0.3 | 0.0 | 1.1 | 0.0 | 1.1 | 1.4 | 0.0 | 0.6 |
| g | Gordonibacter | 0.9 | 0.0 | 0.0 | 1.5 | 0.0 | 0.2 | 1.2 | 0.3 | 0.0 |
| s | Gordonibacter_pamelaeae | 0.9 | 0.0 | 0.0 | 1.5 | 0.0 | 0.2 | 1.2 | 0.3 | 0.0 |
| p | Bacteroidetes | 1.1 | 4.0 | 61.8 | 58.3 | 24.8 | 15.5 | 22.3 | 31.4 | 29.6 |
| c | Bacteroidia | 1.1 | 4.0 | 61.8 | 58.3 | 24.8 | 15.5 | 22.3 | 31.4 | 29.6 |
| o | Bacteroidales | 1.1 | 4.0 | 61.8 | 58.3 | 24.8 | 15.5 | 22.3 | 31.4 | 29.6 |
| f | Bacteroidaceae | 0.6 | 3.8 | 60.0 | 49.2 | 11.3 | 15.1 | 16.6 | 22.5 | 26.5 |
| g | Bacteroides | 0.6 | 3.8 | 60.0 | 49.2 | 11.3 | 15.1 | 16.6 | 22.5 | 26.5 |
| s | Bacteroides_caccae | 0.0 | 0.0 | 0.2 | 15.7 | 1.3 | 0.0 | 1.0 | 0.9 | 0.0 |
| s | Bacteroides_cellulosilyticus | 0.0 | 0.1 | 0.0 | 2.5 | 0.1 | 0.0 | 0.0 | 0.0 | 0.0 |
| s | Bacteroides_coprocola | 0.0 | 0.0 | 0.0 | 0.0 | 0.0 | 0.0 | 0.0 | 0.0 | 0.0 |
| s | Bacteroides_coprophilus | 0.0 | 0.0 | 0.0 | 0.0 | 0.0 | 0.0 | 0.0 | 0.0 | 0.0 |
| s | Bacteroides_eggerthii | 0.0 | 0.0 | 0.0 | 0.0 | 0.6 | 0.0 | 0.0 | 0.0 | 0.0 |
| s | Bacteroides_finegoldii | 0.0 | 0.0 | 0.0 | 0.0 | 0.3 | 0.0 | 0.0 | 0.0 | 0.0 |
| s | Bacteroides_fragilis | 0.0 | 0.0 | 0.0 | 0.0 | 0.0 | 3.2 | 0.0 | 0.0 | 5.6 |
| s | Bacteroides_intestinalis | 0.1 | 0.0 | 0.0 | 0.0 | 1.9 | 0.0 | 0.0 | 0.0 | 0.0 |
| s | Bacteroides_ovatus | 0.0 | 0.1 | 1.3 | 0.2 | 0.0 | 0.0 | 0.0 | 0.2 | 0.0 |
| s | Bacteroides_pectinophilus | 0.0 | 0.0 | 0.0 | 0.0 | 0.0 | 0.0 | 0.0 | 0.0 | 0.0 |
| s | Bacteroides_plebeius | 0.0 | 0.0 | 0.0 | 0.0 | 0.0 | 0.0 | 0.0 | 0.0 | 0.0 |
| s | Bacteroides_stercoris | 0.0 | 0.0 | 0.0 | 0.0 | 0.1 | 0.0 | 0.0 | 2.6 | 0.0 |
| s | Bacteroides_thetaiotaomicron | 0.0 | 0.0 | 1.9 | 0.0 | 0.5 | 0.0 | 0.0 | 0.0 | 0.0 |
| s | Bacteroides_unclassified | 0.1 | 1.7 | 50.3 | 0.0 | 0.0 | 11.9 | 15.6 | 12.0 | 20.8 |
| s | Bacteroides_uniformis | 0.0 | 0.1 | 0.0 | 16.3 | 1.1 | 0.0 | 0.0 | 0.1 | 0.0 |
| s | Bacteroides_vulgatus | 0.3 | 1.7 | 6.3 | 8.1 | 5.1 | 0.0 | 0.0 | 5.6 | 0.0 |
| s | Bacteroides_xylanisolvens | 0.0 | 0.0 | 0.1 | 6.4 | 0.2 | 0.0 | 0.0 | 1.1 | 0.0 |
| f | Porphyromonadaceae | 0.1 | 0.1 | 0.0 | 8.9 | 0.6 | 0.0 | 1.2 | 1.9 | 0.0 |
| g | Odoribacter | 0.0 | 0.0 | 0.0 | 0.0 | 0.3 | 0.0 | 0.2 | 1.0 | 0.0 |
| s | Odoribacter_splanchnicus | 0.0 | 0.0 | 0.0 | 0.0 | 0.3 | 0.0 | 0.2 | 1.0 | 0.0 |
| g | Parabacteroides | 0.1 | 0.0 | 0.0 | 8.9 | 0.3 | 0.0 | 1.0 | 0.9 | 0.0 |
| s | Parabacteroides_distasonis | 0.0 | 0.0 | 0.0 | 0.0 | 0.0 | 0.0 | 0.0 | 0.0 | 0.0 |
| s | Parabacteroides_johnsonii | 0.0 | 0.0 | 0.0 | 0.0 | 0.0 | 0.0 | 0.0 | 0.0 | 0.0 |
| s | Parabacteroides_merdae | 0.1 | 0.0 | 0.0 | 5.8 | 0.3 | 0.0 | 0.7 | 0.6 | 0.0 |
| s | Parabacteroides_unclassified | 0.0 | 0.0 | 0.0 | 3.1 | 0.0 | 0.0 | 0.3 | 0.3 | 0.0 |
| g | Porphyromonas | 0.0 | 0.0 | 0.0 | 0.0 | 0.0 | 0.0 | 0.0 | 0.0 | 0.0 |
| s | Porphyromonas_asaccharolytica | 0.0 | 0.0 | 0.0 | 0.0 | 0.0 | 0.0 | 0.0 | 0.0 | 0.0 |
| f | Prevotellaceae | 0.0 | 0.0 | 0.0 | 0.0 | 10.4 | 0.4 | 0.0 | 0.0 | 3.1 |
| g | Prevotella | 0.0 | 0.0 | 0.0 | 0.0 | 10.4 | 0.4 | 0.0 | 0.0 | 3.1 |
| s | Prevotella_copri | 0.0 | 0.0 | 0.0 | 0.0 | 10.4 | 0.0 | 0.0 | 0.0 | 0.0 |
| f | Rikenellaceae | 0.4 | 0.1 | 1.7 | 0.2 | 2.4 | 0.0 | 4.5 | 6.9 | 0.0 |
| g | Alistipes | 0.4 | 0.1 | 1.7 | 0.2 | 2.4 | 0.0 | 4.5 | 6.9 | 0.0 |
| s | Alistipes_putredinis | 0.3 | 0.0 | 0.0 | 0.0 | 1.8 | 0.0 | 4.2 | 6.1 | 0.0 |
| s | Alistipes_shahii | 0.1 | 0.0 | 0.1 | 0.0 | 0.6 | 0.0 | 0.3 | 0.8 | 0.0 |
| s | Alistipes_unclassified | 0.0 | 0.1 | 1.6 | 0.2 | 0.0 | 0.0 | 0.0 | 0.0 | 0.0 |
| p | Firmicutes | 78.6 | 86.3 | 16.3 | 38.4 | 67.9 | 78.1 | 36.2 | 64.9 | 51.3 |
| c | Bacilli | 3.2 | 1.9 | 4.7 | 0.9 | 0.3 | 10.1 | 1.4 | 0.4 | 3.6 |
| o | Lactobacillales | 3.2 | 1.9 | 4.7 | 0.8 | 0.3 | 10.1 | 1.4 | 0.4 | 3.6 |
| f | Enterococcaceae | 0.0 | 0.0 | 1.4 | 0.0 | 0.0 | 0.1 | 0.0 | 0.0 | 0.2 |
| g | Enterococcus | 0.0 | 0.0 | 1.4 | 0.0 | 0.0 | 0.1 | 0.0 | 0.0 | 0.2 |
| s | Enterococcus_casseliflavus | 0.0 | 0.0 | 1.4 | 0.0 | 0.0 | 0.0 | 0.0 | 0.0 | 0.0 |
| s | Enterococcus_faecalis | 0.0 | 0.0 | 0.0 | 0.0 | 0.0 | 0.1 | 0.0 | 0.0 | 0.2 |
| s | Enterococcus_faecium | 0.0 | 0.0 | 0.0 | 0.0 | 0.0 | 0.0 | 0.0 | 0.0 | 0.0 |
| s | Enterococcus_unclassified | 0.0 | 0.0 | 0.0 | 0.0 | 0.0 | 0.0 | 0.0 | 0.0 | 0.0 |
| f | Lactobacillaceae | 0.2 | 0.0 | 2.9 | 0.3 | 0.0 | 9.6 | 0.0 | 0.0 | 3.4 |
| g | Lactobacillus | 0.2 | 0.0 | 2.9 | 0.3 | 0.0 | 9.5 | 0.0 | 0.0 | 2.7 |
| s | Lactobacillus_casei | 0.0 | 0.0 | 1.6 | 0.0 | 0.0 | 0.0 | 0.0 | 0.0 | 0.0 |
| s | Lactobacillus_crispatus | 0.0 | 0.0 | 0.0 | 0.2 | 0.0 | 0.0 | 0.0 | 0.0 | 0.0 |
| s | Lactobacillus_fermentum | 0.0 | 0.0 | 0.2 | 0.0 | 0.0 | 0.1 | 0.0 | 0.0 | 0.0 |
| s | Lactobacillus_passeri | 0.1 | 0.0 | 0.1 | 0.0 | 0.0 | 1.0 | 0.0 | 0.0 | 0.1 |
| s | Lactobacillus_paracasei | 0.0 | 0.0 | 1.0 | 0.0 | 0.0 | 0.0 | 0.0 | 0.0 | 0.0 |
| s | Lactobacillus_reuteri | 0.0 | 0.0 | 0.0 | 0.0 | 0.0 | 0.0 | 0.0 | 0.0 | 0.0 |
| s | Lactobacillus_ruminis | 0.0 | 0.0 | 0.0 | 0.0 | 0.0 | 0.0 | 0.0 | 0.0 | 0.0 |
| s | Lactobacillus_salivarius | 0.0 | 0.0 | 0.0 | 0.1 | 0.0 | 7.9 | 0.0 | 0.0 | 2.1 |
| f | Streptococcaceae | 2.9 | 1.8 | 0.2 | 0.4 | 0.3 | 0.4 | 1.4 | 0.4 | 0.0 |
| g | Lactococcus | 1.3 | 1.0 | 0.2 | 0.0 | 0.0 | 0.0 | 0.0 | 0.0 | 0.0 |
| s | Lactococcus_lactis | 1.3 | 1.0 | 0.2 | 0.0 | 0.0 | 0.0 | 0.0 | 0.0 | 0.0 |
| g | Streptococcus | 1.6 | 0.8 | 0.1 | 0.4 | 0.3 | 0.4 | 1.3 | 0.4 | 0.0 |
| s | Streptococcus_anginosus | 0.0 | 0.0 | 0.0 | 0.0 | 0.0 | 0.0 | 0.0 | 0.0 | 0.0 |
| s | Streptococcus_infantarius | 0.0 | 0.0 | 0.0 | 0.0 | 0.0 | 0.0 | 0.0 | 0.0 | 0.0 |
| s | Streptococcus_mutans | 0.5 | 0.0 | 0.0 | 0.0 | 0.0 | 0.0 | 0.0 | 0.0 | 0.0 |
| s | Streptococcus_parasanguinis | 0.1 | 0.1 | 0.0 | 0.2 | 0.1 | 0.0 | 0.0 | 0.2 | 0.0 |
| s | Streptococcus_salivarius | 0.7 | 0.4 | 0.0 | 0.1 | 0.0 | 0.0 | 0.0 | 0.1 | 0.0 |
| s | Streptococcus_thermophilus | 0.2 | 0.2 | 0.0 | 0.0 | 0.0 | 0.0 | 1.3 | 0.0 | 0.0 |
| s | Streptococcus_vestibularis | 0.2 | 0.1 | 0.0 | 0.1 | 0.2 | 0.3 | 0.0 | 0.0 | 0.0 |
| c | Clostridia | 65.4 | 66.2 | 0.7 | 33.9 | 65.5 | 53.6 | 28.7 | 64.3 | 41.7 |
| o | Clostridiales | 65.4 | 66.2 | 0.7 | 33.9 | 65.5 | 53.6 | 28.7 | 64.3 | 41.7 |
| f | Clostridiaceae | 11.7 | 33.3 | 0.0 | 2.9 | 0.2 | 20.3 | 8.4 | 0.3 | 20.6 |
| g | Clostridium | 11.7 | 33.3 | 0.0 | 2.7 | 0.2 | 20.3 | 8.4 | 0.2 | 20.6 |
| s | Clostridium_asparagiforme | 0.0 | 0.0 | 0.0 | 0.5 | 0.0 | 0.4 | 0.0 | 0.0 | 1.1 |
| s | Clostridium_bartlettii | 7.8 | 2.2 | 0.0 | 0.0 | 0.0 | 1.8 | 0.0 | 0.0 | 3.3 |
| s | Clostridium_bolteae | 0.0 | 0.2 | 0.0 | 0.6 | 0.0 | 9.9 | 5.1 | 0.0 | 10.5 |
| s | Clostridium_cf | 0.1 | 0.1 | 0.0 | 0.2 | 0.0 | 0.0 | 0.0 | 0.0 | 0.0 |
| s | Clostridium_hathewayi | 0.0 | 0.0 | 0.0 | 0.2 | 0.0 | 1.5 | 0.0 | 0.0 | 2.8 |
| s | Clostridium_hiranonis | 0.0 | 0.0 | 0.0 | 0.0 | 0.0 | 0.0 | 0.0 | 0.0 | 0.0 |
| s | Clostridium_leptum | 1.6 | 22.5 | 0.0 | 0.2 | 0.0 | 0.0 | 0.1 | 0.0 | 0.0 |
| s | Clostridium_nexile | 0.0 | 8.0 | 0.0 | 0.0 | 0.0 | 0.0 | 0.0 | 0.1 | 0.0 |
| s | Clostridium_perfringens | 0.0 | 0.1 | 0.0 | 0.0 | 0.0 | 0.3 | 0.0 | 0.0 | 0.1 |
| s | Clostridium_scindens | 2.1 | 0.1 | 0.0 | 0.6 | 0.0 | 2.5 | 2.7 | 0.0 | 0.7 |
| s | Clostridium_symbiosum | 0.0 | 0.1 | 0.0 | 0.2 | 0.0 | 4.0 | 0.5 | 0.0 | 2.1 |
| f | Clostridiales_Family_XI_Incertae_Sedis | 3.6 | 0.1 | 0.0 | 0.0 | 0.0 | 0.2 | 0.0 | 0.0 | 0.0 |
| g | Anaerococcus | 1.2 | 0.0 | 0.0 | 0.0 | 0.0 | 0.0 | 0.0 | 0.0 | 0.0 |
| s | Anaerococcus_unclassified | 0.7 | 0.0 | 0.0 | 0.0 | 0.0 | 0.0 | 0.0 | 0.0 | 0.0 |
| g | Clostridiales_Family_XI_Incertae_Sedis_unclassified | 0.6 | 0.0 | 0.0 | 0.0 | 0.0 | 0.1 | 0.0 | 0.0 | 0.0 |
| g | Finegoldia | 1.1 | 0.0 | 0.0 | 0.0 | 0.0 | 0.0 | 0.0 | 0.0 | 0.0 |
| s | Finegoldia_magna | 1.1 | 0.0 | 0.0 | 0.0 | 0.0 | 0.0 | 0.0 | 0.0 | 0.0 |
| g | Parvimonas | 0.0 | 0.0 | 0.0 | 0.0 | 0.0 | 0.0 | 0.0 | 0.0 | 0.0 |
| s | Parvimonas_micros | 0.0 | 0.0 | 0.0 | 0.0 | 0.0 | 0.0 | 0.0 | 0.0 | 0.0 |
| g | Peptoniphilus | 0.8 | 0.0 | 0.0 | 0.0 | 0.0 | 0.0 | 0.0 | 0.0 | 0.0 |
| s | Peptoniphilus_harei | 0.0 | 0.0 | 0.0 | 0.0 | 0.0 | 0.0 | 0.0 | 0.0 | 0.0 |
| s | Peptoniphilus_unclassified | 0.7 | 0.0 | 0.0 | 0.0 | 0.0 | 0.0 | 0.0 | 0.0 | 0.0 |
| f | Clostridiales_uncl | 0.4 | 0.2 | 0.7 | 1.1 | 0.3 | 0.1 | 0.3 | 0.4 | 0.0 |
| g | Blautia | 0.4 | 0.2 | 0.7 | 1.1 | 0.3 | 0.1 | 0.3 | 0.4 | 0.0 |
| s | Blautia_hansenii | 0.0 | 0.0 | 0.0 | 0.0 | 0.0 | 0.0 | 0.0 | 0.0 | 0.0 |
| s | Blautia_hydrogenotrophica | 0.0 | 0.0 | 0.0 | 0.2 | 0.0 | 0.0 | 0.3 | 0.0 | 0.0 |
| s | Blautia_unclassified | 0.4 | 0.2 | 0.7 | 0.9 | 0.3 | 0.1 | 0.0 | 0.4 | 0.0 |
| f | Eubacteriaceae | 11.7 | 1.8 | 0.0 | 20.8 | 13.0 | 31.6 | 0.0 | 6.0 | 18.7 |
| g | Eubacterium | 11.5 | 1.8 | 0.0 | 20.5 | 13.0 | 0.0 | 0.0 | 5.9 | 0.0 |
| s | Eubacterium_eligens | 0.1 | 0.0 | 0.0 | 4.1 | 4.1 | 0.0 | 0.0 | 0.3 | 0.0 |
| s | Eubacterium_hallii | 3.5 | 1.4 | 0.0 | 0.0 | 3.0 | 0.0 | 0.0 | 2.5 | 0.0 |
| s | Eubacterium_limosum | 0.3 | 0.0 | 0.0 | 0.0 | 0.0 | 0.0 | 0.0 | 0.0 | 0.0 |
| s | Eubacterium_rectale | 7.6 | 0.4 | 0.0 | 0.0 | 0.5 | 0.0 | 0.0 | 3.2 | 0.0 |
| s | Eubacterium_siraeum | 0.0 | 0.0 | 0.0 | 16.4 | 5.3 | 0.0 | 0.0 | 0.0 | 0.0 |
| s | Eubacterium_ventriosum | 0.0 | 0.0 | 0.0 | 0.0 | 0.0 | 0.0 | 0.0 | 0.0 | 0.0 |
| g | Pseudoramibacter | 0.0 | 0.0 | 0.0 | 0.0 | 0.0 | 31.6 | 0.0 | 0.0 | 18.6 |
| s | Pseudoramibacter_alactolyticus | 0.0 | 0.0 | 0.0 | 0.0 | 0.0 | 31.6 | 0.0 | 0.0 | 18.6 |
| f | Lachnospiraceae | 11.3 | 18.5 | 0.0 | 0.8 | 32.0 | 0.1 | 0.4 | 22.5 | 0.0 |
| g | Anaerostipes | 0.1 | 0.0 | 0.0 | 0.2 | 0.0 | 0.0 | 0.4 | 0.0 | 0.0 |
| s | Anaerostipes_caccae | 0.1 | 0.0 | 0.0 | 0.2 | 0.0 | 0.0 | 0.4 | 0.0 | 0.0 |
| g | Butyrivibrio | 0.0 | 0.0 | 0.0 | 0.0 | 25.0 | 0.0 | 0.0 | 0.0 | 0.0 |
| s | Butyrivibrio_crossotus | 0.0 | 0.0 | 0.0 | 0.0 | 25.0 | 0.0 | 0.0 | 0.0 | 0.0 |
| g | Coprococcus | 2.5 | 0.0 | 0.0 | 0.2 | 2.6 | 0.0 | 0.0 | 1.7 | 0.0 |
| s | Coprococcus_catus | 2.5 | 0.0 | 0.0 | 0.0 | 0.5 | 0.0 | 0.0 | 0.0 | 0.0 |
| s | Coprococcus_comes | 0.0 | 0.0 | 0.0 | 0.2 | 2.0 | 0.0 | 0.0 | 1.7 | 0.0 |
| g | Dorea | 8.0 | 0.0 | 0.0 | 0.4 | 1.5 | 0.0 | 0.0 | 2.9 | 0.0 |
| s | Dorea_formicigenerans | 0.5 | 0.0 | 0.0 | 0.4 | 0.6 | 0.0 | 0.0 | 1.2 | 0.0 |
| s | Dorea_longicatena | 7.4 | 0.0 | 0.0 | 0.0 | 1.0 | 0.0 | 0.0 | 1.7 | 0.0 |
| g | Roseburia | 0.7 | 18.5 | 0.0 | 0.0 | 2.9 | 0.0 | 0.0 | 17.9 | 0.0 |
| s | Roseburia_intestinalis | 0.5 | 17.9 | 0.0 | 0.0 | 1.1 | 0.0 | 0.0 | 17.6 | 0.0 |
| s | Roseburia_inulinivorans | 0.3 | 0.6 | 0.0 | 0.0 | 1.8 | 0.0 | 0.0 | 0.3 | 0.0 |
| f | Ruminococcaceae | 26.7 | 12.4 | 0.0 | 8.3 | 20.0 | 1.4 | 19.6 | 35.1 | 2.4 |
| g | Anaerotruncus | 0.2 | 0.0 | 0.0 | 0.0 | 0.1 | 0.6 | 0.1 | 0.0 | 1.7 |
| s | Anaerotruncus_colihominis | 0.2 | 0.0 | 0.0 | 0.0 | 0.1 | 0.6 | 0.1 | 0.0 | 1.7 |
| g | Faecalibacterium | 4.4 | 1.8 | 0.0 | 0.9 | 6.3 | 0.0 | 0.0 | 21.6 | 0.0 |
| s | Faecalibacterium_cf | 1.8 | 0.0 | 0.0 | 0.0 | 0.8 | 0.0 | 0.0 | 1.2 | 0.0 |
| s | Faecalibacterium_prausnitzii | 2.6 | 0.9 | 0.0 | 0.7 | 5.5 | 0.0 | 0.0 | 20.4 | 0.0 |
| s | Faecalibacterium_unclassified | 0.0 | 0.9 | 0.0 | 0.2 | 0.0 | 0.0 | 0.0 | 0.0 | 0.0 |
| s | Ruminococcus_bromii | 0.0 | 0.0 | 0.0 | 0.0 | 6.7 | 0.0 | 0.0 | 0.0 | 0.1 |
| s | Ruminococcus_gnavus | 0.8 | 10.2 | 0.0 | 0.9 | 0.0 | 0.0 | 0.2 | 0.1 | 0.0 |
| s | Ruminococcus_lactaris | 4.7 | 0.0 | 0.0 | 1.0 | 0.0 | 0.0 | 0.0 | 10.0 | 0.0 |
| s | Ruminococcus_obeum | 11.3 | 0.2 | 0.0 | 0.3 | 1.2 | 0.0 | 19.2 | 0.8 | 0.0 |
| s | Ruminococcus_torques | 5.2 | 0.2 | 0.0 | 5.2 | 5.6 | 0.6 | 0.0 | 2.6 | 0.5 |
| c | Erysipelotrichi | 0.6 | 14.1 | 2.0 | 3.6 | 0.0 | 0.1 | 6.1 | 0.1 | 0.1 |
| o | Erysipelotrichales | 0.6 | 14.1 | 2.0 | 3.6 | 0.0 | 0.1 | 6.1 | 0.1 | 0.1 |
| f | Erysipelotrichaceae | 0.6 | 14.1 | 2.0 | 3.6 | 0.0 | 0.1 | 6.1 | 0.1 | 0.1 |
| g | Catenibacterium | 0.0 | 0.0 | 0.0 | 0.0 | 0.0 | 0.0 | 0.0 | 0.0 | 0.0 |
| s | Catenibacterium_mitsuokai | 0.0 | 0.0 | 0.0 | 0.0 | 0.0 | 0.0 | 0.0 | 0.0 | 0.0 |
| g | Coprobacillus | 0.4 | 13.9 | 0.1 | 3.6 | 0.0 | 0.1 | 6.1 | 0.0 | 0.0 |
| s | Coprobacillus_bacterium | 0.4 | 13.9 | 0.1 | 3.6 | 0.0 | 0.1 | 6.1 | 0.0 | 0.0 |
| g | Holdemania | 0.2 | 0.2 | 1.9 | 0.0 | 0.0 | 0.0 | 0.0 | 0.1 | 0.0 |
| s | Holdemania filiformis | 0.2 | 0.2 | 1.9 | 0.0 | 0.0 | 0.0 | 0.0 | 0.1 | 0.0 |
| c | Negativicutes | 9.4 | 4.1 | 8.9 | 0.0 | 2.1 | 14.3 | 0.0 | 0.0 | 5.9 |
| o | Selenomonadales | 9.4 | 4.1 | 8.9 | 0.0 | 2.1 | 14.3 | 0.0 | 0.0 | 5.9 |
| f | Acidaminococcaceae | 0.0 | 0.0 | 0.0 | 0.0 | 2.1 | 0.0 | 0.0 | 0.0 | 0.0 |
| g | Acidaminococcaceae_unclassified | 0.0 | 0.0 | 0.0 | 0.0 | 0.0 | 0.0 | 0.0 | 0.0 | 0.0 |
| g | Phascolarctobacterium | 0.0 | 0.0 | 0.0 | 0.0 | 2.1 | 0.0 | 0.0 | 0.0 | 0.0 |
| s | Phascolarctobacterium_unclassified | 0.0 | 0.0 | 0.0 | 0.0 | 2.1 | 0.0 | 0.0 | 0.0 | 0.0 |
| f | Veillonellaceae | 9.4 | 4.1 | 8.9 | 0.0 | 0.0 | 14.3 | 0.0 | 0.0 | 5.9 |
| g | Dialister | 9.4 | 4.1 | 0.6 | 0.0 | 0.0 | 14.3 | 0.0 | 0.0 | 5.9 |
| s | Dialister_invisus | 9.4 | 4.1 | 0.6 | 0.0 | 0.0 | 14.3 | 0.0 | 0.0 | 5.9 |
| g | Megamonas | 0.0 | 0.0 | 0.0 | 0.0 | 0.0 | 0.0 | 0.0 | 0.0 | 0.0 |
| s | Megamonas_hypermegale | 0.0 | 0.0 | 0.0 | 0.0 | 0.0 | 0.0 | 0.0 | 0.0 | 0.0 |
| g | Veillonella | 0.0 | 0.0 | 8.3 | 0.0 | 0.0 | 0.0 | 0.0 | 0.0 | 0.0 |
| s | Veillonella_atypica | 0.0 | 0.0 | 0.0 | 0.0 | 0.0 | 0.0 | 0.0 | 0.0 | 0.0 |
| s | Veillonella_parvula | 0.0 | 0.0 | 3.0 | 0.0 | 0.0 | 0.0 | 0.0 | 0.0 | 0.0 |
| s | Veillonella_unclassified | 0.0 | 0.0 | 5.2 | 0.0 | 0.0 | 0.0 | 0.0 | 0.0 | 0.0 |
| p | Fusobacteria | 0.0 | 0.0 | 0.2 | 0.0 | 0.0 | 0.0 | 0.0 | 0.0 | 0.0 |
| c | Fusobacteria | 0.0 | 0.0 | 0.2 | 0.0 | 0.0 | 0.0 | 0.0 | 0.0 | 0.0 |
| o | Fusobacteriales | 0.0 | 0.0 | 0.2 | 0.0 | 0.0 | 0.0 | 0.0 | 0.0 | 0.0 |
| f | Fusobacteriaceae | 0.0 | 0.0 | 0.2 | 0.0 | 0.0 | 0.0 | 0.0 | 0.0 | 0.0 |
| g | Fusobacterium | 0.0 | 0.0 | 0.2 | 0.0 | 0.0 | 0.0 | 0.0 | 0.0 | 0.0 |
| s | Fusobacterium_ulcerans | 0.0 | 0.0 | 0.0 | 0.0 | 0.0 | 0.0 | 0.0 | 0.0 | 0.0 |
| p | Proteobacteria | 0.0 | 0.7 | 21.2 | 0.6 | 1.5 | 0.8 | 0.4 | 0.5 | 0.8 |
| c | Betaproteobacteria | 0.0 | 0.0 | 0.0 | 0.0 | 1.4 | 0.0 | 0.0 | 0.0 | 0.0 |
| o | Burkholderiales | 0.0 | 0.0 | 0.0 | 0.0 | 1.4 | 0.0 | 0.0 | 0.0 | 0.0 |
| f | Sutterellaceae | 0.0 | 0.0 | 0.0 | 0.0 | 1.4 | 0.0 | 0.0 | 0.0 | 0.0 |
| g | Sutterella | 0.0 | 0.0 | 0.0 | 0.0 | 1.4 | 0.0 | 0.0 | 0.0 | 0.0 |
| s | Sutterella_wadsworthensis | 0.0 | 0.0 | 0.0 | 0.0 | 1.4 | 0.0 | 0.0 | 0.0 | 0.0 |
| c | Deltaproteobacteria | 0.0 | 0.0 | 0.0 | 0.0 | 0.1 | 0.1 | 0.4 | 0.4 | 0.1 |
| o | Desulfovibrionales | 0.0 | 0.0 | 0.0 | 0.0 | 0.1 | 0.1 | 0.4 | 0.4 | 0.1 |
| f | Desulfovibrionaceae | 0.0 | 0.0 | 0.0 | 0.0 | 0.1 | 0.1 | 0.4 | 0.4 | 0.1 |
| g | Bilophila | 0.0 | 0.0 | 0.0 | 0.0 | 0.0 | 0.0 | 0.4 | 0.4 | 0.0 |
| s | Bilophila_wadsworthia | 0.0 | 0.0 | 0.0 | 0.0 | 0.0 | 0.0 | 0.4 | 0.4 | 0.0 |
| g | Desulfovibrio | 0.0 | 0.0 | 0.0 | 0.0 | 0.1 | 0.1 | 0.0 | 0.0 | 0.1 |
| s | Desulfovibrio_piper | 0.0 | 0.0 | 0.0 | 0.0 | 0.0 | 0.0 | 0.0 | 0.0 | 0.0 |
| c | Gammaproteobacteria | 0.0 | 0.7 | 21.2 | 0.6 | 0.0 | 0.1 | 0.0 | 0.1 | 0.2 |
| o | Enterobacteriales | 0.0 | 0.7 | 21.2 | 0.6 | 0.0 | 0.1 | 0.0 | 0.1 | 0.2 |
| f | Enterobacteriaceae | 0.0 | 0.7 | 21.2 | 0.6 | 0.0 | 0.1 | 0.0 | 0.1 | 0.2 |
| g | Enterobacter | 0.0 | 0.0 | 0.0 | 0.0 | 0.0 | 0.0 | 0.0 | 0.0 | 0.0 |
| s | Enterobacter_cloacae | 0.0 | 0.0 | 0.0 | 0.0 | 0.0 | 0.0 | 0.0 | 0.0 | 0.0 |
| g | Escherichia | 0.0 | 0.1 | 19.6 | 0.4 | 0.0 | 0.1 | 0.0 | 0.1 | 0.1 |
| s | Escherichia_coli | 0.0 | 0.1 | 19.6 | 0.4 | 0.0 | 0.1 | 0.0 | 0.1 | 0.1 |
| s | Escherichia_unclassified | 0.0 | 0.0 | 0.0 | 0.0 | 0.0 | 0.0 | 0.0 | 0.0 | 0.0 |
| g | Klebsiella | 0.0 | 0.0 | 1.3 | 0.0 | 0.0 | 0.0 | 0.0 | 0.0 | 0.0 |
| s | Klebsiella_pneumoniae | 0.0 | 0.0 | 1.3 | 0.0 | 0.0 | 0.0 | 0.0 | 0.0 | 0.0 |
| s | Klebsiella_unclassified | 0.0 | 0.0 | 0.0 | 0.0 | 0.0 | 0.0 | 0.0 | 0.0 | 0.0 |
| p | Verrucomicrobia | 0.8 | 0.2 | 0.3 | 0.0 | 3.8 | 2.0 | 24.3 | 0.0 | 1.6 |
| c | Verrucomicrobiae | 0.8 | 0.2 | 0.3 | 0.0 | 3.8 | 2.0 | 24.3 | 0.0 | 1.6 |
| o | Verrucomicrobiales | 0.8 | 0.2 | 0.3 | 0.0 | 3.8 | 2.0 | 24.3 | 0.0 | 1.6 |
| f | Verrucomicrobiaceae | 0.8 | 0.2 | 0.3 | 0.0 | 3.8 | 2.0 | 24.3 | 0.0 | 1.6 |
| g | Akkermansia | 0.8 | 0.2 | 0.3 | 0.0 | 3.8 | 2.0 | 24.3 | 0.0 | 1.6 |
| s | Akkermansia_muciniphila | 0.8 | 0.2 | 0.3 | 0.0 | 3.8 | 2.0 | 24.3 | 0.0 | 1.6 |

| Rank | Name | P10 | P11 | P12 | P13 | P14 | P15 | P16 | P17 |
|---|---|---|---|---|---|---|---|---|---|
| k | Archaea | 5.1 | 0.0 | 0.0 | 0.0 | 2.4 | 0.0 | 2.2 | 0.0 |
| p | Euryarchaeota | 5.1 | 0.0 | 0.0 | 0.0 | 2.4 | 0.0 | 2.2 | 0.0 |
| c | Methanobacteria | 5.1 | 0.0 | 0.0 | 0.0 | 2.4 | 0.0 | 2.2 | 0.0 |
| o | Methanobacteriales | 5.1 | 0.0 | 0.0 | 0.0 | 2.4 | 0.0 | 2.2 | 0.0 |
| f | Methanobacteriaceae | 5.1 | 0.0 | 0.0 | 0.0 | 2.4 | 0.0 | 2.2 | 0.0 |
| g | Methanobrevibacter | 5.1 | 0.0 | 0.0 | 0.0 | 2.4 | 0.0 | 2.2 | 0.0 |
| s | Methanobrevibacter_smithii | 5.1 | 0.0 | 0.0 | 0.0 | 2.4 | 0.0 | 2.2 | 0.0 |
| k | Bacteria | 94.9 | 100.0 | 100.0 | 100.0 | 97.6 | 100.0 | 97.8 | 100.0 |
| p | Actinobacteria | 2.6 | 3.0 | 10.2 | 2.3 | 4.9 | 20.4 | 1.4 | 0.7 |
| c | Actinobacteria | 2.6 | 3.0 | 10.2 | 2.3 | 4.9 | 20.4 | 1.4 | 0.7 |
| o | Actinomycetales | 0.1 | 0.0 | 0.0 | 0.0 | 0.2 | 0.1 | 0.1 | 0.0 |
| f | Corynebacteriaceae | 0.0 | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 | 0.0 |
| s | Corynebacterium_amycolatum | 0.0 | 0.0 | 0.0 | 0.0 | 0.0 | 0.0 | 0.0 | 0.0 |
| s | Corynebacterium_striatum | 0.0 | 0.0 | 0.0 | 0.0 | 0.0 | 0.0 | 0.0 | 0.0 |
| o | Bifidobacteriales | 0.5 | 2.5 | 5.7 | 1.8 | 0.0 | 0.0 | 0.1 | 0.0 |
| f | Bifidobacteriaceae | 0.5 | 2.5 | 5.7 | 1.8 | 0.0 | 0.0 | 0.1 | 0.0 |
| g | Bifidobacterium | 0.5 | 2.5 | 5.7 | 1.8 | 0.0 | 0.0 | 0.1 | 0.0 |
| s | Bifidobacterium_adolescentis | 0.4 | 0.0 | 0.0 | 0.0 | 0.0 | 0.0 | 0.0 | 0.0 |
| s | Bifidobacterium_animalis | 0.0 | 0.0 | 0.0 | 0.0 | 0.0 | 0.0 | 0.0 | 0.0 |
| s | Bifidobacterium_bifidum | 0.0 | 0.0 | 0.0 | 0.0 | 0.0 | 0.0 | 0.0 | 0.0 |
| s | Bifidobacterium_breve | 0.0 | 0.0 | 0.0 | 0.0 | 0.0 | 0.0 | 0.0 | 0.0 |
| s | Bifidobacterium_catenulatum | 0.0 | 0.0 | 0.0 | 0.0 | 0.0 | 0.0 | 0.0 | 0.0 |
| s | Bifidobacterium_dentium | 0.0 | 0.1 | 0.2 | 1.2 | 0.0 | 0.0 | 0.1 | 0.0 |
| s | Bifidobacterium_longum | 0.0 | 0.4 | 5.5 | 0.6 | 0.0 | 0.0 | 0.0 | 0.0 |
| s | Bifidobacterium_pseudocatenulatum | 0.0 | 2.1 | 0.0 | 0.0 | 0.0 | 0.0 | 0.0 | 0.0 |
| o | Coriobacteriales | 2.0 | 0.5 | 4.5 | 0.5 | 4.7 | 20.3 | 1.1 | 0.6 |
| f | Coriobacteriaceae | 2.0 | 0.5 | 4.5 | 0.5 | 4.7 | 20.3 | 1.1 | 0.6 |
| g | Collinsella | 1.9 | 0.3 | 4.2 | 0.0 | 3.8 | 16.6 | 1.1 | 0.0 |
| s | Collinsella_aerofaciens | 1.9 | 0.0 | 4.2 | 0.0 | 0.0 | 16.6 | 1.1 | 0.0 |
| s | Collinsella_intestinalis | 0.0 | 0.0 | 0.0 | 0.0 | 3.8 | 0.0 | 0.0 | 0.0 |
| g | Eggerthella | 0.1 | 0.1 | 0.3 | 0.4 | 0.9 | 2.7 | 0.0 | 0.6 |
| s | Eggerthella_lenta | 0.1 | 0.1 | 0.3 | 0.4 | 0.9 | 2.7 | 0.0 | 0.6 |
| g | Gordonibacter | 0.1 | 0.1 | 0.0 | 0.0 | 0.1 | 1.1 | 0.0 | 0.1 |
| s | Gordonibacter_pamelaeae | 0.1 | 0.1 | 0.0 | 0.0 | 0.1 | 1.1 | 0.0 | 0.1 |
| p | Bacteroidetes | 23.0 | 56.3 | 4.1 | 7.2 | 22.2 | 31.2 | 20.3 | 35.7 |
| c | Bacteroidia | 23.0 | 56.3 | 4.1 | 7.2 | 22.2 | 31.2 | 20.3 | 35.7 |
| o | Bacteroidales | 23.0 | 56.3 | 4.1 | 7.2 | 22.2 | 31.2 | 20.3 | 35.7 |
| f | Bacteroidaceae | 6.5 | 39.9 | 4.0 | 5.9 | 14.6 | 31.2 | 11.5 | 35.1 |
| g | Bacteroides | 6.5 | 39.9 | 4.0 | 5.9 | 14.6 | 31.2 | 11.5 | 35.1 |
| s | Bacteroides_caccae | 0.4 | 0.0 | 0.0 | 0.9 | 0.0 | 0.0 | 0.1 | 0.0 |
| s | Bacteroides_cellulosilyticus | 0.7 | 0.0 | 0.1 | 0.0 | 1.4 | 0.0 | 0.1 | 0.0 |
| s | Bacteroides_coprocola | 0.0 | 0.0 | 0.0 | 0.0 | 0.0 | 0.0 | 0.0 | 0.0 |
| s | Bacteroides_coprophilus | 0.0 | 0.0 | 0.0 | 0.0 | 0.0 | 0.0 | 4.7 | 0.0 |
| s | Bacteroides_eggerthii | 0.0 | 0.0 | 0.0 | 0.0 | 0.0 | 0.0 | 0.0 | 0.0 |
| s | Bacteroides_finegoldii | 0.1 | 0.0 | 0.0 | 0.0 | 0.0 | 0.0 | 1.5 | 0.0 |
| s | Bacteroides_fragilis | 0.0 | 0.0 | 0.0 | 1.6 | 0.0 | 0.2 | 0.2 | 0.0 |
| s | Bacteroides_intestinalis | 0.1 | 0.0 | 0.0 | 0.0 | 0.0 | 0.0 | 0.5 | 0.0 |
| s | Bacteroides_ovatus | 0.1 | 0.0 | 0.1 | 0.1 | 0.7 | 0.0 | 0.0 | 1.7 |
| s | Bacteroides_pectinophilus | 0.0 | 0.0 | 0.0 | 0.0 | 0.0 | 0.0 | 0.0 | 0.0 |
| s | Bacteroides_plebeius | 0.5 | 0.0 | 0.0 | 0.0 | 0.0 | 0.0 | 0.0 | 0.0 |
| s | Bacteroides_stercoris | 0.2 | 0.0 | 0.0 | 0.0 | 0.0 | 0.0 | 0.3 | 0.0 |
| s | Bacteroides_thetaiotaomicron | 0.0 | 0.3 | 0.0 | 0.1 | 0.0 | 0.0 | 0.0 | 0.0 |
| s | Bacteroides_unclassified | 1.2 | 30.6 | 2.0 | 3.2 | 8.7 | 26.9 | 0.0 | 19.7 |
| s | Bacteroides_uniformis | 1.0 | 2.0 | 0.0 | 0.0 | 1.8 | 4.1 | 0.1 | 0.0 |
| s | Bacteroides_vulgatus | 2.0 | 4.0 | 1.7 | 0.0 | 1.7 | 0.0 | 3.6 | 13.7 |
| s | Bacteroides_xylanisolvens | 0.1 | 2.9 | 0.0 | 0.1 | 0.2 | 0.0 | 0.1 | 0.0 |
| f | Porphyromonadaceae | 3.6 | 12.0 | 0.1 | 1.1 | 2.7 | 0.0 | 3.4 | 0.0 |
| g | Odoribacter | 1.1 | 0.0 | 0.0 | 0.2 | 0.8 | 0.0 | 2.0 | 0.0 |
| s | Odoribacter_splanchnicus | 1.1 | 0.0 | 0.0 | 0.2 | 0.8 | 0.0 | 2.0 | 0.0 |
| g | Parabacteroides | 2.4 | 12.0 | 0.0 | 0.9 | 1.8 | 0.0 | 0.7 | 0.0 |
| s | Parabacteroides_distasonis | 0.0 | 0.0 | 0.0 | 0.0 | 0.3 | 0.0 | 0.0 | 0.0 |
| s | Parabacteroides_johnsonii | 0.0 | 0.0 | 0.0 | 0.0 | 1.2 | 0.0 | 0.0 | 0.0 |
| s | Parabacteroides_merdae | 1.7 | 9.1 | 0.0 | 0.7 | 0.0 | 0.0 | 0.6 | 0.0 |
| s | Parabacteroides_unclassified | 0.7 | 2.9 | 0.0 | 0.2 | 0.4 | 0.0 | 0.1 | 0.0 |
| g | Porphyromonas | 0.0 | 0.0 | 0.0 | 0.0 | 0.1 | 0.0 | 0.7 | 0.0 |
| s | Porphyromonas_asaccharolytica | 0.0 | 0.0 | 0.0 | 0.0 | 0.1 | 0.0 | 0.7 | 0.0 |
| f | Prevotellaceae | 0.0 | 0.0 | 0.0 | 0.0 | 0.3 | 0.0 | 0.0 | 0.0 |
| g | Prevotella | 0.0 | 0.0 | 0.0 | 0.0 | 0.3 | 0.0 | 0.0 | 0.0 |
| s | Prevotella_copri | 0.0 | 0.0 | 0.0 | 0.0 | 0.0 | 0.0 | 0.0 | 0.0 |
| f | Rikenellaceae | 12.9 | 4.4 | 0.1 | 0.1 | 4.6 | 0.0 | 5.4 | 0.6 |
| g | Alistipes | 12.9 | 4.4 | 0.1 | 0.1 | 4.6 | 0.0 | 5.4 | 0.6 |
| s | Alistipes_putredinis | 11.5 | 4.3 | 0.0 | 0.0 | 3.7 | 0.0 | 1.9 | 0.0 |
| s | Alistipes_shahii | 1.4 | 0.1 | 0.0 | 0.0 | 0.8 | 0.0 | 3.3 | 0.1 |
| s | Alistipes_unclassified | 0.0 | 0.0 | 0.0 | 0.1 | 0.0 | 0.0 | 0.2 | 0.5 |
| p | Firmicutes | 67.1 | 39.8 | 85.1 | 90.4 | 52.6 | 43.1 | 59.0 | 61.2 |
| c | Bacilli | 2.6 | 0.7 | 1.5 | 69.4 | 0.8 | 0.6 | 0.6 | 0.8 |
| o | Lactobacillales | 2.6 | 0.7 | 1.5 | 69.4 | 0.8 | 0.6 | 0.6 | 0.8 |
| f | Enterococcaceae | 0.1 | 0.0 | 0.0 | 0.0 | 0.0 | 0.0 | 0.0 | 0.0 |
| g | Enterococcus | 0.1 | 0.0 | 0.0 | 0.0 | 0.0 | 0.0 | 0.0 | 0.0 |
| s | Enterococcus_casseliflavus | 0.0 | 0.0 | 0.0 | 0.0 | 0.0 | 0.0 | 0.0 | 0.0 |
| s | Enterococcus_faecalis | 0.1 | 0.0 | 0.0 | 0.0 | 0.0 | 0.0 | 0.0 | 0.0 |
| s | Enterococcus_faecium | 0.0 | 0.0 | 0.0 | 0.0 | 0.0 | 0.0 | 0.0 | 0.0 |
| s | Enterococcus_unclassified | 0.0 | 0.0 | 0.0 | 0.0 | 0.0 | 0.0 | 0.0 | 0.0 |
| f | Lactobacillaceae | 0.4 | 0.6 | 0.1 | 65.5 | 0.1 | 0.1 | 0.4 | 0.0 |
| g | Lactobacillus | 0.4 | 0.6 | 0.1 | 65.5 | 0.1 | 0.1 | 0.4 | 0.0 |
| s | Lactobacillus_casei | 0.0 | 0.0 | 0.0 | 0.1 | 0.0 | 0.0 | 0.0 | 0.0 |
| s | Lactobacillus_crispatus | 0.0 | 0.0 | 0.0 | 0.1 | 0.0 | 0.0 | 0.0 | 0.0 |
| s | Lactobacillus_fermentum | 0.1 | 0.0 | 0.0 | 5.6 | 0.1 | 0.0 | 0.0 | 0.0 |
| s | Lactobacillus_passeri | 0.1 | 0.5 | 0.0 | 12.2 | 0.0 | 0.0 | 0.1 | 0.0 |
| s | Lactobacillus_paracasei | 0.0 | 0.0 | 0.0 | 0.0 | 0.0 | 0.0 | 0.0 | 0.0 |
| s | Lactobacillus_reuteri | 0.0 | 0.0 | 0.0 | 0.0 | 0.0 | 0.0 | 0.0 | 0.0 |
| s | Lactobacillus_ruminis | 0.0 | 0.0 | 0.0 | 0.0 | 0.0 | 0.0 | 0.0 | 0.0 |
| s | Lactobacillus_salivarius | 0.0 | 0.0 | 0.0 | 47.2 | 0.0 | 0.1 | 0.3 | 0.0 |
| f | Streptococcaceae | 2.2 | 0.1 | 1.4 | 3.9 | 0.6 | 0.5 | 0.2 | 0.8 |
| g | Lactococcus | 0.0 | 0.0 | 0.8 | 0.0 | 0.2 | 0.2 | 0.0 | 0.0 |
| s | Lactococcus_lactis | 0.0 | 0.0 | 0.8 | 0.0 | 0.2 | 0.2 | 0.0 | 0.0 |
| g | Streptococcus | 2.1 | 0.1 | 0.6 | 3.9 | 0.5 | 0.4 | 0.2 | 0.8 |
| s | Streptococcus_anginosus | 0.0 | 0.0 | 0.0 | 0.0 | 0.1 | 0.0 | 0.0 | 0.0 |
| s | Streptococcus_infantarius | 0.0 | 0.0 | 0.0 | 0.0 | 0.0 | 0.0 | 0.0 | 0.0 |
| s | Streptococcus_mutans | 0.4 | 0.0 | 0.0 | 1.0 | 0.0 | 0.0 | 0.0 | 0.0 |
| s | Streptococcus_parasanguinis | 0.2 | 0.0 | 0.1 | 0.7 | 0.1 | 0.0 | 0.0 | 0.2 |
| s | Streptococcus_salivarius | 0.4 | 0.1 | 0.2 | 1.7 | 0.1 | 0.0 | 0.2 | 0.5 |
| s | Streptococcus_thermophilus | 0.2 | 0.0 | 0.2 | 0.1 | 0.0 | 0.3 | 0.0 | 0.1 |
| s | Streptococcus_vestibularis | 0.0 | 0.0 | 0.1 | 0.4 | 0.1 | 0.0 | 0.0 | 0.0 |
| c | Clostridia | 63.0 | 39.1 | 66.6 | 20.5 | 49.7 | 37.9 | 57.9 | 51.2 |
| o | Clostridiales | 63.0 | 39.1 | 66.6 | 20.5 | 49.7 | 37.9 | 57.9 | 51.2 |
| f | Clostridiaceae | 2.0 | 4.6 | 33.6 | 0.9 | 15.7 | 14.7 | 0.2 | 6.6 |
| g | Clostridium | 2.0 | 4.6 | 33.6 | 0.9 | 15.7 | 14.7 | 0.2 | 6.6 |
| s | Clostridium_asparagiforme | 0.0 | 0.2 | 0.0 | 0.0 | 0.2 | 0.7 | 0.0 | 0.4 |
| s | Clostridium_bartlettii | 0.0 | 0.0 | 2.0 | 0.5 | 0.2 | 4.3 | 0.0 | 0.0 |
| s | Clostridium_bolteae | 0.1 | 0.5 | 0.2 | 0.1 | 10.9 | 1.1 | 0.0 | 2.4 |
| s | Clostridium_cf | 0.0 | 0.0 | 0.0 | 0.0 | 0.0 | 0.5 | 0.0 | 0.1 |
| s | Clostridium_hathewayi | 0.0 | 0.1 | 0.0 | 0.0 | 1.5 | 0.1 | 0.1 | 0.2 |
| s | Clostridium_hiranonis | 0.0 | 0.0 | 0.0 | 0.0 | 0.0 | 0.0 | 0.0 | 0.0 |
| s | Clostridium_leptum | 1.2 | 3.1 | 22.5 | 0.0 | 0.2 | 3.8 | 0.1 | 0.0 |
| s | Clostridium_nexile | 0.3 | 0.1 | 8.6 | 0.0 | 0.4 | 0.0 | 0.0 | 1.8 |
| s | Clostridium_perfringens | 0.0 | 0.0 | 0.1 | 0.0 | 0.0 | 0.0 | 0.0 | 0.0 |
| s | Clostridium_scindens | 0.3 | 0.4 | 0.1 | 0.1 | 1.6 | 2.2 | 0.0 | 0.8 |
| s | Clostridium_symbiosum | 0.0 | 0.2 | 0.1 | 0.1 | 0.6 | 1.7 | 0.0 | 1.0 |
| f | Clostridiales_Family_XI_Incertae_Sedis | 0.0 | 0.0 | 0.0 | 0.0 | 1.6 | 0.1 | 0.0 | 0.7 |
| g | Anaerococcus | 0.0 | 0.0 | 0.0 | 0.0 | 0.5 | 0.0 | 0.0 | 0.1 |
| s | Anaerococcus_unclassified | 0.0 | 0.0 | 0.0 | 0.0 | 0.4 | 0.0 | 0.0 | 0.0 |
| g | Clostridiales_Family_XI_Incertae_Sedis_unclassified | 0.0 | 0.0 | 0.0 | 0.0 | 0.3 | 0.1 | 0.0 | 0.6 |
| g | Finegoldia | 0.0 | 0.0 | 0.0 | 0.0 | 0.0 | 0.0 | 0.0 | 0.0 |
| s | Finegoldia_magna | 0.0 | 0.0 | 0.0 | 0.0 | 0.0 | 0.0 | 0.0 | 0.0 |
| g | Parvimonas | 0.0 | 0.0 | 0.0 | 0.0 | 0.0 | 0.0 | 0.0 | 0.0 |
| s | Parvimonas_micros | 0.0 | 0.0 | 0.0 | 0.0 | 0.0 | 0.0 | 0.0 | 0.0 |
| g | Peptoniphilus | 0.0 | 0.0 | 0.0 | 0.0 | 0.7 | 0.0 | 0.0 | 0.0 |
| s | Peptoniphilus_harei | 0.0 | 0.0 | 0.0 | 0.0 | 0.0 | 0.0 | 0.0 | 0.0 |
| s | Peptoniphilus_unclassified | 0.0 | 0.0 | 0.0 | 0.0 | 0.6 | 0.0 | 0.0 | 0.0 |
| f | Clostridiales_uncl | 0.2 | 2.6 | 0.2 | 0.6 | 0.5 | 3.7 | 0.2 | 0.6 |
| g | Blautia | 0.2 | 2.6 | 0.2 | 0.6 | 0.5 | 3.7 | 0.2 | 0.6 |
| s | Blautia_hansenii | 0.0 | 2.6 | 0.0 | 0.6 | 0.0 | 0.0 | 0.0 | 0.4 |
| s | Blautia_hydrogenotrophica | 0.1 | 0.0 | 0.0 | 0.0 | 0.5 | 3.1 | 0.0 | 0.0 |
| s | Blautia_unclassified | 0.1 | 0.0 | 0.2 | 0.0 | 0.0 | 0.5 | 0.2 | 0.3 |
| f | Eubacteriaceae | 14.5 | 1.1 | 1.6 | 8.7 | 6.2 | 3.9 | 11.1 | 0.0 |
| g | Eubacterium | 14.4 | 1.1 | 1.6 | 8.7 | 6.2 | 3.9 | 11.1 | 0.0 |
| s | Eubacterium_eligens | 2.4 | 0.0 | 0.0 | 0.0 | 0.0 | 3.9 | 2.9 | 0.0 |
| s | Eubacterium_hallii | 8.9 | 0.0 | 1.2 | 1.3 | 0.0 | 0.0 | 1.4 | 0.0 |
| s | Eubacterium_limosum | 0.0 | 0.7 | 0.0 | 0.0 | 0.6 | 0.0 | 0.0 | 0.0 |
| s | Eubacterium_rectale | 2.9 | 0.0 | 0.3 | 7.4 | 5.5 | 0.0 | 6.5 | 0.0 |
| s | Eubacterium_siraeum | 0.0 | 0.0 | 0.0 | 0.0 | 0.1 | 0.0 | 0.1 | 0.0 |
| s | Eubacterium_ventriosum | 0.2 | 0.3 | 0.0 | 0.0 | 0.0 | 0.0 | 0.2 | 0.0 |
| g | Pseudoramibacter | 0.0 | 0.0 | 0.0 | 0.0 | 0.0 | 0.0 | 0.0 | 0.0 |
| s | Pseudoramibacter_alactolyticus | 0.0 | 0.0 | 0.0 | 0.0 | 0.0 | 0.0 | 0.0 | 0.0 |
| f | Lachnospiraceae | 8.9 | 7.6 | 19.6 | 2.1 | 0.2 | 2.2 | 14.1 | 0.1 |
| g | Anaerostipes | 0.0 | 0.1 | 0.0 | 0.0 | 0.2 | 1.5 | 0.0 | 0.1 |
| s | Anaerostipes_caccae | 0.0 | 0.1 | 0.0 | 0.0 | 0.2 | 1.5 | 0.0 | 0.1 |
| g | Butyrivibrio | 0.0 | 0.0 | 0.0 | 0.0 | 0.0 | 0.0 | 8.8 | 0.0 |
| s | Butyrivibrio_crossotus | 0.0 | 0.0 | 0.0 | 0.0 | 0.0 | 0.0 | 8.8 | 0.0 |
| g | Coprococcus | 0.8 | 0.7 | 0.0 | 0.0 | 0.0 | 0.0 | 0.5 | 0.0 |
| s | Coprococcus_catus | 0.6 | 0.0 | 0.0 | 0.0 | 0.0 | 0.0 | 0.3 | 0.0 |
| s | Coprococcus_comes | 0.2 | 0.7 | 0.0 | 0.0 | 0.0 | 0.0 | 0.2 | 0.0 |
| g | Dorea | 7.5 | 0.0 | 0.0 | 1.8 | 0.0 | 0.0 | 3.1 | 0.0 |
| s | Dorea_formicigenerans | 1.4 | 0.0 | 0.0 | 0.0 | 0.0 | 0.0 | 0.9 | 0.0 |
| s | Dorea_longicatena | 6.1 | 0.0 | 0.0 | 1.8 | 0.0 | 0.0 | 2.2 | 0.0 |
| g | Roseburia | 0.6 | 6.9 | 19.6 | 0.3 | 0.0 | 0.7 | 1.7 | 0.0 |
| s | Roseburia_intestinalis | 0.3 | 3.8 | 19.0 | 0.0 | 0.0 | 0.7 | 0.0 | 0.0 |
| s | Roseburia_inulinivorans | 0.3 | 3.1 | 0.6 | 0.2 | 0.0 | 0.0 | 1.6 | 0.0 |
| f | Ruminococcaceae | 37.3 | 23.1 | 11.7 | 8.2 | 25.5 | 13.3 | 32.3 | 43.1 |
| g | Anaerotruncus | 0.1 | 0.1 | 0.0 | 0.1 | 0.1 | 0.1 | 0.0 | 0.1 |
| s | Anaerotruncus_colihominis | 0.1 | 0.1 | 0.0 | 0.1 | 0.1 | 0.1 | 0.0 | 0.1 |
| g | Faecalibacterium | 6.7 | 18.3 | 2.1 | 0.6 | 0.0 | 1.1 | 22.1 | 20.5 |
| s | Faecalibacterium_cf | 1.2 | 0.0 | 0.0 | 0.2 | 0.0 | 0.0 | 0.3 | 18.5 |
| s | Faecalibacterium_prausnitzii | 5.4 | 18.2 | 1.0 | 0.2 | 0.0 | 1.0 | 21.8 | 0.7 |
| s | Faecalibacterium_unclassified | 0.0 | 0.1 | 1.1 | 0.1 | 0.0 | 0.1 | 0.0 | 1.3 |
| s | Ruminococcus_bromii | 26.7 | 0.0 | 0.0 | 2.6 | 22.9 | 0.1 | 4.6 | 10.0 |
| s | Ruminococcus_gnavus | 0.0 | 0.1 | 9.3 | 1.0 | 0.3 | 0.1 | 0.1 | 3.5 |
| s | Ruminococcus_lactaris | 0.0 | 0.0 | 0.0 | 0.3 | 0.0 | 0.0 | 2.1 | 0.0 |
| s | Ruminococcus_obeum | 2.5 | 0.3 | 0.2 | 0.0 | 0.0 | 0.1 | 1.2 | 0.0 |
| s | Ruminococcus_torques | 1.2 | 4.3 | 0.1 | 3.6 | 2.2 | 11.6 | 2.1 | 9.0 |
| c | Erysipelotrichi | 0.6 | 0.1 | 12.9 | 0.1 | 2.2 | 0.7 | 0.4 | 1.2 |
| o | Erysipelotrichales | 0.6 | 0.1 | 12.9 | 0.1 | 2.2 | 0.7 | 0.4 | 1.2 |
| f | Erysipelotrichaceae | 0.6 | 0.1 | 12.9 | 0.1 | 2.2 | 0.7 | 0.4 | 1.2 |
| g | Catenibacterium | 0.0 | 0.0 | 0.0 | 0.0 | 0.0 | 0.0 | 0.1 | 0.0 |
| s | Catenibacterium_mitsuokai | 0.0 | 0.0 | 0.0 | 0.0 | 0.0 | 0.0 | 0.1 | 0.0 |
| g | Coprobacillus | 0.1 | 0.1 | 12.6 | 0.1 | 1.7 | 0.3 | 0.3 | 1.2 |
| s | Coprobacillus_bacterium | 0.1 | 0.1 | 12.6 | 0.1 | 1.7 | 0.3 | 0.3 | 1.2 |
| g | Holdemania | 0.5 | 0.0 | 0.3 | 0.0 | 0.0 | 0.4 | 0.1 | 0.0 |
| s | Holdemania filiformis | 0.5 | 0.0 | 0.3 | 0.0 | 0.0 | 0.4 | 0.1 | 0.0 |
| c | Negativicutes | 1.0 | 0.0 | 4.1 | 0.4 | 0.0 | 3.9 | 0.1 | 8.1 |
| o | Selenomonadales | 1.0 | 0.0 | 4.1 | 0.4 | 0.0 | 3.9 | 0.1 | 8.1 |
| f | Acidaminococcaceae | 1.0 | 0.0 | 0.0 | 0.0 | 0.0 | 0.1 | 0.0 | 0.0 |
| g | Acidaminococcaceae_unclassified | 0.0 | 0.0 | 0.0 | 0.0 | 0.0 | 0.1 | 0.0 | 0.0 |
| g | Phascolarctobacterium | 0.7 | 0.0 | 0.0 | 0.0 | 0.0 | 0.0 | 0.0 | 0.0 |
| s | Phascolarctobacterium_unclassified | 0.7 | 0.0 | 0.0 | 0.0 | 0.0 | 0.0 | 0.0 | 0.0 |
| f | Veillonellaceae | 0.0 | 0.0 | 4.1 | 0.4 | 0.0 | 3.8 | 0.1 | 8.1 |
| g | Dialister | 0.0 | 0.0 | 4.0 | 0.3 | 0.0 | 3.8 | 0.0 | 8.0 |
| s | Dialister_invisus | 0.0 | 0.0 | 4.0 | 0.3 | 0.0 | 3.8 | 0.0 | 8.0 |
| g | Megamonas | 0.0 | 0.0 | 0.0 | 0.0 | 0.0 | 0.0 | 0.0 | 0.0 |
| s | Megamonas_hypermegale | 0.0 | 0.0 | 0.0 | 0.0 | 0.0 | 0.0 | 0.0 | 0.0 |
| g | Veillonella | 0.0 | 0.0 | 0.0 | 0.0 | 0.0 | 0.0 | 0.1 | 0.0 |
| s | Veillonella_atypica | 0.0 | 0.0 | 0.0 | 0.0 | 0.0 | 0.0 | 0.0 | 0.0 |
| s | Veillonella_parvula | 0.0 | 0.0 | 0.0 | 0.0 | 0.0 | 0.0 | 0.0 | 0.0 |
| s | Veillonella_unclassified | 0.0 | 0.0 | 0.0 | 0.0 | 0.0 | 0.0 | 0.0 | 0.0 |
| p | Fusobacteria | 0.0 | 0.0 | 0.0 | 0.0 | 0.1 | 0.0 | 0.0 | 0.0 |
| c | Fusobacteria | 0.0 | 0.0 | 0.0 | 0.0 | 0.1 | 0.0 | 0.0 | 0.0 |
| o | Fusobacteriales | 0.0 | 0.0 | 0.0 | 0.0 | 0.1 | 0.0 | 0.0 | 0.0 |
| f | Fusobacteriaceae | 0.0 | 0.0 | 0.0 | 0.0 | 0.1 | 0.0 | 0.0 | 0.0 |
| g | Fusobacterium | 0.0 | 0.0 | 0.0 | 0.0 | 0.1 | 0.0 | 0.0 | 0.0 |
| s | Fusobacterium_ulcerans | 0.0 | 0.0 | 0.0 | 0.0 | 0.1 | 0.0 | 0.0 | 0.0 |
| p | Proteobacteria | 1.8 | 0.8 | 0.6 | 0.1 | 1.7 | 5.2 | 2.6 | 2.3 |
| c | Betaproteobacteria | 0.0 | 0.0 | 0.0 | 0.0 | 0.0 | 0.0 | 0.0 | 0.0 |
| o | Burkholderiales | 0.0 | 0.0 | 0.0 | 0.0 | 0.0 | 0.0 | 0.0 | 0.0 |
| f | Sutterellaceae | 0.0 | 0.0 | 0.0 | 0.0 | 0.0 | 0.0 | 0.0 | 0.0 |
| g | Sutterella | 0.0 | 0.0 | 0.0 | 0.0 | 0.0 | 0.0 | 0.0 | 0.0 |
| s | Sutterella_wadsworthensis | 0.0 | 0.0 | 0.0 | 0.0 | 0.0 | 0.0 | 0.0 | 0.0 |
| c | Deltaproteobacteria | 0.8 | 0.8 | 0.0 | 0.0 | 0.1 | 4.3 | 1.2 | 0.0 |
| o | Desulfovibrionales | 0.8 | 0.8 | 0.0 | 0.0 | 0.1 | 4.3 | 1.2 | 0.0 |
| f | Desulfovibrionaceae | 0.8 | 0.8 | 0.0 | 0.0 | 0.1 | 4.3 | 1.2 | 0.0 |
| g | Bilophila | 0.6 | 0.8 | 0.0 | 0.0 | 0.0 | 4.2 | 0.1 | 0.0 |
| s | Bilophila_wadsworthia | 0.6 | 0.8 | 0.0 | 0.0 | 0.0 | 4.2 | 0.1 | 0.0 |
| g | Desulfovibrio | 0.2 | 0.0 | 0.0 | 0.0 | 0.1 | 0.1 | 1.0 | 0.0 |
| s | Desulfovibrio_piper | 0.0 | 0.0 | 0.0 | 0.0 | 0.0 | 0.0 | 0.8 | 0.0 |
| c | Gammaproteobacteria | 1.0 | 0.0 | 0.6 | 0.1 | 1.6 | 0.9 | 1.5 | 2.3 |
| o | Enterobacteriales | 1.0 | 0.0 | 0.6 | 0.1 | 1.6 | 0.9 | 1.4 | 2.3 |
| f | Enterobacteriaceae | 1.0 | 0.0 | 0.6 | 0.1 | 1.6 | 0.9 | 1.4 | 2.3 |
| g | Enterobacter | 0.0 | 0.0 | 0.0 | 0.0 | 0.0 | 0.0 | 0.0 | 0.0 |
| s | Enterobacter_cloacae | 0.0 | 0.0 | 0.0 | 0.0 | 0.0 | 0.0 | 0.0 | 0.0 |
| g | Escherichia | 0.9 | 0.0 | 0.0 | 0.1 | 1.4 | 0.9 | 1.4 | 2.3 |
| s | Escherichia_coli | 0.9 | 0.0 | 0.0 | 0.1 | 1.4 | 0.9 | 1.4 | 2.3 |
| s | Escherichia_unclassified | 0.0 | 0.0 | 0.0 | 0.0 | 0.0 | 0.0 | 0.0 | 0.0 |
| g | Klebsiella | 0.0 | 0.0 | 0.0 | 0.0 | 0.0 | 0.0 | 0.0 | 0.0 |
| s | Klebsiella_pneumoniae | 0.0 | 0.0 | 0.0 | 0.0 | 0.0 | 0.0 | 0.0 | 0.0 |
| s | Klebsiella_unclassified | 0.0 | 0.0 | 0.0 | 0.0 | 0.0 | 0.0 | 0.0 | 0.0 |
| p | Verrucomicrobia | 0.2 | 0.0 | 0.0 | 0.0 | 16.0 | 0.0 | 14.2 | 0.0 |
| c | Verrucomicrobiae | 0.2 | 0.0 | 0.0 | 0.0 | 16.0 | 0.0 | 14.2 | 0.0 |
| o | Verrucomicrobiales | 0.2 | 0.0 | 0.0 | 0.0 | 16.0 | 0.0 | 14.2 | 0.0 |
| f | Verrucomicrobiaceae | 0.2 | 0.0 | 0.0 | 0.0 | 16.0 | 0.0 | 14.2 | 0.0 |
| g | Akkermansia | 0.2 | 0.0 | 0.0 | 0.0 | 16.0 | 0.0 | 14.2 | 0.0 |
| s | Akkermansia_muciniphila | 0.2 | 0.0 | 0.0 | 0.0 | 16.0 | 0.0 | 14.2 | 0.0 |

Rank: k = kingdom
p = phylum
c = class
o = order
f = family
g = genus
s = species

TABLE 2B

| Rank | Name | P18 | P19 | P20 | P21 | P22 | P23 | P24 | P25 | P26 | P27 |
|---|---|---|---|---|---|---|---|---|---|---|---|
| k | Archaea | 0.0 | 0.3 | 0.7 | 0.0 | 2.0 | 3.1 | 0.0 | 0.4 | 0.0 | 0.0 |
| p | Euryarchaeota | 0.0 | 0.3 | 0.7 | 0.0 | 2.0 | 3.1 | 0.0 | 0.4 | 0.0 | 0.0 |
| c | Methanobacteria | 0.0 | 0.3 | 0.7 | 0.0 | 2.0 | 3.1 | 0.0 | 0.4 | 0.0 | 0.0 |
| o | Methanobacteriales | 0.0 | 0.3 | 0.7 | 0.0 | 2.0 | 3.1 | 0.0 | 0.4 | 0.0 | 0.0 |
| f | Methanobacteriaceae | 0.0 | 0.3 | 0.7 | 0.0 | 2.0 | 3.1 | 0.0 | 0.4 | 0.0 | 0.0 |
| g | Methanobrevibacter | 0.0 | 0.3 | 0.7 | 0.0 | 2.0 | 3.1 | 0.0 | 0.4 | 0.0 | 0.0 |
| s | Methanobrevibacter_{—} | 0.0 | 0.3 | 0.7 | 0.0 | 2.0 | 3.1 | 0.0 | 0.4 | 0.0 | 0.0 |
| | smithii | | | | | | | | | | |
| k | Bacteria | 100.0 | 99.7 | 99.3 | 100.0 | 98.0 | 96.9 | 100.0 | 99.6 | 100.0 | 100.0 |
| p | Actinobacteria | 2.2 | 0.1 | 1.3 | 16.6 | 5.7 | 5.0 | 2.0 | 0.6 | 9.2 | 10.6 |
| c | Actinobacteria | 2.2 | 0.1 | 1.3 | 16.6 | 5.7 | 5.0 | 2.0 | 0.6 | 9.2 | 10.6 |
| o | Actinomycetales | 0.0 | 0.0 | 0.0 | 0.0 | 0.0 | 0.0 | 0.0 | 0.0 | 0.0 | 0.0 |
| f | Corynebacteriaceae | 0.0 | 0.0 | 0.0 | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 | 0.0 | 0.0 | 0.0 |
| s | Corynebacterium_{—} | 0.0 | 0.0 | 0.0 | 0.0 | 0.0 | 0.0 | 0.0 | 0.0 | 0.0 | 0.0 |
| | amycolatum | | | | | | | | | | |
| s | Corynebacterium_{—} | 0.0 | 0.0 | 0.0 | 0.0 | 0.0 | 0.0 | 0.0 | 0.0 | 0.0 | 0.0 |
| | striatum | | | | | | | | | | |
| o | Bifidobacteriales | 0.1 | 0.0 | 0.0 | 11.7 | 2.5 | 1.3 | 0.1 | 0.0 | 4.3 | 7.9 |
| f | Bifidobacteriaceae | 0.1 | 0.0 | 0.0 | 11.7 | 2.5 | 1.3 | 0.1 | 0.0 | 4.3 | 7.9 |
| g | Bifidobacterium | 0.1 | 0.0 | 0.0 | 11.7 | 2.5 | 1.3 | 0.1 | 0.0 | 4.3 | 7.9 |
| s | Bifidobacterium_{—} | 0.0 | 0.0 | 0.0 | 0.0 | 2.5 | 0.1 | 0.0 | 0.0 | 1.0 | 4.1 |
| | adolescentis | | | | | | | | | | |
| s | Bifidobacterium_{—} | 0.0 | 0.0 | 0.0 | 0.0 | 0.0 | 0.0 | 0.0 | 0.0 | 0.0 | 0.0 |
| | animalis | | | | | | | | | | |
| s | Bifidobacterium_{—} | 0.0 | 0.0 | 0.0 | 0.0 | 0.0 | 0.0 | 0.0 | 0.0 | 3.4 | 3.9 |
| | bifidum | | | | | | | | | | |
| s | Bifidobacterium_{—} | 0.0 | 0.0 | 0.0 | 0.0 | 0.0 | 0.1 | 0.0 | 0.0 | 0.0 | 0.0 |
| | breve | | | | | | | | | | |
| s | Bifidobacterium_{—} | 0.0 | 0.0 | 0.0 | 0.0 | 0.0 | 0.0 | 0.0 | 0.0 | 0.0 | 0.0 |
| | catenulatum | | | | | | | | | | |
| s | Bifidobacterium_{—} | 0.0 | 0.0 | 0.0 | 0.0 | 0.0 | 0.0 | 0.1 | 0.0 | 0.0 | 0.0 |
| | dentium | | | | | | | | | | |
| s | Bifidobacterium_{—} | 0.0 | 0.0 | 0.0 | 5.1 | 0.0 | 1.1 | 0.0 | 0.0 | 0.0 | 0.0 |
| | longum | | | | | | | | | | |
| s | Bifidobacterium_{—} | 0.1 | 0.0 | 0.0 | 6.6 | 0.0 | 0.0 | 0.0 | 0.0 | 0.0 | 0.0 |
| | pseudocatenulatum | | | | | | | | | | |
| o | Coriobacteriales | 2.1 | 0.1 | 1.2 | 4.8 | 3.3 | 3.6 | 1.9 | 0.6 | 4.9 | 2.6 |
| f | Coriobacteriaceae | 2.1 | 0.1 | 1.2 | 4.8 | 3.3 | 3.6 | 1.9 | 0.6 | 4.9 | 2.6 |
| g | Collinsella | 1.7 | 0.0 | 1.0 | 4.1 | 3.0 | 2.6 | 1.3 | 0.0 | 4.5 | 2.1 |
| s | Collinsella_{—} | 0.0 | 0.0 | 1.0 | 4.1 | 3.0 | 2.6 | 1.3 | 0.0 | 4.5 | 2.1 |
| | aerofaciens | | | | | | | | | | |
| s | Collinsella_{—} | 1.7 | 0.0 | 0.0 | 0.0 | 0.0 | 0.0 | 0.0 | 0.0 | 0.0 | 0.0 |
| | intestinalis | | | | | | | | | | |
| g | Eggerthella | 0.3 | 0.1 | 0.1 | 0.3 | 0.2 | 0.9 | 0.5 | 0.4 | 0.3 | 0.4 |
| s | Eggerthella_{—}lenta | 0.3 | 0.1 | 0.1 | 0.3 | 0.2 | 0.9 | 0.5 | 0.4 | 0.3 | 0.4 |
| g | Gordonibacter | 0.1 | 0.0 | 0.0 | 0.4 | 0.1 | 0.0 | 0.0 | 0.2 | 0.1 | 0.1 |
| s | Gordonibacter_{—} | 0.1 | 0.0 | 0.0 | 0.4 | 0.1 | 0.0 | 0.0 | 0.2 | 0.1 | 0.1 |
| | pamelaeae | | | | | | | | | | |
| p | Bacteroidetes | 37.5 | 21.3 | 36.9 | 16.3 | 10.0 | 26.4 | 21.7 | 45.4 | 1.0 | 46.9 |
| c | Bacteroidia | 37.5 | 21.3 | 36.9 | 16.3 | 10.0 | 26.4 | 21.7 | 45.4 | 1.0 | 46.9 |
| o | Bacteroidales | 37.5 | 21.3 | 36.9 | 16.3 | 10.0 | 26.4 | 21.7 | 45.4 | 1.0 | 46.9 |
| f | Bacteroidaceae | 33.1 | 18.0 | 20.3 | 14.6 | 7.0 | 12.8 | 6.4 | 19.4 | 0.2 | 31.8 |
| g | Bacteroides | 33.1 | 18.0 | 20.3 | 14.6 | 7.0 | 12.8 | 6.4 | 19.4 | 0.2 | 31.8 |
| s | Bacteroides_{—} | 0.0 | 0.0 | 11.7 | 0.5 | 0.0 | 0.0 | 1.0 | 2.7 | 0.0 | 4.6 |
| | caccae | | | | | | | | | | |
| s | Bacteroides_{—} | 0.9 | 0.0 | 0.1 | 0.0 | 0.2 | 0.0 | 0.1 | 1.1 | 0.0 | 0.1 |
| | cellulosilyticus | | | | | | | | | | |
| s | Bacteroides_{—} | 10.7 | 7.7 | 0.0 | 0.0 | 0.0 | 0.0 | 0.0 | 0.0 | 0.0 | 0.0 |
| | coprocola | | | | | | | | | | |
| s | Bacteroides_{—} | 1.4 | 0.0 | 0.0 | 0.0 | 0.0 | 0.0 | 0.0 | 0.0 | 0.0 | 0.0 |
| | coprophilus | | | | | | | | | | |
| s | Bacteroides_{—} | 0.0 | 0.0 | 0.0 | 0.0 | 2.3 | 0.0 | 0.0 | 0.0 | 0.0 | 0.0 |
| | eggerthii | | | | | | | | | | |
| s | Bacteroides_{—} | 1.4 | 0.0 | 0.0 | 0.1 | 0.0 | 0.0 | 0.0 | 0.0 | 0.0 | 0.0 |
| | finegoldii | | | | | | | | | | |
| s | Bacteroides_{—} | 0.4 | 0.0 | 0.0 | 0.0 | 0.0 | 0.1 | 0.0 | 0.0 | 0.0 | 0.0 |
| | fragilis | | | | | | | | | | |
| s | Bacteroides_{—} | 0.0 | 0.0 | 1.9 | 0.0 | 0.0 | 2.8 | 0.0 | 0.0 | 0.0 | 7.2 |
| | intestinalis | | | | | | | | | | |
| s | Bacteroides_{—} | 0.5 | 0.2 | 0.6 | 0.7 | 0.0 | 0.0 | 0.3 | 0.6 | 0.0 | 0.1 |
| | ovatus | | | | | | | | | | |
| s | Bacteroides_{—} | 0.0 | 0.0 | 0.0 | 0.0 | 0.0 | 0.0 | 0.0 | 0.0 | 0.0 | 0.0 |
| | pectinophilus | | | | | | | | | | |
| s | Bacteroides_{—} | 0.0 | 8.6 | 0.0 | 0.0 | 0.0 | 0.0 | 0.0 | 0.0 | 0.0 | 0.0 |
| | plebeius | | | | | | | | | | |
| s | Bacteroides_{—} | 3.1 | 0.3 | 0.0 | 3.8 | 0.0 | 0.0 | 0.5 | 0.2 | 0.0 | 0.4 |
| | stercoris | | | | | | | | | | |
| s | Bacteroides_{—} | 0.0 | 0.0 | 0.3 | 0.4 | 0.0 | 0.6 | 0.1 | 0.4 | 0.0 | 0.6 |
| | thetaiotaomicron | | | | | | | | | | |
| s | Bacteroides_{—} | 4.7 | 0.0 | 0.0 | 0.0 | 0.0 | 3.8 | 2.5 | 10.5 | 0.2 | 0.0 |
| | unclassified | | | | | | | | | | |
| s | Bacteroides_{—} | 0.0 | 0.0 | 0.0 | 4.3 | 0.1 | 0.0 | 0.0 | 0.2 | 0.0 | 7.2 |
| | uniformis | | | | | | | | | | |
| s | Bacteroides_{—} | 8.9 | 1.1 | 4.5 | 4.7 | 4.3 | 5.2 | 1.6 | 3.0 | 0.0 | 10.6 |
| | vulgatus | | | | | | | | | | |
| s | Bacteroides_{—} | 1.2 | 0.0 | 1.2 | 0.2 | 0.0 | 0.3 | 0.3 | 0.5 | 0.0 | 0.9 |
| | xylanisolvens | | | | | | | | | | |
| f | Porphyromonadaceae | 3.6 | 0.7 | 4.6 | 1.2 | 1.0 | 0.6 | 0.7 | 6.9 | 0.8 | 8.1 |
| g | Odoribacter | 0.0 | 0.4 | 1.6 | 0.4 | 0.9 | 0.6 | 0.3 | 1.7 | 0.8 | 0.6 |
| s | Odoribacter_{—} | 0.0 | 0.4 | 1.6 | 0.4 | 0.9 | 0.6 | 0.3 | 1.7 | 0.8 | 0.6 |
| | splanchnicus | | | | | | | | | | |
| g | Parabacteroides | 3.6 | 0.3 | 1.9 | 0.9 | 0.0 | 0.0 | 0.5 | 5.3 | 0.0 | 7.5 |
| s | Parabacteroides_{—} | 0.0 | 0.0 | 0.0 | 0.2 | 0.0 | 0.0 | 0.0 | 0.1 | 0.0 | 0.0 |
| | distasonis | | | | | | | | | | |
| s | Parabacteroides_{—} | 0.0 | 0.0 | 0.0 | 0.0 | 0.0 | 0.0 | 0.0 | 0.0 | 0.0 | 0.0 |
| | johnsoniiii | | | | | | | | | | |
| s | Parabacteroides_{—} | 2.7 | 0.3 | 1.5 | 0.7 | 0.0 | 0.0 | 0.5 | 3.5 | 0.0 | 5.6 |
| | merdae | | | | | | | | | | |
| s | Parabacteroides_{—} | 0.9 | 0.0 | 0.4 | 0.0 | 0.0 | 0.0 | 0.0 | 1.7 | 0.0 | 1.9 |
| | unclassified | | | | | | | | | | |
| g | Porphyromonas | 0.0 | 0.0 | 1.0 | 0.0 | 0.0 | 0.0 | 0.0 | 0.0 | 0.0 | 0.0 |
| s | Porphyromonas_{—} | 0.0 | 0.0 | 1.0 | 0.0 | 0.0 | 0.0 | 0.0 | 0.0 | 0.0 | 0.0 |
| | asaccharolytica | | | | | | | | | | |
| f | Prevotellaceae | 0.0 | 0.0 | 0.0 | 0.0 | 0.0 | 0.0 | 14.4 | 0.0 | 0.0 | 0.0 |
| g | Prevotella | 0.0 | 0.0 | 0.0 | 0.0 | 0.0 | 0.0 | 14.4 | 0.0 | 0.0 | 0.0 |
| s | Prevotella_{—}copri | 0.0 | 0.0 | 0.0 | 0.0 | 0.0 | 0.0 | 14.4 | 0.0 | 0.0 | 0.0 |
| f | Rikenellaceae | 0.9 | 2.6 | 12.1 | 0.4 | 2.0 | 13.1 | 0.2 | 19.1 | 0.0 | 7.0 |
| g | Alistipes | 0.9 | 2.6 | 12.1 | 0.4 | 2.0 | 13.1 | 0.2 | 19.1 | 0.0 | 7.0 |
| s | Alistipes_{—} | 0.0 | 2.1 | 10.6 | 0.0 | 0.8 | 7.3 | 0.0 | 18.7 | 0.0 | 5.0 |
| | putredinis | | | | | | | | | | |
| s | Alistipes_{—} | 0.6 | 0.5 | 1.5 | 0.2 | 1.3 | 0.7 | 0.2 | 0.3 | 0.0 | 2.0 |
| | shahii | | | | | | | | | | |
| s | Alistipes_{—} | 0.4 | 0.0 | 0.0 | 0.2 | 0.0 | 5.1 | 0.0 | 0.0 | 0.0 | 0.0 |
| | unclassified | | | | | | | | | | |
| p | Firmicutes | 60.1 | 76.9 | 53.5 | 46.9 | 66.5 | 63.0 | 75.7 | 45.7 | 82.3 | 42.2 |
| c | Bacilli | 0.4 | 0.2 | 1.3 | 3.2 | 0.1 | 0.7 | 0.5 | 0.1 | 12.4 | 5.3 |
| o | Lactobacillales | 0.4 | 0.2 | 1.3 | 3.2 | 0.1 | 0.7 | 0.5 | 0.1 | 12.4 | 5.3 |
| f | Enterococcaceae | 0.0 | 0.0 | 0.8 | 0.0 | 0.0 | 0.0 | 0.0 | 0.0 | 0.0 | 0.0 |
| g | Enterococcus | 0.0 | 0.0 | 0.8 | 0.0 | 0.0 | 0.0 | 0.0 | 0.0 | 0.0 | 0.0 |
| s | Enterococcus_{—} | 0.0 | 0.0 | 0.7 | 0.0 | 0.0 | 0.0 | 0.0 | 0.0 | 0.0 | 0.0 |
| | casseliflavus | | | | | | | | | | |
| s | Enterococcus_{—} | 0.0 | 0.0 | 0.0 | 0.0 | 0.0 | 0.0 | 0.0 | 0.0 | 0.0 | 0.0 |
| | faecalis | | | | | | | | | | |
| s | Enterococcus_{—} | 0.0 | 0.0 | 0.0 | 0.0 | 0.0 | 0.0 | 0.0 | 0.0 | 0.0 | 0.0 |
| | faecium | | | | | | | | | | |
| s | Enterococcus_{—} | 0.0 | 0.0 | 0.1 | 0.0 | 0.0 | 0.0 | 0.0 | 0.0 | 0.0 | 0.0 |
| | unclassified | | | | | | | | | | |
| f | Lactobacillaceae | 0.2 | 0.0 | 0.0 | 0.0 | 0.0 | 0.0 | 0.0 | 0.0 | 0.1 | 0.0 |
| g | Lactobacillus | 0.2 | 0.0 | 0.0 | 0.0 | 0.0 | 0.0 | 0.0 | 0.0 | 0.1 | 0.0 |
| s | Lactobacillus_{—} | 0.0 | 0.0 | 0.0 | 0.0 | 0.0 | 0.0 | 0.0 | 0.0 | 0.1 | 0.0 |
| | casei | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | crispatus | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | fermentum | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | gasseri | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | paracasei | | | | | | | | | | |
| s | Lactobacillus_{—} | 0.1 | 0.0 | 0.0 | 0.0 | 0.0 | 0.0 | 0.0 | 0.0 | 0.0 | 0.0 |
| | reuteri | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | ruminis | | | | | | | | | | |
| s | Lactobacillus_{—} | 0.0 | 0.0 | 0.0 | 0.0 | 0.0 | 0.0 | 0.0 | 0.0 | 0.0 | 0.0 |
| | salivarius | | | | | | | | | | |
| f | Streptococcaceae | 0.2 | 0.2 | 0.5 | 3.2 | 0.1 | 0.7 | 0.4 | 0.1 | 12.3 | 5.3 |
| g | Lactococcus | 0.0 | 0.0 | 0.0 | 0.0 | 0.0 | 0.0 | 0.0 | 0.0 | 0.0 | 0.0 |
| s | Lactococcus_{—} | 0.0 | 0.0 | 0.0 | 0.0 | 0.0 | 0.0 | 0.0 | 0.0 | 0.0 | 0.0 |
| | lactis | | | | | | | | | | |
| g | Streptococcus | 0.2 | 0.2 | 0.5 | 3.2 | 0.1 | 0.7 | 0.4 | 0.1 | 12.3 | 5.2 |
| s | Streptococcus_{—} | 0.0 | 0.0 | 0.0 | 0.0 | 0.0 | 0.0 | 0.1 | 0.0 | 0.0 | 0.1 |
| | anginosus | | | | | | | | | | |
| s | Streptococcus_{—} | 0.0 | 0.0 | 0.0 | 0.0 | 0.0 | 0.0 | 0.0 | 0.0 | 9.8 | 0.0 |
| | infantarius | | | | | | | | | | |
| s | Streptococcus_{—} | 0.0 | 0.0 | 0.0 | 0.0 | 0.0 | 0.0 | 0.1 | 0.0 | 0.0 | 0.0 |
| | mutans | | | | | | | | | | |
| s | Streptococcus_{—} | 0.1 | 0.0 | 0.0 | 0.2 | 0.0 | 0.0 | 0.2 | 0.0 | 0.1 | 1.0 |
| | parasanguinis | | | | | | | | | | |
| s | Streptococcus_{—} | 0.1 | 0.0 | 0.2 | 0.1 | 0.0 | 0.0 | 0.1 | 0.1 | 0.0 | 3.6 |
| | salivarius | | | | | | | | | | |
| s | Streptococcus_{—} | 0.0 | 0.1 | 0.0 | 2.4 | 0.0 | 0.7 | 0.0 | 0.0 | 2.0 | 0.1 |
| | thermophilus | | | | | | | | | | |
| s | Streptococcus_{—} | 0.1 | 0.0 | 0.0 | 0.0 | 0.0 | 0.0 | 0.0 | 0.0 | 0.0 | 0.1 |
| | vestibularis | | | | | | | | | | |
| c | Clostridia | 58.9 | 73.7 | 51.9 | 43.5 | 61.9 | 51.5 | 69.3 | 33.4 | 68.0 | 35.9 |
| o | Clostridiales | 58.9 | 73.7 | 51.9 | 43.5 | 61.9 | 51.5 | 69.3 | 33.4 | 68.0 | 35.9 |
| f | Clostridiaceae | 6.1 | 0.7 | 7.8 | 10.0 | 3.0 | 2.1 | 14.5 | 1.7 | 1.2 | 3.9 |
| g | Clostridium | 6.1 | 0.7 | 7.8 | 10.0 | 3.0 | 2.1 | 14.5 | 1.7 | 0.9 | 3.9 |
| s | Clostridium_{—} | 0.2 | 0.0 | 0.0 | 0.2 | 0.0 | 0.1 | 0.0 | 0.4 | 0.1 | 0.3 |
| | asparagiforme | | | | | | | | | | |
| s | Clostridium_{—} | 4.6 | 0.2 | 0.2 | 1.9 | 0.0 | 0.3 | 14.2 | 0.3 | 0.1 | 0.6 |
| | bartlettii | | | | | | | | | | |
| s | Clostridium_{—} | 0.1 | 0.0 | 0.0 | 0.5 | 0.1 | 0.1 | 0.1 | 0.3 | 0.1 | 0.9 |
| | bolteae | | | | | | | | | | |
| s | Clostridium_cf | 0.0 | 0.0 | 0.0 | 0.0 | 0.0 | 0.2 | 0.0 | 0.2 | 0.0 | 0.2 |
| s | Clostridium_{—} | 0.0 | 0.0 | 0.2 | 0.0 | 0.1 | 0.0 | 0.0 | 0.0 | 0.0 | 1.0 |
| | hathewayi | | | | | | | | | | |
| s | Clostridium_{—} | 0.0 | 0.0 | 0.0 | 0.0 | 0.0 | 0.0 | 0.0 | 0.0 | 0.0 | 0.0 |
| | hiranonis | | | | | | | | | | |
| s | Clostridium_{—} | 0.8 | 0.3 | 1.8 | 7.1 | 2.4 | 1.0 | 0.0 | 0.2 | 0.0 | 0.4 |
| | leptum | | | | | | | | | | |
| s | Clostridium_{—} | 0.0 | 0.1 | 5.2 | 0.0 | 0.4 | 0.2 | 0.0 | 0.0 | 0.0 | 0.1 |
| | nexile | | | | | | | | | | |
| s | Clostridium_{—} | 0.0 | 0.0 | 0.0 | 0.0 | 0.0 | 0.2 | 0.0 | 0.0 | 0.3 | 0.0 |
| | perfringens | | | | | | | | | | |
| s | Clostridium_{—} | 0.2 | 0.0 | 0.0 | 0.4 | 0.0 | 0.0 | 0.0 | 0.1 | 0.2 | 0.1 |
| | scindens | | | | | | | | | | |
| s | Clostridium_{—} | 0.1 | 0.0 | 0.3 | 0.0 | 0.0 | 0.0 | 0.2 | 0.1 | 0.1 | 0.5 |
| | symbiosum | | | | | | | | | | |
| f | Clostridiales_{—} | 0.0 | 0.0 | 1.1 | 0.0 | 0.0 | 0.0 | 0.0 | 0.0 | 0.0 | 0.0 |
| | Family_XI_{—} | | | | | | | | | | |
| | Incertae_{—}Sedis | | | | | | | | | | |
| g | Anaerococcus | 0.0 | 0.0 | 0.2 | 0.0 | 0.0 | 0.0 | 0.0 | 0.0 | 0.0 | 0.0 |
| s | Anaerococcus_{—} | 0.0 | 0.0 | 0.1 | 0.0 | 0.0 | 0.0 | 0.0 | 0.0 | 0.0 | 0.0 |
| | unclassified | | | | | | | | | | |
| g | Clostridiales_{—} | 0.0 | 0.0 | 0.0 | 0.0 | 0.0 | 0.0 | 0.0 | 0.0 | 0.0 | 0.0 |
| | Family_XI_{—} | | | | | | | | | | |
| | Incertae_{—}Sedis_{—} | | | | | | | | | | |
| | unclassified | | | | | | | | | | |
| g | Finegoldia | 0.0 | 0.0 | 0.0 | 0.0 | 0.0 | 0.0 | 0.0 | 0.0 | 0.0 | 0.0 |
| s | Finegoldia_{—} | 0.0 | 0.0 | 0.0 | 0.0 | 0.0 | 0.0 | 0.0 | 0.0 | 0.0 | 0.0 |
| | magna | | | | | | | | | | |
| g | Parvimonas | 0.0 | 0.0 | 0.5 | 0.0 | 0.0 | 0.0 | 0.0 | 0.0 | 0.0 | 0.0 |
| s | Parvimonas_{—} | 0.0 | 0.0 | 0.5 | 0.0 | 0.0 | 0.0 | 0.0 | 0.0 | 0.0 | 0.0 |
| | micros | | | | | | | | | | |
| g | Peptoniphilus | 0.0 | 0.0 | 0.4 | 0.0 | 0.0 | 0.0 | 0.0 | 0.0 | 0.0 | 0.0 |
| s | Peptoniphilus_{—} | 0.0 | 0.0 | 0.0 | 0.0 | 0.0 | 0.0 | 0.0 | 0.0 | 0.0 | 0.0 |
| | harei | | | | | | | | | | |
| s | Peptoniphilus_{—} | 0.0 | 0.0 | 0.3 | 0.0 | 0.0 | 0.0 | 0.0 | 0.0 | 0.0 | 0.0 |
| | unclassified | | | | | | | | | | |
| f | Clostridiales_{—} | 0.2 | 0.1 | 0.1 | 0.3 | 0.0 | 0.2 | 0.1 | 0.1 | 1.1 | 0.2 |
| | uncl | | | | | | | | | | |
| g | Blautia | 0.2 | 0.1 | 0.1 | 0.3 | 0.0 | 0.2 | 0.1 | 0.1 | 1.1 | 0.2 |
| s | Blautia_{—} | 0.0 | 0.0 | 0.0 | 0.0 | 0.0 | 0.0 | 0.0 | 0.0 | 0.7 | 0.0 |
| | hansenii | | | | | | | | | | |
| s | Blautia_{—} | 0.0 | 0.0 | 0.1 | 0.0 | 0.0 | 0.1 | 0.0 | 0.0 | 0.0 | 0.0 |
| | hydrogenotrophica | | | | | | | | | | |
| s | Blautia_{—} | 0.1 | 0.1 | 0.0 | 0.3 | 0.0 | 0.1 | 0.1 | 0.1 | 0.4 | 0.2 |
| | unclassified | | | | | | | | | | |
| f | Eubacteriaceae | 6.1 | 22.6 | 0.4 | 12.0 | 8.4 | 17.9 | 2.3 | 15.2 | 51.5 | 5.0 |
| g | Eubacterium | 6.0 | 22.6 | 0.4 | 12.0 | 8.4 | 17.9 | 2.2 | 15.2 | 51.5 | 5.0 |
| s | Eubacterium_{—} | 0.0 | 0.0 | 0.0 | 0.0 | 5.0 | 1.6 | 0.0 | 7.2 | 6.5 | 1.0 |
| | eligens | | | | | | | | | | |
| s | Eubacterium_{—} | 5.9 | 1.5 | 0.3 | 10.0 | 2.0 | 0.7 | 0.9 | 0.3 | 0.0 | 3.7 |
| | hallii | | | | | | | | | | |
| s | Eubacterium_{—} | 0.0 | 0.0 | 0.0 | 0.0 | 0.0 | 0.2 | 0.0 | 0.0 | 0.0 | 0.0 |
| | limosum | | | | | | | | | | |
| s | Eubacterium_{—} | 0.0 | 14.7 | 0.0 | 2.0 | 0.0 | 11.3 | 0.7 | 7.7 | 45.0 | 0.2 |
| | rectale | | | | | | | | | | |
| s | Eubacterium_{—} | 0.0 | 6.0 | 0.0 | 0.0 | 1.4 | 4.1 | 0.6 | 0.0 | 0.0 | 0.0 |
| | siraeum | | | | | | | | | | |
| s | Eubacterium_{—} | 0.0 | 0.4 | 0.0 | 0.0 | 0.0 | 0.0 | 0.0 | 0.0 | 0.0 | 0.0 |
| | ventriosum | | | | | | | | | | |
| g | Pseudoramibacter | 0.0 | 0.0 | 0.0 | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 | 0.0 | 0.0 | 0.0 |
| | alactolyticus | | | | | | | | | | |
| f | Lachnospiraceae | 12.4 | 12.0 | 4.3 | 0.1 | 7.8 | 0.9 | 19.4 | 3.6 | 7.8 | 0.2 |
| g | Anaerostipes | 0.1 | 0.0 | 0.0 | 0.0 | 0.0 | 0.1 | 0.0 | 0.0 | 0.0 | 0.2 |
| s | Anaerostipes_{—} | 0.1 | 0.0 | 0.0 | 0.0 | 0.0 | 0.1 | 0.0 | 0.0 | 0.0 | 0.2 |
| | caccae | | | | | | | | | | |
| g | Butyrivibrio | 0.0 | 0.0 | 0.0 | 0.0 | 0.0 | 0.0 | 0.0 | 0.0 | 0.0 | 0.0 |
| s | Butyrivibrio_{—} | 0.0 | 0.0 | 0.0 | 0.0 | 0.0 | 0.0 | 0.0 | 0.0 | 0.0 | 0.0 |
| | crossotus | | | | | | | | | | |
| g | Coprococcus | 0.4 | 2.0 | 2.7 | 0.0 | 3.1 | 0.0 | 1.2 | 0.6 | 1.2 | 0.0 |
| s | Coprococcus_{—}catus | 0.4 | 0.7 | 0.3 | 0.0 | 1.5 | 0.0 | 0.0 | 0.0 | 1.2 | 0.0 |
| s | Coprococcus_{—}comes | 0.0 | 1.4 | 2.4 | 0.0 | 1.6 | 0.0 | 1.2 | 0.6 | 0.0 | 0.0 |
| g | Dorea | 1.0 | 4.7 | 0.2 | 0.0 | 4.6 | 0.2 | 9.0 | 1.7 | 3.2 | 0.0 |
| s | Dorea_{—} | 1.0 | 0.5 | 0.2 | 0.0 | 0.8 | 0.2 | 2.9 | 0.2 | 0.0 | 0.0 |
| | formicigenerans | | | | | | | | | | |
| s | Dorea_{—}longicatena | 0.0 | 4.2 | 0.0 | 0.0 | 3.7 | 0.0 | 6.1 | 1.4 | 3.2 | 0.0 |
| g | Roseburia | 11.0 | 5.2 | 1.5 | 0.1 | 0.2 | 0.6 | 9.3 | 1.3 | 3.4 | 0.0 |
| s | Roseburia_{—} | 9.8 | 4.2 | 0.9 | 0.0 | 0.0 | 0.6 | 1.3 | 0.4 | 1.9 | 0.0 |
| | intestinalis | | | | | | | | | | |
| s | Roseburia_{—} | 1.2 | 1.0 | 0.6 | 0.1 | 0.1 | 0.0 | 8.0 | 0.9 | 1.5 | 0.0 |
| | inulinivorans | | | | | | | | | | |
| f | Ruminococcaceae | 34.1 | 38.3 | 38.2 | 21.0 | 42.7 | 30.4 | 32.9 | 12.7 | 6.5 | 26.6 |
| g | Anaerotruncus | 0.0 | 0.0 | 0.1 | 0.1 | 0.1 | 0.0 | 0.0 | 0.1 | 0.2 | 0.4 |
| s | Anaerotruncus_{—} | 0.0 | 0.0 | 0.1 | 0.1 | 0.1 | 0.0 | 0.0 | 0.1 | 0.2 | 0.4 |
| | colihominis | | | | | | | | | | |
| g | Faecalibacterium | 7.6 | 13.2 | 1.3 | 16.0 | 8.7 | 2.1 | 12.5 | 12.3 | 0.0 | 12.8 |
| s | Faecalibacterium_{—}cf | 3.0 | 1.9 | 0.1 | 8.5 | 0.1 | 0.1 | 4.5 | 5.8 | 0.0 | 8.1 |
| s | Faecalibacterium_{—} | 3.0 | 11.3 | 1.2 | 6.6 | 8.5 | 1.8 | 5.0 | 4.7 | 0.0 | 2.9 |
| | prausnitzii | | | | | | | | | | |
| s | Faecalibacterium_{—} | 1.7 | 0.0 | 0.0 | 0.9 | 0.2 | 0.2 | 2.9 | 1.8 | 0.0 | 1.7 |
| | unclassified | | | | | | | | | | |
| s | Ruminococcus_{—}bromii | 11.8 | 20.2 | 32.3 | 0.1 | 31.8 | 13.8 | 0.1 | 0.0 | 0.0 | 8.4 |
| s | Ruminococcus_{—}gnavus | 13.8 | 0.0 | 0.1 | 0.0 | 0.0 | 10.6 | 0.3 | 0.0 | 0.0 | 0.0 |
| s | Ruminococcus_{—}lactaris | 0.7 | 0.0 | 0.0 | 3.9 | 0.0 | 0.0 | 0.0 | 0.0 | 0.0 | 0.0 |
| s | Ruminococcus_{—}obeum | 0.0 | 0.7 | 1.7 | 0.8 | 1.8 | 0.0 | 0.3 | 0.1 | 0.0 | 1.0 |
| s | Ruminococcus_{—}torques | 0.1 | 4.0 | 2.7 | 0.1 | 0.2 | 3.9 | 19.7 | 0.1 | 6.3 | 3.9 |
| c | Erysipelotrichi | 0.2 | 0.0 | 0.4 | 0.1 | 3.7 | 7.9 | 2.5 | 0.2 | 0.0 | 0.3 |
| o | Erysipelotrichales | 0.2 | 0.0 | 0.4 | 0.1 | 3.7 | 7.9 | 2.5 | 0.2 | 0.0 | 0.3 |
| f | Erysipelotrichaceae | 0.2 | 0.0 | 0.4 | 0.1 | 3.7 | 7.9 | 2.5 | 0.2 | 0.0 | 0.3 |
| g | Catenibacterium | 0.0 | 0.0 | 0.0 | 0.0 | 3.5 | 0.0 | 0.0 | 0.0 | 0.0 | 0.0 |
| s | Catenibacterium_{—} | 0.0 | 0.0 | 0.0 | 0.0 | 3.5 | 0.0 | 0.0 | 0.0 | 0.0 | 0.0 |
| | mitsuokai | | | | | | | | | | |
| g | Coprobacillus | 0.0 | 0.0 | 0.2 | 0.1 | 0.0 | 7.9 | 2.4 | 0.1 | 0.0 | 0.1 |
| s | Coprobacillus_{—} | 0.0 | 0.0 | 0.2 | 0.1 | 0.0 | 7.9 | 2.4 | 0.1 | 0.0 | 0.1 |
| | bacterium | | | | | | | | | | |
| g | Holdemania | 0.1 | 0.0 | 0.0 | 0.0 | 0.1 | 0.0 | 0.0 | 0.1 | 0.0 | 0.1 |
| s | Holdemania_{—} | 0.1 | 0.0 | 0.0 | 0.0 | 0.1 | 0.0 | 0.0 | 0.1 | 0.0 | 0.1 |
| | filiformis | | | | | | | | | | |
| c | Negativicutes | 0.6 | 2.9 | 0.0 | 0.1 | 0.7 | 2.8 | 3.5 | 12.0 | 1.9 | 0.7 |
| o | Selenomonadales | 0.6 | 2.9 | 0.0 | 0.1 | 0.7 | 2.8 | 3.5 | 12.0 | 1.9 | 0.7 |
| f | Acidaminococcaceae | 0.0 | 0.0 | 0.0 | 0.0 | 0.3 | 0.0 | 3.4 | 0.0 | 0.0 | 0.0 |
| g | Acidaminococcaceae_{—} | 0.0 | 0.0 | 0.0 | 0.0 | 0.0 | 0.0 | 0.0 | 0.0 | 0.0 | 0.0 |
| | unclassified | | | | | | | | | | |
| g | Phascolarctobacterium | 0.0 | 0.0 | 0.0 | 0.0 | 0.0 | 0.0 | 3.4 | 0.0 | 0.0 | 0.0 |
| s | Phascolarctobacterium | 0.0 | 0.0 | 0.0 | 0.0 | 0.0 | 0.0 | 3.4 | 0.0 | 0.0 | 0.0 |
| | unclassified | | | | | | | | | | |
| f | Veillonellaceae | 0.6 | 2.9 | 0.0 | 0.1 | 0.4 | 2.8 | 0.1 | 12.0 | 1.9 | 0.7 |
| g | Dialister | 0.0 | 2.4 | 0.0 | 0.0 | 0.4 | 2.8 | 0.0 | 12.0 | 1.9 | 0.7 |
| s | Dialister_{—} | 0.0 | 2.4 | 0.0 | 0.0 | 0.4 | 2.8 | 0.0 | 12.0 | 1.9 | 0.7 |
| | invisus | | | | | | | | | | |
| g | Megamonas | 0.0 | 0.0 | 0.0 | 0.0 | 0.0 | 0.0 | 0.0 | 0.0 | 0.0 | 0.0 |
| s | Megamonas_{—} | 0.0 | 0.0 | 0.0 | 0.0 | 0.0 | 0.0 | 0.0 | 0.0 | 0.0 | 0.0 |
| | hypermegale | | | | | | | | | | |
| g | Veillonella | 0.6 | 0.5 | 0.0 | 0.1 | 0.0 | 0.0 | 0.1 | 0.0 | 0.0 | 0.0 |
| s | Veillonella_{—} | 0.0 | 0.0 | 0.0 | 0.0 | 0.0 | 0.0 | 0.0 | 0.0 | 0.0 | 0.0 |
| | atypica | | | | | | | | | | |
| s | Veillonella_{—} | 0.6 | 0.0 | 0.0 | 0.0 | 0.0 | 0.0 | 0.0 | 0.0 | 0.0 | 0.0 |
| | parvula | | | | | | | | | | |
| s | Veillonella_{—} | 0.0 | 0.3 | 0.0 | 0.0 | 0.0 | 0.0 | 0.0 | 0.0 | 0.0 | 0.0 |
| | unclassified | | | | | | | | | | |
| p | Fusobacteria | 0.0 | 0.0 | 0.0 | 0.0 | 0.0 | 0.0 | 0.0 | 0.0 | 0.0 | 0.0 |
| c | Fusobacteria | 0.0 | 0.0 | 0.0 | 0.0 | 0.0 | 0.0 | 0.0 | 0.0 | 0.0 | 0.0 |
| o | Fusobacteriales | 0.0 | 0.0 | 0.0 | 0.0 | 0.0 | 0.0 | 0.0 | 0.0 | 0.0 | 0.0 |
| f | Fusobacteriaceae | 0.0 | 0.0 | 0.0 | 0.0 | 0.0 | 0.0 | 0.0 | 0.0 | 0.0 | 0.0 |
| g | Fusobacterium | 0.0 | 0.0 | 0.0 | 0.0 | 0.0 | 0.0 | 0.0 | 0.0 | 0.0 | 0.0 |
| s | Fusobacterium_{—} | 0.0 | 0.0 | 0.0 | 0.0 | 0.0 | 0.0 | 0.0 | 0.0 | 0.0 | 0.0 |
| | ulcerans | | | | | | | | | | |
| p | Proteobacteria | 0.2 | 1.4 | 7.6 | 1.0 | 15.8 | 1.8 | 0.6 | 0.7 | 7.4 | 0.3 |
| c | Betaproteobacteria | 0.0 | 0.0 | 0.0 | 0.0 | 0.4 | 0.0 | 0.0 | 0.0 | 0.0 | 0.0 |
| o | Burkholderiales | 0.0 | 0.0 | 0.0 | 0.0 | 0.4 | 0.0 | 0.0 | 0.0 | 0.0 | 0.0 |
| f | Sutterellaceae | 0.0 | 0.0 | 0.0 | 0.0 | 0.4 | 0.0 | 0.0 | 0.0 | 0.0 | 0.0 |
| g | Sutterella | 0.0 | 0.0 | 0.0 | 0.0 | 0.4 | 0.0 | 0.0 | 0.0 | 0.0 | 0.0 |
| s | Sutterella_{—} | 0.0 | 0.0 | 0.0 | 0.0 | 0.4 | 0.0 | 0.0 | 0.0 | 0.0 | 0.0 |
| | wadsworthensis | | | | | | | | | | |
| c | Deltaproteobacteria | 0.2 | 0.0 | 0.3 | 0.0 | 0.1 | 0.3 | 0.5 | 0.7 | 0.0 | 0.3 |
| o | Desulfovibrionales | 0.2 | 0.0 | 0.3 | 0.0 | 0.1 | 0.3 | 0.5 | 0.7 | 0.0 | 0.3 |
| f | Desulfovibrionaceae | 0.2 | 0.0 | 0.3 | 0.0 | 0.1 | 0.3 | 0.5 | 0.7 | 0.0 | 0.3 |
| g | Bilophila | 0.2 | 0.0 | 0.2 | 0.0 | 0.1 | 0.3 | 0.4 | 0.6 | 0.0 | 0.2 |
| s | Bilophila_{—} | 0.2 | 0.0 | 0.2 | 0.0 | 0.1 | 0.3 | 0.4 | 0.6 | 0.0 | 0.2 |
| | wadsworthia | | | | | | | | | | |
| g | Desulfovibrio | 0.0 | 0.0 | 0.1 | 0.0 | 0.0 | 0.0 | 0.0 | 0.0 | 0.0 | 0.0 |
| s | Desulfovibrio_{—} | 0.0 | 0.0 | 0.0 | 0.0 | 0.0 | 0.0 | 0.0 | 0.0 | 0.0 | 0.0 |
| | piger | | | | | | | | | | |
| c | Gammaproteobacteria | 0.0 | 1.4 | 7.3 | 1.0 | 15.3 | 1.5 | 0.1 | 0.0 | 7.2 | 0.0 |
| o | Enterobacteriales | 0.0 | 1.4 | 7.3 | 1.0 | 15.3 | 1.5 | 0.0 | 0.0 | 7.2 | 0.0 |
| f | Enterobacteriaceae | 0.0 | 1.4 | 7.3 | 1.0 | 15.3 | 1.5 | 0.0 | 0.0 | 7.2 | 0.0 |
| g | Enterobacter | 0.0 | 1.4 | 0.0 | 0.0 | 0.4 | 0.0 | 0.0 | 0.0 | 0.0 | 0.0 |
| s | Enterobacter_{—} | 0.0 | 1.4 | 0.0 | 0.0 | 0.1 | 0.0 | 0.0 | 0.0 | 0.0 | 0.0 |
| | cloacae | | | | | | | | | | |
| g | Escherichia | 0.0 | 0.0 | 5.7 | 1.0 | 1.2 | 1.3 | 0.0 | 0.0 | 0.0 | 0.0 |
| s | Escherichia_{—} | 0.0 | 0.0 | 3.8 | 1.0 | 1.2 | 1.3 | 0.0 | 0.0 | 0.0 | 0.0 |
| | coli | | | | | | | | | | |
| s | Escherichia_{—} | 0.0 | 0.0 | 1.9 | 0.0 | 0.0 | 0.0 | 0.0 | 0.0 | 0.0 | 0.0 |
| | unclassified | | | | | | | | | | |
| g | Klebsiella | 0.0 | 0.0 | 1.6 | 0.0 | 13.7 | 0.0 | 0.0 | 0.0 | 7.2 | 0.0 |
| s | Klebsiella_{—} | 0.0 | 0.0 | 1.6 | 0.0 | 10.9 | 0.0 | 0.0 | 0.0 | 6.1 | 0.0 |
| | pneumoniae | | | | | | | | | | |
| s | Klebsiella_{—} | 0.0 | 0.0 | 0.0 | 0.0 | 2.9 | 0.0 | 0.0 | 0.0 | 1.1 | 0.0 |
| | unclassified | | | | | | | | | | |
| p | Verrucomicrobia | 0.0 | 0.0 | 0.0 | 19.3 | 0.0 | 0.8 | 0.0 | 7.2 | 0.0 | 0.0 |
| c | Verrucomicrobiae | 0.0 | 0.0 | 0.0 | 19.3 | 0.0 | 0.8 | 0.0 | 7.2 | 0.0 | 0.0 |
| o | Verrucomicrobiales | 0.0 | 0.0 | 0.0 | 19.3 | 0.0 | 0.8 | 0.0 | 7.2 | 0.0 | 0.0 |
| f | Verrucomicrobiaceae | 0.0 | 0.0 | 0.0 | 19.3 | 0.0 | 0.8 | 0.0 | 7.2 | 0.0 | 0.0 |
| g | Akkermansia | 0.0 | 0.0 | 0.0 | 19.3 | 0.0 | 0.8 | 0.0 | 7.2 | 0.0 | 0.0 |
| s | Akkermansia_{—} | 0.0 | 0.0 | 0.0 | 19.3 | 0.0 | 0.8 | 0.0 | 7.2 | 0.0 | 0.0 |
| | muciniphila | | | | | | | | | | |

| Rank | Name | P28 | P29 | P30 | P31 | P32 | P33 | P34 |
|---|---|---|---|---|---|---|---|---|
| k | Archaea | 0.0 | 0.0 | 0.0 | 0.6 | 0.0 | 0.0 | 0.0 |
| p | Euryarchaeota | 0.0 | 0.0 | 0.0 | 0.6 | 0.0 | 0.0 | 0.0 |
| c | Methanobacteria | 0.0 | 0.0 | 0.0 | 0.6 | 0.0 | 0.0 | 0.0 |
| o | Methanobacteriales | 0.0 | 0.0 | 0.0 | 0.6 | 0.0 | 0.0 | 0.0 |
| f | Methanobacteriaceae | 0.0 | 0.0 | 0.0 | 0.6 | 0.0 | 0.0 | 0.0 |
| g | Methanobrevibacter | 0.0 | 0.0 | 0.0 | 0.6 | 0.0 | 0.0 | 0.0 |
| s | Methanobrevibacter_{—} | 0.0 | 0.0 | 0.0 | 0.6 | 0.0 | 0.0 | 0.0 |
| | smithii | | | | | | | |
| k | Bacteria | 100.0 | 100.0 | 100.0 | 99.4 | 100.0 | 100.0 | 100.0 |
| p | Actinobacteria | 4.0 | 5.8 | 15.6 | 1.4 | 14.3 | 0.5 | 10.3 |
| c | Actinobacteria | 4.0 | 5.8 | 15.6 | 1.4 | 14.3 | 0.5 | 10.3 |
| o | Actinomycetales | 0.1 | 0.1 | 0.0 | 0.0 | 0.2 | 0.0 | 0.0 |
| f | Corynebacteriaceae | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 |
| s | Corynebacterium_{—} | 0.0 | 0.0 | 0.0 | 0.0 | 0.0 | 0.0 | 0.0 |
| | amycolatum | | | | | | | |
| s | Corynebacterium_{—} | 0.0 | 0.0 | 0.0 | 0.0 | 0.0 | 0.0 | 0.0 |
| | striatum | | | | | | | |
| o | Bifidobacteriales | 0.0 | 0.1 | 11.3 | 0.6 | 0.3 | 0.0 | 8.8 |
| f | Bifidobacteriaceae | 0.0 | 0.1 | 11.3 | 0.6 | 0.3 | 0.0 | 8.8 |
| g | Bifidobacterium | 0.0 | 0.1 | 11.3 | 0.6 | 0.3 | 0.0 | 8.8 |
| s | Bifidobacterium_{—} | 0.0 | 0.0 | 8.3 | 0.0 | 0.0 | 0.0 | 8.2 |
| | adolescentis | | | | | | | |
| s | Bifidobacterium_{—} | 0.0 | 0.0 | 0.0 | 0.0 | 0.2 | 0.0 | 0.0 |
| | animalis | | | | | | | |
| s | Bifidobacterium_{—} | 0.0 | 0.0 | 0.0 | 0.0 | 0.0 | 0.0 | 0.5 |
| | bifidum | | | | | | | |
| s | Bifidobacterium_{—} | 0.0 | 0.0 | 0.0 | 0.0 | 0.0 | 0.0 | 0.0 |
| | breve | | | | | | | |
| s | Bifidobacterium_{—} | 0.0 | 0.0 | 1.8 | 0.0 | 0.0 | 0.0 | 0.0 |
| | catenulatum | | | | | | | |
| s | Bifidobacterium_{—} | 0.0 | 0.0 | 0.0 | 0.0 | 0.0 | 0.0 | 0.0 |
| | dentium | | | | | | | |
| s | Bifidobacterium_{—} | 0.0 | 0.0 | 1.2 | 0.6 | 0.1 | 0.0 | 0.1 |
| | longum | | | | | | | |
| s | Bifidobacterium_{—} | 0.0 | 0.0 | 0.0 | 0.0 | 0.0 | 0.0 | 0.0 |
| | pseudocatenulatum | | | | | | | |
| o | Coriobacteriales | 3.9 | 5.7 | 4.3 | 0.8 | 13.8 | 0.5 | 1.5 |
| f | Coriobacteriaceae | 3.9 | 5.7 | 4.3 | 0.8 | 13.8 | 0.5 | 1.5 |
| g | Collinsella | 0.0 | 4.9 | 4.1 | 0.0 | 4.4 | 0.0 | 1.2 |
| s | Collinsella_{—} | 0.0 | 4.9 | 4.1 | 0.0 | 4.4 | 0.0 | 1.2 |
| | aerofaciens | | | | | | | |
| s | Collinsella_{—} | 0.0 | 0.0 | 0.0 | 0.0 | 0.0 | 0.0 | 0.0 |
| | intestinalis | | | | | | | |
| g | Eggerthella | 2.6 | 0.8 | 0.1 | 0.5 | 8.7 | 0.0 | 0.2 |
| s | Eggerthella_{—}lenta | 2.6 | 0.8 | 0.1 | 0.5 | 8.7 | 0.0 | 0.2 |
| g | Gordonibacter | 1.3 | 0.0 | 0.0 | 0.4 | 0.6 | 0.5 | 0.1 |
| s | Gordonibacter_{—} | 1.3 | 0.0 | 0.0 | 0.4 | 0.6 | 0.5 | 0.1 |
| | pamelaeae | | | | | | | |
| p | Bacteroidetes | 45.5 | 21.8 | 46.9 | 39.1 | 6.6 | 41.2 | 20.6 |
| c | Bacteroidia | 45.5 | 21.8 | 46.9 | 39.1 | 6.6 | 41.2 | 20.6 |
| o | Bacteroidales | 45.5 | 21.8 | 46.9 | 39.1 | 6.6 | 41.2 | 20.6 |
| f | Bacteroidaceae | 24.9 | 15.9 | 40.9 | 20.8 | 3.9 | 23.9 | 16.4 |
| g | Bacteroides | 24.9 | 15.9 | 40.9 | 20.8 | 3.9 | 23.9 | 16.4 |
| s | Bacteroides_{—} | 0.0 | 0.0 | 0.0 | 0.2 | 1.6 | 1.8 | 0.5 |
| | caccae | | | | | | | |
| s | Bacteroides_{—} | 0.0 | 5.5 | 0.1 | 0.7 | 0.1 | 0.0 | 0.3 |
| | cellulosilyticus | | | | | | | |
| s | Bacteroides_{—} | 0.0 | 0.0 | 0.0 | 0.0 | 0.0 | 0.0 | 0.0 |
| | coprocola | | | | | | | |
| s | Bacteroides_{—} | 0.0 | 0.0 | 0.0 | 0.0 | 0.0 | 0.0 | 0.0 |
| | coprophilus | | | | | | | |
| s | Bacteroides_{—} | 10.5 | 0.0 | 0.1 | 0.0 | 0.7 | 0.0 | 0.0 |
| | eggerthii | | | | | | | |
| s | Bacteroides_{—} | 0.0 | 0.0 | 0.0 | 0.0 | 0.0 | 0.0 | 0.4 |
| | finegoldii | | | | | | | |
| s | Bacteroides_{—} | 0.1 | 0.1 | 0.0 | 0.0 | 0.0 | 0.0 | 0.0 |
| | fragilis | | | | | | | |
| s | Bacteroides_{—} | 0.0 | 0.0 | 0.0 | 0.0 | 0.9 | 0.0 | 0.0 |
| | intestinalis | | | | | | | |
| s | Bacteroides_{—} | 0.4 | 0.0 | 0.6 | 0.5 | 0.2 | 0.5 | 0.7 |
| | ovatus | | | | | | | |
| s | Bacteroides_{—} | 0.0 | 0.0 | 0.0 | 0.0 | 0.0 | 0.0 | 0.0 |
| | pectinophilus | | | | | | | |
| s | Bacteroides_{—} | 0.0 | 0.0 | 0.0 | 0.0 | 0.0 | 0.0 | 9.4 |
| | plebeius | | | | | | | |
| s | Bacteroides_{—} | 0.0 | 2.9 | 21.0 | 10.0 | 0.1 | 0.0 | 1.7 |
| | stercoris | | | | | | | |
| s | Bacteroides_{—} | 0.0 | 0.2 | 0.0 | 0.0 | 0.0 | 0.0 | 0.0 |
| | thetaiotaomicron | | | | | | | |
| s | Bacteroides_{—} | 10.8 | 0.3 | 14.4 | 9.3 | 0.0 | 2.3 | 0.0 |
| | unclassified | | | | | | | |
| s | Bacteroides_{—} | 0.3 | 6.5 | 0.0 | 0.0 | 0.1 | 0.0 | 0.4 |
| | uniformis | | | | | | | |
| s | Bacteroides_{—} | 2.7 | 0.0 | 3.4 | 0.0 | 0.1 | 19.1 | 2.5 |
| | vulgatus | | | | | | | |
| s | Bacteroides_{—} | 0.1 | 0.3 | 1.4 | 0.0 | 0.1 | 0.3 | 0.5 |
| | xylanisolvens | | | | | | | |
| f | Porphyromonadaceae | 8.9 | 0.6 | 5.5 | 2.6 | 0.7 | 8.4 | 3.1 |
| g | Odoribacter | 1.1 | 0.0 | 0.0 | 0.3 | 0.5 | 2.1 | 0.0 |
| s | Odoribacter_{—} | 1.1 | 0.0 | 0.0 | 0.3 | 0.5 | 2.1 | 0.0 |
| | splanchnicus | | | | | | | |
| g | Parabacteroides | 7.8 | 0.6 | 5.5 | 2.3 | 0.2 | 6.3 | 3.1 |
| s | Parabacteroides_{—} | 5.2 | 0.0 | 0.0 | 0.0 | 0.0 | 0.0 | 0.0 |
| | distasonis | | | | | | | |
| s | Parabacteroides_{—} | 2.6 | 0.0 | 0.0 | 0.0 | 0.0 | 0.9 | 0.0 |
| | johnsoniiii | | | | | | | |
| s | Parabacteroides_{—} | 0.0 | 0.4 | 4.2 | 1.6 | 0.1 | 3.2 | 3.0 |
| | merdae | | | | | | | |
| s | Parabacteroides_{—} | 0.0 | 0.2 | 1.3 | 0.7 | 0.1 | 2.2 | 0.0 |
| | unclassified | | | | | | | |
| g | Porphyromonas | 0.0 | 0.0 | 0.0 | 0.0 | 0.0 | 0.0 | 0.0 |
| s | Porphyromonas_{—} | 0.0 | 0.0 | 0.0 | 0.0 | 0.0 | 0.0 | 0.0 |
| | asaccharolytica | | | | | | | |
| f | Prevotellaceae | 0.0 | 0.0 | 0.0 | 0.0 | 0.0 | 0.0 | 0.0 |
| g | Prevotella | 0.0 | 0.0 | 0.0 | 0.0 | 0.0 | 0.0 | 0.0 |
| s | Prevotella_{—}copri | 0.0 | 0.0 | 0.0 | 0.0 | 0.0 | 0.0 | 0.0 |
| f | Rikenellaceae | 11.7 | 5.2 | 0.5 | 15.7 | 1.9 | 8.9 | 1.2 |
| g | Alistipes | 11.7 | 5.2 | 0.5 | 15.7 | 1.9 | 8.9 | 1.2 |
| s | Alistipes_{—} | 9.2 | 4.3 | 0.0 | 13.3 | 0.0 | 6.9 | 0.0 |
| | putredinis | | | | | | | |
| s | Alistipes_{—} | 2.5 | 0.9 | 0.3 | 2.4 | 0.4 | 1.9 | 0.9 |
| | shahii | | | | | | | |
| s | Alistipes_{—} | 0.0 | 0.0 | 0.1 | 0.0 | 1.6 | 0.0 | 0.2 |
| | unclassified | | | | | | | |
| p | Firmicutes | 49.9 | 43.6 | 36.3 | 25.7 | 68.1 | 53.7 | 54.5 |
| c | Bacilli | 1.0 | 9.8 | 1.1 | 0.1 | 23.0 | 0.1 | 0.1 |
| o | Lactobacillales | 1.0 | 9.8 | 1.1 | 0.1 | 22.8 | 0.1 | 0.1 |
| f | Enterococcaceae | 0.0 | 0.0 | 0.0 | 0.0 | 0.0 | 0.0 | 0.0 |
| g | Enterococcus | 0.0 | 0.0 | 0.0 | 0.0 | 0.0 | 0.0 | 0.0 |
| s | Enterococcus_{—} | 0.0 | 0.0 | 0.0 | 0.0 | 0.0 | 0.0 | 0.0 |
| | casseliflavus | | | | | | | |
| s | Enterococcus_{—} | 0.0 | 0.0 | 0.0 | 0.0 | 0.0 | 0.0 | 0.0 |
| | faecalis | | | | | | | |
| s | Enterococcus_{—} | 0.0 | 0.0 | 0.0 | 0.0 | 0.0 | 0.0 | 0.0 |
| | faecium | | | | | | | |
| s | Enterococcus_{—} | 0.0 | 0.0 | 0.0 | 0.0 | 0.0 | 0.0 | 0.0 |
| | unclassified | | | | | | | |
| f | Lactobacillaceae | 0.0 | 4.2 | 0.0 | 0.0 | 0.7 | 0.0 | 0.0 |
| g | Lactobacillus | 0.0 | 4.2 | 0.0 | 0.0 | 0.7 | 0.0 | 0.0 |
| s | Lactobacillus_{—} | 0.0 | 0.0 | 0.0 | 0.0 | 0.4 | 0.0 | 0.0 |
| | casei | | | | | | | |
| s | Lactobacillus_{—} | 0.0 | 0.0 | 0.0 | 0.0 | 0.0 | 0.0 | 0.0 |
| | crispatus | | | | | | | |
| s | Lactobacillus_{—} | 0.0 | 0.0 | 0.0 | 0.0 | 0.0 | 0.0 | 0.0 |
| | fermentum | | | | | | | |
| s | Lactobacillus_{—} | 0.0 | 0.1 | 0.0 | 0.0 | 0.0 | 0.0 | 0.0 |
| | gasseri | | | | | | | |
| s | Lactobacillus_{—} | 0.0 | 0.0 | 0.0 | 0.0 | 0.3 | 0.0 | 0.0 |
| | paracasei | | | | | | | |
| s | Lactobacillus_{—} | 0.0 | 1.6 | 0.0 | 0.0 | 0.0 | 0.0 | 0.0 |
| | reuteri | | | | | | | |
| s | Lactobacillus_{—} | 0.0 | 0.0 | 0.0 | 0.0 | 0.0 | 0.0 | 0.0 |
| | ruminis | | | | | | | |
| s | Lactobacillus_{—} | 0.0 | 2.5 | 0.0 | 0.0 | 0.0 | 0.0 | 0.0 |
| | salivarius | | | | | | | |
| f | Streptococcaceae | 1.0 | 5.6 | 1.1 | 0.1 | 22.0 | 0.1 | 0.1 |
| g | Lactococcus | 0.0 | 0.0 | 0.0 | 0.0 | 0.1 | 0.0 | 0.0 |
| s | Lactococcus_{—} | 0.0 | 0.0 | 0.0 | 0.0 | 0.1 | 0.0 | 0.0 |
| | lactis | | | | | | | |
| g | Streptococcus | 1.0 | 5.6 | 1.1 | 0.1 | 21.9 | 0.1 | 0.1 |
| s | Streptococcus_{—} | 0.0 | 0.1 | 0.0 | 0.0 | 0.8 | 0.0 | 0.0 |
| | anginosus | | | | | | | |
| s | Streptococcus_{—} | 0.0 | 0.0 | 0.0 | 0.0 | 0.0 | 0.0 | 0.0 |
| | infantarius | | | | | | | |
| s | Streptococcus_{—} | 0.0 | 0.1 | 0.0 | 0.0 | 0.0 | 0.0 | 0.0 |
| | mutans | | | | | | | |
| s | Streptococcus_{—} | 0.3 | 2.3 | 0.3 | 0.0 | 0.5 | 0.0 | 0.0 |
| | parasanguinis | | | | | | | |
| s | Streptococcus_{—} | 0.3 | 1.8 | 0.2 | 0.0 | 0.3 | 0.1 | 0.0 |
| | salivarius | | | | | | | |
| s | Streptococcus_{—} | 0.3 | 0.1 | 0.0 | 0.0 | 19.5 | 0.0 | 0.0 |
| | thermophilus | | | | | | | |
| s | Streptococcus_{—} | 0.0 | 1.0 | 0.2 | 0.0 | 0.5 | 0.0 | 0.0 |
| | vestibularis | | | | | | | |
| c | Clostridia | 44.5 | 33.2 | 32.2 | 25.1 | 36.9 | 53.4 | 54.2 |
| o | Clostridiales | 44.5 | 33.2 | 32.2 | 25.1 | 36.9 | 53.4 | 54.2 |
| f | Clostridiaceae | 11.2 | 6.9 | 0.5 | 3.1 | 10.2 | 0.1 | 2.7 |
| g | Clostridium | 11.0 | 6.9 | 0.5 | 3.1 | 10.2 | 0.1 | 2.7 |
| s | Clostridium_{—} | 1.0 | 0.2 | 0.1 | 0.3 | 0.1 | 0.0 | 0.1 |
| | asparagiforme | | | | | | | |
| s | Clostridium_{—} | 0.0 | 0.0 | 0.1 | 0.0 | 1.2 | 0.0 | 1.6 |
| | bartlettii | | | | | | | |
| s | Clostridium_{—} | 4.1 | 3.7 | 0.1 | 0.7 | 0.4 | 0.0 | 0.2 |
| | bolteae | | | | | | | |
| s | Clostridium_cf | 1.4 | 0.0 | 0.0 | 0.0 | 0.0 | 0.0 | 0.0 |
| s | Clostridium_{—} | 0.0 | 0.1 | 0.0 | 0.1 | 5.3 | 0.0 | 0.2 |
| | hathewayi | | | | | | | |
| s | Clostridium_{—} | 0.0 | 0.0 | 0.0 | 0.0 | 0.0 | 0.0 | 0.0 |
| | hiranonis | | | | | | | |
| s | Clostridium_{—} | 0.6 | 0.1 | 0.1 | 0.0 | 0.6 | 0.0 | 0.2 |
| | leptum | | | | | | | |
| s | Clostridium_{—} | 0.0 | 0.0 | 0.0 | 0.0 | 0.0 | 0.0 | 0.0 |
| | nexile | | | | | | | |
| s | Clostridium_{—} | 0.0 | 0.7 | 0.0 | 0.0 | 0.0 | 0.0 | 0.2 |
| | perfringens | | | | | | | |
| s | Clostridium_{—} | 3.5 | 1.3 | 0.0 | 1.6 | 2.2 | 0.0 | 0.2 |
| | scindens | | | | | | | |
| s | Clostridium_{—} | 0.2 | 0.8 | 0.1 | 0.3 | 0.3 | 0.0 | 0.1 |
| | symbiosum | | | | | | | |
| f | Clostridiales_{—} | 0.0 | 0.0 | 0.0 | 0.0 | 1.3 | 0.0 | 0.0 |
| | Family_XI_{—} | | | | | | | |
| | Incertae_{—}Sedis | | | | | | | |
| g | Anaerococcus | 0.0 | 0.0 | 0.0 | 0.0 | 0.4 | 0.0 | 0.0 |
| s | Anaerococcus_{—} | 0.0 | 0.0 | 0.0 | 0.0 | 0.2 | 0.0 | 0.0 |
| | unclassified | | | | | | | |
| g | Clostridiales_{—} | 0.0 | 0.0 | 0.0 | 0.0 | 0.3 | 0.0 | 0.0 |
| | Family_XI_{—} | | | | | | | |
| | Incertae_{—}Sedis_{—} | | | | | | | |
| | unclassified | | | | | | | |
| g | Finegoldia | 0.0 | 0.0 | 0.0 | 0.0 | 0.1 | 0.0 | 0.0 |
| s | Finegoldia_{—} | 0.0 | 0.0 | 0.0 | 0.0 | 0.1 | 0.0 | 0.0 |
| | magna | | | | | | | |
| g | Parvimonas | 0.0 | 0.0 | 0.0 | 0.0 | 0.0 | 0.0 | 0.0 |
| s | Parvimonas_{—} | 0.0 | 0.0 | 0.0 | 0.0 | 0.0 | 0.0 | 0.0 |
| | micros | | | | | | | |
| g | Peptoniphilus | 0.0 | 0.0 | 0.0 | 0.0 | 0.5 | 0.0 | 0.0 |
| s | Peptoniphilus_{—} | 0.0 | 0.0 | 0.0 | 0.0 | 0.1 | 0.0 | 0.0 |
| | harei | | | | | | | |
| s | Peptoniphilus_{—} | 0.0 | 0.0 | 0.0 | 0.0 | 0.4 | 0.0 | 0.0 |
| | unclassified | | | | | | | |
| f | Clostridiales_{—} | 1.4 | 5.8 | 0.2 | 0.2 | 3.6 | 0.2 | 0.2 |
| | uncl | | | | | | | |
| g | Blautia | 1.4 | 5.8 | 0.2 | 0.2 | 3.6 | 0.2 | 0.2 |
| s | Blautia_{—} | 0.7 | 5.8 | 0.1 | 0.0 | 0.0 | 0.0 | 0.0 |
| | hansenii | | | | | | | |
| s | Blautia_{—} | 0.2 | 0.0 | 0.0 | 0.1 | 3.6 | 0.0 | 0.0 |
| | hydrogenotrophica | | | | | | | |
| s | Blautia_{—} | 0.4 | 0.0 | 0.1 | 0.1 | 0.0 | 0.2 | 0.2 |
| | unclassified | | | | | | | |
| f | Eubacteriaceae | 8.5 | 4.6 | 15.9 | 1.0 | 3.4 | 11.1 | 9.9 |
| g | Eubacterium | 8.5 | 4.6 | 15.9 | 1.0 | 3.4 | 11.1 | 9.8 |
| s | Eubacterium_{—} | 8.4 | 0.2 | 0.0 | 0.0 | 0.0 | 0.0 | 1.6 |
| | eligens | | | | | | | |
| s | Eubacterium_{—} | 0.0 | 4.1 | 1.9 | 0.0 | 3.3 | 3.7 | 0.6 |
| | hallii | | | | | | | |
| s | Eubacterium_{—} | 0.0 | 0.0 | 0.0 | 0.0 | 0.0 | 0.0 | 0.0 |
| | limosum | | | | | | | |
| s | Eubacterium_{—} | 0.0 | 0.0 | 12.1 | 0.9 | 0.0 | 6.5 | 6.2 |
| | rectale | | | | | | | |
| s | Eubacterium_{—} | 0.0 | 0.0 | 1.9 | 0.0 | 0.0 | 0.0 | 0.0 |
| | siraeum | | | | | | | |
| s | Eubacterium_{—} | 0.1 | 0.3 | 0.0 | 0.0 | 0.0 | 0.9 | 1.4 |
| | ventriosum | | | | | | | |
| g | Pseudoramibacter | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 |
| | alactolyticus | | | | | | | |
| f | Lachnospiraceae | 3.2 | 4.2 | 6.1 | 2.2 | 4.2 | 6.8 | 1.2 |
| g | Anaerostipes | 2.9 | 0.1 | 0.0 | 0.0 | 0.1 | 0.1 | 0.0 |
| s | Anaerostipes_{—} | 2.9 | 0.1 | 0.0 | 0.0 | 0.1 | 0.1 | 0.0 |
| | caccae | | | | | | | |
| g | Butyrivibrio | 0.0 | 0.0 | 0.0 | 0.0 | 0.0 | 0.0 | 0.0 |
| s | Butyrivibrio_{—} | 0.0 | 0.0 | 0.0 | 0.0 | 0.0 | 0.0 | 0.0 |
| | crossotus | | | | | | | |
| g | Coprococcus | 0.0 | 0.0 | 2.1 | 0.5 | 2.6 | 3.6 | 0.0 |
| s | Coprococcus_{—}catus | 0.0 | 0.0 | 0.6 | 0.0 | 1.3 | 1.1 | 0.0 |
| s | Coprococcus_{—}comes | 0.0 | 0.0 | 1.5 | 0.5 | 1.3 | 2.4 | 0.0 |
| g | Dorea | 0.1 | 0.0 | 0.3 | 0.2 | 1.4 | 3.2 | 0.9 |
| s | Dorea_{—} | 0.0 | 0.0 | 0.3 | 0.0 | 0.2 | 1.6 | 0.0 |
| | formicigenerans | | | | | | | |
| s | Dorea_{—}longicatena | 0.0 | 0.0 | 0.0 | 0.2 | 1.2 | 1.6 | 0.9 |
| g | Roseburia | 0.2 | 4.1 | 3.6 | 1.6 | 0.0 | 0.0 | 0.3 |
| s | Roseburia_{—} | 0.2 | 4.1 | 0.0 | 1.0 | 0.0 | 0.0 | 0.1 |
| | intestinalis | | | | | | | |
| s | Roseburia_{—} | 0.0 | 0.0 | 3.6 | 0.6 | 0.0 | 0.0 | 0.2 |
| | inulinivorans | | | | | | | |
| f | Ruminococcaceae | 20.2 | 11.7 | 9.5 | 18.5 | 14.2 | 35.3 | 40.2 |
| g | Anaerotruncus | 0.4 | 0.2 | 0.1 | 0.2 | 0.0 | 0.0 | 0.1 |
| s | Anaerotruncus_{—} | 0.4 | 0.2 | 0.1 | 0.2 | 0.0 | 0.0 | 0.1 |
| | colihominis | | | | | | | |
| g | Faecalibacterium | 0.0 | 0.0 | 4.8 | 5.6 | 0.1 | 11.1 | 5.8 |
| s | Faecalibacterium_{—}cf | 0.0 | 0.0 | 0.8 | 3.5 | 0.0 | 2.6 | 0.5 |
| s | Faecalibacterium_{—} | 0.0 | 0.0 | 2.6 | 0.6 | 0.1 | 8.4 | 4.9 |
| | prausnitzii | | | | | | | |
| s | Faecalibacterium_{—} | 0.0 | 0.0 | 1.4 | 1.6 | 0.0 | 0.1 | 0.3 |
| | unclassified | | | | | | | |
| s | Ruminococcus_{—}bromii | 0.0 | 0.0 | 0.0 | 10.4 | 0.0 | 0.0 | 27.5 |
| s | Ruminococcus_{—}gnavus | 0.6 | 5.1 | 0.1 | 0.1 | 0.6 | 0.6 | 0.2 |
| s | Ruminococcus_{—}lactaris | 0.0 | 0.0 | 0.6 | 0.0 | 0.0 | 0.0 | 2.5 |
| s | Ruminococcus_{—}obeum | 0.0 | 0.1 | 0.1 | 2.0 | 2.0 | 14.3 | 0.0 |
| s | Ruminococcus_{—}torques | 19.2 | 6.3 | 3.8 | 0.2 | 11.5 | 9.3 | 4.1 |
| c | Erysipelotrichi | 4.4 | 0.5 | 0.0 | 0.5 | 8.2 | 0.1 | 0.2 |
| o | Erysipelotrichales | 4.4 | 0.5 | 0.0 | 0.5 | 8.2 | 0.1 | 0.2 |
| f | Erysipelotrichaceae | 4.4 | 0.5 | 0.0 | 0.5 | 8.2 | 0.1 | 0.2 |
| g | Catenibacterium | 0.0 | 0.0 | 0.0 | 0.0 | 0.0 | 0.0 | 0.0 |
| s | Catenibacterium_{—} | 0.0 | 0.0 | 0.0 | 0.0 | 0.0 | 0.0 | 0.0 |
| | mitsuokai | | | | | | | |
| g | Coprobacillus | 4.4 | 0.3 | 0.0 | 0.4 | 8.1 | 0.0 | 0.0 |
| s | Coprobacillus_{—} | 4.4 | 0.3 | 0.0 | 0.4 | 8.1 | 0.0 | 0.0 |
| | bacterium | | | | | | | |
| g | Holdemania | 0.0 | 0.3 | 0.0 | 0.1 | 0.0 | 0.1 | 0.1 |
| s | Holdemania_{—} | 0.0 | 0.3 | 0.0 | 0.1 | 0.0 | 0.1 | 0.1 |
| | filiformis | | | | | | | |
| c | Negativicutes | 0.0 | 0.0 | 2.9 | 0.0 | 0.0 | 0.1 | 0.0 |
| o | Selenomonadales | 0.0 | 0.0 | 2.9 | 0.0 | 0.0 | 0.1 | 0.0 |
| f | Acidaminococcaceae | 0.0 | 0.0 | 0.0 | 0.0 | 0.0 | 0.0 | 0.0 |
| g | Acidaminococcaceae_{—} | 0.0 | 0.0 | 0.0 | 0.0 | 0.0 | 0.0 | 0.0 |
| | unclassified | | | | | | | |
| g | Phascolarctobacterium | 0.0 | 0.0 | 0.0 | 0.0 | 0.0 | 0.0 | 0.0 |
| s | Phascolarctobacterium | 0.0 | 0.0 | 0.0 | 0.0 | 0.0 | 0.0 | 0.0 |
| | unclassified | | | | | | | |
| f | Veillonellaceae | 0.0 | 0.0 | 2.9 | 0.0 | 0.0 | 0.1 | 0.0 |
| g | Dialister | 0.0 | 0.0 | 2.8 | 0.0 | 0.0 | 0.0 | 0.0 |
| s | Dialister_{—} | 0.0 | 0.0 | 2.8 | 0.0 | 0.0 | 0.0 | 0.0 |
| | invisus | | | | | | | |
| g | Megamonas | 0.0 | 0.0 | 0.0 | 0.0 | 0.0 | 0.0 | 0.0 |
| s | Megamonas_{—} | 0.0 | 0.0 | 0.0 | 0.0 | 0.0 | 0.0 | 0.0 |
| | hypermegale | | | | | | | |
| g | Veillonella | 0.0 | 0.0 | 0.1 | 0.0 | 0.0 | 0.1 | 0.0 |
| s | Veillonella_{—} | 0.0 | 0.0 | 0.0 | 0.0 | 0.0 | 0.0 | 0.0 |
| | atypica | | | | | | | |
| s | Veillonella_{—} | 0.0 | 0.0 | 0.1 | 0.0 | 0.0 | 0.1 | 0.0 |
| | parvula | | | | | | | |
| s | Veillonella_{—} | 0.0 | 0.0 | 0.0 | 0.0 | 0.0 | 0.0 | 0.0 |
| | unclassified | | | | | | | |
| p | Fusobacteria | 0.0 | 5.4 | 0.0 | 0.0 | 0.0 | 0.0 | 0.0 |
| c | Fusobacteria | 0.0 | 5.4 | 0.0 | 0.0 | 0.0 | 0.0 | 0.0 |
| o | Fusobacteriales | 0.0 | 5.4 | 0.0 | 0.0 | 0.0 | 0.0 | 0.0 |
| f | Fusobacteriaceae | 0.0 | 5.4 | 0.0 | 0.0 | 0.0 | 0.0 | 0.0 |
| g | Fusobacterium | 0.0 | 5.4 | 0.0 | 0.0 | 0.0 | 0.0 | 0.0 |
| s | Fusobacterium_{—} | 0.0 | 5.4 | 0.0 | 0.0 | 0.0 | 0.0 | 0.0 |
| | ulcerans | | | | | | | |
| p | Proteobacteria | 0.5 | 1.4 | 1.1 | 1.7 | 10.5 | 4.6 | 14.5 |
| c | Betaproteobacteria | 0.0 | 0.0 | 0.0 | 0.0 | 0.0 | 0.0 | 0.0 |
| o | Burkholderiales | 0.0 | 0.0 | 0.0 | 0.0 | 0.0 | 0.0 | 0.0 |
| f | Sutterellaceae | 0.0 | 0.0 | 0.0 | 0.0 | 0.0 | 0.0 | 0.0 |
| g | Sutterella | 0.0 | 0.0 | 0.0 | 0.0 | 0.0 | 0.0 | 0.0 |
| s | Sutterella_{—} | 0.0 | 0.0 | 0.0 | 0.0 | 0.0 | 0.0 | 0.0 |
| | wadsworthensis | | | | | | | |
| c | Deltaproteobacteria | 0.5 | 0.6 | 0.8 | 1.7 | 0.5 | 0.0 | 0.3 |
| o | Desulfovibrionales | 0.5 | 0.6 | 0.8 | 1.7 | 0.5 | 0.0 | 0.3 |
| f | Desulfovibrionaceae | 0.5 | 0.6 | 0.8 | 1.7 | 0.5 | 0.0 | 0.3 |
| g | Bilophila | 0.4 | 0.6 | 0.8 | 1.2 | 0.1 | 0.0 | 0.3 |
| s | Bilophila_{—} | 0.4 | 0.6 | 0.8 | 1.2 | 0.1 | 0.0 | 0.3 |
| | wadsworthia | | | | | | | |
| g | Desulfovibrio | 0.0 | 0.0 | 0.0 | 0.4 | 0.4 | 0.0 | 0.0 |
| s | Desulfovibrio_{—} | 0.0 | 0.0 | 0.0 | 0.0 | 0.4 | 0.0 | 0.0 |
| | piger | | | | | | | |
| c | Gammaproteobacteria | 0.1 | 0.8 | 0.3 | 0.0 | 10.0 | 4.6 | 14.2 |
| o | Enterobacteriales | 0.1 | 0.8 | 0.0 | 0.0 | 10.0 | 4.6 | 14.1 |
| f | Enterobacteriaceae | 0.1 | 0.8 | 0.0 | 0.0 | 10.0 | 4.6 | 14.1 |
| g | Enterobacter | 0.0 | 0.0 | 0.0 | 0.0 | 0.0 | 0.0 | 0.0 |
| s | Enterobacter_{—} | 0.0 | 0.0 | 0.0 | 0.0 | 0.0 | 0.0 | 0.0 |
| | cloacae | | | | | | | |
| g | Escherichia | 0.1 | 0.0 | 0.0 | 0.0 | 9.5 | 4.6 | 0.2 |
| s | Escherichia_{—} | 0.1 | 0.0 | 0.0 | 0.0 | 9.5 | 2.9 | 0.2 |
| | coli | | | | | | | |
| s | Escherichia_{—} | 0.0 | 0.0 | 0.0 | 0.0 | 0.0 | 1.7 | 0.0 |
| | unclassified | | | | | | | |
| g | Klebsiella | 0.0 | 0.8 | 0.0 | 0.0 | 0.4 | 0.0 | 14.0 |
| s | Klebsiella_{—} | 0.0 | 0.6 | 0.0 | 0.0 | 0.4 | 0.0 | 2.5 |
| | pneumoniae | | | | | | | |
| s | Klebsiella_{—} | 0.0 | 0.1 | 0.0 | 0.0 | 0.0 | 0.0 | 11.5 |
| | unclassified | | | | | | | |
| p | Verrucomicrobia | 0.0 | 22.0 | 0.1 | 31.4 | 0.5 | 0.0 | 0.0 |
| c | Verrucomicrobiae | 0.0 | 22.0 | 0.1 | 31.4 | 0.5 | 0.0 | 0.0 |
| o | Verrucomicrobiales | 0.0 | 22.0 | 0.1 | 31.4 | 0.5 | 0.0 | 0.0 |
| f | Verrucomicrobiaceae | 0.0 | 22.0 | 0.1 | 31.4 | 0.5 | 0.0 | 0.0 |
| g | Akkermansia | 0.0 | 22.0 | 0.1 | 31.4 | 0.5 | 0.0 | 0.0 |
| s | Akkermansia_{—} | 0.0 | 22.0 | 0.1 | 31.4 | 0.5 | 0.0 | 0.0 |
| | muciniphila | | | | | | | |

Rank: k = kingdom p = phylum c = class o = order f = family g = genus s = species

TABLE 2C

| Rank | Name | P35 | P36 | P37 | P38 | P39 | P40 | P41 | P42 | P43 | P44 | P45 |
|---|---|---|---|---|---|---|---|---|---|---|---|---|
| k | Archaea | 0.0 | 8.0 | 0.0 | 10.2 | 0.0 | 0.0 | 0.0 | 0.0 | 23.4 | 0.0 | 0.0 |
| p | Euryarchaeota | 0.0 | 8.0 | 0.0 | 10.2 | 0.0 | 0.0 | 0.0 | 0.0 | 23.4 | 0.0 | 0.0 |
| c | Methanobacteria | 0.0 | 8.0 | 0.0 | 10.2 | 0.0 | 0.0 | 0.0 | 0.0 | 23.4 | 0.0 | 0.0 |
| o | Methanobacteriales | 0.0 | 8.0 | 0.0 | 10.2 | 0.0 | 0.0 | 0.0 | 0.0 | 23.4 | 0.0 | 0.0 |
| f | Methanobacteriaceae | 0.0 | 8.0 | 0.0 | 10.2 | 0.0 | 0.0 | 0.0 | 0.0 | 23.4 | 0.0 | 0.0 |
| g | Methanobrevibacter | 0.0 | 8.0 | 0.0 | 10.2 | 0.0 | 0.0 | 0.0 | 0.0 | 23.4 | 0.0 | 0.0 |
| s | Methanobrevibacter_{—} | 0.0 | 8.0 | 0.0 | 10.2 | 0.0 | 0.0 | 0.0 | 0.0 | 23.4 | 0.0 | 0.0 |
| | smithii | | | | | | | | | | | |
| k | Bacteria | 100.0 | 92.0 | 100.0 | 89.8 | 100.0 | 100.0 | 100.0 | 100.0 | 76.6 | 100.0 | 100.0 |
| p | Actinobacteria | 1.7 | 1.9 | 3.1 | 8.9 | 21.8 | 30.5 | 0.9 | 4.5 | 0.1 | 1.4 | 1.2 |
| c | Actinobacteria | 1.7 | 1.9 | 3.1 | 8.9 | 21.8 | 30.5 | 0.9 | 4.5 | 0.1 | 1.4 | 1.2 |
| o | Actinomycetales | 0.0 | 1.6 | 0.0 | 0.2 | 0.0 | 0.0 | 0.0 | 0.0 | 0.0 | 0.0 | 0.1 |
| f | Corynebacteriaceae | 0.0 | 1.6 | 0.0 | 0.0 | 0.0 | 0.0 | 0.0 | 0.0 | 0.0 | 0.0 | 0.0 |
| g | Corynebacterium | 0.0 | 1.6 | 0.0 | 0.0 | 0.0 | 0.0 | 0.0 | 0.0 | 0.0 | 0.0 | 0.0 |
| s | Corynebacterium_{—} | 0.0 | 0.0 | 0.0 | 0.0 | 0.0 | 0.0 | 0.0 | 0.0 | 0.0 | 0.0 | 0.0 |
| | amycolatum | | | | | | | | | | | |
| s | Corynebacterium_{—} | 0.0 | 1.5 | 0.0 | 0.0 | 0.0 | 0.0 | 0.0 | 0.0 | 0.0 | 0.0 | 0.0 |
| | striatum | | | | | | | | | | | |
| o | Bifidobacteriales | 0.2 | 0.0 | 2.7 | 3.3 | 0.9 | 26.6 | 0.0 | 2.8 | 0.0 | 0.0 | 0.0 |
| f | Bifidobacteriaceae | 0.2 | 0.0 | 2.7 | 3.3 | 0.9 | 26.6 | 0.0 | 2.8 | 0.0 | 0.0 | 0.0 |
| g | Bifidobacterium | 0.2 | 0.0 | 2.7 | 3.3 | 0.9 | 26.6 | 0.0 | 2.8 | 0.0 | 0.0 | 0.0 |
| s | Bifidobacterium_{—} | 0.0 | 0.0 | 0.0 | 0.0 | 0.9 | 20.2 | 0.0 | 1.4 | 0.0 | 0.0 | 0.0 |
| | adolescentis | | | | | | | | | | | |
| s | Bifidobacterium_{—} | 0.0 | 0.0 | 0.0 | 3.3 | 0.0 | 0.0 | 0.0 | 0.0 | 0.0 | 0.0 | 0.0 |
| | animalis | | | | | | | | | | | |
| s | Bifidobacterium_{—} | 0.0 | 0.0 | 2.7 | 0.0 | 0.0 | 0.0 | 0.0 | 0.0 | 0.0 | 0.0 | 0.0 |
| | bifidum | | | | | | | | | | | |
| s | Bifidobacterium_{—} | 0.0 | 0.0 | 0.0 | 0.0 | 0.0 | 0.0 | 0.0 | 0.0 | 0.0 | 0.0 | 0.0 |
| | breve | | | | | | | | | | | |
| s | Bifidobacterium_{—} | 0.0 | 0.0 | 0.0 | 0.0 | 0.0 | 3.5 | 0.0 | 0.0 | 0.0 | 0.0 | 0.0 |
| | catenulatum | | | | | | | | | | | |
| s | Bifidobacterium_{—} | 0.0 | 0.0 | 0.0 | 0.0 | 0.0 | 0.0 | 0.0 | 0.0 | 0.0 | 0.0 | 0.0 |
| | dentium | | | | | | | | | | | |
| s | Bifidobacterium_{—} | 0.2 | 0.0 | 0.0 | 0.0 | 0.0 | 2.8 | 0.0 | 1.2 | 0.0 | 0.0 | 0.0 |
| | longum | | | | | | | | | | | |
| s | Bifidobacterium_{—} | 0.0 | 0.0 | 0.0 | 0.0 | 0.0 | 0.0 | 0.0 | 0.1 | 0.0 | 0.0 | 0.0 |
| | pseudocatenulatum | | | | | | | | | | | |
| o | Coriobacteriales | 1.5 | 0.3 | 0.4 | 5.5 | 21.0 | 3.9 | 0.9 | 1.7 | 0.0 | 1.4 | 1.1 |
| f | Coriobacteriaceae | 1.5 | 0.3 | 0.4 | 5.5 | 21.0 | 3.9 | 0.9 | 1.7 | 0.0 | 1.4 | 1.1 |
| g | Collinsella | 0.8 | 0.3 | 0.0 | 5.2 | 19.7 | 0.0 | 0.0 | 1.4 | 0.0 | 0.0 | 0.0 |
| s | Collinsella_{—} | 0.8 | 0.3 | 0.0 | 5.2 | 19.7 | 0.0 | 0.0 | 1.3 | 0.0 | 0.0 | 0.0 |
| | aerofaciens | | | | | | | | | | | |
| s | Collinsella_{—} | 0.0 | 0.0 | 0.0 | 0.0 | 0.0 | 0.0 | 0.0 | 0.0 | 0.0 | 0.0 | 0.0 |
| | intestinalis | | | | | | | | | | | |
| g | Eggerthella | 0.5 | 0.0 | 0.3 | 0.3 | 0.7 | 3.3 | 0.0 | 0.4 | 0.0 | 1.4 | 1.0 |
| s | Eggerthella_{—} | 0.5 | 0.0 | 0.3 | 0.3 | 0.7 | 3.3 | 0.0 | 0.4 | 0.0 | 1.4 | 1.0 |
| | lenta | | | | | | | | | | | |
| g | Gordonibacter | 0.2 | 0.0 | 0.1 | 0.0 | 0.6 | 0.6 | 0.9 | 0.0 | 0.0 | 0.0 | 0.1 |
| s | Gordonibacter_{—} | 0.2 | 0.0 | 0.1 | 0.0 | 0.6 | 0.6 | 0.9 | 0.0 | 0.0 | 0.0 | 0.1 |
| | pamelaeae | | | | | | | | | | | |
| p | Bacteroidetes | 35.5 | 78.2 | 23.7 | 37.1 | 13.7 | 10.6 | 27.9 | 41.8 | 54.7 | 54.4 | 1.2 |
| c | Bacteroidia | 35.5 | 78.2 | 23.7 | 37.1 | 13.7 | 10.6 | 27.9 | 41.8 | 54.7 | 54.4 | 1.2 |
| o | Bacteroidales | 35.5 | 78.2 | 23.7 | 37.1 | 13.7 | 10.6 | 27.9 | 41.8 | 54.7 | 54.4 | 1.2 |
| f | Bacteroidaceae | 18.6 | 49.4 | 22.8 | 31.0 | 12.6 | 6.8 | 17.9 | 26.6 | 48.6 | 54.4 | 0.8 |
| g | Bacteroides | 18.6 | 49.4 | 22.8 | 31.0 | 12.6 | 6.8 | 17.9 | 26.6 | 48.6 | 54.4 | 0.8 |
| s | Bacteroides_{—} | 1.0 | 0.1 | 0.0 | 6.4 | 6.6 | 0.0 | 2.7 | 0.0 | 0.0 | 0.0 | 0.2 |
| | caccae | | | | | | | | | | | |
| s | Bacteroides_{—} | 0.0 | 0.0 | 0.0 | 0.0 | 0.1 | 0.0 | 0.0 | 0.0 | 0.0 | 0.0 | 0.0 |
| | cellulosilyticus | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 |
| | coprocola | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 |
| | coprophilus | | | | | | | | | | | |
| s | Bacteroides_{—} | 0.0 | 0.0 | 0.0 | 0.0 | 1.8 | 0.0 | 0.0 | 0.0 | 0.0 | 0.0 | 0.0 |
| | eggerthii | | | | | | | | | | | |
| s | Bacteroides_{—} | 0.0 | 0.0 | 0.0 | 0.0 | 0.0 | 0.5 | 0.0 | 0.0 | 11.4 | 0.0 | 0.0 |
| | finegoldii | | | | | | | | | | | |
| s | Bacteroides_{—} | 0.0 | 0.0 | 0.1 | 0.0 | 0.0 | 0.0 | 0.0 | 0.1 | 0.0 | 0.0 | 0.0 |
| | fragilis | | | | | | | | | | | |
| s | Bacteroides_{—} | 0.0 | 0.3 | 0.0 | 0.0 | 0.0 | 0.1 | 0.0 | 11.7 | 0.0 | 0.0 | 0.0 |
| | intestinalis | | | | | | | | | | | |
| s | Bacteroides_{—} | 0.2 | 0.0 | 0.0 | 1.0 | 0.1 | 0.1 | 0.3 | 0.2 | 0.0 | 6.3 | 0.1 |
| | ovatus | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 |
| | pectinophilus | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 |
| | plebeius | | | | | | | | | | | |
| s | Bacteroides_{—} | 0.0 | 0.0 | 0.0 | 2.0 | 0.8 | 4.2 | 0.0 | 14.2 | 31.3 | 17.6 | 0.1 |
| | stercoris | | | | | | | | | | | |
| s | Bacteroides_{—} | 0.0 | 1.1 | 7.3 | 0.0 | 0.0 | 0.0 | 0.0 | 0.0 | 0.0 | 0.5 | 0.0 |
| | thetaiotaomicron | | | | | | | | | | | |
| s | Bacteroides_{—} | 0.0 | 47.2 | 15.4 | 0.0 | 1.9 | 0.3 | 3.0 | 0.0 | 0.0 | 0.0 | 0.0 |
| | unclassified | | | | | | | | | | | |
| s | Bacteroides_{—} | 0.6 | 0.0 | 0.0 | 1.4 | 0.2 | 0.1 | 0.0 | 0.3 | 0.0 | 19.2 | 0.1 |
| | uniformis | | | | | | | | | | | |
| s | Bacteroides_{—} | 16.1 | 0.1 | 0.0 | 19.2 | 0.9 | 1.3 | 11.5 | 0.0 | 5.9 | 5.7 | 0.4 |
| | vulgatus | | | | | | | | | | | |
| s | Bacteroides_{—} | 0.5 | 0.4 | 0.0 | 0.9 | 0.1 | 0.1 | 0.5 | 0.0 | 0.0 | 4.9 | 0.0 |
| | xylanisolvens | | | | | | | | | | | |
| f | Porphyromonadaceae | 6.6 | 14.7 | 0.7 | 2.3 | 0.5 | 0.4 | 5.9 | 8.7 | 4.4 | 0.0 | 0.1 |
| g | Odoribacter | 2.3 | 8.2 | 0.7 | 1.7 | 0.1 | 0.1 | 0.6 | 1.0 | 0.8 | 0.0 | 0.0 |
| s | Odoribacter_{—} | 2.3 | 8.2 | 0.7 | 1.7 | 0.1 | 0.1 | 0.6 | 1.0 | 0.8 | 0.0 | 0.0 |
| | splanchnicus | | | | | | | | | | | |
| g | Parabacteroides | 4.3 | 6.5 | 0.0 | 0.0 | 0.4 | 0.3 | 5.3 | 7.7 | 3.6 | 0.0 | 0.1 |
| s | Parabacteroides_{—} | 0.0 | 0.0 | 0.0 | 0.0 | 0.0 | 0.0 | 0.0 | 0.0 | 0.0 | 0.0 | 0.0 |
| | distasonis | | | | | | | | | | | |
| s | Parabacteroides_{—} | 0.0 | 0.6 | 0.0 | 0.0 | 0.0 | 0.0 | 0.7 | 0.0 | 0.0 | 0.0 | 0.0 |
| | johnsonii | | | | | | | | | | | |
| s | Parabacteroides_{—} | 3.8 | 5.0 | 0.0 | 0.0 | 0.3 | 0.2 | 2.7 | 6.4 | 2.5 | 0.0 | 0.1 |
| | merdae | | | | | | | | | | | |
| s | Parabacteroides_{—} | 0.4 | 1.0 | 0.0 | 0.0 | 0.1 | 0.0 | 1.8 | 1.3 | 1.1 | 0.0 | 0.0 |
| | unclassified | | | | | | | | | | | |
| g | Porphyromonas | 0.0 | 0.0 | 0.0 | 0.6 | 0.0 | 0.0 | 0.0 | 0.0 | 0.0 | 0.0 | 0.0 |
| s | Porphyromonas_{—} | 0.0 | 0.0 | 0.0 | 0.6 | 0.0 | 0.0 | 0.0 | 0.0 | 0.0 | 0.0 | 0.0 |
| | asaccharolytica | | | | | | | | | | | |
| f | Prevotellaceae | 0.0 | 0.0 | 0.0 | 0.0 | 0.0 | 0.1 | 0.0 | 0.0 | 0.0 | 0.0 | 0.0 |
| g | Prevotella | 0.0 | 0.0 | 0.0 | 0.0 | 0.0 | 0.1 | 0.0 | 0.0 | 0.0 | 0.0 | 0.0 |
| s | Prevotella_{—}copri | 0.0 | 0.0 | 0.0 | 0.0 | 0.0 | 0.0 | 0.0 | 0.0 | 0.0 | 0.0 | 0.0 |
| f | Rikenellaceae | 10.3 | 14.1 | 0.2 | 3.8 | 0.7 | 3.3 | 4.1 | 6.6 | 1.7 | 0.0 | 0.3 |
| g | Alistipes | 10.3 | 14.1 | 0.2 | 3.8 | 0.7 | 3.3 | 4.1 | 6.6 | 1.7 | 0.0 | 0.3 |
| s | Alistipes_{—} | 8.0 | 5.4 | 0.0 | 3.2 | 0.6 | 2.7 | 2.2 | 4.9 | 1.7 | 0.0 | 0.3 |
| | putredinis | | | | | | | | | | | |
| s | Alistipes_shahii | 2.4 | 3.8 | 0.0 | 0.6 | 0.1 | 0.6 | 1.9 | 1.6 | 0.0 | 0.0 | 0.0 |
| s | Alistipes_{—} | 0.0 | 4.9 | 0.2 | 0.0 | 0.0 | 0.0 | 0.0 | 0.0 | 0.0 | 0.0 | 0.0 |
| | unclassified | | | | | | | | | | | |
| p | Firmicutes | 50.6 | 9.5 | 65.4 | 43.0 | 63.9 | 56.5 | 63.3 | 51.3 | 17.8 | 44.1 | 90.8 |
| c | Bacilli | 2.1 | 5.1 | 2.6 | 11.0 | 1.7 | 1.9 | 0.5 | 0.1 | 10.0 | 0.6 | 8.8 |
| o | Lactobacillales | 2.1 | 5.1 | 2.6 | 11.0 | 1.7 | 1.9 | 0.5 | 0.1 | 10.0 | 0.6 | 8.8 |
| f | Enterococcaceae | 0.1 | 4.5 | 0.0 | 0.0 | 0.0 | 0.0 | 0.1 | 0.0 | 9.6 | 0.0 | 2.4 |
| g | Enterococcus | 0.1 | 4.5 | 0.0 | 0.0 | 0.0 | 0.0 | 0.1 | 0.0 | 9.6 | 0.0 | 2.4 |
| s | Enterococcus_{—} | 0.0 | 0.0 | 0.0 | 0.0 | 0.0 | 0.0 | 0.0 | 0.0 | 0.0 | 0.0 | 0.0 |
| | casseliflavus | | | | | | | | | | | |
| s | Enterococcus_{—}faecalis | 0.0 | 4.5 | 0.0 | 0.0 | 0.0 | 0.0 | 0.0 | 0.0 | 9.6 | 0.0 | 0.0 |
| s | Enterococcus_{—}faecium | 0.0 | 0.0 | 0.0 | 0.0 | 0.0 | 0.0 | 0.0 | 0.0 | 0.0 | 0.0 | 0.0 |
| s | Enterococcus_{—} | 0.1 | 0.0 | 0.0 | 0.0 | 0.0 | 0.0 | 0.1 | 0.0 | 0.0 | 0.0 | 2.4 |
| | unclassified | | | | | | | | | | | |
| f | Lactobacillaceae | 0.3 | 0.6 | 0.0 | 1.8 | 0.0 | 0.3 | 0.0 | 0.0 | 0.0 | 0.0 | 0.3 |
| g | Lactobacillus | 0.3 | 0.6 | 0.0 | 1.8 | 0.0 | 0.3 | 0.0 | 0.0 | 0.0 | 0.0 | 0.3 |
| s | Lactobacillus_{—} | 0.0 | 0.0 | 0.0 | 0.0 | 0.0 | 0.0 | 0.0 | 0.0 | 0.0 | 0.0 | 0.0 |
| | casei | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 |
| | crispatus | | | | | | | | | | | |
| s | Lactobacillus_{—} | 0.0 | 0.0 | 0.0 | 0.5 | 0.0 | 0.2 | 0.0 | 0.0 | 0.0 | 0.0 | 0.2 |
| | fermentum | | | | | | | | | | | |
| s | Lactobacillus_{—} | 0.0 | 0.6 | 0.0 | 0.9 | 0.0 | 0.0 | 0.0 | 0.0 | 0.0 | 0.0 | 0.0 |
| | gasseri | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 |
| | paracasei | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 |
| | reuteri | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 |
| | ruminis | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 |
| | salivarius | | | | | | | | | | | |
| f | Streptococcaceae | 1.3 | 0.0 | 2.6 | 9.2 | 1.7 | 1.6 | 0.4 | 0.1 | 0.4 | 0.6 | 6.1 |
| g | Lactococcus | 0.1 | 0.0 | 0.0 | 0.3 | 0.2 | 0.0 | 0.0 | 0.1 | 0.0 | 0.1 | 0.0 |
| s | Lactococcus_{—} | 0.1 | 0.0 | 0.0 | 0.3 | 0.2 | 0.0 | 0.0 | 0.1 | 0.0 | 0.1 | 0.0 |
| | lactis | | | | | | | | | | | |
| g | Streptococcus | 1.2 | 0.0 | 2.6 | 8.9 | 1.4 | 1.6 | 0.4 | 0.0 | 0.3 | 0.5 | 6.1 |
| s | Streptococcus_{—} | 0.0 | 0.0 | 0.0 | 0.0 | 0.0 | 0.0 | 0.0 | 0.0 | 0.0 | 0.0 | 0.0 |
| | anginosus | | | | | | | | | | | |
| s | Streptococcus_{—} | 0.0 | 0.0 | 2.3 | 0.0 | 0.0 | 0.0 | 0.0 | 0.0 | 0.0 | 0.0 | 4.8 |
| | infantarius | | | | | | | | | | | |
| s | Streptococcus_{—} | 0.0 | 0.0 | 0.0 | 3.2 | 0.0 | 1.0 | 0.0 | 0.0 | 0.0 | 0.0 | 0.0 |
| | mutans | | | | | | | | | | | |
| s | Streptococcus_{—} | 0.5 | 0.0 | 0.0 | 0.8 | 0.1 | 0.1 | 0.0 | 0.0 | 0.0 | 0.2 | 0.4 |
| | parasanguinis | | | | | | | | | | | |
| s | Streptococcus_{—} | 0.2 | 0.0 | 0.1 | 1.0 | 0.3 | 0.4 | 0.2 | 0.0 | 0.0 | 0.1 | 0.6 |
| | salivarius | | | | | | | | | | | |
| s | Streptococcus_{—} | 0.0 | 0.0 | 0.0 | 0.4 | 0.9 | 0.0 | 0.1 | 0.0 | 0.0 | 0.0 | 0.1 |
| | thermophilus | | | | | | | | | | | |
| s | Streptococcus_{—} | 0.0 | 0.0 | 0.0 | 3.6 | 0.1 | 0.1 | 0.0 | 0.0 | 0.0 | 0.1 | 0.0 |
| | vestibularis | | | | | | | | | | | |
| c | Clostridia | 48.2 | 1.9 | 56.2 | 27.8 | 61.9 | 51.4 | 62.7 | 49.4 | 6.8 | 40.5 | 79.7 |
| o | Clostridiales | 48.2 | 1.9 | 56.2 | 27.8 | 61.9 | 51.4 | 62.7 | 49.4 | 6.8 | 40.5 | 79.7 |
| f | Clostridiaceae | 0.6 | 0.6 | 4.4 | 2.6 | 2.5 | 7.6 | 0.1 | 1.6 | 0.0 | 1.0 | 2.2 |
| g | Clostridium | 0.6 | 0.5 | 4.4 | 2.6 | 2.5 | 7.6 | 0.1 | 1.6 | 0.0 | 1.0 | 2.2 |
| s | Clostridium_{—} | 0.0 | 0.0 | 0.3 | 0.5 | 0.0 | 0.1 | 0.0 | 0.1 | 0.0 | 0.2 | 0.1 |
| | asparagiforme | | | | | | | | | | | |
| s | Clostridium_{—} | 0.2 | 0.0 | 0.1 | 0.1 | 0.0 | 4.6 | 0.0 | 0.0 | 0.0 | 0.0 | 0.0 |
| | bartlettii | | | | | | | | | | | |
| s | Clostridium_{—} | 0.0 | 0.0 | 1.4 | 1.3 | 0.9 | 0.2 | 0.0 | 0.3 | 0.0 | 0.3 | 0.0 |
| | bolteae | | | | | | | | | | | |
| s | Clostridium_{—} | 0.0 | 0.0 | 0.1 | 0.0 | 0.1 | 0.0 | 0.0 | 0.0 | 0.0 | 0.0 | 0.0 |
| | cf | | | | | | | | | | | |
| s | Clostridium_{—} | 0.0 | 0.0 | 0.2 | 0.2 | 0.2 | 0.1 | 0.0 | 0.1 | 0.0 | 0.0 | 0.0 |
| | hathewayi | | | | | | | | | | | |
| s | Clostridium_{—} | 0.0 | 0.0 | 0.0 | 0.0 | 0.0 | 0.0 | 0.0 | 0.0 | 0.0 | 0.0 | 0.0 |
| | hiranonis | | | | | | | | | | | |
| s | Clostridium_{—} | 0.0 | 0.5 | 0.3 | 0.0 | 1.2 | 0.5 | 0.1 | 0.8 | 0.0 | 0.2 | 0.3 |
| | leptum | | | | | | | | | | | |
| s | Clostridium_{—} | 0.0 | 0.0 | 0.0 | 0.0 | 0.0 | 0.0 | 0.0 | 0.0 | 0.0 | 0.0 | 0.0 |
| | nexile | | | | | | | | | | | |
| s | Clostridium_{—} | 0.3 | 0.0 | 0.0 | 0.0 | 0.0 | 0.0 | 0.0 | 0.0 | 0.0 | 0.0 | 1.8 |
| | perfringens | | | | | | | | | | | |
| s | Clostridium_{—} | 0.1 | 0.0 | 1.7 | 0.3 | 0.0 | 1.9 | 0.0 | 0.2 | 0.0 | 0.0 | 0.0 |
| | scindens | | | | | | | | | | | |
| s | Clostridium_{—} | 0.0 | 0.0 | 0.2 | 0.0 | 0.0 | 0.1 | 0.0 | 0.1 | 0.0 | 0.2 | 0.0 |
| | symbiosum | | | | | | | | | | | |
| f | Clostridiales_{—} | 0.0 | 0.7 | 0.0 | 0.0 | 0.0 | 0.2 | 0.0 | 1.5 | 6.7 | 0.0 | 0.0 |
| | Family_XI_{—} | | | | | | | | | | | |
| | Incertae_{—}Sedis | | | | | | | | | | | |
| g | Anaerococcus | 0.0 | 0.2 | 0.0 | 0.0 | 0.0 | 0.0 | 0.0 | 0.1 | 0.4 | 0.0 | 0.0 |
| s | Anaerococcus_{—} | 0.0 | 0.2 | 0.0 | 0.0 | 0.0 | 0.0 | 0.0 | 0.0 | 0.4 | 0.0 | 0.0 |
| | unclassified | | | | | | | | | | | |
| g | Clostridiales_{—} | 0.0 | 0.1 | 0.0 | 0.0 | 0.0 | 0.0 | 0.0 | 0.0 | 1.4 | 0.0 | 0.0 |
| | Family_XI_{—} | | | | | | | | | | | |
| | Incertae_{—}Sedis__unclassified | | | | | | | | | | | |
| g | Finegoldia | 0.0 | 0.1 | 0.0 | 0.0 | 0.0 | 0.1 | 0.0 | 0.0 | 0.0 | 0.0 | 0.0 |
| s | Finegoldia_{—} | 0.0 | 0.1 | 0.0 | 0.0 | 0.0 | 0.1 | 0.0 | 0.0 | 0.0 | 0.0 | 0.0 |
| | magna | | | | | | | | | | | |
| g | Parvimonas | 0.0 | 0.0 | 0.0 | 0.0 | 0.0 | 0.0 | 0.0 | 1.1 | 0.0 | 0.0 | 0.0 |
| s | Parvimonas_{—} | 0.0 | 0.0 | 0.0 | 0.0 | 0.0 | 0.0 | 0.0 | 1.1 | 0.0 | 0.0 | 0.0 |
| | micros | | | | | | | | | | | |
| g | Peptoniphilus | 0.0 | 0.3 | 0.0 | 0.0 | 0.0 | 0.1 | 0.0 | 0.3 | 4.8 | 0.0 | 0.0 |
| s | Peptoniphilus_{—} | 0.0 | 0.1 | 0.0 | 0.0 | 0.0 | 0.0 | 0.0 | 0.0 | 2.6 | 0.0 | 0.0 |
| | harei | | | | | | | | | | | |
| s | Peptoniphilus_{—} | 0.0 | 0.1 | 0.0 | 0.0 | 0.0 | 0.1 | 0.0 | 0.3 | 2.3 | 0.0 | 0.0 |
| | unclassified | | | | | | | | | | | |
| f | Clostridiales_{—} | 0.9 | 0.0 | 0.4 | 0.2 | 1.8 | 0.1 | 0.2 | 0.3 | 0.0 | 0.2 | 0.2 |
| | uncl | | | | | | | | | | | |
| g | Blautia | 0.9 | 0.0 | 0.4 | 0.2 | 1.8 | 0.1 | 0.2 | 0.3 | 0.0 | 0.2 | 0.2 |
| s | Blautia_{—} | 0.0 | 0.0 | 0.3 | 0.0 | 0.5 | 0.0 | 0.0 | 0.2 | 0.0 | 0.0 | 0.0 |
| | hansenii | | | | | | | | | | | |
| s | Blautia_{—} | 0.3 | 0.0 | 0.1 | 0.0 | 1.3 | 0.0 | 0.0 | 0.0 | 0.0 | 0.0 | 0.1 |
| | hydrogenotrophica | | | | | | | | | | | |
| s | Blautia_{—} | 0.6 | 0.0 | 0.0 | 0.2 | 0.0 | 0.1 | 0.2 | 0.1 | 0.0 | 0.2 | 0.1 |
| | unclassified | | | | | | | | | | | |
| f | Eubacteriaceae | 17.3 | 0.1 | 0.5 | 1.2 | 15.6 | 16.3 | 24.1 | 19.6 | 0.0 | 28.4 | 26.9 |
| g | Eubacterium | 17.3 | 0.1 | 0.5 | 1.2 | 15.5 | 16.3 | 24.1 | 19.5 | 0.0 | 28.4 | 26.9 |
| s | Eubacterium_{—} | 2.2 | 0.0 | 0.0 | 0.0 | 4.0 | 2.4 | 0.0 | 0.0 | 0.0 | 0.0 | 0.2 |
| | eligens | | | | | | | | | | | |
| s | Eubacterium_{—} | 1.7 | 0.0 | 0.0 | 0.3 | 2.7 | 6.6 | 2.2 | 9.2 | 0.0 | 0.9 | 2.0 |
| | hallii | | | | | | | | | | | |
| s | Eubacterium_{—} | 0.0 | 0.0 | 0.5 | 0.0 | 0.0 | 0.2 | 0.0 | 0.0 | 0.0 | 0.0 | 0.0 |
| | limosum | | | | | | | | | | | |
| s | Eubacterium_{—} | 6.4 | 0.1 | 0.0 | 0.9 | 4.0 | 7.1 | 21.4 | 10.3 | 0.0 | 27.5 | 23.2 |
| | rectale | | | | | | | | | | | |
| s | Eubacterium_{—} | 6.6 | 0.0 | 0.0 | 0.0 | 0.0 | 0.0 | 0.0 | 0.0 | 0.0 | 0.0 | 0.0 |
| | siraeum | | | | | | | | | | | |
| s | Eubacterium_{—} | 0.3 | 0.0 | 0.0 | 0.0 | 4.7 | 0.0 | 0.5 | 0.0 | 0.0 | 0.0 | 1.4 |
| | ventriosum | | | | | | | | | | | |
| g | Pseudoramibacter | 0.0 | 0.0 | 0.0 | 0.0 | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 | 0.0 | 0.0 | 0.0 | 0.0 |
| | alactolyticus | | | | | | | | | | | |
| f | Lachnospiraceae | 14.4 | 0.1 | 3.4 | 12.6 | 14.6 | 5.3 | 6.8 | 8.2 | 0.0 | 4.8 | 33.5 |
| g | Anaerostipes | 0.0 | 0.0 | 0.0 | 0.0 | 0.0 | 0.1 | 0.0 | 0.0 | 0.0 | 0.0 | 0.0 |
| s | Anaerostipes_{—} | 0.0 | 0.0 | 0.0 | 0.0 | 0.0 | 0.1 | 0.0 | 0.0 | 0.0 | 0.0 | 0.0 |
| | caccae | | | | | | | | | | | |
| g | Butyrivibrio | 0.0 | 0.0 | 0.0 | 0.0 | 0.0 | 0.0 | 0.0 | 0.0 | 0.0 | 0.0 | 0.0 |
| s | Butyrivibrio_{—} | 0.0 | 0.0 | 0.0 | 0.0 | 0.0 | 0.0 | 0.0 | 0.0 | 0.0 | 0.0 | 0.0 |
| | crossotus | | | | | | | | | | | |
| g | Coprococcus | 4.1 | 0.0 | 0.8 | 0.5 | 0.1 | 2.1 | 4.2 | 0.3 | 0.0 | 1.6 | 4.9 |
| s | Coprococcus_{—} | 1.5 | 0.0 | 0.8 | 0.3 | 0.0 | 2.1 | 0.9 | 0.3 | 0.0 | 0.0 | 0.9 |
| | catus | | | | | | | | | | | |
| s | Coprococcus_{—} | 2.5 | 0.0 | 0.0 | 0.2 | 0.1 | 0.0 | 3.3 | 0.0 | 0.0 | 1.6 | 4.0 |
| | comes | | | | | | | | | | | |
| g | Dorea | 4.5 | 0.0 | 0.0 | 0.2 | 10.3 | 2.2 | 2.6 | 4.4 | 0.0 | 2.2 | 13.7 |
| s | Dorea_{—} | 1.0 | 0.0 | 0.0 | 0.0 | 4.5 | 0.5 | 1.4 | 0.4 | 0.0 | 1.1 | 2.6 |
| | formicigenerans | | | | | | | | | | | |
| s | Dorea_{—} | 3.4 | 0.0 | 0.0 | 0.2 | 5.8 | 1.6 | 1.2 | 4.0 | 0.0 | 1.1 | 11.1 |
| | longicatena | | | | | | | | | | | |
| g | Roseburia | 5.9 | 0.0 | 2.6 | 11.8 | 4.2 | 1.0 | 0.0 | 3.5 | 0.0 | 1.0 | 14.9 |
| s | Roseburia_{—} | 4.8 | 0.0 | 1.8 | 0.0 | 0.4 | 0.3 | 0.0 | 2.8 | 0.0 | 1.0 | 14.9 |
| | intestinalis | | | | | | | | | | | |
| s | Roseburia_{—} | 1.1 | 0.0 | 0.8 | 11.8 | 3.9 | 0.7 | 0.0 | 0.7 | 0.0 | 0.0 | 0.0 |
| | inulinivorans | | | | | | | | | | | |
| f | Ruminococcaceae | 15.0 | 0.4 | 47.4 | 11.1 | 27.4 | 21.8 | 31.5 | 18.3 | 0.1 | 6.1 | 16.9 |
| g | Anaerotruncus | 0.0 | 0.0 | 0.0 | 0.0 | 0.1 | 0.1 | 0.0 | 0.0 | 0.0 | 0.0 | 0.0 |
| s | Anaerotruncus_{—} | 0.0 | 0.0 | 0.0 | 0.0 | 0.1 | 0.1 | 0.0 | 0.0 | 0.0 | 0.0 | 0.0 |
| | colihominis | | | | | | | | | | | |
| g | Faecalibacterium | 3.9 | 0.0 | 0.0 | 0.7 | 3.5 | 2.7 | 11.6 | 9.2 | 0.0 | 0.0 | 8.5 |
| s | Faecalibacterium_cf | 0.6 | 0.0 | 0.0 | 0.0 | 0.0 | 0.9 | 1.4 | 2.1 | 0.0 | 0.0 | 1.4 |
| s | Faecalibacterium_{—} | 2.9 | 0.0 | 0.0 | 0.6 | 3.5 | 1.9 | 10.0 | 6.8 | 0.0 | 0.0 | 6.5 |
| | prausnitzii | | | | | | | | | | | |
| s | Faecalibacterium_{—} | 0.5 | 0.0 | 0.0 | 0.0 | 0.0 | 0.0 | 0.3 | 0.3 | 0.0 | 0.0 | 0.7 |
| | unclassified | | | | | | | | | | | |
| s | Ruminococcus_{—} | 0.0 | 0.0 | 41.0 | 0.0 | 0.0 | 0.0 | 0.0 | 0.0 | 0.0 | 0.0 | 0.0 |
| | bromii | | | | | | | | | | | |
| s | Ruminococcus_{—} | 0.6 | 0.0 | 3.6 | 0.0 | 0.0 | 1.9 | 0.1 | 0.1 | 0.0 | 0.4 | 4.7 |
| | gnavus | | | | | | | | | | | |
| s | Ruminococcus_{—} | 0.0 | 0.0 | 0.0 | 5.8 | 0.0 | 0.0 | 0.0 | 0.0 | 0.0 | 4.1 | 1.4 |
| | lactaris | | | | | | | | | | | |
| s | Ruminococcus_{—} | 4.7 | 0.1 | 2.3 | 0.8 | 5.9 | 3.8 | 12.5 | 5.5 | 0.0 | 1.5 | 1.1 |
| | obeum | | | | | | | | | | | |
| s | Ruminococcus_{—} | 5.7 | 0.3 | 0.4 | 3.6 | 17.8 | 13.2 | 7.3 | 3.5 | 0.1 | 0.1 | 1.2 |
| | torques | | | | | | | | | | | |
| c | Erysipelotrichi | 0.2 | 2.6 | 2.3 | 0.0 | 0.3 | 1.7 | 0.0 | 0.3 | 0.8 | 0.5 | 0.1 |
| o | Erysipelotrichales | 0.2 | 2.6 | 2.3 | 0.0 | 0.3 | 1.7 | 0.0 | 0.3 | 0.8 | 0.5 | 0.1 |
| f | Erysipelotrichaceae | 0.2 | 2.6 | 2.3 | 0.0 | 0.3 | 1.7 | 0.0 | 0.3 | 0.8 | 0.5 | 0.1 |
| g | Catenibacterium | 0.0 | 0.0 | 0.0 | 0.0 | 0.0 | 0.0 | 0.0 | 0.0 | 0.0 | 0.0 | 0.0 |
| s | Catenibacterium_{—} | 0.0 | 0.0 | 0.0 | 0.0 | 0.0 | 0.0 | 0.0 | 0.0 | 0.0 | 0.0 | 0.0 |
| | mitsuokai | | | | | | | | | | | |
| g | Coprobacillus | 0.0 | 2.6 | 2.3 | 0.0 | 0.2 | 0.5 | 0.0 | 0.0 | 0.8 | 0.5 | 0.0 |
| s | Coprobacillus_{—} | 0.0 | 2.6 | 2.3 | 0.0 | 0.2 | 0.5 | 0.0 | 0.0 | 0.8 | 0.5 | 0.0 |
| | bacterium | | | | | | | | | | | |
| g | Holdemania | 0.2 | 0.0 | 0.0 | 0.0 | 0.2 | 0.9 | 0.0 | 0.2 | 0.0 | 0.0 | 0.1 |
| s | Holdemania_{—} | 0.2 | 0.0 | 0.0 | 0.0 | 0.2 | 0.9 | 0.0 | 0.2 | 0.0 | 0.0 | 0.1 |
| | filiformis | | | | | | | | | | | |
| c | Negativicutes | 0.0 | 0.0 | 4.3 | 4.2 | 0.0 | 1.5 | 0.0 | 1.4 | 0.2 | 2.5 | 2.3 |
| o | Selenomonadales | 0.0 | 0.0 | 4.3 | 4.2 | 0.0 | 1.5 | 0.0 | 1.4 | 0.2 | 2.5 | 2.3 |
| f | Acidaminococcaceae | 0.0 | 0.0 | 0.0 | 0.0 | 0.0 | 0.0 | 0.0 | 0.0 | 0.0 | 0.0 | 0.0 |
| g | Acidaminococcaceae_{—} | 0.0 | 0.0 | 0.0 | 0.0 | 0.0 | 0.0 | 0.0 | 0.0 | 0.0 | 0.0 | 0.0 |
| | unclassified | | | | | | | | | | | |
| g | Phascolarctobacterium | 0.0 | 0.0 | 0.0 | 0.0 | 0.0 | 0.0 | 0.0 | 0.0 | 0.0 | 0.0 | 0.0 |
| s | Phascolarctobacterium_{—} | 0.0 | 0.0 | 0.0 | 0.0 | 0.0 | 0.0 | 0.0 | 0.0 | 0.0 | 0.0 | 0.0 |
| | unclassified | | | | | | | | | | | |
| f | Veillonellaceae | 0.0 | 0.0 | 4.3 | 4.2 | 0.0 | 1.5 | 0.0 | 1.4 | 0.2 | 2.5 | 2.3 |
| g | Dialister | 0.0 | 0.0 | 4.3 | 3.9 | 0.0 | 1.5 | 0.0 | 0.0 | 0.0 | 2.5 | 0.0 |
| s | Dialister_{—} | 0.0 | 0.0 | 4.3 | 3.9 | 0.0 | 1.5 | 0.0 | 0.0 | 0.0 | 2.5 | 0.0 |
| | invisus | | | | | | | | | | | |
| g | Megamonas | 0.0 | 0.0 | 0.0 | 0.0 | 0.0 | 0.0 | 0.0 | 0.0 | 0.0 | 0.0 | 0.0 |
| s | Megamonas_{—} | 0.0 | 0.0 | 0.0 | 0.0 | 0.0 | 0.0 | 0.0 | 0.0 | 0.0 | 0.0 | 0.0 |
| | hypermegale | | | | | | | | | | | |
| g | Veillonella | 0.0 | 0.0 | 0.0 | 0.3 | 0.0 | 0.0 | 0.0 | 0.0 | 0.2 | 0.0 | 2.3 |
| s | Veillonella_{—} | 0.0 | 0.0 | 0.0 | 0.1 | 0.0 | 0.0 | 0.0 | 0.0 | 0.0 | 0.0 | 2.3 |
| | atypica | | | | | | | | | | | |
| s | Veillonella_{—} | 0.0 | 0.0 | 0.0 | 0.1 | 0.0 | 0.0 | 0.0 | 0.0 | 0.0 | 0.0 | 0.0 |
| | parvula | | | | | | | | | | | |
| s | Veillonella_{—} | 0.0 | 0.0 | 0.0 | 0.0 | 0.0 | 0.0 | 0.0 | 0.0 | 0.1 | 0.0 | 0.0 |
| | unclassified | | | | | | | | | | | |
| p | Fusobacteria | 0.0 | 0.0 | 0.0 | 0.0 | 0.0 | 0.0 | 0.0 | 0.1 | 0.0 | 0.0 | 0.0 |
| c | Fusobacteria | 0.0 | 0.0 | 0.0 | 0.0 | 0.0 | 0.0 | 0.0 | 0.1 | 0.0 | 0.0 | 0.0 |
| o | Fusobacteriales | 0.0 | 0.0 | 0.0 | 0.0 | 0.0 | 0.0 | 0.0 | 0.1 | 0.0 | 0.0 | 0.0 |
| f | Fusobacteriaceae | 0.0 | 0.0 | 0.0 | 0.0 | 0.0 | 0.0 | 0.0 | 0.1 | 0.0 | 0.0 | 0.0 |
| g | Fusobacterium | 0.0 | 0.0 | 0.0 | 0.0 | 0.0 | 0.0 | 0.0 | 0.1 | 0.0 | 0.0 | 0.0 |
| s | Fusobacterium_{—} | 0.0 | 0.0 | 0.0 | 0.0 | 0.0 | 0.0 | 0.0 | 0.0 | 0.0 | 0.0 | 0.0 |
| | ulcerans | | | | | | | | | | | |
| p | Proteobacteria | 0.7 | 2.4 | 0.7 | 0.7 | 0.4 | 0.2 | 7.8 | 2.1 | 3.9 | 0.1 | 6.8 |
| c | Betaproteobacteria | 0.0 | 0.0 | 0.0 | 0.0 | 0.1 | 0.0 | 0.0 | 0.0 | 0.0 | 0.0 | 0.0 |
| o | Burkholderiales | 0.0 | 0.0 | 0.0 | 0.0 | 0.1 | 0.0 | 0.0 | 0.0 | 0.0 | 0.0 | 0.0 |
| f | Sutterellaceae | 0.0 | 0.0 | 0.0 | 0.0 | 0.1 | 0.0 | 0.0 | 0.0 | 0.0 | 0.0 | 0.0 |
| g | Sutterella | 0.0 | 0.0 | 0.0 | 0.0 | 0.1 | 0.0 | 0.0 | 0.0 | 0.0 | 0.0 | 0.0 |
| s | Sutterella_{—} | 0.0 | 0.0 | 0.0 | 0.0 | 0.1 | 0.0 | 0.0 | 0.0 | 0.0 | 0.0 | 0.0 |
| | wadsworthensis | | | | | | | | | | | |
| c | Deltaproteobacteria | 0.3 | 0.0 | 0.4 | 0.6 | 0.0 | 0.1 | 0.0 | 0.3 | 0.0 | 0.0 | 0.1 |
| o | Desulfovibrionales | 0.3 | 0.0 | 0.4 | 0.6 | 0.0 | 0.1 | 0.0 | 0.3 | 0.0 | 0.0 | 0.1 |
| f | Desulfovibrionaceae | 0.3 | 0.0 | 0.4 | 0.6 | 0.0 | 0.1 | 0.0 | 0.3 | 0.0 | 0.0 | 0.1 |
| g | Bilophila | 0.3 | 0.0 | 0.3 | 0.3 | 0.0 | 0.1 | 0.0 | 0.3 | 0.0 | 0.0 | 0.1 |
| s | Bilophila_{—} | 0.3 | 0.0 | 0.3 | 0.3 | 0.0 | 0.1 | 0.0 | 0.3 | 0.0 | 0.0 | 0.1 |
| | wadsworthia | | | | | | | | | | | |
| g | Desulfovibrio | 0.0 | 0.0 | 0.1 | 0.3 | 0.0 | 0.0 | 0.0 | 0.0 | 0.0 | 0.0 | 0.0 |
| s | Desulfovibrio_{—} | 0.0 | 0.0 | 0.0 | 0.0 | 0.0 | 0.0 | 0.0 | 0.0 | 0.0 | 0.0 | 0.0 |
| | piger | | | | | | | | | | | |
| c | Gammaproteobacteria | 0.4 | 2.4 | 0.3 | 0.0 | 0.3 | 0.1 | 7.8 | 1.8 | 3.9 | 0.1 | 6.7 |
| o | Enterobacteriales | 0.4 | 2.4 | 0.3 | 0.0 | 0.3 | 0.1 | 7.8 | 1.7 | 3.9 | 0.1 | 6.7 |
| f | Enterobacteriaceae | 0.4 | 2.4 | 0.3 | 0.0 | 0.3 | 0.1 | 7.8 | 1.7 | 3.9 | 0.1 | 6.7 |
| g | Enterobacter | 0.1 | 0.1 | 0.0 | 0.0 | 0.0 | 0.0 | 0.0 | 0.0 | 0.0 | 0.0 | 6.5 |
| s | Enterobacter_{—} | 0.0 | 0.1 | 0.0 | 0.0 | 0.0 | 0.0 | 0.0 | 0.0 | 0.0 | 0.0 | 6.5 |
| | cloacae | | | | | | | | | | | |
| g | Escherichia | 0.2 | 1.5 | 0.3 | 0.0 | 0.3 | 0.1 | 5.9 | 0.0 | 3.9 | 0.1 | 0.0 |
| s | Escherichia_{—} | 0.2 | 1.5 | 0.3 | 0.0 | 0.3 | 0.1 | 3.3 | 0.0 | 3.9 | 0.1 | 0.0 |
| | coli | | | | | | | | | | | |
| s | Escherichia_{—} | 0.0 | 0.0 | 0.0 | 0.0 | 0.0 | 0.0 | 2.6 | 0.0 | 0.0 | 0.0 | 0.0 |
| | unclassified | | | | | | | | | | | |
| g | Klebsiella | 0.2 | 0.0 | 0.0 | 0.0 | 0.0 | 0.0 | 2.0 | 1.7 | 0.0 | 0.0 | 0.0 |
| s | Klebsiella_{—} | 0.1 | 0.0 | 0.0 | 0.0 | 0.0 | 0.0 | 0.0 | 1.5 | 0.0 | 0.0 | 0.0 |
| | pneumoniae | | | | | | | | | | | |
| s | Klebsiella_{—} | 0.0 | 0.0 | 0.0 | 0.0 | 0.0 | 0.0 | 1.9 | 0.2 | 0.0 | 0.0 | 0.0 |
| | unclassified | | | | | | | | | | | |
| p | Verrucomicrobia | 11.5 | 0.0 | 7.1 | 0.0 | 0.1 | 2.2 | 0.0 | 0.2 | 0.0 | 0.0 | 0.0 |
| c | Verrucomicrobiae | 11.5 | 0.0 | 7.1 | 0.0 | 0.1 | 2.2 | 0.0 | 0.2 | 0.0 | 0.0 | 0.0 |
| o | Verrucomicrobiales | 11.5 | 0.0 | 7.1 | 0.0 | 0.1 | 2.2 | 0.0 | 0.2 | 0.0 | 0.0 | 0.0 |
| f | Verrucomicrobiaceae | 11.5 | 0.0 | 7.1 | 0.0 | 0.1 | 2.2 | 0.0 | 0.2 | 0.0 | 0.0 | 0.0 |
| g | Akkermansia | 11.5 | 0.0 | 7.1 | 0.0 | 0.1 | 2.2 | 0.0 | 0.2 | 0.0 | 0.0 | 0.0 |
| s | Akkermansia_{—} | 11.5 | 0.0 | 7.1 | 0.0 | 0.1 | 2.2 | 0.0 | 0.2 | 0.0 | 0.0 | 0.0 |
| | muciniphila | | | | | | | | | | | |

| Rank | Name | P46 | P47 | P48 | P49 | P50 | P51 | P52 |
|---|---|---|---|---|---|---|---|---|
| k | Archaea | 0.0 | 0.0 | 21.0 | 3.6 | 4.4 | 2.4 | 2.5 |
| p | Euryarchaeota | 0.0 | 0.0 | 21.0 | 3.6 | 4.4 | 2.4 | 2.5 |
| c | Methanobacteria | 0.0 | 0.0 | 21.0 | 3.6 | 4.4 | 2.4 | 2.5 |
| o | Methanobacteriales | 0.0 | 0.0 | 21.0 | 3.6 | 4.4 | 2.4 | 2.5 |
| f | Methanobacteriaceae | 0.0 | 0.0 | 21.0 | 3.6 | 4.4 | 2.4 | 2.5 |
| g | Methanobrevibacter | 0.0 | 0.0 | 21.0 | 3.5 | 4.4 | 2.4 | 2.5 |
| s | Methanobrevibacter_{—} | 0.0 | 0.0 | 21.0 | 3.5 | 4.4 | 2.4 | 2.5 |
| | smithii | | | | | | | |
| k | Bacteria | 100.0 | 100.0 | 79.0 | 96.4 | 95.6 | 97.6 | 97.5 |
| p | Actinobacteria | 2.5 | 8.2 | 22.0 | 4.1 | 5.7 | 5.4 | 35.5 |
| c | Actinobacteria | 2.5 | 8.2 | 22.0 | 4.1 | 5.7 | 5.4 | 35.5 |
| o | Actinomycetales | 0.1 | 0.1 | 0.0 | 0.0 | 0.0 | 0.3 | 0.0 |
| f | Corynebacteriaceae | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 |
| s | Corynebacterium_{—} | 0.0 | 0.0 | 0.0 | 0.0 | 0.0 | 0.0 | 0.0 |
| | amycolatum | | | | | | | |
| s | Corynebacterium_{—} | 0.0 | 0.0 | 0.0 | 0.0 | 0.0 | 0.0 | 0.0 |
| | striatum | | | | | | | |
| o | Bifidobacteriales | 0.0 | 2.8 | 14.9 | 0.0 | 1.6 | 0.0 | 29.1 |
| f | Bifidobacteriaceae | 0.0 | 2.8 | 14.9 | 0.0 | 1.6 | 0.0 | 29.1 |
| g | Bifidobacterium | 0.0 | 2.7 | 14.9 | 0.0 | 1.6 | 0.0 | 29.1 |
| s | Bifidobacterium_{—} | 0.0 | 0.0 | 14.8 | 0.0 | 0.1 | 0.0 | 12.6 |
| | adolescentis | | | | | | | |
| s | Bifidobacterium_{—} | 0.0 | 0.0 | 0.0 | 0.0 | 0.0 | 0.0 | 0.0 |
| | animalis | | | | | | | |
| s | Bifidobacterium_{—} | 0.0 | 0.0 | 0.0 | 0.0 | 0.0 | 0.0 | 6.0 |
| | bifidum | | | | | | | |
| s | Bifidobacterium_{—} | 0.0 | 0.0 | 0.0 | 0.0 | 0.1 | 0.0 | 0.0 |
| | breve | | | | | | | |
| s | Bifidobacterium_{—} | 0.0 | 0.0 | 0.0 | 0.0 | 0.0 | 0.0 | 0.2 |
| | catenulatum | | | | | | | |
| s | Bifidobacterium_{—} | 0.0 | 0.4 | 0.1 | 0.0 | 0.0 | 0.0 | 0.0 |
| | dentium | | | | | | | |
| s | Bifidobacterium_{—} | 0.0 | 1.9 | 0.0 | 0.0 | 1.4 | 0.0 | 9.5 |
| | longum | | | | | | | |
| s | Bifidobacterium_{—} | 0.0 | 0.5 | 0.0 | 0.0 | 0.0 | 0.0 | 0.0 |
| | pseudocatenulatum | | | | | | | |
| o | Coriobacteriales | 2.5 | 5.3 | 7.1 | 4.1 | 4.1 | 5.1 | 6.4 |
| f | Coriobacteriaceae | 2.5 | 5.3 | 7.1 | 4.1 | 4.1 | 5.1 | 6.4 |
| g | Collinsella | 0.0 | 4.7 | 7.0 | 3.5 | 3.0 | 4.1 | 6.4 |
| s | Collinsella_{—} | 0.0 | 4.7 | 7.0 | 3.5 | 3.0 | 0.0 | 6.4 |
| | aerofaciens | | | | | | | |
| s | Collinsella_{—} | 0.0 | 0.0 | 0.0 | 0.0 | 0.0 | 4.1 | 0.0 |
| | intestinalis | | | | | | | |
| g | Eggerthella | 1.8 | 0.6 | 0.1 | 0.6 | 1.0 | 1.0 | 0.0 |
| s | Eggerthella_{—} | 1.8 | 0.6 | 0.1 | 0.6 | 1.0 | 1.0 | 0.0 |
| | lenta | | | | | | | |
| g | Gordonibacter | 0.7 | 0.0 | 0.0 | 0.0 | 0.0 | 0.0 | 0.0 |
| s | Gordonibacter_{—} | 0.7 | 0.0 | 0.0 | 0.0 | 0.0 | 0.0 | 0.0 |
| | pamelaeae | | | | | | | |
| p | Bacteroidetes | 32.1 | 23.1 | 26.0 | 8.3 | 20.1 | 20.3 | 34.2 |
| c | Bacteroidia | 32.1 | 23.1 | 26.0 | 8.3 | 20.1 | 20.3 | 34.2 |
| o | Bacteroidales | 32.1 | 23.1 | 26.0 | 8.3 | 20.1 | 20.3 | 34.2 |
| f | Bacteroidaceae | 32.1 | 22.8 | 12.2 | 6.8 | 9.0 | 12.0 | 5.1 |
| g | Bacteroides | 32.1 | 22.8 | 12.2 | 6.8 | 9.0 | 12.0 | 5.1 |
| s | Bacteroides_{—} | 0.0 | 2.2 | 1.9 | 0.0 | 0.0 | 0.0 | 0.3 |
| | caccae | | | | | | | |
| s | Bacteroides_{—} | 0.0 | 0.0 | 0.0 | 0.5 | 0.0 | 1.6 | 0.0 |
| | cellulosilyticus | | | | | | | |
| s | Bacteroides_{—} | 0.0 | 0.0 | 0.0 | 0.0 | 0.0 | 0.0 | 0.6 |
| | coprocola | | | | | | | |
| s | Bacteroides_{—} | 0.0 | 0.0 | 0.0 | 0.0 | 0.0 | 0.0 | 1.5 |
| | coprophilus | | | | | | | |
| s | Bacteroides_{—} | 0.0 | 0.0 | 0.3 | 0.0 | 0.0 | 0.0 | 0.0 |
| | eggerthii | | | | | | | |
| s | Bacteroides_{—} | 0.0 | 0.0 | 0.0 | 0.0 | 0.0 | 0.0 | 0.0 |
| | finegoldii | | | | | | | |
| s | Bacteroides_{—} | 0.9 | 0.0 | 0.1 | 0.0 | 0.1 | 0.0 | 0.0 |
| | fragilis | | | | | | | |
| s | Bacteroides_{—} | 0.0 | 0.0 | 0.0 | 0.0 | 2.0 | 0.0 | 0.0 |
| | intestinalis | | | | | | | |
| s | Bacteroides_{—} | 2.0 | 0.1 | 0.1 | 0.1 | 0.0 | 0.6 | 0.0 |
| | ovatus | | | | | | | |
| s | Bacteroides_{—} | 0.0 | 0.0 | 0.0 | 0.0 | 0.0 | 0.0 | 0.0 |
| | pectinophilus | | | | | | | |
| s | Bacteroides_{—} | 0.0 | 0.0 | 0.0 | 0.0 | 0.0 | 0.0 | 0.8 |
| | plebeius | | | | | | | |
| s | Bacteroides_{—} | 0.0 | 0.0 | 2.7 | 0.8 | 0.0 | 0.0 | 0.3 |
| | stercoris | | | | | | | |
| s | Bacteroides_{—} | 0.0 | 0.0 | 0.1 | 1.1 | 0.4 | 0.0 | 0.0 |
| | thetaiotaomicron | | | | | | | |
| s | Bacteroides_{—} | 22.6 | 15.5 | 4.2 | 3.5 | 3.3 | 6.3 | 0.0 |
| | unclassified | | | | | | | |
| s | Bacteroides_{—} | 4.2 | 1.1 | 0.6 | 0.0 | 0.0 | 1.3 | 0.1 |
| | uniformis | | | | | | | |
| s | Bacteroides_{—} | 0.0 | 3.1 | 2.0 | 0.0 | 3.1 | 2.0 | 1.4 |
| | vulgatus | | | | | | | |
| s | Bacteroides_{—} | 2.4 | 0.8 | 0.1 | 0.3 | 0.2 | 0.3 | 0.1 |
| | xylanisolvens | | | | | | | |
| f | Porphyromonadaceae | 0.0 | 0.0 | 6.3 | 1.0 | 0.5 | 3.0 | 2.6 |
| g | Odoribacter | 0.0 | 0.0 | 3.6 | 0.7 | 0.5 | 0.8 | 0.3 |
| s | Odoribacter_{—} | 0.0 | 0.0 | 3.6 | 0.7 | 0.5 | 0.8 | 0.3 |
| | splanchnicus | | | | | | | |
| g | Parabacteroides | 0.0 | 0.0 | 2.8 | 0.3 | 0.0 | 1.8 | 2.3 |
| s | Parabacteroides_{—} | 0.0 | 0.0 | 0.1 | 0.0 | 0.0 | 0.2 | 0.0 |
| | distasonis | | | | | | | |
| s | Parabacteroides_{—} | 0.0 | 0.0 | 0.0 | 0.0 | 0.0 | 1.2 | 0.0 |
| | johnsonii | | | | | | | |
| s | Parabacteroides_{—} | 0.0 | 0.0 | 2.5 | 0.2 | 0.0 | 0.0 | 1.6 |
| | merdae | | | | | | | |
| s | Parabacteroides_{—} | 0.0 | 0.0 | 0.1 | 0.0 | 0.0 | 0.4 | 0.7 |
| | unclassified | | | | | | | |
| g | Porphyromonas | 0.0 | 0.0 | 0.0 | 0.0 | 0.0 | 0.3 | 0.0 |
| s | Porphyromonas_{—} | 0.0 | 0.0 | 0.0 | 0.0 | 0.0 | 0.3 | 0.0 |
| | asaccharolytica | | | | | | | |
| f | Prevotellaceae | 0.0 | 0.0 | 0.0 | 0.2 | 0.0 | 0.3 | 25.4 |
| g | Prevotella | 0.0 | 0.0 | 0.0 | 0.2 | 0.0 | 0.3 | 25.4 |
| s | Prevotella_{—} | 0.0 | 0.0 | 0.0 | 0.2 | 0.0 | 0.0 | 25.4 |
| | copri | | | | | | | |
| f | Rikenellaceae | 0.0 | 0.3 | 7.5 | 0.5 | 10.6 | 5.0 | 1.0 |
| g | Alistipes | 0.0 | 0.3 | 7.5 | 0.5 | 10.6 | 5.0 | 1.0 |
| s | Alistipes_{—} | 0.0 | 0.0 | 2.6 | 0.0 | 6.8 | 4.1 | 0.8 |
| | putredinis | | | | | | | |
| s | Alistipes_shahii | 0.0 | 0.0 | 4.9 | 0.4 | 0.4 | 0.9 | 0.2 |
| s | Alistipes_{—} | 0.0 | 0.3 | 0.0 | 0.0 | 3.4 | 0.0 | 0.0 |
| | unclassified | | | | | | | |
| p | Firmicutes | 59.0 | 68.1 | 27.1 | 37.0 | 67.5 | 53.7 | 25.5 |
| c | Bacilli | 2.1 | 15.9 | 0.1 | 1.3 | 0.5 | 0.6 | 8.8 |
| o | Lactobacillales | 2.1 | 15.9 | 0.1 | 1.3 | 0.5 | 0.6 | 8.8 |
| f | Enterococcaceae | 2.1 | 0.0 | 0.0 | 0.1 | 0.0 | 0.0 | 0.0 |
| g | Enterococcus | 2.1 | 0.0 | 0.0 | 0.1 | 0.0 | 0.0 | 0.0 |
| s | Enterococcus_{—} | 0.0 | 0.0 | 0.0 | 0.0 | 0.0 | 0.0 | 0.0 |
| | casseliflavus | | | | | | | |
| s | Enterococcus_{—} | 0.0 | 0.0 | 0.0 | 0.1 | 0.0 | 0.0 | 0.0 |
| | faecalis | | | | | | | |
| s | Enterococcus_{—} | 2.1 | 0.0 | 0.0 | 0.0 | 0.0 | 0.0 | 0.0 |
| | faecium | | | | | | | |
| s | Enterococcus_{—} | 0.0 | 0.0 | 0.0 | 0.0 | 0.0 | 0.0 | 0.0 |
| | unclassified | | | | | | | |
| f | Lactobacillaceae | 0.0 | 7.3 | 0.0 | 0.4 | 0.0 | 0.1 | 8.7 |
| g | Lactobacillus | 0.0 | 7.3 | 0.0 | 0.3 | 0.0 | 0.1 | 8.7 |
| s | Lactobacillus_{—} | 0.0 | 0.0 | 0.0 | 0.0 | 0.0 | 0.0 | 0.0 |
| | casei | | | | | | | |
| s | Lactobacillus_{—} | 0.0 | 0.1 | 0.0 | 0.0 | 0.0 | 0.0 | 0.0 |
| | crispatus | | | | | | | |
| s | Lactobacillus_{—} | 0.0 | 1.5 | 0.0 | 0.1 | 0.0 | 0.1 | 0.0 |
| | fermentum | | | | | | | |
| s | Lactobacillus_{—} | 0.0 | 0.0 | 0.0 | 0.1 | 0.0 | 0.0 | 0.0 |
| | gasseri | | | | | | | |
| s | Lactobacillus_{—} | 0.0 | 0.0 | 0.0 | 0.0 | 0.0 | 0.0 | 0.0 |
| | paracasei | | | | | | | |
| s | Lactobacillus_{—} | 0.0 | 0.0 | 0.0 | 0.0 | 0.0 | 0.0 | 0.0 |
| | reuteri | | | | | | | |
| s | Lactobacillus_{—} | 0.0 | 1.3 | 0.0 | 0.0 | 0.0 | 0.0 | 8.7 |
| | ruminis | | | | | | | |
| s | Lactobacillus_{—} | 0.0 | 4.3 | 0.0 | 0.0 | 0.0 | 0.0 | 0.0 |
| | salivarius | | | | | | | |
| f | Streptococcaceae | 0.0 | 8.5 | 0.1 | 0.8 | 0.5 | 0.5 | 0.0 |
| g | Lactococcus | 0.0 | 0.3 | 0.0 | 0.0 | 0.0 | 0.1 | 0.0 |
| s | Lactococcus_{—} | 0.0 | 0.3 | 0.0 | 0.0 | 0.0 | 0.1 | 0.0 |
| | lactis | | | | | | | |
| g | Streptococcus | 0.0 | 8.2 | 0.1 | 0.8 | 0.5 | 0.4 | 0.0 |
| s | Streptococcus_{—} | 0.0 | 2.0 | 0.0 | 0.0 | 0.0 | 0.1 | 0.0 |
| | anginosus | | | | | | | |
| s | Streptococcus_{—} | 0.0 | 0.0 | 0.0 | 0.0 | 0.0 | 0.0 | 0.0 |
| | infantarius | | | | | | | |
| s | Streptococcus_{—} | 0.0 | 0.2 | 0.1 | 0.0 | 0.0 | 0.0 | 0.0 |
| | mutans | | | | | | | |
| s | Streptococcus_{—} | 0.0 | 0.4 | 0.0 | 0.3 | 0.0 | 0.1 | 0.0 |
| | parasanguinis | | | | | | | |
| s | Streptococcus_{—} | 0.0 | 4.0 | 0.0 | 0.0 | 0.0 | 0.1 | 0.0 |
| | salivarius | | | | | | | |
| s | Streptococcus_{—} | 0.0 | 0.2 | 0.0 | 0.3 | 0.4 | 0.0 | 0.0 |
| | thermophilus | | | | | | | |
| s | Streptococcus_{—} | 0.0 | 0.6 | 0.0 | 0.2 | 0.0 | 0.1 | 0.0 |
| | vestibularis | | | | | | | |
| c | Clostridia | 56.1 | 25.8 | 26.7 | 29.4 | 54.9 | 51.2 | 12.3 |
| o | Clostridiales | 56.1 | 25.8 | 26.7 | 29.4 | 54.9 | 51.2 | 12.3 |
| f | Clostridiaceae | 5.6 | 2.1 | 10.2 | 6.2 | 2.2 | 13.7 | 0.5 |
| g | Clostridium | 5.6 | 2.1 | 10.2 | 6.2 | 2.2 | 13.7 | 0.5 |
| s | Clostridium_{—} | 0.6 | 0.1 | 0.0 | 0.9 | 0.0 | 0.2 | 0.0 |
| | asparagiforme | | | | | | | |
| s | Clostridium_{—} | 0.4 | 0.0 | 2.7 | 0.0 | 0.3 | 0.2 | 0.1 |
| | bartlettii | | | | | | | |
| s | Clostridium_{—} | 2.1 | 1.0 | 0.0 | 1.4 | 0.0 | 9.2 | 0.0 |
| | bolteae | | | | | | | |
| s | Clostridium_{—} | 0.0 | 0.0 | 0.0 | 0.1 | 0.2 | 0.0 | 0.0 |
| | cf | | | | | | | |
| s | Clostridium_{—} | 0.1 | 0.1 | 0.6 | 1.4 | 0.0 | 1.5 | 0.0 |
| | hathewayi | | | | | | | |
| s | Clostridium_{—} | 0.0 | 0.0 | 0.0 | 0.0 | 0.0 | 0.0 | 0.0 |
| | hiranonis | | | | | | | |
| s | Clostridium_{—} | 2.4 | 0.0 | 0.7 | 0.0 | 0.8 | 0.1 | 0.4 |
| | leptum | | | | | | | |
| s | Clostridium_{—} | 0.0 | 0.0 | 2.1 | 0.0 | 0.6 | 0.4 | 0.0 |
| | nexile | | | | | | | |
| s | Clostridium_{—} | 0.0 | 0.1 | 4.0 | 0.0 | 0.2 | 0.0 | 0.0 |
| | perfringens | | | | | | | |
| s | Clostridium_{—} | 0.0 | 0.4 | 0.0 | 2.0 | 0.0 | 1.5 | 0.0 |
| | scindens | | | | | | | |
| s | Clostridium_{—} | 0.0 | 0.4 | 0.0 | 0.2 | 0.0 | 0.5 | 0.0 |
| | symbiosum | | | | | | | |
| f | Clostridiales_{—} | 1.4 | 0.0 | 0.0 | 0.0 | 0.0 | 7.0 | 0.0 |
| | Family_XI_{—} | | | | | | | |
| | Incertae_{—}Sedis | | | | | | | |
| g | Anaerococcus | 0.3 | 0.0 | 0.0 | 0.0 | 0.0 | 2.7 | 0.0 |
| s | Anaerococcus_{—} | 0.2 | 0.0 | 0.0 | 0.0 | 0.0 | 2.0 | 0.0 |
| | unclassified | | | | | | | |
| g | Clostridiales_{—} | 0.4 | 0.0 | 0.0 | 0.0 | 0.0 | 1.6 | 0.0 |
| | Family_XI_{—} | | | | | | | |
| | Incertae_{—}Sedis_{—} | | | | | | | |
| | unclassified | | | | | | | |
| g | Finegoldia | 0.2 | 0.0 | 0.0 | 0.0 | 0.0 | 0.2 | 0.0 |
| s | Finegoldia_{—} | 0.2 | 0.0 | 0.0 | 0.0 | 0.0 | 0.2 | 0.0 |
| | magna | | | | | | | |
| g | Parvimonas | 0.0 | 0.0 | 0.0 | 0.0 | 0.0 | 0.0 | 0.0 |
| s | Parvimonas_{—} | 0.0 | 0.0 | 0.0 | 0.0 | 0.0 | 0.0 | 0.0 |
| | micros | | | | | | | |
| g | Peptoniphilus | 0.5 | 0.0 | 0.0 | 0.0 | 0.0 | 2.5 | 0.0 |
| s | Peptoniphilus_{—} | 0.2 | 0.0 | 0.0 | 0.0 | 0.0 | 0.2 | 0.0 |
| | harei | | | | | | | |
| s | Peptoniphilus_{—} | 0.3 | 0.0 | 0.0 | 0.0 | 0.0 | 2.2 | 0.0 |
| | unclassified | | | | | | | |
| f | Clostridiales_{—} | 0.9 | 0.5 | 0.1 | 0.1 | 0.1 | 0.4 | 0.1 |
| | uncl | | | | | | | |
| g | Blautia | 0.9 | 0.5 | 0.1 | 0.1 | 0.1 | 0.4 | 0.1 |
| s | Blautia_{—} | 0.6 | 0.4 | 0.0 | 0.0 | 0.0 | 0.0 | 0.0 |
| | hansenii | | | | | | | |
| s | Blautia_{—} | 0.0 | 0.0 | 0.0 | 0.0 | 0.1 | 0.4 | 0.0 |
| | hydrogenotrophica | | | | | | | |
| s | Blautia_{—} | 0.4 | 0.1 | 0.1 | 0.1 | 0.0 | 0.0 | 0.1 |
| | unclassified | | | | | | | |
| f | Eubacteriaceae | 0.9 | 13.1 | 5.2 | 1.9 | 15.0 | 5.9 | 2.1 |
| g | Eubacterium | 0.9 | 13.1 | 5.1 | 1.8 | 15.0 | 5.8 | 2.1 |
| s | Eubacterium_{—} | 0.3 | 0.0 | 0.0 | 0.0 | 1.0 | 0.0 | 0.1 |
| | eligens | | | | | | | |
| s | Eubacterium_{—} | 0.6 | 0.0 | 0.0 | 0.0 | 0.7 | 0.0 | 1.2 |
| | hallii | | | | | | | |
| s | Eubacterium_{—} | 0.0 | 0.0 | 0.3 | 0.1 | 0.2 | 0.7 | 0.0 |
| | limosum | | | | | | | |
| s | Eubacterium_{—} | 0.0 | 13.1 | 0.1 | 0.0 | 9.4 | 5.1 | 0.9 |
| | rectale | | | | | | | |
| s | Eubacterium_{—} | 0.0 | 0.0 | 4.6 | 1.7 | 3.7 | 0.1 | 0.0 |
| | siraeum | | | | | | | |
| s | Eubacterium_{—} | 0.0 | 0.0 | 0.0 | 0.0 | 0.0 | 0.0 | 0.0 |
| | ventriosum | | | | | | | |
| g | Pseudoramibacter | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 |
| | alactolyticus | | | | | | | |
| f | Lachnospiraceae | 22.1 | 1.1 | 4.2 | 3.2 | 0.7 | 0.2 | 2.6 |
| g | Anaerostipes | 0.3 | 0.0 | 0.0 | 0.1 | 0.1 | 0.2 | 0.0 |
| s | Anaerostipes_{—} | 0.3 | 0.0 | 0.0 | 0.1 | 0.1 | 0.2 | 0.0 |
| | caccae | | | | | | | |
| g | Butyrivibrio | 0.0 | 0.0 | 4.1 | 0.0 | 0.0 | 0.0 | 0.0 |
| s | Butyrivibrio_{—} | 0.0 | 0.0 | 4.1 | 0.0 | 0.0 | 0.0 | 0.0 |
| | crossotus | | | | | | | |
| g | Coprococcus | 0.0 | 0.0 | 0.0 | 1.6 | 0.0 | 0.0 | 0.4 |
| s | Coprococcus_{—} | 0.0 | 0.0 | 0.0 | 0.0 | 0.0 | 0.0 | 0.1 |
| | catus | | | | | | | |
| s | Coprococcus_{—} | 0.0 | 0.0 | 0.0 | 1.6 | 0.0 | 0.0 | 0.3 |
| | comes | | | | | | | |
| g | Dorea | 0.0 | 1.0 | 0.0 | 0.1 | 0.2 | 0.0 | 1.9 |
| s | Dorea_{—} | 0.0 | 1.0 | 0.0 | 0.1 | 0.2 | 0.0 | 0.9 |
| | formicigenerans | | | | | | | |
| s | Dorea_{—} | 0.0 | 0.0 | 0.0 | 0.0 | 0.0 | 0.0 | 1.0 |
| | longicatena | | | | | | | |
| g | Roseburia | 21.8 | 0.0 | 0.0 | 1.4 | 0.4 | 0.0 | 0.4 |
| s | Roseburia_{—} | 21.7 | 0.0 | 0.0 | 0.0 | 0.4 | 0.0 | 0.1 |
| | intestinalis | | | | | | | |
| s | Roseburia_{—} | 0.0 | 0.0 | 0.0 | 1.4 | 0.0 | 0.0 | 0.3 |
| | inulinivorans | | | | | | | |
| f | Ruminococcaceae | 25.2 | 9.0 | 7.1 | 17.9 | 36.9 | 24.0 | 7.0 |
| g | Anaerotruncus | 0.3 | 0.0 | 0.0 | 0.3 | 0.0 | 0.1 | 0.0 |
| s | Anaerotruncus_{—} | 0.3 | 0.0 | 0.0 | 0.3 | 0.0 | 0.1 | 0.0 |
| | colihominis | | | | | | | |
| g | Faecalibacterium | 0.0 | 0.1 | 0.5 | 1.9 | 1.5 | 0.0 | 5.0 |
| s | Faecalibacterium_cf | 0.0 | 0.0 | 0.0 | 0.0 | 0.0 | 0.0 | 0.9 |
| s | Faecalibacterium_{—} | 0.0 | 0.0 | 0.4 | 1.9 | 1.5 | 0.0 | 4.1 |
| | prausnitzii | | | | | | | |
| s | Faecalibacterium_{—} | 0.0 | 0.1 | 0.0 | 0.0 | 0.0 | 0.0 | 0.0 |
| | unclassified | | | | | | | |
| s | Ruminococcus_{—} | 7.2 | 0.0 | 0.1 | 5.1 | 19.2 | 21.5 | 0.5 |
| | bromii | | | | | | | |
| s | Ruminococcus_{—} | 6.3 | 2.0 | 0.2 | 0.1 | 10.3 | 0.3 | 0.0 |
| | gnavus | | | | | | | |
| s | Ruminococcus_{—} | 0.0 | 0.0 | 0.0 | 0.0 | 0.0 | 0.0 | 0.0 |
| | lactaris | | | | | | | |
| s | Ruminococcus_{—} | 0.0 | 0.0 | 2.0 | 5.9 | 0.0 | 0.0 | 1.1 |
| | obeum | | | | | | | |
| s | Ruminococcus_{—} | 11.3 | 6.9 | 4.3 | 4.5 | 5.8 | 2.1 | 0.4 |
| | torques | | | | | | | |
| c | Erysipelotrichi | 0.8 | 0.8 | 0.3 | 4.8 | 10.2 | 1.8 | 1.6 |
| o | Erysipelotrichales | 0.8 | 0.8 | 0.3 | 4.8 | 10.2 | 1.8 | 1.6 |
| f | Erysipelotrichaceae | 0.8 | 0.8 | 0.3 | 4.8 | 10.2 | 1.8 | 1.6 |
| g | Catenibacterium | 0.0 | 0.0 | 0.0 | 3.2 | 0.0 | 0.0 | 1.6 |
| s | Catenibacterium_{—} | 0.0 | 0.0 | 0.0 | 3.2 | 0.0 | 0.0 | 1.6 |
| | mitsuokai | | | | | | | |
| g | Coprobacillus | 0.8 | 0.7 | 0.1 | 1.6 | 10.2 | 1.4 | 0.0 |
| s | Coprobacillus_{—} | 0.8 | 0.7 | 0.1 | 1.6 | 10.2 | 1.4 | 0.0 |
| | bacterium | | | | | | | |
| g | Holdemania | 0.0 | 0.1 | 0.1 | 0.0 | 0.0 | 0.0 | 0.0 |
| s | Holdemania_{—} | 0.0 | 0.1 | 0.1 | 0.0 | 0.0 | 0.0 | 0.0 |
| | filiformis | | | | | | | |
| c | Negativicutes | 0.0 | 25.7 | 0.0 | 1.6 | 1.9 | 0.0 | 2.9 |
| o | Selenomonadales | 0.0 | 25.7 | 0.0 | 1.6 | 1.9 | 0.0 | 2.9 |
| f | Acidaminococcaceae | 0.0 | 0.0 | 0.0 | 1.6 | 0.0 | 0.0 | 2.9 |
| g | Acidaminococcaceae_{—} | 0.0 | 0.0 | 0.0 | 0.0 | 0.0 | 0.0 | 0.0 |
| | unclassified | | | | | | | |
| g | Phascolarctobacterium | 0.0 | 0.0 | 0.0 | 1.6 | 0.0 | 0.0 | 2.9 |
| s | Phascolarctobacterium_{—} | 0.0 | 0.0 | 0.0 | 1.6 | 0.0 | 0.0 | 2.9 |
| | unclassified | | | | | | | |
| f | Veillonellaceae | 0.0 | 25.7 | 0.0 | 0.0 | 1.9 | 0.0 | 0.0 |
| g | Dialister | 0.0 | 2.1 | 0.0 | 0.0 | 1.9 | 0.0 | 0.0 |
| s | Dialister_{—} | 0.0 | 2.1 | 0.0 | 0.0 | 1.9 | 0.0 | 0.0 |
| | invisus | | | | | | | |
| g | Megamonas | 0.0 | 23.2 | 0.0 | 0.0 | 0.0 | 0.0 | 0.0 |
| s | Megamonas_{—} | 0.0 | 23.2 | 0.0 | 0.0 | 0.0 | 0.0 | 0.0 |
| | hypermegale | | | | | | | |
| g | Veillonella | 0.0 | 0.4 | 0.0 | 0.0 | 0.0 | 0.0 | 0.0 |
| s | Veillonella_{—} | 0.0 | 0.0 | 0.0 | 0.0 | 0.0 | 0.0 | 0.0 |
| | atypica | | | | | | | |
| s | Veillonella_{—} | 0.0 | 0.3 | 0.0 | 0.0 | 0.0 | 0.0 | 0.0 |
| | parvula | | | | | | | |
| s | Veillonella_{—} | 0.0 | 0.1 | 0.0 | 0.0 | 0.0 | 0.0 | 0.0 |
| | unclassified | | | | | | | |
| p | Fusobacteria | 0.0 | 0.0 | 0.0 | 0.0 | 0.0 | 0.1 | 0.0 |
| c | Fusobacteria | 0.0 | 0.0 | 0.0 | 0.0 | 0.0 | 0.1 | 0.0 |
| o | Fusobacteriales | 0.0 | 0.0 | 0.0 | 0.0 | 0.0 | 0.1 | 0.0 |
| f | Fusobacteriaceae | 0.0 | 0.0 | 0.0 | 0.0 | 0.0 | 0.1 | 0.0 |
| g | Fusobacterium | 0.0 | 0.0 | 0.0 | 0.0 | 0.0 | 0.1 | 0.0 |
| s | Fusobacterium_{—} | 0.0 | 0.0 | 0.0 | 0.0 | 0.0 | 0.1 | 0.0 |
| | ulcerans | | | | | | | |
| p | Proteobacteria | 6.4 | 0.5 | 2.9 | 2.3 | 1.7 | 1.6 | 2.3 |
| c | Betaproteobacteria | 0.0 | 0.0 | 0.0 | 0.1 | 0.0 | 0.0 | 0.9 |
| o | Burkholderiales | 0.0 | 0.0 | 0.0 | 0.1 | 0.0 | 0.0 | 0.9 |
| f | Sutterellaceae | 0.0 | 0.0 | 0.0 | 0.1 | 0.0 | 0.0 | 0.9 |
| g | Sutterella | 0.0 | 0.0 | 0.0 | 0.1 | 0.0 | 0.0 | 0.9 |
| s | Sutterella_{—} | 0.0 | 0.0 | 0.0 | 0.1 | 0.0 | 0.0 | 0.9 |
| | wadsworthensis | | | | | | | |
| c | Deltaproteobacteria | 1.3 | 0.4 | 0.4 | 1.2 | 0.3 | 0.1 | 0.0 |
| o | Desulfovibrionales | 1.3 | 0.4 | 0.4 | 1.2 | 0.3 | 0.1 | 0.0 |
| f | Desulfovibrionaceae | 1.3 | 0.4 | 0.4 | 1.2 | 0.3 | 0.1 | 0.0 |
| g | Bilophila | 1.3 | 0.4 | 0.1 | 0.0 | 0.3 | 0.0 | 0.0 |
| s | Bilophila_{—} | 1.3 | 0.4 | 0.1 | 0.0 | 0.3 | 0.0 | 0.0 |
| | wadsworthia | | | | | | | |
| g | Desulfovibrio | 0.0 | 0.0 | 0.3 | 1.2 | 0.0 | 0.1 | 0.0 |
| s | Desulfovibrio_{—} | 0.0 | 0.0 | 0.2 | 1.0 | 0.0 | 0.0 | 0.0 |
| | piger | | | | | | | |
| c | Gammaproteobacteria | 5.1 | 0.1 | 2.5 | 1.0 | 1.4 | 1.5 | 1.4 |
| o | Enterobacteriales | 5.1 | 0.0 | 2.5 | 1.0 | 1.4 | 1.5 | 1.4 |
| f | Enterobacteriaceae | 5.1 | 0.0 | 2.5 | 1.0 | 1.4 | 1.5 | 1.4 |
| g | Enterobacter | 0.0 | 0.0 | 0.0 | 0.0 | 0.0 | 0.0 | 0.0 |
| s | Enterobacter_{—} | 0.0 | 0.0 | 0.0 | 0.0 | 0.0 | 0.0 | 0.0 |
| | cloacae | | | | | | | |
| g | Escherichia | 5.1 | 0.0 | 2.4 | 1.0 | 1.3 | 1.2 | 1.4 |
| s | Escherichia_{—} | 5.1 | 0.0 | 2.4 | 1.0 | 1.3 | 1.2 | 1.4 |
| | coli | | | | | | | |
| s | Escherichia_{—} | 0.0 | 0.0 | 0.0 | 0.0 | 0.0 | 0.0 | 0.0 |
| | unclassified | | | | | | | |
| g | Klebsiella | 0.0 | 0.0 | 0.2 | 0.0 | 0.0 | 0.0 | 0.0 |
| s | Klebsiella_{—} | 0.0 | 0.0 | 0.1 | 0.0 | 0.0 | 0.0 | 0.0 |
| | pneumoniae | | | | | | | |
| s | Klebsiella_{—} | 0.0 | 0.0 | 0.0 | 0.0 | 0.0 | 0.0 | 0.0 |
| | unclassified | | | | | | | |
| p | Verrucomicrobia | 0.0 | 0.0 | 1.0 | 44.7 | 0.6 | 16.5 | 0.0 |
| c | Verrucomicrobiae | 0.0 | 0.0 | 1.0 | 44.7 | 0.6 | 16.5 | 0.0 |
| o | Verrucomicrobiales | 0.0 | 0.0 | 1.0 | 44.7 | 0.6 | 16.5 | 0.0 |
| f | Verrucomicrobiaceae | 0.0 | 0.0 | 1.0 | 44.7 | 0.6 | 16.5 | 0.0 |
| g | Akkermansia | 0.0 | 0.0 | 1.0 | 44.7 | 0.6 | 16.5 | 0.0 |
| s | Akkermansia_{—} | 0.0 | 0.0 | 1.0 | 44.7 | 0.6 | 16.5 | 0.0 |
| | muciniphila | | | | | | | |

Rank: k = kingdom p = phylum c = class o = order f = family g = genus s = species

TABLE 2D

| Rank | Name | P53 | P54 | P55 | P56 | P57 | P58 | P59 | P60 | P61 | P62 | P63 | P64 |
|---|---|---|---|---|---|---|---|---|---|---|---|---|---|
| k | Archaea | 0.4 | 0.0 | 0.0 | 1.3 | 3.5 | 0.0 | 0.0 | 0.0 | 0.0 | 0.0 | 0.0 | 0.0 |
| p | Euryarchaeota | 0.4 | 0.0 | 0.0 | 1.3 | 3.5 | 0.0 | 0.0 | 0.0 | 0.0 | 0.0 | 0.0 | 0.0 |
| c | Methanobacteria | 0.4 | 0.0 | 0.0 | 1.3 | 3.5 | 0.0 | 0.0 | 0.0 | 0.0 | 0.0 | 0.0 | 0.0 |
| o | Methanobacteriales | 0.4 | 0.0 | 0.0 | 1.3 | 3.5 | 0.0 | 0.0 | 0.0 | 0.0 | 0.0 | 0.0 | 0.0 |
| f | Methanobacteriaceae | 0.4 | 0.0 | 0.0 | 1.3 | 3.5 | 0.0 | 0.0 | 0.0 | 0.0 | 0.0 | 0.0 | 0.0 |
| g | Methanobrevibacter | 0.4 | 0.0 | 0.0 | 1.3 | 3.0 | 0.0 | 0.0 | 0.0 | 0.0 | 0.0 | 0.0 | 0.0 |
| s | Methanobrevibacter_{—} | 0.4 | 0.0 | 0.0 | 1.3 | 3.0 | 0.0 | 0.0 | 0.0 | 0.0 | 0.0 | 0.0 | 0.0 |
| | smithii | | | | | | | | | | | | |
| k | Bacteria | 99.6 | 100.0 | 100.0 | 98.7 | 96.5 | 100.0 | 100.0 | 100.0 | 100.0 | 100.0 | 100.0 | 100.0 |
| p | Actinobacteria | 2.9 | 7.2 | 5.6 | 8.4 | 1.6 | 7.3 | 1.2 | 8.8 | 1.5 | 5.8 | 2.0 | 8.0 |
| c | Actinobacteria | 2.9 | 7.2 | 5.6 | 8.4 | 1.6 | 7.3 | 1.2 | 8.8 | 1.5 | 5.8 | 2.0 | 8.0 |
| o | Actinomycetales | 0.3 | 0.1 | 0.0 | 0.1 | 0.0 | 0.0 | 0.2 | 0.1 | 0.0 | 0.1 | 0.0 | 0.1 |
| f | Corynebacteriaceae | 0.0 | 0.0 | 0.0 | 0.0 | 0.0 | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 | 0.0 | 0.0 | 0.0 | 0.0 | 0.0 |
| s | corynebacterium_{—} | 0.0 | 0.0 | 0.0 | 0.0 | 0.0 | 0.0 | 0.0 | 0.0 | 0.0 | 0.0 | 0.0 | 0.0 |
| | amycolatum | | | | | | | | | | | | |
| s | corynebacterium_{—} | 0.0 | 0.0 | 0.0 | 0.0 | 0.0 | 0.0 | 0.0 | 0.0 | 0.0 | 0.0 | 0.0 | 0.0 |
| | striatum | | | | | | | | | | | | |
| o | Bifidobacteriales | 0.0 | 2.4 | 0.3 | 3.7 | 0.1 | 5.2 | 0.0 | 4.3 | 0.0 | 2.3 | 0.3 | 1.5 |
| f | Bifidobacteriaceae | 0.0 | 2.4 | 0.3 | 3.7 | 0.1 | 5.2 | 0.0 | 4.3 | 0.0 | 2.3 | 0.3 | 1.5 |
| g | Bifidobacterium | 0.0 | 2.4 | 0.3 | 3.7 | 0.1 | 5.2 | 0.0 | 4.3 | 0.0 | 2.3 | 0.3 | 1.5 |
| s | Bifidobacterium_{—} | 0.0 | 0.6 | 0.0 | 0.0 | 0.0 | 0.6 | 0.0 | 0.0 | 0.0 | 0.0 | 0.1 | 0.0 |
| | adolescentis | | | | | | | | | | | | |
| s | Bifidobacterium_{—} | 0.0 | 0.0 | 0.0 | 0.0 | 0.1 | 0.0 | 0.0 | 0.0 | 0.0 | 0.0 | 0.0 | 0.0 |
| | animalis | | | | | | | | | | | | |
| s | Bifidobacterium_{—} | 0.0 | 0.0 | 0.0 | 0.0 | 0.0 | 4.5 | 0.0 | 0.0 | 0.0 | 0.0 | 0.0 | 0.0 |
| | bifidum | | | | | | | | | | | | |
| s | Bifidobacterium_{—} | 0.0 | 0.0 | 0.0 | 0.0 | 0.0 | 0.0 | 0.0 | 0.0 | 0.0 | 0.0 | 0.0 | 0.0 |
| | breve | | | | | | | | | | | | |
| s | bifidobacterium_{—} | 0.0 | 0.0 | 0.0 | 0.0 | 0.0 | 0.0 | 0.0 | 0.0 | 0.0 | 0.0 | 0.0 | 0.0 |
| | catenulatum | | | | | | | | | | | | |
| s | bifidobacterium_{—} | 0.0 | 0.0 | 0.0 | 0.0 | 0.0 | 0.0 | 0.0 | 0.1 | 0.0 | 0.0 | 0.0 | 0.1 |
| | dentium | | | | | | | | | | | | |
| s | bifidobacterium_{—} | 0.0 | 1.7 | 0.3 | 1.9 | 0.0 | 0.0 | 0.0 | 4.2 | 0.0 | 2.2 | 0.2 | 1.4 |
| | longum | | | | | | | | | | | | |
| s | bifidobacterium_{—} | 0.0 | 0.1 | 0.0 | 1.7 | 0.1 | 0.0 | 0.0 | 0.0 | 0.0 | 0.0 | 0.0 | 0.0 |
| | pseudocatenulatum | | | | | | | | | | | | |
| o | coriobacteriales | 2.6 | 4.6 | 5.2 | 4.7 | 1.4 | 2.0 | 1.0 | 4.3 | 1.5 | 3.5 | 1.7 | 6.3 |
| f | coriobacteriaceae | 0.0 | 4.6 | 5.2 | 4.7 | 1.4 | 2.0 | 1.0 | 4.3 | 1.5 | 3.5 | 1.7 | 6.3 |
| g | collinsella | 0.0 | 4.4 | 4.0 | 4.6 | 1.4 | 1.9 | 0.0 | 4.2 | 0.0 | 0.0 | 1.6 | 6.0 |
| s | collinsella_{—} | 0.0 | 4.3 | 4.0 | 4.6 | 1.4 | 1.9 | 0.0 | 4.2 | 0.0 | 0.0 | 1.6 | 6.0 |
| | aerofaciens | | | | | | | | | | | | |
| s | collinsella_{—} | 0.0 | 0.0 | 0.0 | 0.0 | 0.0 | 0.0 | 0.0 | 0.0 | 0.0 | 0.0 | 0.0 | 0.0 |
| | intestinalis | | | | | | | | | | | | |
| g | eggerthella | 2.2 | 0.1 | 0.8 | 0.0 | 0.0 | 0.1 | 1.0 | 0.1 | 1.0 | 3.5 | 0.0 | 0.2 |
| s | eggerthella_{—} | 2.2 | 0.1 | 0.8 | 0.0 | 0.0 | 0.1 | 1.0 | 0.1 | 1.0 | 3.5 | 0.0 | 0.2 |
| | lenta | | | | | | | | | | | | |
| g | gordonibacter | 0.3 | 0.2 | 0.4 | 0.0 | 0.0 | 0.1 | 0.0 | 0.0 | 0.5 | 0.0 | 0.1 | 0.1 |
| s | gordonibacter_{—} | 0.3 | 0.2 | 0.4 | 0.0 | 0.0 | 0.1 | 0.0 | 0.0 | 0.5 | 0.0 | 0.1 | 0.1 |
| | pamelaeae | | | | | | | | | | | | |
| p | Bacteroidetes | 45.3 | 27.2 | 45.6 | 14.5 | 31.1 | 49.6 | 20.1 | 43.2 | 25.0 | 56.6 | 27.3 | 50.7 |
| c | Bacteroidia | 45.3 | 27.2 | 45.6 | 14.5 | 31.1 | 49.6 | 20.1 | 43.2 | 25.0 | 56.6 | 27.3 | 50.7 |
| o | Bacteroidales | 45.3 | 27.2 | 45.6 | 14.5 | 31.1 | 49.6 | 20.1 | 43.2 | 25.0 | 56.6 | 27.3 | 50.7 |
| f | Bacteroidaceae | 21.4 | 15.9 | 36.8 | 13.2 | 20.7 | 9.0 | 9.2 | 32.8 | 20.6 | 45.9 | 11.0 | 35.6 |
| g | Bacteroides | 21.4 | 15.9 | 36.8 | 13.2 | 20.7 | 9.0 | 9.2 | 32.8 | 20.6 | 45.9 | 11.0 | 35.6 |
| s | Bacteroides_{—} | 0.0 | 1.1 | 0.0 | 0.9 | 0.7 | 0.1 | 0.7 | 2.4 | 4.2 | 0.0 | 0.5 | 0.0 |
| | caccae | | | | | | | | | | | | |
| s | Bacteroides_{—} | | 0.2 | 0.0 | 0.0 | 2.0 | 0.1 | 0.6 | 0.5 | 0.0 | 0.0 | 0.3 | 0.1 |
| | cellulosilyticus | 0.1 | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 | 0.0 |
| | coprocola | | | | | | | | | | | | |
| s | Bacteroides_{—} | 0.0 | 0.0 | 0.0 | 0.0 | 0.0 | 0.0 | 0.0 | 0.0 | 0.0 | 0.0 | 0.0 | 0.0 |
| | coprophilus | | | | | | | | | | | | |
| s | Bacteroides_{—} | 0.0 | 0.2 | 0.0 | 0.0 | 0.0 | 0.0 | 0.0 | 0.0 | 0.0 | 0.0 | 0.0 | 0.0 |
| | eggerthi | | | | | | | | | | | | |
| s | Bacteroides_{—} | 0.0 | 1.9 | 1.5 | 1.2 | 0.0 | 0.3 | 0.0 | 0.8 | 0.0 | 0.0 | 2.2 | 0.0 |
| | finegoldii | | | | | | | | | | | | |
| s | Bacteroides_{—} | 0.4 | 0.1 | 0.0 | 0.0 | 0.1 | 0.0 | 0.0 | 0.0 | 0.0 | 0.0 | 0.0 | 0.0 |
| | fragilis | | | | | | | | | | | | |
| s | Bacteroides_{—} | 0.0 | 0.0 | 0.0 | 0.0 | 7.0 | 0.0 | 0.0 | 0.0 | 1.8 | 0.0 | 0.0 | 1.4 |
| | intestinalis | | | | | | | | | | | | |
| s | Bacteroides_{—} | 1.3 | 0.1 | 1.1 | 0.1 | 0.1 | 0.1 | 0.9 | 0.1 | 0.4 | 0.0 | 0.1 | 0.9 |
| | ovatus | | | | | | | | | | | | |
| s | Bacteroides_{—} | 0.0 | 0.0 | 0.0 | 0.0 | 3.0 | 0.0 | 0.0 | 0.0 | 0.0 | 0.0 | 0.0 | 0.0 |
| | pectinophilus | | | | | | | | | | | | |
| s | Bacteroides_{—} | 0.0 | 0.0 | 4.8 | 0.0 | 0.0 | 7.5 | 0.0 | 6.8 | 0.0 | 0.0 | 0.0 | 0.0 |
| | plebeius | | | | | | | | | | | | |
| s | Bacteroides_{—} | 10.8 | 8.1 | 18.2 | 2.7 | 0.0 | 0.2 | 0.0 | 0.0 | 0.0 | 0.0 | 0.4 | 13.3 |
| | stercoris | | | | | | | | | | | | |
| s | Bacteroides_{—}thetaiotaomicron | 0.0 | 0.4 | 0.3 | 0.4 | 0.0 | 0.0 | 1.9 | 0.2 | 0.2 | 0.0 | 0.1 | 0.0 |
| s | Bacteroides_{—} | 2.8 | 3.7 | 8.8 | 3.6 | 0.0 | 0.0 | 3.2 | 3.0 | 0.1 | 41.3 | 0.5 | 9.2 |
| | unclassified | | | | | | | | | | | | |
| s | Bacteroides_{—} | 2.0 | 0.0 | 0.1 | 0.2 | 0.1 | 0.0 | 0.0 | 0.3 | 0.6 | 4.6 | 0.1 | 0.0 |
| | uniformis | | | | | | | | | | | | |
| s | Bacteroides_{—} | 2.6 | 0.0 | 1.6 | 2.9 | 7.6 | 0.6 | 1.6 | 18.0 | 12.7 | 0.0 | 6.0 | 10.7 |
| | vulgatus | | | | | | | | | | | | |
| s | Bacteroides_{—} | 1.3 | 0.0 | 0.2 | 1.1 | 0.1 | 0.0 | 0.3 | 0.7 | 0.5 | 0.0 | 0.3 | 0.0 |
| | xylanisolvens | | | | | | | | | | | | |
| f | Porphyromonadaceae | 8.5 | 2.6 | 2.4 | 0.6 | 0.7 | 1.0 | 0.8 | 7.3 | 0.9 | 5.5 | 1.8 | 5.1 |
| g | Odoribacter | 6.7 | 0.6 | 0.5 | 0.0 | 0.5 | 0.3 | 0.7 | 0.0 | 0.9 | 0.0 | 0.6 | 0.0 |
| s | Odoribacter_{—} | 6.7 | 0.6 | 0.5 | 0.0 | 0.5 | 0.3 | 0.7 | 0.0 | 0.9 | 0.0 | 0.6 | 0.0 |
| | splanchnicus | | | | | | | | | | | | |
| g | Parabacteroides | 1.8 | 2.0 | 1.8 | 0.6 | 0.2 | 0.7 | 0.1 | 7.3 | 0.1 | 5.5 | 1.2 | 5.1 |
| s | Parabacteroides_{—} | 0.0 | 0.0 | 0.0 | 0.0 | 0.1 | 0.0 | 0.0 | 0.0 | 0.0 | 0.0 | 0.0 | 0.0 |
| | distasonis | | | | | | | | | | | | |
| s | Parabacteroides_{—} | 0.1 | 0.0 | 0.0 | 0.0 | 0.0 | 0.0 | 0.0 | 0.1 | 0.0 | 0.0 | 0.4 | 0.5 |
| | johnsonii | | | | | | | | | | | | |
| s | Parabacteroides_{—} | 1.4 | 1.5 | 1.5 | 0.5 | 0.0 | 0.6 | 0.0 | 5.7 | 0.0 | 3.8 | 0.5 | 3.8 |
| | merdae | | | | | | | | | | | | |
| s | Parabacteroides_{—} | 0.3 | 0.5 | 0.3 | 0.1 | 0.0 | 0.1 | 0.1 | 1.5 | 0.0 | 1.7 | 0.3 | 0.8 |
| | unclassified | | | | | | | | | | | | |
| g | Porphyromonas | 0.0 | 0.0 | 0.0 | 0.0 | 0.0 | 0.0 | 0.0 | 0.0 | 0.0 | 0.0 | 0.0 | 0.0 |
| s | Porphyromonas_{—} | 0.0 | 0.0 | 0.0 | 0.0 | 0.0 | 0.0 | 0.0 | 0.0 | 0.0 | 0.0 | 0.0 | 0.0 |
| | asaccharolytica | | | | | | | | | | | | |
| f | Prevotellaceae | 0.0 | 0.0 | 0.0 | 0.0 | 0.0 | 39.2 | 0.0 | 0.0 | 0.0 | 0.1 | 6.1 | 0.0 |
| g | Prevotella | 0.0 | 0.0 | 0.0 | 0.0 | 0.0 | 39.2 | 0.0 | 0.0 | 0.0 | 0.1 | 6.1 | 0.0 |
| s | Prevotella_{—} | 0.0 | 0.0 | 0.0 | 0.0 | 0.0 | 39.2 | 0.0 | 0.0 | 0.0 | 0.0 | 6.1 | 0.0 |
| | copri | | | | | | | | | | | | |
| f | Rikenellaceae | 15.4 | 8.7 | 6.4 | 0.7 | 9.7 | 0.5 | 10.0 | 3.1 | 3.4 | 5.2 | 8.4 | 9.9 |
| g | Alistipes | 15.4 | 8.7 | 6.4 | 0.7 | 9.7 | 0.5 | 10.0 | 3.1 | 3.4 | 5.2 | 8.4 | 9.9 |
| s | Alistipes_{—} | 13.5 | 8.0 | 5.7 | 0.0 | 7.6 | 0.5 | 5.3 | 0.0 | 0.0 | 0.0 | 7.4 | 9.0 |
| | putredinis | | | | | | | | | | | | |
| s | Alistipes_{—} | 1.0 | 0.7 | 0.8 | 0.1 | 2.0 | 0.0 | 4.8 | 2.5 | 0.7 | 0.6 | 1.0 | 0.9 |
| | shahii | | | | | | | | | | | | |
| s | Alistipes_{—} | 0.8 | 0.0 | 0.0 | 0.5 | 0.0 | 0.0 | 0.0 | 0.6 | 2.8 | 4.5 | 0.0 | 0.0 |
| | unclassified | | | | | | | | | | | | |
| p | Firmicutes | 36.5 | 60.7 | 48.0 | 75.1 | 63.5 | 41.6 | 78.6 | 45.9 | 67.9 | 25.6 | 63.8 | 40.9 |
| c | Bacilli | 2.2 | 2.4 | 0.3 | 6.2 | 0.1 | 0.3 | 41.8 | 5.3 | 0.1 | 0.6 | 0.2 | 3.4 |
| o | Lactobacillales | 2.2 | 2.4 | 0.3 | 6.1 | 0.1 | 0.3 | 41.8 | 5.3 | 0.1 | 0.6 | 0.2 | 3.4 |
| f | Enterococcaceae | 0.0 | 0.0 | 0.0 | 0.0 | 0.0 | 0.0 | 1.5 | 0.0 | 0.0 | 0.0 | 0.0 | 0.0 |
| g | Enterococcus | 0.0 | 0.0 | 0.0 | 0.0 | 0.0 | 0.0 | 1.5 | 0.0 | 0.0 | 0.0 | 0.0 | 0.0 |
| s | Enterococcus_{—} | 0.0 | 0.0 | 0.0 | 0.0 | 0.0 | 0.0 | 0.0 | 0.0 | 0.0 | 0.0 | 0.0 | 0.0 |
| | casseliflavus | | | | | | | | | | | | |
| s | Enterococcus_{—} | 0.0 | 0.0 | 0.0 | 0.0 | 0.0 | 0.0 | 1.5 | 0.0 | 0.0 | 0.0 | 0.0 | 0.0 |
| | faecalis | | | | | | | | | | | | |
| s | Enterococcus_{—} | 0.0 | 0.0 | 0.0 | 0.0 | 0.0 | 0.0 | 0.0 | 0.0 | 0.0 | 0.0 | 0.0 | 0.0 |
| | faecium | | | | | | | | | | | | |
| s | Enterococcus_{—} | 0.0 | 0.0 | 0.0 | 0.0 | 0.0 | 0.0 | 0.0 | 0.0 | 0.0 | 0.0 | 0.0 | 0.0 |
| | unclassified | | | | | | | | | | | | |
| f | Lactobacillaceae | 0.0 | 1.7 | 0.0 | 3.5 | 0.1 | 0.0 | 14.3 | 0.8 | 0.0 | 0.0 | 0.0 | 0.1 |
| g | Lactobacillus | 0.0 | 1.7 | 0.0 | 3.5 | 0.1 | 0.0 | 14.3 | 0.8 | 0.0 | 0.0 | 0.0 | 0.1 |
| s | Lactobacillus_{—} | 0.0 | 0.0 | 0.0 | 0.0 | 0.0 | 0.0 | 0.0 | 0.0 | 0.0 | 0.0 | 0.0 | 0.0 |
| | casei | | | | | | | | | | | | |
| s | Lactobacillus_{—} | 0.0 | 0.6 | 0.0 | 0.0 | 0.0 | 0.0 | 1.8 | 0.0 | 0.0 | 0.0 | 0.0 | 0.0 |
| | crispatus | | | | | | | | | | | | |
| s | Lactobacillus_{—} | 0.0 | 0.9 | 0.0 | 0.0 | 0.0 | 0.0 | 11.3 | 0.1 | 0.0 | 0.0 | 0.0 | 0.0 |
| | fermentum | | | | | | | | | | | | |
| s | Lactobacillus_{—} | 0.0 | 0.1 | 0.0 | 0.0 | 0.0 | 0.0 | 1.2 | 0.5 | 0.0 | 0.0 | 0.0 | 0.1 |
| | gasseri | | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 | 0.0 |
| | paracasei | | | | | | | | | | | | |
| s | Lactobacillus_{—} | 0.0 | 0.0 | 0.0 | 0.0 | 0.0 | 0.0 | 0.0 | 0.0 | 0.0 | 0.0 | 0.0 | 0.0 |
| | reuteri | | | | | | | | | | | | |
| s | Lactobacillus_{—} | 0.0 | 0.0 | 0.0 | 3.4 | 0.0 | 0.0 | 0.0 | 0.0 | 0.0 | 0.0 | 0.0 | 0.0 |
| | ruminis | | | | | | | | | | | | |
| s | Lactobacillus_{—} | 0.0 | 0.0 | 0.0 | 0.1 | 0.0 | 0.0 | 0.0 | 0.0 | 0.0 | 0.0 | 0.0 | 0.0 |
| | salivarius | | | | | | | | | | | | |
| f | Streptococcaceae | 2.1 | 0.7 | 0.3 | 2.6 | 0.0 | 0.3 | 26.0 | 4.5 | 0.1 | 0.6 | 0.1 | 3.3 |
| g | Lactococcus | 0.5 | 0.0 | 0.1 | 0.1 | 0.0 | 0.0 | 0.2 | 0.0 | 0.0 | 0.0 | 0.0 | 0.0 |
| s | Lactococcus_{—} | 0.5 | 0.0 | 0.1 | 0.1 | 0.0 | 0.0 | 0.2 | 0.0 | 0.0 | 0.0 | 0.0 | 0.0 |
| | lactis | | | | | | | | | | | | |
| g | Streptococcus | 1.6 | 0.7 | 0.2 | 2.5 | 0.0 | 0.3 | 25.8 | 4.5 | 0.1 | 0.5 | 0.1 | 3.2 |
| s | Streptococcus_{—} | 0.0 | 0.1 | 0.0 | 0.0 | 0.0 | 0.0 | 0.0 | 0.0 | 0.0 | 0.0 | 0.0 | 0.0 |
| | anginosus | | | | | | | | | | | | |
| s | Streptococcus_{—} | 0.0 | 0.0 | 0.0 | 0.0 | 0.0 | 0.0 | 0.0 | 0.0 | 0.0 | 0.0 | 0.0 | 0.0 |
| | infantarius | | | | | | | | | | | | |
| s | Streptococcus_{—} | 0.0 | 0.0 | 0.0 | 0.2 | 0.0 | 0.0 | 0.0 | 0.3 | 0.0 | 0.0 | 0.0 | 0.1 |
| | mutans | | | | | | | | | | | | |
| s | Streptococcus_{—} | 1.1 | 0.3 | 0.1 | 0.8 | 0.0 | 0.1 | 8.4 | 3.1 | 0.0 | 0.1 | 0.0 | 1.7 |
| | parasanguinis | | | | | | | | | | | | |
| s | Streptococcus_{—} | 0.3 | 0.2 | 0.1 | 1.1 | 0.0 | 0.1 | 14.7 | 0.5 | 0.1 | 0.2 | 0.1 | 0.6 |
| | salivarius | | | | | | | | | | | | |
| s | Streptococcus_{—} | 0.1 | 0.0 | 0.0 | 0.0 | 0.0 | 0.1 | 2.3 | 0.2 | 0.0 | 0.1 | 0.0 | 0.0 |
| | thermophilus | | | | | | | | | | | | |
| s | Streptococcus_{—} | 0.1 | 0.1 | 0.0 | 0.2 | 0.0 | 0.0 | 0.3 | 0.3 | 0.0 | 0.0 | 0.0 | 0.1 |
| | vestibularis | | | | | | | | | | | | |
| c | Clostridia | 34.3 | 57.7 | 45.2 | 61.8 | 63.3 | 41.2 | 36.2 | 36.9 | 64.1 | 24.3 | 63.6 | 36.1 |
| o | Clostridiales | 34.3 | 57.7 | 45.2 | 61.8 | 63.3 | 41.2 | 36.2 | 36.9 | 64.1 | 24.3 | 63.6 | 36.1 |
| f | Clostridiaceae | 1.2 | 6.8 | 2.5 | 1.8 | 0.5 | 0.2 | 6.3 | 4.7 | 3.2 | 9.3 | 1.0 | 3.7 |
| g | Clostridium | 1.2 | 6.8 | 2.5 | 1.8 | 0.5 | 0.1 | 6.3 | 4.7 | 3.2 | 9.3 | 1.0 | 3.7 |
| s | Clostridium_{—} | 0.0 | 0.1 | 0.3 | 0.0 | 0.0 | 0.0 | 0.1 | 2.3 | 0.1 | 0.9 | 0.1 | 0.1 |
| | asparagiforme | | | | | | | | | | | | |
| s | Clostridium_{—} | 0.0 | 2.2 | 0.0 | 1.5 | 0.4 | 0.1 | 0.0 | 0.0 | 0.0 | 0.1 | 0.7 | 1.5 |
| | bartlettii | | | | | | | | | | | | |
| s | Clostridium_{—} | 0.1 | 0.1 | 0.9 | 0.0 | 0.0 | 0.0 | 0.2 | 0.6 | 0.6 | 3.1 | 0.0 | 0.5 |
| | bolteae | | | | | | | | | | | | |
| s | Clostridium_{—} | 0.0 | 0.1 | 0.0 | 0.0 | 0.0 | 0.0 | 0.0 | 0.0 | 0.1 | 0.0 | 0.1 | 0.1 |
| | cf | | | | | | | | | | | | |
| s | Clostridium_{—} | 0.2 | 0.0 | 0.2 | 0.0 | 0.0 | 0.0 | 0.9 | 0.1 | 0.0 | 1.4 | 0.0 | 0.0 |
| | hathewayi | | | | | | | | | | | | |
| s | Clostridium_{—} | 0.0 | 0.0 | 0.0 | 0.0 | 0.0 | 0.0 | 0.0 | 0.0 | 0.0 | 0.0 | 0.0 | 0.0 |
| | hiranonis | | | | | | | | | | | | |
| s | Clostridium_{—} | 0.5 | 1.2 | 0.1 | 0.3 | 0.0 | 0.0 | 0.3 | 0.0 | 1.6 | 0.2 | 0.0 | 0.1 |
| | leptum | | | | | | | | | | | | |
| s | Clostridium_{—} | 0.0 | 2.9 | 0.0 | 0.0 | 0.0 | 0.0 | 0.0 | 0.2 | 0.0 | 2.1 | 0.0 | 0.9 |
| | nexile | | | | | | | | | | | | |
| s | Clostridium_{—} | 0.0 | 0.0 | 0.0 | 0.0 | 0.0 | 0.0 | 0.0 | 0.1 | 0.0 | 0.2 | 0.0 | 0.0 |
| | perfringens | | | | | | | | | | | | |
| s | Clostridium_{—} | 0.1 | 0.1 | 0.9 | 0.0 | 0.0 | 0.0 | 4.4 | 1.0 | 0.4 | 0.5 | 0.0 | 0.5 |
| | scindens | | | | | | | | | | | | |
| s | Clostridium_{—} | 0.0 | 0.0 | 0.1 | 0.0 | 0.0 | 0.0 | 0.3 | 0.1 | 0.3 | 0.7 | 0.1 | 0.1 |
| | symbiosum | | | | | | | | | | | | |
| f | Clostridiales_{—} | 0.0 | 0.0 | 0.0 | 0.0 | 0.0 | 0.0 | 0.1 | 0.0 | 0.0 | 0.1 | 0.0 | 0.0 |
| | Family_XI_{—} | | | | | | | | | | | | |
| | Incertae_{—}Sedis | | | | | | | | | | | | |
| g | Anaerococcus | 0.0 | 0.0 | 0.0 | 0.0 | 0.0 | 0.0 | 0.0 | 0.0 | 0.0 | 0.0 | 0.0 | 0.0 |
| s | Anaerococcus_{—} | 0.0 | 0.0 | 0.0 | 0.0 | 0.0 | 0.0 | 0.0 | 0.0 | 0.0 | 0.0 | 0.0 | 0.0 |
| | unclassified | | | | | | | | | | | | |
| g | Clostridiales_{—} | 0.0 | 0.0 | 0.0 | 0.0 | 0.0 | 0.0 | 0.1 | 0.0 | 0.0 | 0.0 | 0.0 | 0.0 |
| | Family_XI_{—} | | | | | | | | | | | | |
| | Incertae_{—}Sedis_{—} | | | | | | | | | | | | |
| | unclassified | | | | | | | | | | | | |
| g | Finegoldia | 0.0 | 0.0 | 0.0 | 0.0 | 0.0 | 0.0 | 0.0 | 0.0 | 0.0 | 0.0 | 0.0 | 0.0 |
| s | Finegoldia_{—} | 0.0 | 0.0 | 0.0 | 0.0 | 0.0 | 0.0 | 0.0 | 0.0 | 0.0 | 0.0 | 0.0 | 0.0 |
| | magna | | | | | | | | | | | | |
| g | Parvimonas | 0.0 | 0.0 | 0.0 | 0.0 | 0.0 | 0.0 | 0.0 | 0.0 | 0.0 | 0.0 | 0.0 | 0.0 |
| s | Parvimonas_{—} | 0.0 | 0.0 | 0.0 | 0.0 | 0.0 | 0.0 | 0.0 | 0.0 | 0.0 | 0.0 | 0.0 | 0.0 |
| | micros | | | | | | | | | | | | |
| g | Peptoniphilus | 0.0 | 0.0 | 0.0 | 0.0 | 0.0 | 0.0 | 0.0 | 0.0 | 0.0 | 0.0 | 0.0 | 0.0 |
| s | Peptoniphilus_{—} | 0.0 | 0.0 | 0.0 | 0.0 | 0.0 | 0.0 | 0.0 | 0.0 | 0.0 | 0.0 | 0.0 | 0.0 |
| | harei | | | | | | | | | | | | |
| s | Peptoniphilus_{—} | 0.0 | 0.0 | 0.0 | 0.0 | 0.0 | 0.0 | 0.0 | 0.0 | 0.0 | 0.0 | 0.0 | 0.0 |
| | unclassified | | | | | | | | | | | | |
| f | Clostridiales_{—} | 2.9 | 0.1 | 0.9 | 0.1 | 0.8 | 0.1 | 9.7 | 0.5 | 1.6 | 1.6 | 0.1 | 0.4 |
| | uncl | | | | | | | | | | | | |
| g | Blautia | 2.9 | 0.1 | 0.9 | 0.1 | 0.8 | 0.1 | 9.7 | 0.5 | 1.6 | 1.6 | 0.1 | 0.4 |
| s | Blautia_{—} | 0.0 | 0.0 | 0.0 | 0.0 | 0.0 | 0.0 | 6.3 | 0.3 | 1.1 | 0.8 | 0.0 | 0.0 |
| | hansenii | | | | | | | | | | | | |
| s | Blautia_{—} | 2.9 | 0.1 | 0.0 | 0.0 | 0.0 | 0.0 | 3.4 | 0.0 | 0.4 | 0.8 | 0.1 | 0.4 |
| | hydrogenotrophica | | | | | | | | | | | | |
| s | Blautia_{—} | 0.0 | 0.0 | 0.8 | 0.1 | 0.8 | 0.1 | 0.0 | 0.2 | 0.0 | 0.0 | 0.1 | 0.0 |
| | unclassified | | | | | | | | | | | | |
| f | Eubacteriaceae | 5.6 | 10.6 | 0.7 | 26.4 | 37.6 | 22.7 | 2.7 | 6.6 | 1.5 | 11.0 | 20.1 | 9.1 |
| g | Eubacterium | 5.6 | 10.6 | 0.7 | 26.4 | 37.6 | 22.7 | 2.5 | 6.6 | 1.5 | 11.0 | 20.1 | 9.1 |
| s | Eubacterium_{—} | 1.9 | 0.4 | 0.0 | 0.0 | 0.8 | 7.2 | 0.0 | 0.0 | 0.6 | 0.0 | 1.1 | 0.0 |
| | eligens | | | | | | | | | | | | |
| s | Eubacterium_{—} | 0.2 | 0.3 | 0.7 | 1.3 | 1.6 | 1.7 | 0.0 | 3.9 | 0.4 | 0.0 | 1.0 | 0.0 |
| | hallii | | | | | | | | | | | | |
| s | Eubacterium_{—} | 0.0 | 0.1 | 0.0 | 0.0 | 0.0 | 0.0 | 2.4 | 0.1 | 0.0 | 0.0 | 0.0 | 0.0 |
| | limosum | | | | | | | | | | | | |
| s | Eubacterium_{—} | 0.0 | 8.0 | 0.0 | 25.1 | 6.4 | 13.5 | 0.1 | 2.6 | 0.0 | 11.0 | 16.7 | 7.9 |
| | rectale | | | | | | | | | | | | |
| s | Eubacterium_{—} | 3.4 | 1.9 | 0.0 | 0.0 | 28.5 | 0.2 | 0.1 | 0.0 | 0.4 | 0.0 | 1.2 | 0.0 |
| | siraeum | | | | | | | | | | | | |
| s | Eubacterium_{—} | 0.0 | 0.0 | 0.0 | 0.0 | 0.3 | 0.0 | 0.0 | 0.0 | 0.0 | 0.0 | 0.0 | 1.3 |
| | ventriosum | | | | | | | | | | | | |
| g | Pseudoramibacter | 0.0 | 0.0 | 0.0 | 0.0 | 0.0 | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 | 0.0 | 0.0 | 0.0 | 0.0 | 0.0 |
| | alactolyticus | | | | | | | | | | | | |
| f | Lachnospiraceae | 10.2 | 2.5 | 3.8 | 9.6 | 6.1 | 6.7 | 0.2 | 10.1 | 17.1 | 0.9 | 4.1 | 10.5 |
| g | Anaerostipes | 0.0 | 0.0 | 0.0 | 0.0 | 0.0 | 0.0 | 0.1 | 0.0 | 0.0 | 0.9 | 0.0 | 0.0 |
| s | Anaerostipes_{—} | 0.0 | 0.0 | 0.0 | 0.0 | 0.0 | 0.0 | 0.1 | 0.0 | 0.0 | 0.9 | 0.0 | 0.0 |
| | caccae | | | | | | | | | | | | |
| g | Butyrivibrio | 0.0 | 0.0 | 0.0 | 0.0 | 0.0 | 0.0 | 0.0 | 0.0 | 0.0 | 0.0 | 0.0 | 0.0 |
| s | Butyrivibrio_{—} | 0.0 | 0.0 | 0.0 | 0.0 | 0.0 | 0.0 | 0.0 | 0.0 | 0.0 | 0.0 | 0.0 | 0.0 |
| | crossotus | | | | | | | | | | | | |
| g | Coprococcus | 9.1 | 1.7 | 0.2 | 3.9 | 1.7 | 1.2 | 0.0 | 0.6 | 0.0 | 0.0 | 0.6 | 1.3 |
| s | Coprococcus_{—} | 0.4 | 0.6 | 0.2 | 0.3 | 0.9 | 0.0 | 0.0 | 0.2 | 0.0 | 0.0 | 0.6 | 0.6 |
| | catus | | | | | | | | | | | | |
| s | Coprococcus_{—} | 8.7 | 1.1 | 0.0 | 3.6 | 0.7 | 1.2 | 0.0 | 0.4 | 0.0 | 0.0 | 0.0 | 0.7 |
| | comes | | | | | | | | | | | | |
| g | Dorea | 1.1 | 0.8 | 3.6 | 3.3 | 1.6 | 1.2 | 0.0 | 0.9 | 0.0 | 0.0 | 1.5 | 1.1 |
| s | Dorea_{—} | 0.0 | 0.2 | 3.6 | 0.5 | 0.7 | 0.6 | 0.0 | 0.7 | 0.0 | 0.0 | 1.1 | 0.0 |
| | formicigenerans | | | | | | | | | | | | |
| s | Dorea_{—} | 1.1 | 0.6 | 0.0 | 2.8 | 0.9 | 0.6 | 0.0 | 0.2 | 0.0 | 0.0 | 0.4 | 1.1 |
| | longicatena | | | | | | | | | | | | |
| g | Roseburia | 0.0 | 0.0 | 0.0 | 2.4 | 2.8 | 4.2 | 0.0 | 8.5 | 17.1 | 0.0 | 2.0 | 8.2 |
| s | Roseburia_{—} | 0.0 | 0.0 | 0.0 | 0.8 | 2.7 | 2.2 | 0.0 | 0.0 | 13.9 | 0.0 | 0.4 | 4.0 |
| | intestinalis | | | | | | | | | | | | |
| s | Roseburia_{—} | 0.0 | 0.0 | 0.0 | 1.6 | 0.1 | 2.0 | 0.0 | 8.5 | 3.1 | 0.0 | 1.5 | 4.2 |
| | inulinivorans | | | | | | | | | | | | |
| f | Ruminococcaceae | 14.3 | 37.7 | 37.2 | 23.8 | 18.3 | 11.6 | 17.2 | 15.0 | 40.6 | 1.4 | 38.3 | 12.3 |
| g | Anaerotruncus | 0.0 | 0.0 | 0.1 | 0.0 | 0.0 | 0.0 | 0.4 | 0.4 | 0.0 | 0.0 | 0.1 | 0.0 |
| s | Anaerotruncus_{—} | 0.0 | 0.0 | 0.1 | 0.0 | 0.0 | 0.0 | 0.4 | 0.4 | 0.0 | 0.0 | 0.1 | 0.0 |
| | colihominis | | | | | | | | | | | | |
| g | Faecalibacterium | 0.1 | 5.7 | 6.6 | 20.6 | 13.3 | 10.8 | 0.0 | 6.8 | 1.6 | 0.0 | 21.1 | 2.2 |
| s | Faecalibacterium_{—} | 0.1 | 3.1 | 0.0 | 4.0 | 1.2 | 4.9 | 0.0 | 0.6 | 0.0 | 0.0 | 1.8 | 1.7 |
| | cf | | | | | | | | | | | | |
| s | Faecalibacterium_{—} | 0.0 | 1.9 | 1.1 | 16.5 | 11.3 | 5.0 | 0.0 | 5.5 | 1.3 | 0.0 | 19.3 | 0.2 |
| | prausnitzii | | | | | | | | | | | | |
| s | Faecalibacterium_{—} | 0.0 | 0.7 | 5.4 | 0.0 | 0.8 | 0.9 | 0.0 | 0.6 | 0.3 | 0.0 | 0.0 | 0.3 |
| | unclassified | | | | | | | | | | | | |
| s | Ruminococcus_{—}bromii | 0.0 | 13.6 | 0.0 | 0.0 | 0.0 | 0.0 | 0.0 | 3.2 | 28.7 | 0.0 | 12.3 | 0.0 |
| s | Ruminococcus_{—} | 0.0 | 10.2 | 0.9 | 0.0 | 0.0 | 0.0 | 0.2 | 0.0 | 0.5 | 1.3 | 0.0 | 0.8 |
| | gnavus | | | | | | | | | | | | |
| s | Ruminococcus_{—} | 0.0 | 0.0 | 0.0 | 0.0 | 0.8 | 0.0 | 0.0 | 0.0 | 0.0 | 0.0 | 2.5 | 0.0 |
| | lactaris | | | | | | | | | | | | |
| s | Ruminococcus_{—} | 14.0 | 0.4 | 10.6 | 1.0 | 3.9 | 0.7 | 12.5 | 0.5 | 0.0 | 0.0 | 0.8 | 0.4 |
| | obeum | | | | | | | | | | | | |
| s | Ruminococcus_{—} | 0.0 | 7.7 | 19.0 | 2.2 | 0.1 | 0.0 | 4.1 | 4.1 | 9.7 | 0.0 | 1.6 | 8.9 |
| | torques | | | | | | | | | | | | |
| c | Erysipelotrichi | 0.1 | 0.4 | 2.6 | 0.1 | 0.1 | 0.0 | 0.6 | 0.2 | 0.2 | 0.2 | 0.0 | 0.0 |
| o | Erysipelotrichales | 0.1 | 0.4 | 2.6 | 0.1 | 0.1 | 0.0 | 0.6 | 0.2 | 0.2 | 0.2 | 0.0 | 0.0 |
| f | Erysipelotrichaceae | 0.1 | 0.4 | 2.6 | 0.1 | 0.1 | 0.0 | 0.6 | 0.2 | 0.2 | 0.2 | 0.0 | 0.0 |
| g | Catenibacterium | 0.0 | 0.0 | 0.0 | 0.0 | 0.0 | 0.0 | 0.0 | 0.0 | 0.0 | 0.0 | 0.0 | 0.0 |
| s | Catenibacterium_{—} | 0.0 | 0.0 | 0.0 | 0.0 | 0.0 | 0.0 | 0.0 | 0.0 | 0.0 | 0.0 | 0.0 | 0.0 |
| | mitsuokai | | | | | | | | | | | | |
| g | Coprobacillus | 0.1 | 0.4 | 2.4 | 0.0 | 0.0 | 0.0 | 0.6 | 0.0 | 0.1 | 0.2 | 0.0 | 0.0 |
| s | Coprobacillus_{—} | 0.1 | 0.4 | 2.4 | 0.0 | 0.0 | 0.0 | 0.6 | 0.0 | 0.1 | 0.2 | 0.0 | 0.0 |
| | bacterium | | | | | | | | | | | | |
| g | Holdemania | 0.0 | 0.0 | 0.2 | 0.1 | 0.0 | 0.0 | 0.0 | 0.2 | 0.1 | 0.0 | 0.0 | 0.0 |
| s | Holdemania_{—} | 0.0 | 0.0 | 0.2 | 0.1 | 0.0 | 0.0 | 0.0 | 0.2 | 0.1 | 0.0 | 0.0 | 0.0 |
| | filiformis | | | | | | | | | | | | |
| c | Negativicutes | 0.0 | 0.1 | 0.1 | 7.0 | 0.0 | 0.1 | 0.0 | 3.5 | 3.5 | 0.5 | 0.0 | 1.4 |
| o | Selenomonadales | 0.0 | 0.1 | 0.1 | 7.0 | 0.0 | 0.1 | 0.0 | 3.5 | 3.5 | 0.5 | 0.0 | 1.4 |
| f | Acidaminococcaceae | 0.0 | 0.1 | 0.0 | 0.4 | 0.0 | 0.0 | 0.0 | 0.0 | 0.0 | 0.5 | 0.0 | 0.0 |
| g | Acidaminococcaceae_{—} | 0.0 | 0.1 | 0.0 | 0.4 | 0.0 | 0.0 | 0.0 | 0.0 | 0.0 | 0.5 | 0.0 | 0.0 |
| | unclassified | | | | | | | | | | | | |
| g | Phascolarctobacterium | 0.0 | 0.0 | 0.0 | 0.0 | 0.0 | 0.0 | 0.0 | 0.0 | 0.0 | 0.0 | 0.0 | 0.0 |
| s | Phascolarctobacterium_{—} | 0.0 | 0.0 | 0.0 | 0.0 | 0.0 | 0.0 | 0.0 | 0.0 | 0.0 | 0.0 | 0.0 | 0.0 |
| | unclassified | | | | | | | | | | | | |
| f | Veillonellaceae | 0.0 | 0.0 | 0.1 | 6.6 | 0.0 | 0.1 | 0.0 | 3.5 | 3.5 | 0.0 | 0.0 | 1.4 |
| g | Dialister | 0.0 | 0.0 | 0.0 | 6.6 | 0.0 | 0.0 | 0.0 | 3.0 | 3.5 | 0.0 | 0.0 | 0.0 |
| s | Dialister_{—} | 0.0 | 0.0 | 0.0 | 6.6 | 0.0 | 0.0 | 0.0 | 3.0 | 3.5 | 0.0 | 0.0 | 0.0 |
| | invisus | | | | | | | | | | | | |
| g | Megamonas | 0.0 | 0.0 | 0.0 | 0.0 | 0.0 | 0.0 | 0.0 | 0.0 | 0.0 | 0.0 | 0.0 | 0.0 |
| s | Megamonas_{—} | 0.0 | 0.0 | 0.0 | 0.0 | 0.0 | 0.0 | 0.0 | 0.0 | 0.0 | 0.0 | 0.0 | 0.0 |
| | hypermegale | | | | | | | | | | | | |
| g | Veillonella | 0.0 | 0.0 | 0.0 | 0.0 | 0.0 | 0.1 | 0.0 | 0.5 | 0.0 | 0.0 | 0.0 | 1.3 |
| s | Veillonella_{—} | 0.0 | 0.0 | 0.0 | 0.0 | 0.0 | 0.0 | 0.0 | 0.3 | 0.0 | 0.0 | 0.0 | 0.0 |
| | atypica | | | | | | | | | | | | |
| s | Veillonella_{—} | 0.0 | 0.0 | 0.0 | 0.0 | 0.0 | 0.0 | 0.0 | 0.2 | 0.0 | 0.0 | 0.0 | 0.4 |
| | parvula | | | | | | | | | | | | |
| s | Veillonella_{—} | 0.0 | 0.0 | 0.0 | 0.0 | 0.0 | 0.0 | 0.0 | 0.0 | 0.0 | 0.0 | 0.0 | 0.6 |
| | unclassified | | | | | | | | | | | | |
| p | Fusobacteria | 0.0 | 0.0 | 0.0 | 0.0 | 0.0 | 0.0 | 0.0 | 0.0 | 0.0 | 0.0 | 0.0 | 0.0 |
| c | Fusobacteria | 0.0 | 0.0 | 0.0 | 0.0 | 0.0 | 0.0 | 0.0 | 0.0 | 0.0 | 0.0 | 0.0 | 0.0 |
| o | Fusobacteriales | 0.0 | 0.0 | 0.0 | 0.0 | 0.0 | 0.0 | 0.0 | 0.0 | 0.0 | 0.0 | 0.0 | 0.0 |
| f | Fusobacteriaceae | 0.0 | 0.0 | 0.0 | 0.0 | 0.0 | 0.0 | 0.0 | 0.0 | 0.0 | 0.0 | 0.0 | 0.0 |
| g | Fusobacterium | 0.0 | 0.0 | 0.0 | 0.0 | 0.0 | 0.0 | 0.0 | 0.0 | 0.0 | 0.0 | 0.0 | 0.0 |
| s | Fusobacterium_{—} | 0.0 | 0.0 | 0.0 | 0.0 | 0.0 | 0.0 | 0.0 | 0.0 | 0.0 | 0.0 | 0.0 | 0.0 |
| | ulcerans | | | | | | | | | | | | |
| p | Proteobacteria | 2.3 | 4.9 | 0.8 | 0.7 | 0.3 | 1.5 | 0.0 | 2.1 | 0.5 | 5.3 | 3.5 | 0.5 |
| c | Betaproteobacteria | 0.0 | 0.0 | 0.0 | 0.0 | 0.0 | 1.4 | 0.0 | 0.0 | 0.0 | 0.0 | 0.0 | 0.0 |
| o | Burkholderiales | 0.0 | 0.0 | 0.0 | 0.0 | 0.0 | 1.4 | 0.0 | 0.0 | 0.0 | 0.0 | 0.0 | 0.0 |
| f | Sutterellaceae | 0.0 | 0.0 | 0.0 | 0.0 | 0.0 | 1.4 | 0.0 | 0.0 | 0.0 | 0.0 | 0.0 | 0.0 |
| g | Sutterella | 0.0 | 0.0 | 0.0 | 0.0 | 0.0 | 1.4 | 0.0 | 0.0 | 0.0 | 0.0 | 0.0 | 0.0 |
| s | Sutterella_{—} | 0.0 | 0.0 | 0.0 | 0.0 | 0.0 | 1.4 | 0.0 | 0.0 | 0.0 | 0.0 | 0.0 | 0.0 |
| | wadsworthensis | | | | | | | | | | | | |
| c | Deltaproteobacteria | 1.6 | 0.2 | 0.8 | 0.0 | 0.2 | 0.1 | 0.0 | 1.9 | 0.0 | 0.0 | 1.4 | 0.0 |
| o | Desulfovibrionales | 1.6 | 0.2 | 0.8 | 0.0 | 0.2 | 0.1 | 0.0 | 1.9 | 0.0 | 0.0 | 1.4 | 0.0 |
| f | Desulfovibrionaceae | 1.6 | 0.2 | 0.8 | 0.0 | 0.2 | 0.1 | 0.0 | 1.9 | 0.0 | 0.0 | 1.4 | 0.0 |
| g | Bilophila | 1.4 | 0.1 | 0.8 | 0.0 | 0.2 | 0.1 | 0.0 | 1.9 | 0.0 | 0.0 | 0.5 | 0.0 |
| s | Bilophila_{—} | 1.4 | 0.1 | 0.8 | 0.0 | 0.2 | 0.1 | 0.0 | 1.9 | 0.0 | 0.0 | 0.5 | 0.0 |
| | wadsworthia | | | | | | | | | | | | |
| g | Desulfovibrio | 0.2 | 0.1 | 0.0 | 0.0 | 0.0 | 0.0 | 0.0 | 0.1 | 0.0 | 0.0 | 0.9 | 0.0 |
| s | Desulfovibrio_{—} | 0.0 | 0.0 | 0.0 | 0.0 | 0.0 | 0.0 | 0.0 | 0.0 | 0.0 | 0.0 | 0.7 | 0.0 |
| | piger | | | | | | | | | | | | |
| c | Gammaproteobacteria | 0.7 | 4.7 | 0.0 | 0.7 | 0.1 | 0.1 | 0.0 | 0.2 | 0.5 | 5.3 | 2.1 | 0.5 |
| o | Enterobacteriales | 0.7 | 4.7 | 0.0 | 0.5 | 0.0 | 0.0 | 0.0 | 0.2 | 0.5 | 5.3 | 2.1 | 0.3 |
| f | Enterobacteriaceae | 0.7 | 4.7 | 0.0 | 0.5 | 0.0 | 0.0 | 0.0 | 0.2 | 0.5 | 5.3 | 2.1 | 0.3 |
| g | Enterobacter | 0.0 | 0.0 | 0.0 | 0.1 | 0.0 | 0.0 | 0.0 | 0.1 | 0.0 | 0.0 | 0.0 | 0.0 |
| s | Enterobacter_{—} | 0.0 | 0.0 | 0.0 | 0.1 | 0.0 | 0.0 | 0.0 | 0.1 | 0.0 | 0.0 | 0.0 | 0.0 |
| | cloacae | | | | | | | | | | | | |
| g | Escherichia | 0.7 | 4.3 | 0.0 | 0.3 | 0.0 | 0.0 | 0.0 | 0.0 | 0.5 | 5.1 | 2.1 | 0.2 |
| s | Escherichia_{—} | 0.7 | 4.3 | 0.0 | 0.3 | 0.0 | 0.0 | 0.0 | 0.0 | 0.5 | 3.5 | 2.1 | 0.2 |
| | coli | | | | | | | | | | | | |
| s | Escherichia_{—} | 0.0 | 0.0 | 0.0 | 0.0 | 0.0 | 0.0 | 0.0 | 0.0 | 0.0 | 1.6 | 0.0 | 0.0 |
| | unclassified | | | | | | | | | | | | |
| g | Klebsiella | 0.0 | 0.4 | 0.0 | 0.1 | 0.0 | 0.0 | 0.0 | 0.0 | 0.0 | 0.2 | 0.0 | 0.0 |
| s | Klebsiella_{—} | 0.0 | 0.3 | 0.0 | 0.1 | 0.0 | 0.0 | 0.0 | 0.0 | 0.0 | 0.0 | 0.0 | 0.0 |
| | pneumoniae | | | | | | | | | | | | |
| s | Klebsiella_{—} | 0.0 | 0.1 | 0.0 | 0.0 | 0.0 | 0.0 | 0.0 | 0.0 | 0.0 | 0.2 | 0.0 | 0.0 |
| | unclassified | | | | | | | | | | | | |
| p | Verrucomicrobia | 12.5 | 0.0 | 0.0 | 0.0 | 0.1 | 0.0 | 0.0 | 0.0 | 5.1 | 6.6 | 3.4 | 0.0 |
| c | Verrucomicrobiae | 12.5 | 0.0 | 0.0 | 0.0 | 0.1 | 0.0 | 0.0 | 0.0 | 5.1 | 6.6 | 3.4 | 0.0 |
| o | Verrucomicrobiales | 12.5 | 0.0 | 0.0 | 0.0 | 0.1 | 0.0 | 0.0 | 0.0 | 5.1 | 6.6 | 3.4 | 0.0 |
| f | Verrucomicrobiaceae | 12.5 | 0.0 | 0.0 | 0.0 | 0.1 | 0.0 | 0.0 | 0.0 | 5.1 | 6.6 | 3.4 | 0.0 |
| g | Akkermansia | 12.5 | 0.0 | 0.0 | 0.0 | 0.1 | 0.0 | 0.0 | 0.0 | 5.1 | 6.6 | 3.4 | 0.0 |
| s | Akkermansia_{—} | 12.5 | 0.0 | 0.0 | 0.0 | 0.1 | 0.0 | 0.0 | 0.0 | 5.1 | 6.6 | 3.4 | 0.0 |
| | muciniphila | | | | | | | | | | | | |

| Rank | Name | P65 | P66 | P67 | P68 | P69 | P70 | P71 |
|---|---|---|---|---|---|---|---|---|
| k | Archaea | 0.0 | 5.1 | 0.0 | 0.0 | 0.0 | 0.0 | 0.0 |
| p | Euryarchaeota | 0.0 | 5.1 | 0.0 | 0.0 | 0.0 | 0.0 | 0.0 |
| c | Methanobacteria | 0.0 | 5.1 | 0.0 | 0.0 | 0.0 | 0.0 | 0.0 |
| o | Methanobacteriales | 0.0 | 5.1 | 0.0 | 0.0 | 0.0 | 0.0 | 0.0 |
| f | Methanobacteriaceae | 0.0 | 5.1 | 0.0 | 0.0 | 0.0 | 0.0 | 0.0 |
| g | Methanobrevibacter | 0.0 | 5.1 | 0.0 | 0.0 | 0.0 | 0.0 | 0.0 |
| s | Methanobrevibacter_{—} | 0.0 | 5.1 | 0.0 | 0.0 | 0.0 | 0.0 | 0.0 |
| | smithii | | | | | | | |
| k | Bacteria | 100.0 | 94.9 | 100.0 | 100.0 | 100.0 | 100.0 | 100.0 |
| p | Actinobacteria | 10.6 | 6.9 | 1.6 | 1.7 | 1.6 | 1.2 | 6.5 |
| c | Actinobacteria | 10.6 | 6.9 | 1.6 | 1.7 | 1.6 | 1.2 | 6.5 |
| o | Actinomycetales | 0.0 | 0.0 | 0.0 | 0.0 | 0.0 | 0.0 | 0.1 |
| f | Corynebacteriaceae | 0.0 | 0.0 | 0.0 | 0.0 | 0.0 | 0.0 | 0.0 |
| g | Corynebacterium | 0.0 | 0.0 | 0.0 | 0.0 | 0.0 | 0.0 | 0.0 |
| s | corynebacterium_{—} | 0.0 | 0.0 | 0.0 | 0.0 | 0.0 | 0.0 | 0.0 |
| | amycolatum | | | | | | | |
| s | corynebacterium_{—} | 0.0 | 0.0 | 0.0 | 0.0 | 0.0 | 0.0 | 0.0 |
| | striatum | | | | | | | |
| o | Bifidobacteriales | 0.5 | 1.1 | 0.0 | 0.0 | 0.2 | 0.0 | 2.7 |
| f | Bifidobacteriaceae | 0.5 | 1.1 | 0.0 | 0.0 | 0.2 | 0.0 | 2.7 |
| g | Bifidobacterium | 0.5 | 1.1 | 0.0 | 0.0 | 0.2 | 0.0 | 2.7 |
| s | Bifidobacterium_{—} | 0.0 | 0.0 | 0.0 | 0.0 | 0.0 | 0.0 | 0.8 |
| | adolescentis | | | | | | | |
| s | Bifidobacterium_{—} | 0.0 | 0.0 | 0.0 | 0.0 | 0.2 | 0.0 | 0.0 |
| | animalis | | | | | | | |
| s | Bifidobacterium_{—} | 0.0 | 0.0 | 0.0 | 0.0 | 0.0 | 0.0 | 0.0 |
| | bifidum | | | | | | | |
| s | Bifidobacterium_{—} | 0.0 | 0.0 | 0.0 | 0.0 | 0.0 | 0.0 | 0.0 |
| | breve | | | | | | | |
| s | bifidobacterium_{—} | 0.0 | 0.0 | 0.0 | 0.0 | 0.0 | 0.0 | 0.0 |
| | catenulatum | | | | | | | |
| s | bifidobacterium_{—} | 0.0 | 0.0 | 0.0 | 0.0 | 0.0 | 0.0 | 0.0 |
| | dentium | | | | | | | |
| s | bifidobacterium_{—} | 0.0 | 1.1 | 0.0 | 0.0 | 0.0 | 0.0 | 1.8 |
| | longum | | | | | | | |
| s | bifidobacterium_{—} | 0.0 | 0.0 | 0.0 | 0.0 | 0.0 | 0.0 | 0.1 |
| | pseudocatenulatum | | | | | | | |
| o | coriobacteriales | 10.0 | 5.8 | 1.6 | 1.6 | 1.5 | 1.1 | 3.7 |
| f | coriobacteriaceae | 10.0 | 5.8 | 1.6 | 1.6 | 1.5 | 1.1 | 3.7 |
| g | collinsella | 10.0 | 5.4 | 0.0 | 0.8 | 0.0 | 0.0 | 3.5 |
| s | collinsella_{—} | 0.0 | 5.4 | 0.0 | 0.8 | 0.0 | 0.0 | 3.5 |
| | aerofaciens | | | | | | | |
| s | collinsella_{—} | 9.9 | 0.0 | 0.0 | 0.0 | 0.0 | 0.0 | 0.0 |
| | intestinalis | | | | | | | |
| g | eggerthella | 0.0 | 0.3 | 1.0 | 0.3 | 1.3 | 1.1 | 0.1 |
| s | eggerthella_{—} | 0.0 | 0.3 | 1.0 | 0.3 | 1.3 | 1.1 | 0.1 |
| | lenta | | | | | | | |
| g | gordonibacter | 0.0 | 0.1 | 0.6 | 0.5 | 0.1 | 0.0 | 0.1 |
| s | gordonibacter_{—} | 0.0 | 0.1 | 0.6 | 0.5 | 0.1 | 0.0 | 0.1 |
| | pamelaeae | | | | | | | |
| p | Bacteroidetes | 51.3 | 19.5 | 41.7 | 36.2 | 33.2 | 48.5 | 30.0 |
| c | Bacteroidia | 51.3 | 19.5 | 41.7 | 36.2 | 33.2 | 48.5 | 30.0 |
| o | Bacteroidales | 51.3 | 19.5 | 41.7 | 36.2 | 33.2 | 48.5 | 30.0 |
| f | Bacteroidaceae | 48.0 | 18.1 | 26.5 | 26.5 | 27.6 | 45.9 | 20.3 |
| g | Bacteroides | 48.0 | 18.1 | 26.5 | 26.5 | 27.6 | 45.9 | 20.3 |
| s | Bacteroides_{—} | 0.0 | 0.0 | 3.1 | 0.8 | 0.0 | 0.0 | 0.4 |
| | caccae | | | | | | | |
| s | Bacteroides_{—} | 3.4 | 0.0 | 0.0 | 0.0 | 0.0 | 0.0 | 0.2 |
| | cellulosilyticus | | | | | | | |
| s | Bacteroides_{—} | 0.0 | 0.0 | 0.0 | 0.0 | 0.0 | 0.0 | 0.0 |
| | coprocola | | | | | | | |
| s | Bacteroides_{—} | 0.0 | 0.0 | 0.0 | 0.0 | 0.0 | 0.0 | 0.0 |
| | coprophilus | | | | | | | |
| s | Bacteroides_{—} | 0.0 | 0.0 | 0.0 | 0.0 | 0.0 | 0.0 | 0.2 |
| | eggerthi | | | | | | | |
| s | Bacteroides_{—} | 3.8 | 0.0 | 0.2 | 0.0 | 0.0 | 0.0 | 2.1 |
| | finegoldii | | | | | | | |
| s | Bacteroides_{—} | 0.0 | 0.0 | 0.0 | 0.0 | 0.0 | 0.1 | 0.0 |
| | fragilis | | | | | | | |
| s | Bacteroides_{—} | 0.0 | 0.0 | 0.0 | 6.2 | 10.1 | 0.0 | 0.0 |
| | intestinalis | | | | | | | |
| s | Bacteroides_{—} | 0.0 | 1.3 | 0.5 | 1.1 | 0.0 | 1.3 | 0.1 |
| | ovatus | | | | | | | |
| s | Bacteroides_{—} | 0.0 | 0.0 | 0.0 | 0.0 | 0.0 | 0.0 | 0.0 |
| | pectinophilus | | | | | | | |
| s | Bacteroides_{—} | 8.2 | 0.0 | 7.5 | 0.0 | 0.0 | 0.0 | 0.0 |
| | plebeius | | | | | | | |
| s | Bacteroides_{—} | 5.3 | 0.0 | 3.6 | 1.0 | 6.6 | 9.2 | 12.2 |
| | stercoris | | | | | | | |
| s | Bacteroides_{—}thetaiotaomicron | 6.5 | 0.0 | 0.0 | 0.3 | 4.7 | 8.4 | 0.3 |
| s | Bacteroides_{—} | 5.4 | 11.0 | 6.5 | 0.0 | 6.2 | 19.3 | 4.6 |
| | unclassified | | | | | | | |
| s | Bacteroides_{—} | 4.8 | 0.1 | 1.0 | 2.6 | 0.0 | 0.0 | 0.0 |
| | uniformis | | | | | | | |
| s | Bacteroides_{—} | 7.0 | 4.8 | 3.6 | 14.3 | 0.0 | 6.8 | 0.0 |
| | vulgatus | | | | | | | |
| s | Bacteroides_{—} | 3.0 | 0.8 | 0.1 | 0.1 | 0.0 | 0.6 | 0.0 |
| | xylanisolvens | | | | | | | |
| f | Porphyromonadaceae | 1.4 | 1.3 | 2.7 | 2.5 | 1.6 | 2.6 | 2.2 |
| g | Odoribacter | 0.0 | 0.0 | 2.1 | 0.5 | 0.0 | 0.0 | 0.6 |
| s | Odoribacter_{—} | 0.0 | 0.0 | 2.1 | 0.5 | 0.0 | 0.0 | 0.6 |
| | splanchnicus | | | | | | | |
| g | Parabacteroides | 1.4 | 1.3 | 0.6 | 2.0 | 1.6 | 2.6 | 1.6 |
| s | Parabacteroides_{—} | 1.4 | 0.0 | 0.0 | 0.0 | 0.0 | 0.3 | 0.0 |
| | distasonis | | | | | | | |
| s | Parabacteroides_{—} | 0.0 | 0.9 | 0.4 | 0.0 | 0.6 | 0.0 | 0.0 |
| | johnsonii | | | | | | | |
| s | Parabacteroides_{—} | 0.0 | 0.0 | 0.0 | 1.6 | 0.3 | 2.3 | 1.3 |
| | merdae | | | | | | | |
| s | Parabacteroides_{—} | 0.0 | 0.3 | 0.2 | 0.4 | 0.7 | 0.0 | 0.3 |
| | unclassified | | | | | | | |
| g | Porphyromonas | 0.0 | 0.0 | 0.0 | 0.0 | 0.0 | 0.0 | 0.0 |
| s | Porphyromonas_{—} | 0.0 | 0.0 | 0.0 | 0.0 | 0.0 | 0.0 | 0.0 |
| | asaccharolytica | | | | | | | |
| f | Prevotellaceae | 0.0 | 0.0 | 0.0 | 0.0 | 0.0 | 0.0 | 0.0 |
| g | Prevotella | 0.0 | 0.0 | 0.0 | 0.0 | 0.0 | 0.0 | 0.0 |
| s | Prevotella_{—} | 0.0 | 0.0 | 0.0 | 0.0 | 0.0 | 0.0 | 0.0 |
| | copri | | | | | | | |
| f | Rikenellaceae | 1.8 | 0.1 | 12.5 | 7.2 | 4.0 | 0.1 | 7.5 |
| g | Alistipes | 1.8 | 0.1 | 12.5 | 7.2 | 4.0 | 0.1 | 7.5 |
| s | Alistipes_{—} | 0.0 | 0.0 | 9.2 | 5.9 | 0.0 | 0.0 | 6.9 |
| | putredinis | | | | | | | |
| s | Alistipes_{—} | 0.3 | 0.0 | 3.3 | 1.4 | 4.0 | 0.0 | 0.6 |
| | shahii | | | | | | | |
| s | Alistipes_{—} | 1.4 | 0.1 | 0.0 | 0.0 | 0.0 | 0.0 | 0.0 |
| | unclassified | | | | | | | |
| p | Firmicutes | 35.3 | 66.1 | 54.4 | 61.6 | 64.2 | 37.5 | 55.7 |
| c | Bacilli | 2.9 | 0.6 | 3.9 | 3.2 | 0.3 | 0.1 | 2.4 |
| o | Lactobacillales | 2.9 | 0.6 | 3.9 | 3.2 | 0.3 | 0.1 | 2.4 |
| f | Enterococcaceae | 0.0 | 0.0 | 0.0 | 0.0 | 0.0 | 0.0 | 0.0 |
| g | Enterococcus | 0.0 | 0.0 | 0.0 | 0.0 | 0.0 | 0.0 | 0.0 |
| s | Enterococcus_{—} | 0.0 | 0.0 | 0.0 | 0.0 | 0.0 | 0.0 | 0.0 |
| | casseliflavus | | | | | | | |
| s | Enterococcus_{—} | 0.0 | 0.0 | 0.0 | 0.0 | 0.0 | 0.0 | 0.0 |
| | faecalis | | | | | | | |
| s | Enterococcus_{—} | 0.0 | 0.0 | 0.0 | 0.0 | 0.0 | 0.0 | 0.0 |
| | faecium | | | | | | | |
| s | Enterococcus_{—} | 0.0 | 0.0 | 0.0 | 0.0 | 0.0 | 0.0 | 0.0 |
| | unclassified | | | | | | | |
| f | Lactobacillaceae | 0.7 | 0.1 | 0.1 | 0.0 | 0.0 | 0.0 | 1.4 |
| g | Lactobacillus | 0.7 | 0.1 | 0.1 | 0.0 | 0.0 | 0.0 | 1.4 |
| s | Lactobacillus_{—} | 0.0 | 0.0 | 0.1 | 0.0 | 0.0 | 0.0 | 0.0 |
| | casei | | | | | | | |
| s | Lactobacillus_{—} | 0.0 | 0.0 | 0.0 | 0.0 | 0.0 | 0.0 | 0.5 |
| | crispatus | | | | | | | |
| s | Lactobacillus_{—} | 0.0 | 0.0 | 0.0 | 0.0 | 0.0 | 0.0 | 0.8 |
| | fermentum | | | | | | | |
| s | Lactobacillus_{—} | 0.3 | 0.0 | 0.0 | 0.0 | 0.0 | 0.0 | 0.1 |
| | gasseri | | | | | | | |
| s | Lactobacillus_{—} | 0.0 | 0.0 | 0.0 | 0.0 | 0.0 | 0.0 | 0.0 |
| | paracasei | | | | | | | |
| s | Lactobacillus_{—} | 0.2 | 0.0 | 0.0 | 0.0 | 0.0 | 0.0 | 0.0 |
| | reuteri | | | | | | | |
| s | Lactobacillus_{—} | 0.0 | 0.0 | 0.0 | 0.0 | 0.0 | 0.0 | 0.0 |
| | ruminis | | | | | | | |
| s | Lactobacillus_{—} | 0.0 | 0.0 | 0.0 | 0.0 | 0.0 | 0.0 | 0.0 |
| | salivarius | | | | | | | |
| f | Streptococcaceae | 2.2 | 0.5 | 3.8 | 3.1 | 0.3 | 0.1 | 1.0 |
| g | Lactococcus | 0.0 | 0.0 | 0.0 | 0.0 | 0.0 | 0.0 | 0.0 |
| s | Lactococcus_{—} | 0.0 | 0.0 | 0.0 | 0.0 | 0.0 | 0.0 | 0.0 |
| | lactis | | | | | | | |
| g | Streptococcus | 2.2 | 0.5 | 3.7 | 3.1 | 0.3 | 0.1 | 1.0 |
| s | Streptococcus_{—} | 0.0 | 0.0 | 0.0 | 0.0 | 0.0 | 0.0 | 0.0 |
| | anginosus | | | | | | | |
| s | Streptococcus_{—} | 0.0 | 0.0 | 0.0 | 0.0 | 0.0 | 0.0 | 0.0 |
| | infantarius | | | | | | | |
| s | Streptococcus_{—} | 0.1 | 0.1 | 0.0 | 0.0 | 0.0 | 0.0 | 0.0 |
| | mutans | | | | | | | |
| s | Streptococcus_{—} | 0.6 | 0.1 | 0.1 | 0.3 | 0.1 | 0.0 | 0.4 |
| | parasanguinis | | | | | | | |
| s | Streptococcus_{—} | 0.5 | 0.2 | 0.2 | 2.0 | 0.1 | 0.0 | 0.4 |
| | salivarius | | | | | | | |
| s | Streptococcus_{—} | 0.1 | 0.0 | 3.4 | 0.1 | 0.0 | 0.1 | 0.0 |
| | thermophilus | | | | | | | |
| s | Streptococcus_{—} | 0.8 | 0.0 | 0.0 | 0.1 | 0.1 | 0.0 | 0.1 |
| | vestibularis | | | | | | | |
| c | Clostridia | 11.7 | 64.6 | 50.1 | 58.1 | 60.6 | 37.0 | 52.9 |
| o | Clostridiales | 11.7 | 64.6 | 50.1 | 58.1 | 60.6 | 37.0 | 52.9 |
| f | Clostridiaceae | 10.0 | 1.9 | 3.1 | 0.4 | 3.5 | 18.3 | 5.8 |
| g | Clostridium | 10.0 | 1.9 | 3.1 | 0.4 | 3.3 | 18.3 | 5.8 |
| s | Clostridium_{—} | 0.0 | 0.1 | 0.6 | 0.1 | 0.6 | 0.0 | 0.1 |
| | asparagiforme | | | | | | | |
| s | Clostridium_{—} | 0.0 | 0.0 | 0.0 | 0.0 | 0.0 | 3.5 | 3.4 |
| | bartlettii | | | | | | | |
| s | Clostridium_{—} | 1.5 | 0.3 | 0.9 | 0.0 | 0.8 | 14.6 | 0.1 |
| | bolteae | | | | | | | |
| s | Clostridium_{—} | 0.0 | 0.1 | 0.1 | 0.0 | 0.2 | 0.0 | 0.0 |
| | cf | | | | | | | |
| s | Clostridium_{—} | 0.0 | 0.0 | 0.0 | 0.0 | 0.2 | 0.2 | 0.0 |
| | hathewayi | | | | | | | |
| s | Clostridium_{—} | 4.7 | 0.0 | 0.0 | 0.0 | 0.0 | 0.0 | 0.0 |
| | hiranonis | | | | | | | |
| s | Clostridium_{—} | 3.3 | 0.7 | 0.1 | 0.2 | 0.1 | 0.0 | 1.3 |
| | leptum | | | | | | | |
| s | Clostridium_{—} | 0.0 | 0.0 | 0.0 | 0.0 | 0.0 | 0.0 | 0.6 |
| | nexile | | | | | | | |
| s | Clostridium_{—} | 0.0 | 0.0 | 0.0 | 0.0 | 0.0 | 0.0 | 0.0 |
| | perfringens | | | | | | | |
| s | Clostridium_{—} | 0.3 | 0.5 | 1.2 | 0.0 | 0.7 | 0.0 | 0.1 |
| | scindens | | | | | | | |
| s | Clostridium_{—} | 0.1 | 0.2 | 0.1 | 0.0 | 0.8 | 0.0 | 0.0 |
| | symbiosum | | | | | | | |
| f | Clostridiales_{—} | 0.1 | 0.0 | 0.0 | 0.1 | 0.0 | 0.0 | 0.0 |
| | Family_XI_{—} | | | | | | | |
| | Incertae_{—}Sedis | | | | | | | |
| g | Anaerococcus | 0.0 | 0.0 | 0.0 | 0.0 | 0.0 | 0.0 | 0.0 |
| s | Anaerococcus_{—} | 0.0 | 0.0 | 0.0 | 0.0 | 0.0 | 0.0 | 0.0 |
| | unclassified | | | | | | | |
| g | Clostridiales_{—} | 0.0 | 0.0 | 0.0 | 0.0 | 0.0 | 0.0 | 0.0 |
| | Family_XI_{—} | | | | | | | |
| | Incertae_{—}Sedis_{—} | | | | | | | |
| | unclassified | | | | | | | |
| g | Finegoldia | 0.0 | 0.0 | 0.0 | 0.0 | 0.0 | 0.0 | 0.0 |
| s | Finegoldia_{—} | 0.0 | 0.0 | 0.0 | 0.0 | 0.0 | 0.0 | 0.0 |
| | magna | | | | | | | |
| g | Parvimonas | 0.0 | 0.0 | 0.0 | 0.0 | 0.0 | 0.0 | 0.0 |
| s | Parvimonas_{—} | 0.0 | 0.0 | 0.0 | 0.0 | 0.0 | 0.0 | 0.0 |
| | micros | | | | | | | |
| g | Peptoniphilus | 0.0 | 0.0 | 0.0 | 0.0 | 0.0 | 0.0 | 0.0 |
| s | Peptoniphilus_{—} | 0.0 | 0.0 | 0.0 | 0.0 | 0.0 | 0.0 | 0.0 |
| | harei | | | | | | | |
| s | Peptoniphilus_{—} | 0.0 | 0.0 | 0.0 | 0.0 | 0.0 | 0.0 | 0.0 |
| | unclassified | | | | | | | |
| f | Clostridiales_uncl | 1.0 | 0.2 | 0.1 | 0.7 | 1.2 | 0.1 | 0.1 |
| g | Blautia | 1.0 | 0.2 | 0.1 | 0.7 | 1.2 | 0.1 | 0.1 |
| s | Blautia_{—} | 0.0 | 0.0 | 0.0 | 0.0 | 0.0 | 0.0 | 0.0 |
| | hansenii | | | | | | | |
| s | Blautia_{—} | 0.0 | 0.1 | 0.0 | 0.7 | 1.2 | 0.0 | 0.1 |
| | hydrogenotrophica | | | | | | | |
| s | Blautia_{—} | 1.0 | 0.2 | 0.1 | 0.0 | 0.0 | 0.1 | 0.0 |
| | unclassified | | | | | | | |
| f | Eubacteriaceae | 0.3 | 35.1 | 10.2 | 17.2 | 10.5 | 0.0 | 12.1 |
| g | Eubacterium | 0.3 | 35.1 | 10.2 | 17.2 | 10.4 | 0.0 | 12.1 |
| s | Eubacterium_{—} | 0.0 | 0.0 | 0.0 | 0.0 | 0.0 | 0.0 | 0.7 |
| | eligens | | | | | | | |
| s | Eubacterium_{—} | 0.0 | 0.5 | 0.0 | 1.3 | 0.0 | 0.0 | 0.2 |
| | hallii | | | | | | | |
| s | Eubacterium_{—} | 0.3 | 0.0 | 0.0 | 0.0 | 0.6 | 0.0 | 0.1 |
| | limosum | | | | | | | |
| s | Eubacterium_{—} | 0.0 | 20.4 | 4.9 | 12.9 | 0.0 | 0.0 | 8.4 |
| | rectale | | | | | | | |
| s | Eubacterium_{—} | 0.0 | 14.2 | 5.3 | 3.0 | 9.8 | 0.0 | 2.7 |
| | siraeum | | | | | | | |
| s | Eubacterium_{—} | 0.0 | 0.0 | 0.0 | 0.0 | 0.0 | 0.0 | 0.0 |
| | ventriosum | | | | | | | |
| g | Pseudoramibacter | 0.0 | 0.0 | 0.0 | 0.0 | 0.0 | 0.0 | 0.0 |
| s | Pseudoramibacter_{—} | 0.0 | 0.0 | 0.0 | 0.0 | 0.0 | 0.0 | 0.0 |
| | alactolyticus | | | | | | | |
| f | Lachnospiraceae | 0.0 | 17.1 | 11.5 | 6.5 | 11.7 | 0.2 | 2.2 |
| g | Anaerostipes | 0.0 | 0.0 | 1.0 | 0.0 | 0.0 | 0.1 | 0.0 |
| s | Anaerostipes_{—} | 0.0 | 0.0 | 1.0 | 0.0 | 0.0 | 0.1 | 0.0 |
| | caccae | | | | | | | |
| g | Butyrivibrio | 0.0 | 0.0 | 0.0 | 0.0 | 0.0 | 0.0 | 0.0 |
| s | Butyrivibrio_{—} | 0.0 | 0.0 | 0.0 | 0.0 | 0.0 | 0.0 | 0.0 |
| | crossotus | | | | | | | |
| g | Coprococcus | 0.0 | 2.1 | 8.9 | 0.6 | 0.0 | 0.0 | 1.4 |
| s | Coprococcus_{—} | 0.0 | 0.0 | 3.7 | 0.6 | 0.0 | 0.0 | 0.5 |
| | catus | | | | | | | |
| s | Coprococcus_{—} | 0.0 | 2.1 | 5.2 | 0.0 | 0.0 | 0.0 | 0.9 |
| | comes | | | | | | | |
| g | Dorea | 0.0 | 3.4 | 0.0 | 5.1 | 9.6 | 0.0 | 0.8 |
| s | Dorea_{—} | 0.0 | 1.1 | 0.0 | 1.4 | 5.5 | 0.0 | 0.2 |
| | formicigenerans | | | | | | | |
| s | Dorea_{—} | 0.0 | 2.3 | 0.0 | 3.7 | 4.1 | 0.0 | 0.6 |
| | longicatena | | | | | | | |
| g | Roseburia | 0.0 | 11.6 | 1.6 | 0.8 | 2.0 | 0.0 | 0.0 |
| s | Roseburia_{—} | 0.0 | 8.1 | 1.6 | 0.7 | 0.0 | 0.0 | 0.0 |
| | intestinalis | | | | | | | |
| s | Roseburia_{—} | 0.0 | 3.5 | 0.0 | 0.1 | 2.0 | 0.0 | 0.0 |
| | inulinivorans | | | | | | | |
| f | Ruminococcaceae | 0.3 | 10.3 | 25.1 | 33.2 | 33.8 | 18.4 | 32.6 |
| g | Anaerotruncus | 0.0 | 0.0 | 0.0 | 0.1 | 0.0 | 0.0 | 0.0 |
| s | Anaerotruncus_{—} | 0.0 | 0.0 | 0.0 | 0.1 | 0.0 | 0.0 | 0.0 |
| | colihominis | | | | | | | |
| g | Faecalibacterium | 0.0 | 0.0 | 4.4 | 11.7 | 20.1 | 0.0 | 5.6 |
| s | Faecalibacterium_{—} | 0.0 | 0.0 | 0.8 | 0.7 | 10.7 | 0.0 | 3.1 |
| | cf | | | | | | | |
| s | Faecalibacterium_{—} | 0.0 | 0.0 | 2.9 | 11.0 | 6.6 | 0.0 | 1.9 |
| | prausnitzii | | | | | | | |
| s | Faecalibacterium_{—} | 0.0 | 0.0 | 0.7 | 0.0 | 2.7 | 0.0 | 0.7 |
| | unclassified | | | | | | | |
| s | Ruminococcus_{—}bromii | 0.0 | 0.2 | 0.0 | 11.3 | 0.0 | 0.0 | 13.7 |
| s | Ruminococcus_{—} | 0.2 | 0.0 | 0.0 | 0.0 | 0.0 | 7.5 | 10.3 |
| | gnavus | | | | | | | |
| s | Ruminococcus_{—} | 0.0 | 8.4 | 0.0 | 2.8 | 0.0 | 0.0 | 0.0 |
| | lactaris | | | | | | | |
| s | Ruminococcus_{—} | 0.0 | 1.6 | 5.0 | 2.9 | 0.0 | 0.0 | 0.4 |
| | obeum | | | | | | | |
| s | Ruminococcus_{—} | 0.0 | 0.0 | 15.6 | 4.4 | 13.7 | 10.9 | 2.6 |
| | torques | | | | | | | |
| c | Erysipelotrichi | 1.1 | 0.8 | 0.4 | 0.0 | 2.5 | 0.3 | 0.3 |
| o | Erysipelotrichales | 1.1 | 0.8 | 0.4 | 0.0 | 2.5 | 0.3 | 0.3 |
| f | Erysipelotrichaceae | 1.1 | 0.8 | 0.4 | 0.0 | 2.5 | 0.3 | 0.3 |
| g | Catenibacterium | 0.0 | 0.0 | 0.0 | 0.0 | 0.0 | 0.0 | 0.0 |
| s | Catenibacterium_{—} | 0.0 | 0.0 | 0.0 | 0.0 | 0.0 | 0.0 | 0.0 |
| | mitsuokai | | | | | | | |
| g | Coprobacillus | 1.1 | 0.2 | 0.0 | 0.0 | 1.5 | 0.3 | 0.3 |
| s | Coprobacillus_{—} | 1.1 | 0.2 | 0.0 | 0.0 | 1.5 | 0.3 | 0.3 |
| | bacterium | | | | | | | |
| g | Holdemania | 0.0 | 0.6 | 0.4 | 0.0 | 1.0 | 0.0 | 0.0 |
| s | Holdemania_{—} | 0.0 | 0.6 | 0.4 | 0.0 | 1.0 | 0.0 | 0.0 |
| | filiformis | | | | | | | |
| c | Negativicutes | 19.6 | 0.0 | 0.0 | 0.3 | 0.8 | 0.0 | 0.1 |
| o | Selenomonadales | 19.6 | 0.0 | 0.0 | 0.3 | 0.8 | 0.0 | 0.1 |
| f | Acidaminococcaceae | 1.8 | 0.0 | 0.0 | 0.3 | 0.0 | 0.0 | 0.1 |
| g | Acidaminococcaceae_{—} | 1.8 | 0.0 | 0.0 | 0.3 | 0.0 | 0.0 | 0.1 |
| | unclassified | | | | | | | |
| g | Phascolarctobacterium | 0.0 | 0.0 | 0.0 | 0.0 | 0.0 | 0.0 | 0.0 |
| s | Phascolarctobacterium_{—} | 0.0 | 0.0 | 0.0 | 0.0 | 0.0 | 0.0 | 0.0 |
| | unclassified | | | | | | | |
| f | Veillonellaceae | 17.7 | 0.0 | 0.0 | 0.0 | 0.8 | 0.0 | 0.0 |
| g | Dialister | 0.0 | 0.0 | 0.0 | 0.0 | 0.8 | 0.0 | 0.0 |
| s | Dialister_{—} | 0.0 | 0.0 | 0.0 | 0.0 | 0.8 | 0.0 | 0.0 |
| | invisus | | | | | | | |
| g | Megamonas | 17.7 | 0.0 | 0.0 | 0.0 | 0.0 | 0.0 | 0.0 |
| s | Megamonas_{—} | 17.7 | 0.0 | 0.0 | 0.0 | 0.0 | 0.0 | 0.0 |
| | hypermegale | | | | | | | |
| g | Veillonella | 0.0 | 0.0 | 0.0 | 0.0 | 0.0 | 0.0 | 0.0 |
| s | Veillonella_{—} | 0.0 | 0.0 | 0.0 | 0.0 | 0.0 | 0.0 | 0.0 |
| | atypica | | | | | | | |
| s | Veillonella_{—} | 0.0 | 0.0 | 0.0 | 0.0 | 0.0 | 0.0 | 0.0 |
| | parvula | | | | | | | |
| s | Veillonella_{—} | 0.0 | 0.0 | 0.0 | 0.0 | 0.0 | 0.0 | 0.0 |
| | unclassified | | | | | | | |
| p | Fusobacteria | 0.0 | 0.0 | 0.0 | 0.0 | 0.0 | 0.0 | 0.0 |
| c | Fusobacteria | 0.0 | 0.0 | 0.0 | 0.0 | 0.0 | 0.0 | 0.0 |
| o | Fusobacteriales | 0.0 | 0.0 | 0.0 | 0.0 | 0.0 | 0.0 | 0.0 |
| f | Fusobacteriaceae | 0.0 | 0.0 | 0.0 | 0.0 | 0.0 | 0.0 | 0.0 |
| g | Fusobacterium | 0.0 | 0.0 | 0.0 | 0.0 | 0.0 | 0.0 | 0.0 |
| s | Fusobacterium_{—} | 0.0 | 0.0 | 0.0 | 0.0 | 0.0 | 0.0 | 0.0 |
| | ulcerans | | | | | | | |
| p | Proteobacteria | 1.4 | 0.8 | 2.3 | 0.5 | 1.0 | 12.8 | 7.7 |
| c | Betaproteobacteria | 0.0 | 0.0 | 0.1 | 0.0 | 0.0 | 0.0 | 0.0 |
| o | Burkholderiales | 0.0 | 0.0 | 0.1 | 0.0 | 0.0 | 0.0 | 0.0 |
| f | Sutterellaceae | 0.0 | 0.0 | 0.0 | 0.0 | 0.0 | 0.0 | 0.0 |
| g | Sutterella | 0.0 | 0.0 | 0.0 | 0.0 | 0.0 | 0.0 | 0.0 |
| s | Sutterella_{—} | 0.0 | 0.0 | 0.0 | 0.0 | 0.0 | 0.0 | 0.0 |
| | wadsworthensis | | | | | | | |
| c | Deltaproteobacteria | 1.1 | 0.0 | 1.4 | 0.5 | 1.0 | 0.0 | 0.2 |
| o | Desulfovibrionales | 1.1 | 0.0 | 1.4 | 0.5 | 1.0 | 0.0 | 0.2 |
| f | Desulfovibrionaceae | 1.1 | 0.0 | 1.4 | 0.5 | 1.0 | 0.0 | 0.2 |
| g | Bilophila | 1.1 | 0.0 | 1.2 | 0.5 | 1.0 | 0.0 | 0.1 |
| s | Bilophila_{—} | 1.1 | 0.0 | 1.2 | 0.5 | 1.0 | 0.0 | 0.1 |
| | wadsworthia | | | | | | | |
| g | Desulfovibrio | 0.0 | 0.0 | 0.2 | 0.0 | 0.0 | 0.0 | 0.0 |
| s | Desulfovibrio_{—} | 0.0 | 0.0 | 0.0 | 0.0 | 0.0 | 0.0 | 0.0 |
| | piger | | | | | | | |
| c | Gammaproteobacteria | 0.3 | 0.5 | 0.8 | 0.0 | 0.0 | 12.8 | 7.5 |
| o | Enterobacteriales | 0.3 | 0.5 | 0.8 | 0.0 | 0.0 | 12.8 | 7.5 |
| f | Enterobacteriaceae | 0.3 | 0.5 | 0.8 | 0.0 | 0.0 | 12.8 | 7.5 |
| g | Enterobacter | 0.0 | 0.0 | 0.0 | 0.0 | 0.0 | 0.1 | 0.0 |
| s | Enterobacter_{—} | 0.0 | 0.0 | 0.0 | 0.0 | 0.0 | 0.0 | 0.0 |
| | cloacae | | | | | | | |
| g | Escherichia | 0.0 | 0.5 | 0.8 | 0.0 | 0.0 | 12.4 | 6.7 |
| s | Escherichia_{—} | 0.0 | 0.5 | 0.8 | 0.0 | 0.0 | 12.4 | 6.7 |
| | coli | | | | | | | |
| s | Escherichia_{—} | 0.0 | 0.0 | 0.0 | 0.0 | 0.0 | 0.0 | 0.0 |
| | unclassified | | | | | | | |
| g | Klebsiella | 0.3 | 0.0 | 0.0 | 0.0 | 0.0 | 0.3 | 0.8 |
| s | Klebsiella_{—} | 0.2 | 0.0 | 0.0 | 0.0 | 0.0 | 0.1 | 0.6 |
| | pneumoniae | | | | | | | |
| s | Klebsiella_{—} | 0.0 | 0.0 | 0.0 | 0.0 | 0.0 | 0.2 | 0.1 |
| | unclassified | | | | | | | |
| p | Verrucomicrobia | 1.4 | 1.6 | 0.0 | 0.0 | 0.0 | 0.0 | 0.0 |
| c | Verrucomicrobiae | 1.4 | 1.6 | 0.0 | 0.0 | 0.0 | 0.0 | 0.0 |
| o | Verrucomicrobiales | 1.4 | 1.6 | 0.0 | 0.0 | 0.0 | 0.0 | 0.0 |
| f | Verrucomicrobiaceae | 1.4 | 1.6 | 0.0 | 0.0 | 0.0 | 0.0 | 0.0 |
| g | Akkermansia | 1.4 | 1.6 | 0.0 | 0.0 | 0.0 | 0.0 | 0.0 |
| s | Akkermansia_{—} | 1.4 | 1.6 | 0.0 | 0.0 | 0.0 | 0.0 | 0.0 |
| | muciniphila | | | | | | | |

The patients as set forth in Table 1 were followed through treatment using the present invention and the results tracked as set forth in Tables 3 to 9.

TABLE 3
Repeat Responders + Stable vs. Progressors
For IPI + NIVO (In) Melanoma.

| Responders + Stable | Progressors |
|---|---|
| P7 | P24 |
| P8 | P28 |
| P10 | P30 |
| P14 | P32 |
| P23 | P46 |
| P25 | P53 |
| P34 | P54 |
| P35 | P66 |
| P52 | |
| P55 | |
| P59 | |
| P61 | |
| P63 | |
| P67 | |
| P68 | |
| P69 | |

This repeat analysis was conducted as P52 had a large bone lesion that on resection which, after one week, showed only dead tumor cells, as such, P52 is listed as a responder.

TABLE 4
Second Analysis-By Percent Change In Tumor Size For All
Treated Melanomas (IPI + NIVO, PEMBRO, IPI, NIVO).

| Patient | Percent Change Tumor |
|---|---|
| P25 | −100 |
| P42 | −100 |
| P52 | −100 |
| P55 | −100 |
| P57 | −100 |
| P63 | −100 |
| P69 | −87 |
| P10 | −83 |
| P17 | −70 |
| P39 | −68 |
| P34 | −66 |
| P61 | −65 |
| P14 | −60 |
| P23 | −55 |
| P8 | −55 |
| P58 | −48 |
| P33 | −36 |
| P67 | −34 |
| P68 | −34 |
| P35 | −20 |
| P45 | −14 |
| P59 | −10 |
| P7 | −4 |
| P30 | 0 |
| P44 | 0 |
| P53 | 0 |
| P48 | 0 |
| P46 | +24 |
| P49 | +25 |
| P64 | +31 |
| P56 | +46 |
| P16 | +53 |
| P32 | +60 |
| P66 | +61 |
| P54 | +70 |
| P22 | +81 |
| P28 | +85 |
| P64 | +100 |
| P24 | +136 |

TABLE 5
Responders + Stable vs. Progressors with NIVO (N) in RCC

| Responders + Stable | Progressors |
|---|---|
| P1 | P5 |
| P3 | P11 |
| P6 | P26 |
| P27 | P38 |
| | P43 |
| | P47 |
| | P60 |
| | P62 |

TABLE 6
Severe Autoimmune Toxicities vs. None In All
Treated Melanoma Patients (IN, P, I, N)

| With GR3/4 Toxicities | No Toxicities |
|---|---|
| P7 | P14 |
| P8 | P16 |
| P10 | P17 |
| P23 | P22 |
| P23 | P25 |
| P28 | P32 |
| P30 | P34 |
| P33 | P35 |
| P42 | P39 |
| P53 | P44 |
| P56 | P45 |
| P57 | P46 |
| P58 | P48 |
| P59 | P49 |
| P63 | P52 |
| P66 | P54 |
| P68 | P55 |
| | P61 |
| | P64 |
| | P65 |
| | P67 |

TABLE 7
Severe Autoimmune Toxicities vs. None
In All Treated RCC Patients (N)

| With GR3/4 Toxicities | No Toxicities |
|---|---|
| P5 | P1 |
| P27 | P3 |
| | P6 |
| | P1 |
| | P26 |
| | P38 |
| | P43 |
| | P47 |
| | P60 |
| | P62 |

TABLE 8
Specific Toxicities Among Melanoma Patients

| Pneumonitis | Hepatitis | Colitis | Hypophysitis | Rare | None |
|---|---|---|---|---|---|
| P24 | P7 | P8 | P42 | P10 | P14 |
| P28 | P33 | P23 | P56 | P53 | P16 |
| P30 | P58 | P57 | | P63 | P17 |
| P59 | | P68 | | | P22 |
| P66 | | | | | P25 |
| | | | | | P32 |
| | | | | | P34 |
| | | | | | P35 |
| | | | | | P39 |
| | | | | | P44 |
| | | | | | P45 |
| | | | | | P46 |
| | | | | | P48 |
| | | | | | P49 |
| | | | | | P52 |
| | | | | | P54 |
| | | | | | P55 |
| | | | | | P61 |
| | | | | | P64 |
| | | | | | P65 |
| | | | | | P67 |
| | | | | | P69 |

TABLE 9
Specific Toxicities Among Patients

| Pneumonitis | Rare | None |
|---|---|---|
| P27 | P5 | P1 |
| | | P3 |
| | | P6 |
| | | P11 |
| | | P26 |
| | | P38 |
| | | P43 |
| | | P47 |
| | | P60 |

Using next generation sequencing techniques, the present inventors identified specific species of gut commensal bacteria that were associated with response to immune checkpoint inhibitor therapy (ICT) in melanoma patients. For example, patients receiving ipilumumab (anti-CTLA-4) and nivolumab (anti-PD-1) combination ICT for melanoma who showed a positive response had increased abundance of three specific gut bacterial species (Faecalibacterium prausnitzii, Bacteroides tlretaiotaomicron, and Holdemania filiformis) compared to counterparts with progressive disease. Next, the inventors used a preclinical melanoma model (immunocompetent C57BL/6 mice with B16-F10 melanoma) to investigate whether we could recapitulate our clinical findings and establish causality. Indeed, mice with melanoma who were treated with combination ICT and a precision probiotic cocktail (B. thetaiotaomicron and F. prausnitzii) had smaller tumor growth and increased length of survival compared to mice who were treated with Lactobacillus acidophilus, a probiotic commonly found in yogurt. These data show that administration of a precision probiotic cocktail increases efficacy of ICT (ipilumumab and nivolumab) against melanoma as an adjunctive therapy to ICT that optimize therapeutic efficacy.

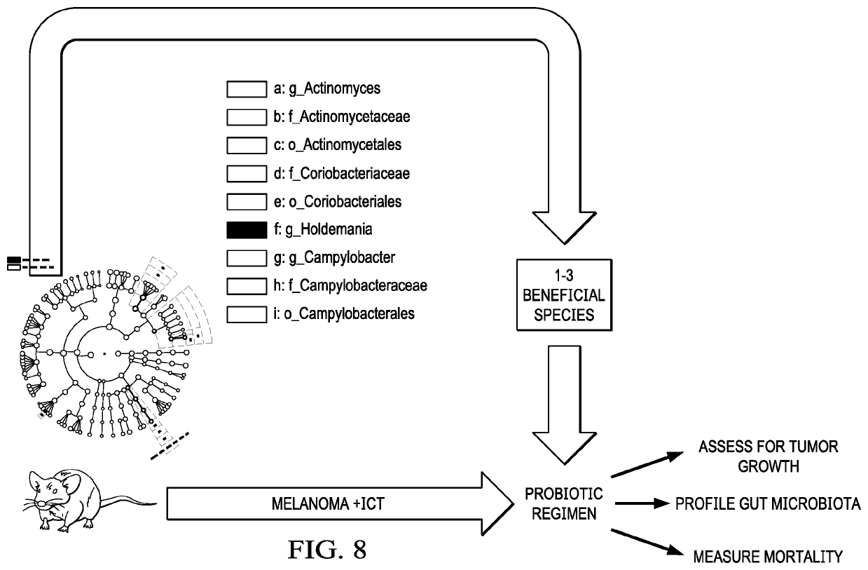

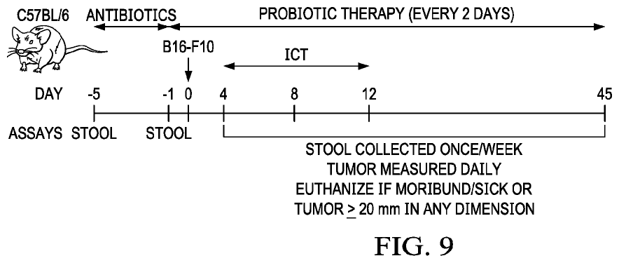

FIG. 8 shows an outline of in vivo studies and expected outcomes of the present invention. FIG. 9 shows an outline of the detailed in vivo studies using the present invention.

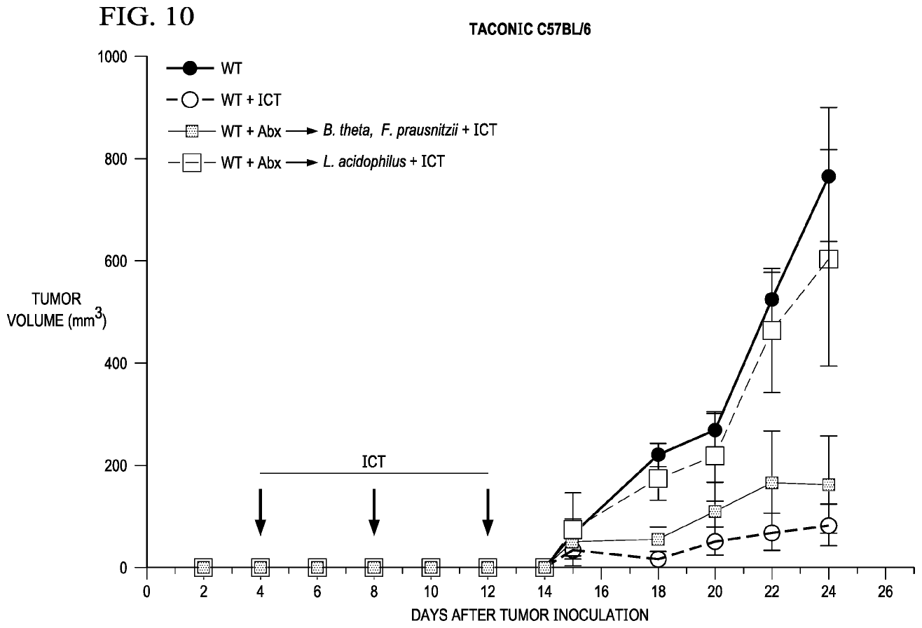

FIG. 10 is a graph that shows the results of the precision probiotic therapy on Taconic C57BL/6 versus control that promotes the activity of immune checkpoint therapy of the present invention. ICT was 200 micrograms anti-PD1 and 200 micrograms anti-CTLA4 IP injections. B16-F10 melanoma cells from C57BL/6, 1×10⁵ cells were injected subcutaneously in the right flank. Probiotic therapy was 2×10⁸ cfu of bacteria administered by oral gavage.

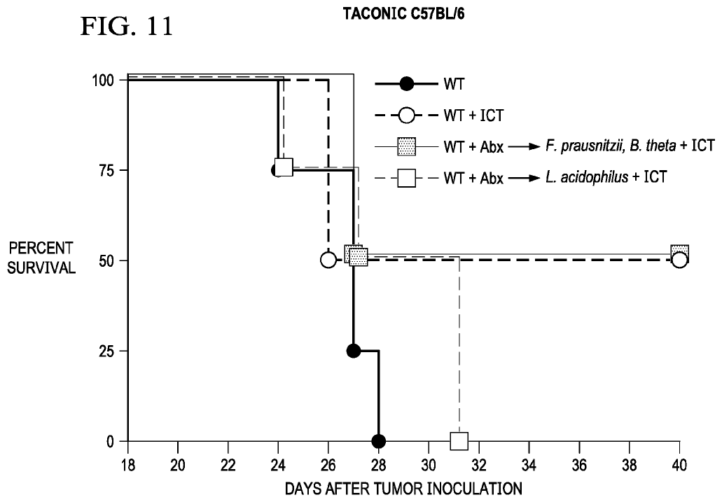

FIG. 11 is a graph that shows the results of the precision probiotic therapy on Taconic C57BL/6 prolongs survival in mice with melanoma that are treated with ICT compared to mice treated with control. It was found that precision probiotic therapy (B. theta, F. prausnitzii) prolongs survival in mice with melanoma that are treated with ICT compared to mice treated with L. acidophilus (probiotic commonly found in yogurt).

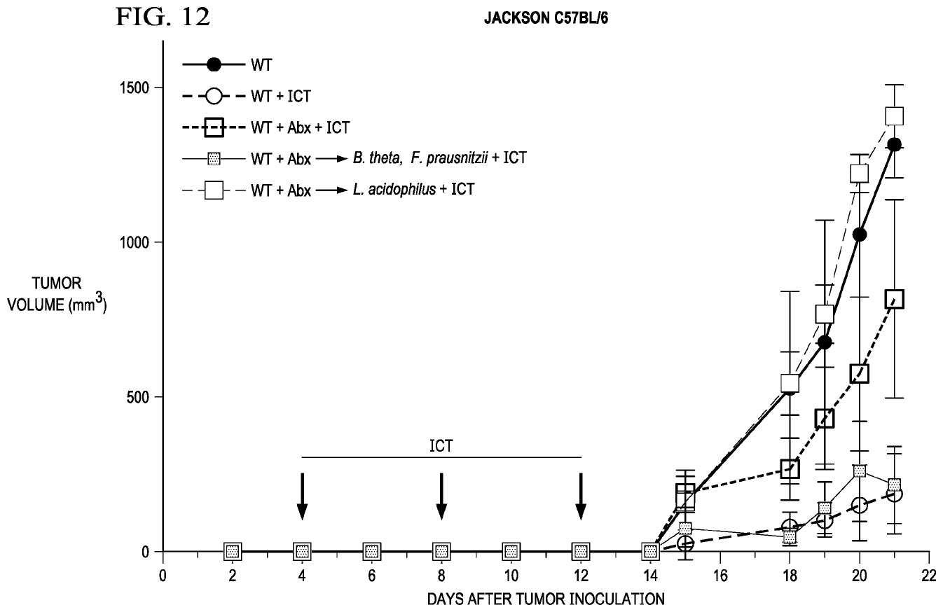

FIG. 12 is a graph that shows the results of the precision probiotic therapy on Jackson C57BL/6 versus control that promotes the activity of immune checkpoint therapy against melanoma using the present invention. It was found that precision probiotic therapy (B. theta, F. prausnitzii) promoted ICT in Mice (Jackson, n=4) with melanoma.

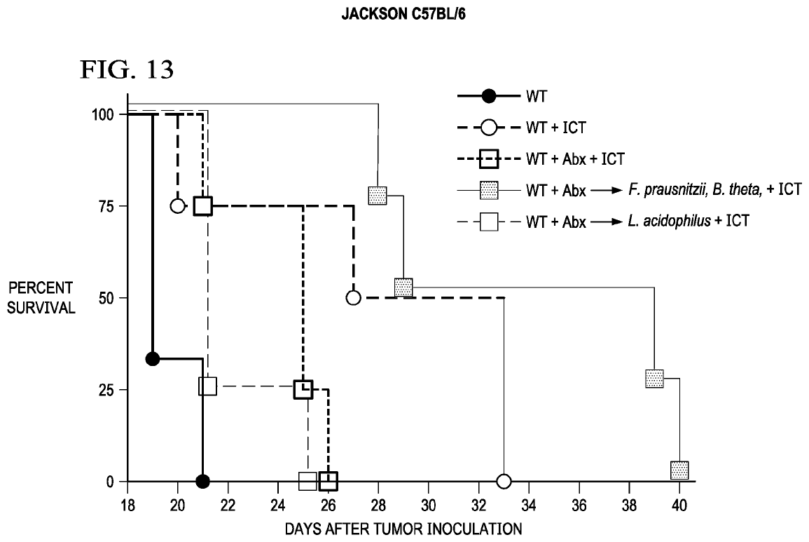

FIG. 13 is a graph that shows the results of the precision probiotic therapy on Jackson C57BL/6 prolongs survival in mice with melanoma that are treated with ICT compared to mice treated with control. These results show that precision probiotic therapy significantly increases the length of survival in mice (Jackson) with melanoma treated with ICT.

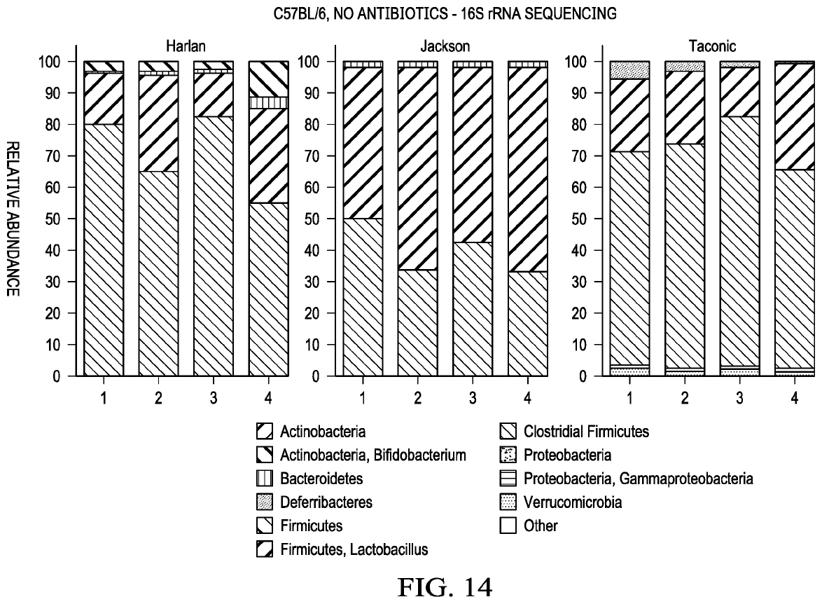

FIG. 14 is a graph that shows that Jackson C57BL/6 mice have decreased relative abundance of Clostridial firmicutes (which include F. prausnitzii, H. filiformis) compared to Taconic C57BL/6 Mice.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] Larkin J, Chiarian-Sileri V, Gonzalez R, Grobb J J, Cowey C L, Lao C D, et al (2015). Combined nivolumab and ipilimumab or monotherapy in untreated melanoma. N Engl J Med 373 (1), 23-34.
[2] Horvat T Z, Adel N G, Dang T O, Momtaz P, Postow M A, Callahan M K, et al (2015). Immune-related adverse events, need for systemic immunosuppression, and effects on survival and time to treatment failure in patients with melanoma treated with ipilimumab at Memorial Sloan Kettering Cancer Center. J Clin Oncol 33 (28), 3192-3198.
[3] Frankel A E, Frenkel E P (2017). Melanoma metamorphoses: advances in biology and therapy. J Cancer Science & Therapy 9 (1), 325-335.
[4] Salerno E P, Bedognetti D, Mauldin I S, Deacon D H, Shea S M, Pinczewski J, et al (2016). Human melanomas and ovarian cancers overexpressing mechanical barrier molecule genes lack immune signatures and have increased patient mortality risk. Oncoimmunol 5 (12), e1240857.
[5] Gibney G T, Weiner L M, Atkins M B (2016). Predictive biomarkers for checkpoint inhibitor-based immunotherapy. Lancet Oncology 17 (12), e542-e551.
[6] Robert E W, Broz M L, Binnewies M, Headley M B, Nelson A E, Wolf D M, et al (2016). Critical role for CD103+/CD141+ dendritic cells bearing CCR7 for tumor antigen trafficking and priming of T cell immunity in melanoma. Cancer Cell 30 (2), 324-336.
[7] Tida N, Dzutseva A, Stewart C A, Smith L, Bouladoux N, Weingarten R A, et al (2013). Commensal bacteria control cancer response to therapy by modulating the tumor microenvironment. Science 342 (6161), 967-970.
[8] Sivan A, Corrales L, Hubert N, Williams J B, Aquino-Michaels K, Earley Z M, et al (2015). Commensal Bifidobacterium promotes antitumor immunity and facilitates anti-PD-L1 efficacy. Science 350 (6264), 1084-1089.
[9] Vetizou M, Pitt J M, Daillere R, Lepage P, Waldschmitt N, Flament C, et al (2015). Anticancer immunotherapy by CTLA-4 blockade relies on the gut microbiota. Science 350 (6264), 1079-1084.
[10] Eisenhauer E A, Therasse P, Bogaerts J, Schwartz L H, Sargent D, Ford R, et al (2009). New response evaluation criteria in solid tumours: revised RECIST guideline (version 1.1). Eur J Cancer 45 (2), 228-247.
[11] Fan D, Coughlin L A, Neubauer M M, Kim J, Kim M S, Zhan X, et al (2015) Activation of HIF-1α and LL-37 by commensal bacteria inhibits Candida albicans colonization. Nat Med 21 (7), 808-814.
[12] Piper H G, Fan D, Coughlin L A, Ho E X, McDaniel M M, Channabasappa N, et al (2016). Severe gut microbiota dysbiosis is associated with poor growth in patients with short bowel syndrome. JPEN J Parenter Enteral Nutr, Epub July 12.
[13] Sinha R, Ahn J, Sampson J N, Shi J, Yu G, Xiong X, et al (2016). Fecal microbiota, fecal metabolome, and colorectal cancer interrelations. PLoS One 11 (3), e0152126.
[14] Dhaven C D, Evans A M, Dai H, Lawton K A (2010). Organization of GC/MS and LC/MS metabolomics data into chemical libraries. J Cheminform 2 (1), 9.
[15] Hodi F S, Chesney J, Pavlick A C, Robert C, Grossmann K F, McDermott D F, et al (2016). Combined nivolumab and ipilimumab versus ipilimumab alone in patients with advanced melanoma: 2-year overall survival outcomes in a multicenter, randomized, controlled, phase 2 trial. Lancet Oncology 17 (11), 1558-1568.
[16] Wolchok J D, Kluger H, Callahan M K, Postow M A, Rizvi N A, Lesokhin A M, et al (2013). Nivolumab plus ipilimumab in advanced melanoma. N Engl J Med 369 (2), 122-133.
[17] Faith J J, Guruge J L, Charbonneau M, Subramanian S, Seedorf H, Goodman A L, et al (2013). The long-term stability of the human gut microbiota. Science 341 (6141), 1237439.
[18] Dethlefsen L, Relman D A (2011). Incomplete recovery and individualized responses of the human distal gut microbiota to repeated antibiotic perturbation. PNAS 108 Suppl 1, 4554-4561.
[19] Gopalakrishnan V, Spencer C, Reuben A, Karpinets T, Hutchinson D, Hoffman K, et al (2017). Association of diversity and composition of the gut microbiome with differential responses to PD-1 based therapy in patients with metastatic melanoma. J Clin Oncol 35 (Suppl 7S), 2.
[20] Chaput N, Lepage P, Coutzac C, Soularue E, Le Roux K, Monot C, et al (2017). Baseline gut microbiota predicts clinical response and colitis in metastatic melanoma patients treated with ipilimumab. Ann Oncol, Epub March 27.
[21] Daillere R, Vetizou M, Waldschmitt N, Yamazaki T, Isnard C, Poirier-Colame V, et al (2016). Enterococcus hirae and Barnesiella intestinihominis facilitate cyclophosphamide-induced therapeutic immunomodulatory effects. Immunity 45 (4), 931-943.
[22] Butcher J P, Malcolm J, Benson R A, Deng D M, Brewer J M, Garside P, et al (2011). Effects of Streptococcus mutans on dendritic cell activation and function. J Dent Res 90 (10), 1221-1227.
[23] Li H, Wang D (2014). Streptococcus mutans wall-associated protein A promotes TLR4-induced dendritic cell maturation. Scand J Immunol 80 (2), 121-126.
[24] Hickey C A, Kuhn K A, Donermeyer D L, Porter N T, Jin C, Cameron E A, et al (2015). Colitogenic Bacteroides thetaiotamicron antigens access host immune cells in a sulfatase-dependent manner via outer membrane vesicles. Cell Host Microbe 17 (5), 672-680.
[25] Berezow A B, Ernst R K, Coats S R, Braham P H, Karimi-Naser L M, Darveau R P (2009). The structurally similar, penta-acylated lipopolysaccharides of Porphyromonas gingivalis and Bacteroides elicit strikingly different innate immune responses.
[26] Selby M J, Engelhardt J J, Johnston R J, Lu L S, Han M, Thudium K, et al (2016). Preclinical development of ipilimumab and nivolumab combination immunotherapy: mouse tumor models, in vitro functional studies, and cynomolgus macaque toxicology. PLoS One 11 (9), e0161779.
[27] Wang H, Hu S, Chen X, Shi H, Chen C, Sun L, et al (2017). cGAS is essential for the antitumor effect of immune checkpoint blockade. PNAS 114 (7), 1637-1642.
[28] McNulty N P, Yatsunenko T, Hsiao A, Faith J J, Muegge B D, Goodman A L, et al (2011). The impact of a consortium of fermented milk strains on the gut microbiome of gnotobiotic mice and monozygotic twins. Sci Transl Med 3 (106), 106ra106.
[29] Derrien M, van Hylckama Vlieg J E (2015). Fate, activity and impact of ingested bacteria within the human gut microbiota. Trends in Microbiology 23 (6), 354-366.
[30] Morais S M, Silva K A, Araujo H, Vieira I G P, Alves D R, Fontenelle R O S, et al (2017). Anacardic acid constituents from cashew nut shell liquid: NMR characterization and the effect of unsaturation on its biological activities. Pharmaceuticals 10 (1), E31.
[31] Hollands A, Corriden R, Gysler G, Dahesh S, Olson J, Raza A S, et al (2016). Natural product anacardic acid from cashew nut shells stimulates neutrophil extracellular trap production and bactericidal activity. J Biol Chem 291 (27), 13964-13973.
[32] Gnanaprakasam J N, Estrada-Muniz E, Vega L (2015). The anacardic 6-pentadecyl salicylic acid induces macrophage activation via the phosphorylation of ERK1/2, JNK, P38 kinases and NF-κB. Int Immunopharmacol 29 (2), 808-817.
[33] Fadelu T, Niedzwiecki D, Zhang S, Ye X, Saltz L, Mayer R J, et al (2017). Nut consumption and survival in stage III colon cancer patients: results from CALGB89803 (Alliance). J Clin Oncol 35 (Suppl), 3517.

1. A method of increasing an efficacy of an immune checkpoint inhibitor comprising:
identifying a human patient in need of treatment for a melanoma;
providing the human patient with an effective amount of the immune checkpoint inhibitor; and
providing the human patient with at least one of: a probiotic bacteria, a prebiotic agent, or a xenobiotic agent, in an amount is effective to increase a potency of the immune checkpoint inhibitor against the melanoma.

2. The method of claim 1, wherein the immune checkpoint inhibitor is selected from at least one of a CTLA-4 inhibitor or a PD1 inhibitor, ipilimumab, nivolumab, ipilimumab plus nivolumab, or pembrolizumab.

3. The method of claim 1, wherein the human patient does not have a BRAF mutation.

4. (canceled)

5. The method of claim 1, wherein the probiotic bacteria are selected from at least one of Faecalibacterium prausnitzii, Holdemania filiformis, Bacteroides thetaiotamicron, Dorea formicigenerans, Bacteroides caccae or Streptococcus parasanguinis.

6. The method of claim 1, wherein the probiotic bacteria is Dorea formicigeneran and is provided to enhance a therapeutic effect of pembrolizumab (PD1); the probiotic bacteria are at least one of:
Faecalibacterium prausnitzii, or Holdemania filiformis, and the probiotic bacteria are provided to enhance a potency of a combination of ipilimumab and nivolumab;
the probiotic bacteria that increase an effectiveness of a combination of the immune checkpoint inhibitors ipilimumab plus nivolumab is selected from at least one of: Faecalibacterium prausnitzii, Holdemania filiformis, or Bacteroides thetaiotamicron;
the probiotic bacteria that increase an effectiveness of the immune checkpoint inhibitor pembrolizumab is Dorea formicigenerans; or
the probiotic bacteria that increase an effectiveness of any immune checkpoint inhibitor is selected from at least one of: Bacteroides caccae or Streptococcus parasanguinis.

7. (canceled)

8. The method of claim 1, wherein the xenobiotic agent is an anacardic acid, a 15:2 anacardic acid, or active derivatives thereof.

9. (canceled)

10. (canceled)

11. (canceled)

12. The method of claim 1, wherein the human patient is further provided with an effective amount of the probiotic bacteria, the prebiotic agent, and an anacardic acid in an amount sufficient to enhance the potency of the immune checkpoint inhibitor selected from at least one of ipilimumab, nivolumab, ipilimumab plus nivolumab, or pembrolizumab.

13. The method of claim 1, further comprising providing a probiotic further comprising one or more bacteria selected from at least one of: Methanobrevibacter smithii, Bacteroides thetaiotamicron, Lactobacillus plantarum, Eubacterium limosum, Faecalibacterium prausnitzii, Holdemania filiformis, Bacteroides thetaiotamicron, Dorea formicigenerans, Bacteroides caccae or Streptococcus parasanguinis in an amount sufficient to enhance the potency of the immune checkpoint inhibitor selected from at least one of ipilimumab, nivolumab, ipilimumab plus nivolumab, or pembrolizumab.

14. The method of claim 1, wherein the melanoma is metastatic melanoma, or is resistant or refractory to the immune checkpoint inhibitor.

15. (canceled)

16. The method of claim 1, wherein the human patient did not receive a concurrent antibiotic or a probiotic therapy.

17. The method of claim 1, wherein the probiotic bacteria is listed as responsive in Tables 2A to 2D.

18. A method of identifying a patient that will respond to a therapy with an immune checkpoint inhibitor comprising:
identifying a subject in need of treatment with the immune checkpoint inhibitor to treat a melanoma;
obtaining a biological sample from the patient that comprises gut intestinal flora; and
determining whether the gut intestinal flora in the biological sample comprises a probiotic bacteria, wherein the presence of at least one of the Faecalibacterium prausnitzii, Holdemania filiformis, Bacteroides thetaiotamicron, Dorea formicigenerans, Bacteroides caccae or Streptococcus parasanguinis, is indicative that the melanoma will respond to the immune checkpoint inhibitor, or will have an improved response to the immune checkpoint inhibitor.

19. The method of claim 18, further comprising providing the patient with:
an amount of Faecalibacterium prausnitzii, Holdemania filiformis, Bacteroides thetaiotamicron, Dorea formicigenerans, Bacteroides caccae or Streptococcus parasanguinis probiotic bacteria sufficient to enhance the response to the immune checkpoint inhibitor;
a combination of the immune checkpoint inhibitors ipilimumab plus nivolumab and the probiotic bacteria is selected from at least one of: Faecalibacterium prausnitzii, Holdemania filiformis or Bacteroides thetaiotamicron;
pembrolizumab and the probiotic bacteria is Dorea formicigenerans; or
an amount of probiotic bacteria that increase the effectiveness of the immune checkpoint inhibitor selected from at least one of least one of ipilimumab, nivolumab, ipilimumab plus nivolumab, or pembrolizumab and the probiotic bacteria is selected from at least one of: Bacteroides caccae or Streptococcus parasanguinis.

20. (canceled)

21. (canceled)

22. (canceled)

23. The method of claim 18, wherein the patient is further provided with an effective amount of the probiotic bacteria and an anacardic acid in an amount sufficient to enhance a potency of the immune checkpoint inhibitor selected from at least one of ipilimumab, nivolumab, ipilimumab plus nivolumab, or pembrolizumab.

24. The method of claim 18, wherein the biological sample is a fecal sample.

25. The method of claim 18, wherein a presence of the probiotic bacteria is determined by at least one of metagenomic shotgun sequencing of bacteria or metabolic LC-MS analysis of metabolites.

26. The method of claim 18, wherein the patient does not have a BRAF mutation.

27. The method of claim 18, wherein the immune checkpoint inhibitor is selected from at least one of CTLA-4 inhibitor, PD1 inhibitor, ipilimumab, nivolumab, ipilimumab plus nivolumab, or pembrolizumab.

28. The method of claim 18, wherein at least one of:
a presence of Dorea formicigeneran is indicative of an enhanced therapeutic effect by pembrolizumab (PD1); a presence of at least one of: Faecalibacterium prausnitzii, Holdemania filiformis or Bacteroides thetaiotamicron, is indicative of an enhanced therapeutic effect by ipilimumab plus nivolumab;
a presence of at least one of: Bacteroides caccae or Streptococcus parasanguinis, is indicative of an enhanced therapeutic effect by an immune checkpoint inhibitor selected from at least one of least one of ipilimumab, nivolumab, ipilimumab plus nivolumab, or pembrolizumab; or
a presence of 15:2 anacardic acid in the biological sample is indicative of enhanced therapeutic effect by the immune checkpoint inhibitor.

29. (canceled)

30. (canceled)

31. (canceled)

32. The method of claim 18, wherein the patient is a human patient, the melanoma is metastatic melanoma; or the melanoma is resistant or refractory to the immune checkpoint inhibitor.

33. (canceled)

34. (canceled)

35. The method of claim 18, wherein the patient did not receive a concurrent antibiotic or a probiotic therapy.

36. The method of claim 18, wherein the probiotic bacteria is selected from the responsive probiotic bacteria in Tables 2A to 2D having a relative response of 1.0 or greater.

37. A composition that increases an efficacy of an immune checkpoint inhibitor for a treatment of cancer comprising:
a first composition comprising at least one of a probiotic bacteria, a prebiotic composition, or an anacardic acid, wherein the first composition is capable of increasing the efficacy of the immune checkpoint inhibitor; and
a second composition comprising a therapeutically effective amount of the immune checkpoint inhibitor, wherein the immune checkpoint inhibitor is selected from at least one of ipilimumab, nivolumab, ipilimumab plus nivolumab, or pembrolizumab.

38. The composition of claim 37, wherein the first and second compositions are provided in a single formulation, are provided concurrently, or are provided separately.

39. The composition of claim 37, wherein at least one of:
the first composition further comprises at least one of Bacteroides caccae, Streptococcus parasanguinis, Dorea formicigenerans, Faecalibacterium prausnitzii, Holdemania filiformis or Bacteroides thetaiotamicron;
the first composition comprises Dorea formicigeneran, when the second composition comprises pembrolizumab;
the first composition comprises at least one of: Bacteroides caccae or Streptococcus parasanguinis, when the second composition comprises at least one of ipilimumab, nivolumab, ipilimumab plus nivolumab, or pembrolizumab;
the first composition consists essentially of Bacteroides caccae or Streptococcus parasanguinis when the second composition is comprises at least one of ipilimumab, nivolumab, ipilimumab plus nivolumab, or pembrolizumab;
the first composition comprises at least one of: Faecalibacterium prausnitzii, Holdemania filiformis or Bacteroides thetaiotamicron, when the second composition is ipilimumab and nivolumab;
the first composition consists essentially of Faecalibacterium prausnitzii, Holdemania filiformis or Bacteroides thetaiotamicron when the second composition is ipilimumab and nivolumab; or
the first composition consists essentially of Dorea formicigeneran when the second composition is pembrolizumab.

40. (canceled)

41. (canceled)

42. (canceled)

43. (canceled)

44. (canceled)

45. (canceled)

46. The composition of claim 37, wherein the probiotic bacteria are selected from those responsive in Tables 2A to 2D.

47. A pharmaceutical composition comprising at least one of an isolated Bacteroides caccae, Streptococcus parasanguinis, Dorea formicigenerans, Faecalibacterium prausnitzii, Holdemania filiformis or Bacteroides thetaiotamicron in an amount effective to increase an effectiveness of an immune checkpoint inhibitor against a cancer.

48. The composition of claim 47, wherein the composition consists essentially of Bacteroides caccae and Streptococcus parasanguinis; consists essentially of Faecalibacterium prausnitzii, Holdemania filiformis and Bacteroides thetaiotamicron; or consists essentially of Dorea formicigenerans.

49. (canceled)

50. (canceled)

51. A method of increasing an efficacy of an immune checkpoint inhibitor against melanoma comprising:
identifying a human patient in need of treatment for melanoma;
providing the human patient with an effective amount of the immune checkpoint inhibitor; and
providing the human patient with at least one of a targeted antibacterial agent in the amount effective to increase the potency of the immune checkpoint inhibitor against the melanoma.

52. The method of claim 51, wherein the antibacterial agent is directed against at least one of actinobacteria, coriobacteriaceae, coriobacteriales, bacteroides eggerthii, parvimonas micrs, parvimonas, Bifidobacterium dentium, or actinomyces viscosues; the antibacterial agent is directed against at least one of lactobacillaceae, lactobacillus, acidaminococcaceae, anaerococcus, atopobium parvulum, anaerococcus vaginalis, peptoniphilus or lactobacillus gasseri, the antibacterial agent is a bacteriophade or the antibacterial agent is an antimicrobial CRISP-Cas system agent.

53. (canceled)

54. (canceled)

55. (canceled)
